(19) United States
(12) Patent Application Publication
(10) Pub. No.: US 2026/0078198 A1
(43) Pub. Date: Mar. 19, 2026

(54) METHODS FOR TREATING CANCER USING SUBCUTANEOUS DOSING OF MOSUNETUZUMAB IN COMBINATION WITH POLATUZUMAB VEDOTIN

(71) Applicants: Genentech, Inc., South San Francisco, CA (US); Hoffmann-La Roche Inc., Little Falls, NJ (US)

(72) Inventors: Iris Tranthuyngan TO, Sunnyvale, CA (US); Jue WANG, San Jose, CA (US); Carol Elaine O'HEAR, Burlingame, CA (US); Yasuhiro OKI, Mountain View, CA (US); Song Ha PHAM, Toronto (CA)

(21) Appl. No.: 19/312,481

(22) Filed: Aug. 28, 2025

Related U.S. Application Data
(60) Provisional application No. 63/688,185, filed on Aug. 28, 2024.
(60) Provisional application No. 63/792,882, filed on Apr. 22, 2025.

(51) Int. Cl. C07K 16/28; A61K 31/135; A61K 31/167; A61K 31/555; A61K 31/573; A61K 31/7068; A61K 39/00; A61K 47/68; A61P 35/00

(52) U.S. Cl. CPC C07K 16/2887; A61K 31/135; A61K 31/167; A61K 31/555; A61K 31/573; A61K 31/7068; A61K 47/68031; A61K 47/6849; A61K 47/6867; A61P 35/00; C07K 16/2809; C07K 16/2866; A61K 2039/507; A61K 2039/54; A61K 2039/545

(57) ABSTRACT

The present invention relates to the treatment of subjects having a CD20-positive cell proliferative disorder (e.g., B cell proliferative disorders, such as a non-Hodgkin's lymphoma (NHL); e.g., an aggressive NHL or a relapsed and/or refractory NHL). More specifically, the invention pertains to the treatment of subjects having a B cell proliferative disorder by administering a combination of mosunetuzumab and polatuzumab vedotin.

### SEQUENCE LISTING

The instant application contains a Sequence Listing which has been submitted electronically in XML format and is hereby incorporated by reference in its entirety. Said XML copy, created on Aug. 14, 2025, is named 50474-359003_Sequence_Listing_8_14_25.xml and is 50,549 bytes in size.

### FIELD OF THE INVENTION

The present invention relates to the treatment of subjects having a CD20-positive cell proliferative disorder (e.g., a B cell proliferative disorder). More specifically, the invention pertains to combination treatment of subjects having a CD20-positive cell proliferative disorder (e.g., a B cell proliferative disorder; e.g., a non-Hodgkin's lymphoma (NHL); e.g., an aggressive NHL), who may have received at least one line of prior therapy, by subcutaneous administration of mosunetuzumab and intravenous administration of polatuzumab vedotin.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Indolent cancers can also severely effect quality of life. Cancer care thus represents a significant and ever-increasing societal burden.

B cell proliferative disorders are a leading cause of cancer-related deaths. For example, non-Hodgkin's lymphoma (NHL) advances quickly and is fatal if untreated. In the United States, B-cell lymphomas constitute approximately 80%-85% of all cases of NHL. Aggressive NHLs include DLBCLs, transformed FLs, and Grade 3b FLs. Up to 40% of patients with DLBCL who are treated in the first-line setting will experience disease progression within 3-4 years (Friedberg 2011), and more than half of the patients treated with second-line therapies do not achieve a complete remission (Gisselbrecht et al. 2010). Furthermore, since the introduction of the monoclonal anti CD20 antibody rituximab, it has become more challenging to find effective therapies for the large proportion of patients with R/R DLBCL who have prior exposure to anti CD20 antibody.

Each year around 3% of FLs transform into higher-grade NHL, most commonly DLBCL (Lossos and Gascoyne, 2011), leading to almost a third of histologic transformation in 10 years. These patients with DLBCL transformed from a previous FL histology and have been treated with the same standard therapies as high-grade lymphomas. Follicular lymphoma Grade 3b is a distinct subgroup of FL that is more in common genetically, immunophenotypically, and clinically with DLBCL than with other indolent FLs, and the coexistence with DLBCL is frequent (Harris and Kluin, 2011). The clinical course of patients with FL Grade 3b is similar to those with DLBCL, and FL Grade 3b is commonly treated as DLBCL (National Comprehensive Cancer Network [NCCN], 2020).

Regardless of the biologic and clinical heterogeneity of B-cell lymphomas, subjects with advanced-stage B-cell malignancies are typically treated, initially, with intensive cytotoxic chemotherapy combined with monoclonal antibodies (mAbs) such as the anti-CD20 mAb, rituximab (Rituxan®, MabThera®). Although durable responses can be achieved in some subjects, the majority of subjects will ultimately experience progressive or relapsed disease. NHL remains an incurable disease with currently available therapies. The addition of rituximab to commonly used induction chemotherapy, including cyclophosphamide, doxorubicin, vincristine, and prednisone (CHOP); cyclophosphamide, vincristine, and prednisone (CVP); fludarabine, cyclophosphamide, and mitoxantrone (FCM); bendamustine; or gemcitabine and oxaliplatin (Zelenetz et al. J. Natl. Compr. Canc. Netw. 2014, 12 (6): 916-946; Forstpointner et al. Blood. 2006, 108 (13): 4003-4008; Mounier et al., Haematologica. 2013, 98 (11): 1726-1731), followed by rituximab maintenance therapy led to prolonged remission and improved subject outcomes. In particular, R-GemOx (rituximab, gemcitabine, and oxaliplatin; see Mounier et al., Haematologica. 2013, 98 (11): 1726-1731) is a recommended regimen in the NCCN Guideline 2021 for subjects with R/R DLBCL who are not candidates for autologous stem cell transplant (ASCT) therapy.

For such subjects, alternative or secondary treatment modalities, such as bispecific antibody-based immunotherapies, may be particularly efficacious. Bispecific antibodies such as mosunetuzumab are capable of simultaneously binding cell surface antigens on cytotoxic cells (e.g., T cells, via binding to CD3) and cancer cells (e.g., B cells, via binding to CD20), with the intent that the bound cytotoxic cell will destroy the bound cancer cell. Antibody drug conjugates are capable of binding to cell-surface epitopes (e.g., targeting CD79b; e.g., polatuzumab vedotin) to promote internalization of the bound drug conjugate for targeted delivery of cytotoxic agents. However, such antibody-based and antibody-drug-conjugate-based immunotherapies may be limited by unwanted effects, including cytokine-driven toxicities (e.g., cytokine release syndrome (CRS)), infusion-related reactions (IRRs), severe tumor lysis syndrome (TLS), and hepatotoxicities.

Thus, there is an unmet need in the field for the development of efficacious methods of combination dosing for the treatment of CD20-positive cell proliferative disorders, including B cell proliferative disorders such as non-Hodgkin's lymphoma (NHL) (e.g., a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma) that achieve a more favorable benefit-risk profile.

### SUMMARY OF THE INVENTION

The present invention relates to methods of treating a subject having a CD20-positive cell proliferative disorder (e.g., a B cell proliferative disorder; e.g., a non-Hodgkin's lymphoma (NHL) (e.g., a large B cell lymphoma (LBCL), a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma; e.g., a relapsed and/or refractory NHL (R/R NHL; e.g., an R/R LBCL, an R/R DLBCL, an R/R FL, or an R/R MCL) or an aggressive NHL (aNHL, e.g., R/R diffuse large DLBCL, R/R HGBL, R/R trFL, and R/R Grade 3b FL)), by subcutaneous administration of mosunetuzumab and intravenous administration of polatuzumab vedotin as a combination treatment, wherein the combination treatment comprising subcutaneous administration of mosunetuzumab in combination with intravenous administration of polatuzumab vedotin exhibits non-inferiority or an improved outcome as measured using pharmacokinetics (PK), efficacy, safety, or a combination thereof, as compared to (i) a reference treatment comprising intravenous administration of mosunetuzumab and intravenous administration of polatuzumab vedotin; (ii) a reference treatment comprising rituximab and polatuzumab vedotin; or (iii) a combination treatment comprising rituximab, gemcitabine, and oxaliplatin (R-GemOx).

In one aspect, the invention features a method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

In some embodiments, the efficacy response is a CRR, and wherein the CRR or reference CRR is the proportion of the subjects receiving the corresponding treatment whose best overall response is a complete response (CR).

In some embodiments, the improved response in CRR is an increase in CRR compared to the reference CRR of between 1% and 41% (e.g., between 1% and 30%, between 1% and 20%, between 1% and 10%, between 10% and 30%, between 10% and 20%, between 15% and 35%, between 15% and 25%, between 30% and 40%, or between 20% and 40%; e.g., about 5%, 10%, 15%, 20%, 21%, 22%, 23%, 25%, 30%, 35%, 40%, or 41%). In some embodiments, the improved response in CRR is an increase in CRR compared to the reference CRR of about 20.8%.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 3% and 47% (e.g., between 3% and 40%, between 3% and 35%, between 3% and 30%, between 3% and 25%, between 3% and 15%, between 3% and 10%, between 5% and 45%, between 5% and 45%, between 10% and 45%, between 15% and 45%, between 25% and 45%, between 30% and 45%, between 35% and 45%, between 40% and 45%, between 15% and 35%, or between 20% and 30%; e.g., about 3%, 5%, 10%, 15%, 20%, 25%, 26%, 30%, 35%, 40% or 45%). In some embodiments, the improved response in ORR is an increase in ORR compared to the reference ORR of about 25.6%.

In some embodiments, the efficacy response is a DOR, and wherein the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the efficacy response in median DOR is non-inferior compared to the reference median DOR. In some embodiments, the efficacy response in the rate of a DOR of 3 months is non-inferior compared to the reference rate of a DOR of 3 months. In some embodiments, the efficacy response in the rate of a DOR of 6 months is non-inferior compared to the reference rate of a DOR of 6 months. In some embodiments, the efficacy response in the rate of a DOR of 9 months is non-inferior compared to the reference rate of a DOR of 9 months.

In some embodiments, the efficacy response is a DOCR, and wherein the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the efficacy response in median DOCR is non-inferior compared to the reference median DOCR. In some embodiments, the efficacy response in the rate of a DOCR of 3 months is non-inferior compared to the reference rate of a DOCR of 3 months. In some embodiments, the efficacy response in the rate of a DOCR of 6 months is non-inferior compared to the reference rate of a DOCR of 6 months. In some embodiments, the efficacy response in the rate of a DOCR of 9 months is non-inferior compared to the reference rate of a DOCR of 9 months.

In some embodiments, the CR or PR determined by PET/computed tomography (CT). In some embodiments, the CR or PR is determined based on the Lugano Response Criteria for Malignant Lymphoma (Cheson et al., 2014).

In one aspect, the invention provides a method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

In some embodiments, the safety response is the rate of AE, and wherein the rate of AE or the reference rate of AE is the rate of AE in the plurality of subjects receiving the corresponding treatment. In some embodiments, the safety response in rate of AE is non-inferior compared to the reference rate of AE. In some embodiments, the safety response in rate of Grade 3-5 AE is non-inferior compared to the reference rate of Grade 3-5 AE. In some embodiments, the Grade of AE is determined based on the National Cancer Institute Common Terminology Criteria for Adverse Events (NCI CTCAE), v5.0.

In some embodiments, the safety response is the rate of SAE, and wherein the rate of SAE or the reference rate of SAE is the rate of SAE in the plurality of subjects receiving the corresponding treatment. In some embodiments, the safety response in rate of SAE is non-inferior compared to the reference rate of SAE.

In some embodiments, the combination treatment comprises subcutaneously administering mosunetuzumab and intravenously administered polatuzumab vedotin according to a dosing regimen comprising at least a first 21-day dosing cycle and a second 21-day dosing cycle, wherein: (a) the first dosing cycle comprises: (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and (ii) an intravenous dose of polatuzumab vedotin administered on Day 1 of the first dosing cycle, wherein the intravenous dose of polatuzumab vedotin is about 1.8 mg/kg; and (b) the second dosing cycle comprises: (i) a first subcutaneous dose (scC2D1) of mosunetuzumab administered on Day 1 of the second dosing cycle, wherein the scC2D1 is about 45 mg; and (ii) an intravenous dose of polatuzumab vedotin administered on Day 1 of the second dosing cycle, wherein the intravenous dose of polatuzumab vedotin is about 1.8 mg/kg.

In some embodiments, the dosing regimen of the combination treatment further comprises one or more additional (e.g., 1, 2, 3, 4, 5, 6, 7, 8, 9, 10, 11, 12, or more) 21-day dosing cycles. In some embodiments, the dosing regimen of the combination treatment comprises six additional 21-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; or (b) a subcutaneous dose of mosunetuzumab administered on Day 1 of the additional dosing cycle, wherein the subcutaneous dose of mosunetuzumab is about 45 mg. In some embodiments, (a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and (b) the next two additional dosing cycles each comprises a subcutaneous dose of mosunetuzumab administered on Day 1 of the additional dosing cycle, wherein the subcutaneous dose of mosunetuzumab is about 45 mg.

In some embodiments, the control treatment comprises intravenously administering 375 mg/m² rituximab, 1000 mg/m² gemcitabine, and 100 mg/m² oxaliplatin for eight 14-day dosing cycles, wherein rituximab, gemcitabine, and oxaliplatin are administered on Day 1 of each dosing cycle.

In one aspect, the invention features a method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

In some embodiments, the efficacy response is a CRR, and wherein the CRR or reference CRR is the proportion of the subjects receiving the corresponding treatment whose best overall response is a complete response (CR). In some embodiments, the improved response in CRR is an increase in CRR compared to the reference CRR of between 1% and 52% (e.g., between 1% and 40%, between 1% and 30%, between 1% and 20%, between 1% and 10%, between 10% and 40%, between 10% and 30%, between 10% and 20%, between 15% and 35%, between 15% and 25%, between 1% and 25%, between 25% and 50%, between 30% and 50%, between 20% and 50%, between 30% and 40%, between 20% and 40%, between 20% and 30%, or between 20% and 25%; e.g., about 5%, 10%, 15%, 20%, 21%, 22%, 23%, 24%, 25%, 30%, 35%, 40%, 45%, 50%, or 52%). In some embodiments, the improved response in CRR is an increase in CRR compared to the reference CRR of about 23%.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 1% and 55% (e.g., between 1% and 50%, between 1% and 40%, between 1% and 30%, between 1% and 20%, between 1% and 10%, between 10% and 40%, between 10% and 30%, between 10% and 20%, between 15% and 35%, between 15% and 25%, between 1% and 25%, between 25% and 50%, between 35% and 55%, between 25% and 35%, between 30% and 50%, between 20% and 50%, between 30% and 40%, between 20% and 40%, between 20% and 30%, between 20% and 25%, or between 25% and 30%; e.g., about 5%, 10%, 15%, 20%, 25%, 26%, 27%, 28%, 29%, 30%, 35%, 40%, 45%, 50%, or 55%). In some embodiments, the improved response in ORR is an increase in ORR compared to the reference ORR of about 28%.

In some embodiments, the efficacy response is a DOR, and wherein the DOR or the reference DOR is measured starting from the time from the first occurrence of a documented CR or PR to disease progression or relapse or death from any cause, whichever occurs first.

In some embodiments, the DOR or the reference DOR is the median DOR of the plurality of subjects receiving the corresponding treatment. In some embodiments, the improvement of the median DOR is an increase in the DOR compared to the reference DOR of between 1 and 21 months (e.g., between 1 month and 5 months, between 5 months and 21 months, between 10 months and 21 months, between 15 months and 21 months, between 5 months and 10 months, between 5 months and 15 months, between 10 months and 21 months, between 10 months and 15 months, or between 15 months and 21 months; e.g., about 1 months, 5 months, 10 months, 15 months, 20 months, or 21 months). In some embodiments, the improvement of the DOR is an increase in the rate of a DOR of 6 months compared to the rate of a reference DOR of 6 months of between 1% and 61% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 18%, 19%, 20%, 21%, 22%, 25%, 30%, 35%, 40%, 45%, 50%, 55%, 60%, or 61%). In some embodiments, the improvement of the DOR is an increase in the rate of a DOR of 6 months compared to the rate of a reference DOR of 6 months of about 20%. In some embodiments, the improvement of the DOR is an increase in the rate of a DOR of 9 months compared to the rate of a reference DOR of 9 months of between 1% and 72% (e.g., between 1% and 70%, between 1% and 60%, between 1% and 50%, between 1% and 40%, between 1% and 30%, between 1% and 20%, between 1% and 10%, between 10% and 70%, between 10% and 60%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 35% and 70%, between 55% and 70%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 30%, 35%, 40%, 45%, 50%, 55%, 60%, 65%, 70%, or 72%). In some embodiments, the improvement of the DOR is an increase in the rate of a DOR of 9 months compared to the rate of a reference DOR of 9 months of about 26%. In some embodiments, the improvement of the DOR is an increase in the rate of a DOR of 12 months compared to the rate of a reference DOR of 12 months of between 1% and 87% (e.g., between 1% and 85%, between 1% and 80%, between 1% and 70%, between 1% and 60%, between 1% and 50%, between 1% and 40%, between 1% and 30%, between 1% and 20%, between 1% and 10%, between 10% and 80%, between 10% and 60%, between between 10% and 40%, between 10% and 30%, between 10% and 20%, between 15% and 35%, between 15% and 25%, between 1% and 25%, between 25% and 50%, between 35% and 55%, between 30% and 80%, between 35% and 70%, between 55% and 70%, between 25% and 35%, between 20% and 50%, between 40% and 60%, between 20% and 60%, between 30% and 40%, between 20% and 40%, between 60% and 80%, between 10% and 50%, between 20% and 50%, between 30% and 50%, or between 35% and 45%; e.g., about 5%, 10%, 15%, 20%, 25%, 26%, 27%, 28%, 30%, 35%, 40%, 45%, 50%, 55%, 60%, 65%, 70%, 75%, 80%, 85%, or 87%). In some embodiments, the improvement of the DOR is an increase in the rate of a DOR of 12 months compared to the rate of a reference DOR of 12 months of about 39%. In some embodiments, the improvement of the DOR is an increase in the rate of a DOR of 18 months compared to the rate of a reference DOR of 18 months of between 43% and 92% (e.g., between 43% and 90%, between 43% and 80%, between 43% and 70%, between 43% and 60%, between 43% and 50%, between 50% and 80%, between 50% and 70%, between 50% and 60%, between 60% and 70%, between 60% and 80%, between 60% and 75%, between 55% and 65%, between 50% and 90%, between 80%, and 90%, between 75% and 90%, or between 65% and 70%; e.g., about 43%, 45%, 50%, 55%, 60%, 65%, 66%, 67%, 68%, 69%, 70%, 75%, 80%, 85%, 90%, or 92%). In some embodiments, the improvement of the DOR is an increase in the rate of a DOR of 18 months compared to the rate of a reference DOR of 18 months of about 67%.

In some embodiments, the CR or PR determined by PET/computed tomography (CT). In some embodiments, the CR or PR is determined based on the Lugano Response Criteria for Malignant Lymphoma (Cheson et al., 2014).

In some embodiments, the efficacy response is a PFS, and wherein the PFS or the reference PFS is measured starting from the time of receiving first dose of mosunetuzumab or polatuzumab vedotin to the time of a first occurrence of disease progression or death from any cause. In some embodiments, the PFS or the reference PFS is the median PFS of the plurality of subjects receiving the corresponding treatment. In some embodiments, the improvement of the median PFS is an increase in the PFS compared to the reference PFS of between 1 and 24 months. In some embodiments, the improvement of the PFS is an increase in the rate of a PFS of 6 months compared to the rate of a reference PFS of 6 months of between 1% and 53% (e.g., between 1% and 50%, between 1% and 40%, between 1% and 30%, between 1% and 20%, between 1% and 10%, between 10% and 40%, between 10% and 30%, between 10% and 20%, between 15% and 35%, between 15% and 25%, between 1% and 25%, between 25% and 50%, between 35% and 53%, between 25% and 35%, between 30% and 50%, between 20% and 50%, between 30% and 40%, between 20% and 40%, between 20% and 30%, between 20% and 25%, or between 25% and 30%; e.g., about 5%, 10%, 15%, 20%, 25%, 26%, 27%, 28%, 29%, 30%, 35%, 40%, 45%, 50%, or 53%). In some embodiments, the improvement of the PFS is an increase in the rate of a PFS of 6 months compared to the rate of a reference PFS of 6 months of about 21%. In some embodiments, the improvement of the PFS is an increase in the rate of a PFS of 9 months compared to the rate of a reference PFS of 9 months of between 1% and 62% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 29%, 30%, 35%, 40%, 45%, 50%, 55%, 60%, or 62%). In some embodiments, the improvement of the PFS is an increase in the rate of a PFS of 9 months compared to the rate of a reference PFS of 9 months of about 28%. In some embodiments, the improvement of the PFS is an increase in the rate of a PFS of 12 months compared to the rate of a reference PFS of 12 months of between 1% and 63% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 29%, 30%, 35%, 40%, 45%, 50%, 55%, 60%, or 63%). In some embodiments, the improvement of the PFS is an increase in the rate of a PFS of 12 months compared to the rate of a reference PFS of 12 months of about 27%. In some embodiments, the improvement of the PFS is an increase in the rate of a PFS of 18 months compared to the rate of a reference PFS of 18 months of between 1% and 68% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 20% and 65%, between 40% and 65%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 29%, 30%, 35%, 40%, 45%, 50%, 55%, 60%, 65%, or 68%). In some embodiments, the improvement of the PFS is an increase in the rate of a PFS of 18 months compared to the rate of a reference PFS of 18 months of about 25%.

In one aspect, the invention features a method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

In some embodiments, (a) the safety response is the rate of AE, and wherein the rate of AE or the reference rate of AE is the rate of AE in the plurality of subjects receiving the corresponding treatment; or (b) the safety response is the rate of SAE, and wherein the rate of SAE or the reference rate of SAE is the rate of SAE in the plurality of subjects receiving the corresponding treatment. In some embodiments, (a) the safety response in rate of AE is non-inferior compared to the reference rate of AE; or (b) the safety response in rate of SAE is non-inferior compared to the reference rate of SAE.

In some embodiments, the control treatment comprises (a) intravenously administering 375 mg/m² rituximab and 1.8 mg/kg polatuzumab vedotin for six 21-day dosing cycles, wherein rituximab and polatuzumab vedotin are administered on Day 1 of each dosing cycle; and (b) intravenously administering 375 mg/m² rituximab for two 21-day dosing cycles, wherein rituximab is administered on Day 1 of each dosing cycle.

In one aspect, the invention features a method of achieving an objective response (OR), a complete response (CR), an overall survival (OS), or a progression free survival (PFS) in a subject having a relapsed and/or refractory non-Hodgkin's lymphoma (R/R NHL), wherein the subject is administered a combination treatment comprising subcutaneously administered mosunetuzumab and intravenously administered polatuzumab vedotin.

In some embodiments, the subject achieves an OR. In some embodiments, the OR is maintained for at least 6, 9, 12, or 18 months.

In some embodiments, the subject achieves a CR.

In some embodiments, the OS is maintained for at least 6, 9, 12, or 18 months.

In some embodiments, the PFS is maintained for at least 6, 9, 12, or 18 months.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall response rate of between 62% and 89% (e.g., between 65% and 85%, between 65% and 75%, or between 75% and 85%; e.g., about 62%, 65%, 70%, 75%, 80%, 85%, or 89%).

In some embodiments, the overall response rate is about 78%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a complete response rate of between 41% and 73% (e.g., between 45% and 70%, between 55% and 70%, between 65% and 70%, between 45% and 55%, between 45% and 65%, or between 55% and 65%; e.g., about 41%, 45%, 50%, 55%, 56%, 57%, 58%, 59%, 60%, 65%, 70%, or 73%).

In some embodiments, the complete response rate is about 58%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall survival rate at 9 months of between 66% and 92% (e.g., between 65% and 90%, between 65% and 80%, between 65% and 75%, between 75% and 85% between 75% and 90%, or between 70% and 85%; e.g., about 62%, 65%, 70%, 75%, 80%, 85%, 90%, or 92%).

In some embodiments, the overall survival rate at 9 months is about 79%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall survival rate at 12 months of between 60% and 88% (e.g., between 65% and 85%, between 65% and 80%, between 65% and 75%, between 75% and 85%, between 75% and 88%, or between 70% and 85%; e.g., about 62%, 65%, 70%, 73%, 74%, 75%, 80%, 85%, or 88%).

In some embodiments, the overall survival rate at 12 months is about 74%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a progression free survival rate at 9 months of between 57% and 87% (e.g., between 60% and 85%, between 65% and 85%, between 65% and 80%, between 60% and 75%, between 75% and 85%, between 75% and 87%, or between 70% and 85%; e.g., about 57%, 60%, 65%, 70%, 71%, 72%, 73%, 74%, 75%, 80%, 85%, or 87%).

In some embodiments, the progression free survival rate at 9 months is about 72%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a progression free survival rate at 12 months of between 47% and 81% (e.g., between 50% and 80%, between 50% and 70%, between 55% and 70%, between 65% and 70%, between 50% and 75%, between 60% and 80%, or between 60% and 65%; e.g., about 47%, 50%, 55%, 60%, 63%, 64%, 65%, 70%, 75%, 80%, or 81%).

In some embodiments, the progression free survival rate at 12 months is about 64%.

In some embodiments, the combination treatment comprises subcutaneously administering mosunetuzumab and intravenously administered polatuzumab vedotin according to a dosing regimen comprising at least a first 21-day dosing cycle and a second 21-day dosing cycle, wherein: (a) the first dosing cycle comprises: (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and (ii) an intravenous dose of polatuzumab vedotin administered on Day 1 of the first dosing cycle, wherein the intravenous dose of polatuzumab vedotin is about 1.8 mg/kg; and (b) the second dosing cycle comprises: (i) a first subcutaneous dose (scC2D1) of mosunetuzumab administered on Day 1 of the second dosing cycle, wherein the scC2D1 is about 45 mg; and (ii) an intravenous dose of polatuzumab vedotin administered on Day 1 of the second dosing cycle, wherein the intravenous dose of polatuzumab vedotin is about 1.8 mg/kg.

In some embodiments, the dosing regimen of the combination treatment further comprises one or more (e.g., 1, 2, 3, 4, 5, 6, 7, 8, 9, 10, 11, 12, or more) additional 21-day dosing cycles. In some embodiments, the dosing regimen of the combination treatment comprises six additional 21-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; or (b) a subcutaneous dose of mosunetuzumab administered on Day 1 of the additional dosing cycle, wherein the subcutaneous dose of mosunetuzumab is about 45 mg.

In some embodiments, (a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and (b) the next two additional dosing cycles each comprises a subcutaneous dose of mosunetuzumab administered on Day 1 of the additional dosing cycle, wherein the subcutaneous dose of mosunetuzumab is about 45 mg.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median overall survival of between 15 and 39 months (e.g., between 15 months and 35 months, between 15 months and 30 months, between 15 months and 25 months, between 15 months and 20 months, between 20 months and 35 months, between 20 months and 30 months, between 20 months and 25 months, between 25 months and 39 months, or between 25 months and 30 months; e.g., about 15, 20, 25, 26, 27, 28, 29, 30, 35, or 39 months). In some embodiments, the median overall survival is about 27 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 6 months of between 73% and 89% (e.g., between 75% and 89%, between 75% and 85%, between 75% and 80%, between 80% and 85%, or between 80% and 89%; e.g., about 73%, 75%, 80%, 81%, 82%, 85%, or 89%). In some embodiments, the overall survival rate at 6 months is about 81%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 9 months of between 61% and 80% (e.g., between 61% and 80%, between 65% and 80%, between 60% and 75%, between 75% and 80%, between 61% and 70%, between 70% and 80%, or between 65% and 75%; e.g., about 61%, 65%, 68%, 69%, 70%, 71%, 72%, 75%, or 80%). In some embodiments, the overall survival rate at 9 months is about 70%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 12 months of between 55% and 75% (e.g., between 55% and 70%, between 55% and 65%, between 55% and 60%, between 60% and 75%, between 65% and 75%, between 70% and 75%, or between 60% and 70%; e.g., about 55%, 60%, 65%, 70%, or 75%). In some embodiments, the overall survival rate at 12 months is about 65%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 18 months of between 45% and 65% (e.g., between 45% and 65%, between 45% and 60%, between 45% and 55%, between 45% and 50%, between 60% and 65%, between 50% and 65%, between 55% and 65%, or between 50% and 60%; e.g., about 45%, 50%, 55%, 60%, or 65%). In some embodiments, the overall survival rate at 18 months is about 55%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 24 months of between 40% and 61% (e.g., between 40% and 60%, between 40% and 55%, between 40% and 50%, between 40% and 45%, between 55% and 60%, between 50% and 60%, or between 45% and 55%; e.g., about 40%, 45%, 50%, 55%, 60%, or 61%). In some embodiments, the overall survival rate at 24 months is about 50%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median progression free survival of between 9 and 27 months (e.g., between 9 months and 25 months, between 15 months and 25 months, between 10 months and 25 months, between 15 months and 20 months, or between 10 months and 20 months; e.g., about 9, 10, 13, 14, 15, 20, 25, or 27 months). In some embodiments, the median progression free survival is about 14 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 6 months of between 51% and 72% (e.g., between 55% and 70%, between 55% and 65%, between 55% and 60%, between 60% and 72%, between 65% and 72%, between 70% and 72%, or between 60% and 70%; e.g., about 51%, 55%, 60%, 61%, 62%, 63%, 64%, 65%, 70%, or 72%). In some embodiments, the progression free survival rate at 6 months is about 62%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 9 months of between 50% and 71% (e.g., between 50% and 70%, between 50% and 65%, between 50% and 60%, between 50% and 55%, between 60% and 71%, between 65% and 71%, between 55% and 70%, or between 55% and 65%; e.g., about 50%, 55%, 60%, 61%, 62%, 63%, 64%, 65%, 70%, or 71%). In some embodiments, the progression free survival rate at 9 months is about 60%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 12 months of between 41% and 64% (e.g., between 45% and 64%, between 45% and 60%, between 45% and 55%, between 45% and 50%, between 60% and 64%, between 50% and 64%, between 55% and 64%, or between 50% and 60%; e.g., about 41%, 45%, 50%, 55%, 60%, or 64%). In some embodiments, the progression free survival rate at 12 months is about 52%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 18 months of between 34% and 57% (e.g., between 35% and 55%, between 35% and 50%, between 35% and 45%, between 35% and 40%, between 40% and 55%, between 40% and 50%, between 40% and 45%, or between 45% and 55%; e.g., about 34%, 35%, 40%, 45%, 46%, 47%, 48%, 50%, 55%, or 57%). In some embodiments, the progression free survival rate at 18 months is about 46%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 24 months of between 28% and 52% (e.g., between 30% and 50%, between 30% and 45%, between 30% and 40%, between 30% and 35%, between 35% and 50%, between 40% and 50%, between 40% and 45%, or between 35% and 45%; e.g., about 28%, 30%, 35%, 40%, 45%, 50%, or 52%). In some embodiments, the progression free survival rate at 24 months is about 40%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median duration of response of between 16 and 39 months (e.g., between 16 months and 35 months, between 16 months and 25 months, between 16 months and 20 months, between 20 months and 35 months, or between 25 months and 30 months; e.g., about 16, 20, 25, 26, 27, 28, 29, 30, 35, or 39 months).

In some embodiments, the median duration of response is about 28 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 78% and 96% (e.g., between 80% and 95%, between 80% and 90%, between 80% and 85%, between 85% and 90%, or between 85% and 95%; e.g., about 78%, 80%, 85%, 86%, 87%, 88%, 89%, 90%, 95%, or 96%) of the population of subjects maintains a durable response for 6 months. In some embodiments, about 87% of the population of subjects maintains a durable response for 6 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 63% and 88% (e.g., between 65% and 85%, between 65% and 80%, between 65% and 75%, between 65% and 70%, between 70% and 75%, between 70% and 80%, between 70% and 85%, or between 75% and 85%; e.g., about 63%, 65%, 70%, 75%, 80%, 85%, or 88%) of the population of subjects maintains a durable response for 9 months. In some embodiments, about 75% of the population of subjects maintains a durable response for 9 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 58% and 84% (e.g., between 60% and 84%, between 65% and 84%, between 65% and 80%, between 60% and 75%, between 65% and 70%, between 65% and 75%, between 70% and 75%, between 70% and 80%, between 70% and 84%, or between 75% and 84%; e.g., about 58%, 60%, 65%, 70%, 71%, 72%, 73%, 75%, 80%, or 84%) of the population of subjects maintains a durable response for 12 months. In some embodiments, about 71% of the population of subjects maintains a durable response for 12 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 46% and 75% (e.g., between 46% and 75%, between 46% and 70%, between 46% and 60%, between 46% and 50%, between 50% and 75%, between 50% and 70%, between 60% and 70%, between 55% and 65%, between 50% and 60%, or between 55% and 75%; e.g., about 46%, 50%, 55%, 60%, 61%, 62%, 63%, 65%, 70%, or 75%) of the population of subjects maintains a durable response for 18 months. In some embodiments, about 61% of the population of subjects maintains a durable response for 18 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 43% and 73% (e.g., between 45% and 73%, between 45% and 70%, between 45% and 60%, between 45% and 50%, between 50% and 73%, between 55% and 70%, between 50% and 70%, between 60% and 70%, between 55% and 65%, between 50% and 60%, or between 55% and 60%; e.g., about 43%, 45%, 50%, 55%, 56%, 57%, 58%, 59%, 60%, 65%, 70%, or 73%) of the population of subjects maintains a durable response for 24 months. In some embodiments, about 58% of the population of subjects maintains a durable response for 24 months.

In some embodiments, the combination treatment comprises intravenously administering mosunetuzumab and intravenously administering polatuzumab vedotin according to a dosing regimen comprising eight 21-day dosing cycle, wherein: (a) the first dosing cycle comprises: (i) a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg; and (ii) an intravenous dose of polatuzumab vedotin administered on Day 1 of the first dosing cycle, wherein the intravenous dose of polatuzumab vedotin is about 1.8 mg/kg; (b) the second dosing cycle comprises: (i) a first intravenous dose (ivC2D1) of mosunetuzumab administered on Day 1 of the second dosing cycle, wherein the ivC2D1 is about 60 mg; and (ii) an intravenous dose of polatuzumab vedotin administered on Day 1 of the second dosing cycle, wherein the intravenous dose of polatuzumab vedotin is about 1.8 mg/kg; (c) the third to sixth dosing cycles each comprises: (i) a first intravenous dose (ivC3D1-ivC6D1) of mosunetuzumab administered on Day 1 of each respective dosing cycle, wherein the ivC3D1-ivC6D1 is each about 30 mg; and (ii) an intravenous dose of polatuzumab vedotin administered on Day 1 of the second dosing cycle, wherein the intravenous dose of polatuzumab vedotin is about 1.8 mg/kg; and (d) the seventh and eighth dosing cycles each comprises a first intravenous dose (ivC7D1-ivC8D1) of mosunetuzumab administered on Day 1 of each respective dosing cycle, wherein the ivC7D1-ivC8D1 is each about 30 mg and does not comprise administration of polatuzumab vedotin.

In some embodiments, the subject has an Eastern Cooperative Oncology Group (ECOG) Performance Status of 0, 1, or 2. In some embodiments, aNHL is a diffuse large B cell lymphoma (DLBCL), a high grade B cell lymphoma (HGBL), a Grade 3b follicular lymphoma (FL), or a transformed FL (trFL). In some embodiments, the R/R NHL is an R/R large B cell lymphoma (LBCL), an R/R diffuse large B cell lymphoma (DLBCL), an R/R follicular lymphoma (FL), or an R/R mantle cell lymphoma (MCL). In some embodiments, the subject has relapsed after or is refractory to one or more prior lines of systemic therapy. In some embodiments, the subject is ineligible for autologous stem cell transplant (ASCT).

In some embodiments, the method comprises administering an additional therapeutic agent. In some embodiments, the additional therapeutic agent comprises a corticosteroid, an antihistamine, an antipyretic, or an IL-6R antagonist. In some embodiments, the additional therapeutic agent is a corticosteroid, and wherein the corticosteroid comprises prednisone, methylprednisolone, or dexamethasone. In some embodiments, the additional therapeutic agent is an antihistamine, and wherein the antihistamine comprises diphenhydramine hydrochloride or equivalent. In some embodiments, the additional therapeutic agent is an antipyretic, and wherein the antipyretic is acetaminophen. In some embodiments, the additional therapeutic agent is an IL-6R antagonist, wherein the IL-6R antagonist is tocilizumab.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the population of subjects has an overall response rate of between 74% and 100% (e.g., between 75% and 100%, between 75% and 90%, between 75% and 80%, between 80% and 100%, between 90% and 100%, between 80% and 90%, or between 85% and 95%; e.g., about 74%, 75%, 80%, 85%, 86%, 87%, 88%, 89%, 90%, 91%, 93%, 94%, 95%, or 100%).

In some embodiments, the overall response rate is about 88%.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the population of subjects has a complete response rate of between 63% and 93% (e.g., between 65% and 93%, between 75% and 93%, between 85% and 93%, between 63% and 80%, between 63% and 70%, between 70% and 80%, between 65% and 75%, or between 75% and 90%; e.g., about 63%, 65%, 68%, 69%, 70%, 71%, 75%, 77%, 78%, 79%, 80%, 81%, 82%, 85%, 90%, 91%, 92%, or 93%).

In some embodiments, the complete response rate is about 79%.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the median time to first response in the population of subjects is between 80 days to 100 days. In some embodiments, the median time to first response in the population of subjects is about 3 months.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the median PFS in the population of subjects is at least 14 months. In some embodiments, the median PFS in the population of subjects is about 19 months.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the median OS in the population of subjects is at least 17 months. In some embodiments, the median OS in the population of subjects is about 21 months.

In some embodiments, the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
   (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
   (i) a first subcutaneous dose (scC2D1) of mosunetuzumab administered on Day 1 of the second dosing cycle, wherein the scC2D1 is about 45 mg; and
   (ii) an intravenous dose of polatuzumab vedotin administered on Day 1 of the second dosing cycle, wherein the intravenous dose of polatuzumab vedotin is about 1.8 mg/kg.

In some embodiments, the dosing regimen of the combination treatment further comprises one or more additional 21-day dosing cycles (e.g., 1, 2, 3, 4, 5, 6, 7, 8, 9, 10, 11, 12, or more). In some embodiments, wherein the dosing regimen of the combination treatment comprises six additional 21-day dosing cycles.

In some embodiments, the MCL is R/R MCL. In some embodiments, the subjects of the population are R/R to Bruton's tyrosine kinase (BTK) inhibitor therapy. In some embodiments, the subjects of the population are R/R to prior chimeric antigen receptor T-cell (CAR T) therapy. In some embodiments, the subject is R/R to 2 or more prior lines of therapy. In some embodiments, the subjects of the population have a high-risk factor comprising a Ki-67 proliferation index ≥50%, a blastoid/pleomorphic variants, or a TP53 mutation.

In one aspect, the invention features a method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a CRR, an ORR, a PFS, or an OS.

In some embodiments, the efficacy response is a CRR, and wherein the CRR or reference CRR is the proportion of the subjects receiving the corresponding treatment whose best overall response is a complete response (CR). In some embodiments, the improved response in CRR is an increase in CRR compared to the reference CRR of between 3% and 44%. In some embodiments, the improved response in CRR is an increase in CRR compared to the reference CRR of about 25%.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 15% and 46%. In some embodiments, the improved response in ORR is an increase in ORR compared to the reference ORR of about 31%.

In some embodiments, the efficacy response is a PFS, and wherein the PFS or the reference PFS is measured starting from the time of receiving first dose of mosunetuzumab or polatuzumab vedotin to the time of a first occurrence of disease progression or death from any cause. In some embodiments, the PFS or the reference PFS is the median PFS of the plurality of subjects receiving the corresponding treatment.

In some embodiments, the improvement of the median PFS is an increase in the PFS compared to the reference PFS of between 1 and 15 months. In some embodiments, the improvement of the median PFS is an increase in the PFS compared to the reference PFS of 7 months.

In some embodiments, the improvement of the PFS is an increase in the rate of a PFS of 3 months compared to the rate of a reference PFS of 3 months of between 7% and 37%. In some embodiments, the improvement of the PFS is an increase in the rate of a PFS of 3 months compared to the rate of a reference PFS of 3 months of about 22%.

In some embodiments, the improvement of the PFS is an increase in the rate of a PFS of 6 months compared to the rate of a reference PFS of 6 months of between 11% and 44%. In some embodiments, the improvement of the PFS is an increase in the rate of a PFS of 6 months compared to the rate of a reference PFS of 6 months of about 27%.

In some embodiments, the improvement of the PFS is an increase in the rate of a PFS of 9 months compared to the rate of a reference PFS of 9 months of between 11% and 45%. In some embodiments, the improvement of the PFS is an increase in the rate of a PFS of 9 months compared to the rate of a reference PFS of 9 months of about 28%.

In some embodiments, the improvement of the PFS is an increase in the rate of a PFS of 12 months compared to the rate of a reference PFS of 12 months of between 10% and 44%. In some embodiments, the improvement of the PFS is an increase in the rate of a PFS of 12 months compared to the rate of a reference PFS of 12 months of about 27%.

In some embodiments, the improvement of the PFS is an increase in the rate of a PFS of 18 months compared to the rate of a reference PFS of 18 months of between 5% and 39%. In some embodiments, the improvement of the PFS is an increase in the rate of a PFS of 18 months compared to the rate of a reference PFS of 18 months of about 22%.

In some embodiments, the improvement of the PFS is an increase in the rate of a PFS of 24 months compared to the rate of a reference PFS of 24 months of between 5% and 38%. In some embodiments, the improvement of the PFS is an increase in the rate of a PFS of 24 months compared to the rate of a reference PFS of 24 months of about 21%.

In some embodiments, the efficacy response is an OS, and wherein the OS or the reference OS is measured starting from the time of receiving first dose of mosunetuzumab or polatuzumab vedotin to the time of a first occurrence of disease progression or death from any cause. In some embodiments, the OS or the reference OS is the median OS of the plurality of subjects receiving the corresponding treatment.

In some embodiments, the improvement of the median OS is an increase in the OS compared to the reference OS. In some embodiments, the improvement of the median OS is an increase in the OS compared to the reference OS of 7.4 months.

In some embodiments, the improvement of the OS is an increase in the rate of a OS of 6 months compared to the rate of a reference OS of 6 months of between 1% and 26%. In some embodiments, the improvement of the OS is an increase in the rate of a OS of 6 months compared to the rate of a reference OS of 6 months of about 12%.

In some embodiments, the improvement of the OS is an increase in the rate of a OS of 9 months compared to the rate of a reference OS of 9 months of between 1% and 24%. In some embodiments, the improvement of the OS is an increase in the rate of a OS of 9 months compared to the rate of a reference OS of 9 months of about 9%.

In some embodiments, the improvement of the OS is an increase in the rate of a OS of 12 months compared to the rate of a reference OS of 12 months of between 1% and 27%. In some embodiments, the improvement of the OS is an increase in the rate of a OS of 12 months compared to the rate of a reference OS of 12 months of about 11%.

In some embodiments, the improvement of the OS is an increase in the rate of a OS of 18 months compared to the rate of a reference OS of 18 months of between 1% and 28%. In some embodiments, the improvement of the OS is an increase in the rate of a OS of 18 months compared to the rate of a reference OS of 18 months of about 11%.

In some embodiments, the improvement of the OS is an increase in the rate of a OS of 24 months compared to the rate of a reference OS of 24 months of between 1% and 29%. In some embodiments, the improvement of the OS is an increase in the rate of a OS of 24 months compared to the rate of a reference OS of 24 months of about 13%.

In some embodiments, the CR or PR determined by PET/computed tomography (CT). In some embodiments, the CR or PR is determined based on the Lugano Response Criteria for Malignant Lymphoma (Cheson et al., 2014).

In one aspect, the invention features a method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered R-GemOx, and wherein the safety response is the rate of AE or the rate of SAE.

In some embodiments, the safety response is the rate of AE, and wherein the rate of AE or the reference rate of AE is the rate of AE in the plurality of subjects receiving the corresponding treatment.

In some embodiments, the safety response in rate of AE is non-inferior compared to the reference rate of AE. In some embodiments, the safety response in rate of Grade 3-5 AE is non-inferior compared to the reference rate of Grade 3-5 AE. In some embodiments, the Grade of AE is determined based on the National Cancer Institute Common Terminology Criteria for Adverse Events (NCI CTCAE), v5.0.

In some embodiments, the safety response is the rate of SAE, and wherein the rate of SAE or the reference rate of SAE is the rate of SAE in the plurality of subjects receiving the corresponding treatment. In some embodiments, the safety response in rate of SAE is non-inferior compared to the reference rate of SAE.

In some embodiments, the combination treatment comprises subcutaneously administering mosunetuzumab and intravenously administered polatuzumab vedotin according to a dosing regimen comprising at least a first 21-day dosing cycle and a second 21-day dosing cycle, wherein: (a) the first dosing cycle comprises: (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the cC1D2 is about 45 mg, and the scC1D3 is about 45 mg; and (ii) an intravenous dose of polatuzumab vedotin administered on Day 1 of the first dosing cycle, wherein the intravenous dose of polatuzumab vedotin is about 1.8 mg/kg; and (b) the second dosing cycle comprises: (i) a first subcutaneous dose (scC2D1) of mosunetuzumab administered on Day 1 of the second dosing cycle, wherein the scC2D1 is about 45 mg; and (ii) an intravenous dose of polatuzumab vedotin administered on Day 1 of the second dosing cycle, wherein the intravenous dose of polatuzumab vedotin is about 1.8 mg/kg. In some embodiments, the dosing regimen of the combination treatment further comprises six additional 21-day dosing cycles.

In some embodiments, (a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and (b) the next two additional dosing cycles each comprises a subcutaneous dose of mosunetuzumab administered on Day 1 of the additional dosing cycle, wherein the subcutaneous dose of mosunetuzumab is about 45 mg.

In some embodiments, the control treatment comprises intravenously administering 375 mg/m² rituximab, 1000 mg/m² gemcitabine, and 100 mg/m² oxaliplatin for eight 14-day dosing cycles, wherein rituximab, gemcitabine, and oxaliplatin are administered on Day 1 of each dosing cycle.

### BRIEF DESCRIPTION OF THE DRAWINGS

FIG. 1 is a schematic of the study design described in Example 1. 2L=second-line; DLBCL=diffuse large B cell lymphoma; CRR=complete response rate; FL=follicular lymphoma; M=mosunetuzumab; NOS=not otherwise specified; OS=overall survival; ORR=objective response rate; P =polatuzumab vedotin; PFS=progression-free survival; Q2W=every 2 weeks (i.e., 14-day dosing cycles); Q3W=every 3 weeks (i.e., 21-day dosing cycles); R GemOx=rituximab, gemcitabine, and oxaliplatin; R/R=relapsed and/or refractory.

FIG. 2A is a schematic of the dosing regimen of Arm A (mosunetuzumab+polatuzumab vedotin arm) described in Example 1. Treatment comprises eight total dosing cycles (Cycles 1-8). Mosunetuzumab is administered with the following doses and timings: 5 mg subcutaneously on Cycle 1, Day 1; 45 mg on Cycle 1, Day 8; Cycle 1, Day 15; and Day 1 of Cycles 2-8. Polatuzumab vedotin is administered intravenously with the following dose and timing: 1.8 mg/kg on Day 1 of Cycles 1-6. Each dosing cycle is 21 days.

FIG. 2B is a schematic of the dosing regimen of Arm B (R-GemOx arm) described in Example 1. Treatment comprises eight total dosing cycles (Cycles 1-8). Rituximab 375 mg/m² is administered IV on Day 1 of Cycles 1-8. Gemcitabine 1000 mg/m² is administered IV on Day 1 of Cycles 1-8. Oxaliplatin 100 mg/m² is administered IV on Day 1 of Cycles 1-8. Each dosing cycle is 14 days.

FIG. 3A shows the study design described in Example 2. R/R=relapsed and/or refractory; DLBCL=diffuse large B cell lymphoma; FL=follicular lymphoma; MCL=mantle cell lymphoma; IV=intravenous; SC=subcutaneous; Pola=polatuzumab vedotin.

FIG. 3B shows the study treatment duration and follow-up duration described in Example 2.

FIG. 4 shows decision-making regarding treatment and re-treatment of patients in Groups A, B, and C, and Arms I, J, and K for the study described in Example 2.

FIG. 5 shows the patient demographics and baseline characteristics of patients described in Example 3.

FIG. 6 is a Kaplan-Meier plot showing duration of response of patients in Arm J, as well as summary statistics described in Example 3.

FIG. 7 is a Kaplan-Meier plot showing progression-free survival of patients in Arm J, as well as summary statistics described in Example 3.

FIG. 8 is a Kaplan-Meier plot showing overall survival of patients in Arm J, as well as summary statistics described in Example 3.

FIG. 9 summarizes the adverse events experienced by patients in Group A or Arm J described in Example 3.

FIG. 10 summarizes the adverse events experienced by patients in Arms L or M described in Example 3.

FIG. 11 summarizes the serious adverse events experienced by patients in Group A or Arm J described in Example 3.

FIG. 12 summarizes the serious adverse events experienced by patients in Arms L or M described in Example 3.

FIG. 13 summarizes the hematological adverse events experienced by patients in Group A or Arm J described in Example 3.

FIG. 14 summarizes the hematological adverse events experienced by patients in Arms L or M described in Example 3.

FIG. 15A-FIG. 15F summarizes various adverse events of special interest described in Example 3, i.e., AST/ALT/total bilirubin elevation and cytokine release syndrome (FIG. 15A); febrile neutropenia and immune effector cell-associated neurotoxicity syndrome (ICANs) events (FIG. 15B); infections/infestations and neurological AE (FIG. 15C); neutropenia and pleural effusion (FIG. 15D); pneumonitis/interstitial lung disease and rash (FIG. 15E); and tumor flare events (FIG. 15F).

FIG. 16 summarizes death events and primary causes described in Example 3.

FIG. 17A-FIG. 17C summarizes patient demographics (FIG. 17A) and baseline characteristics (FIG. 17B and FIG. 17C) of patients described in Example 4.

FIG. 18 describes summary statistics of the complete response (CR), partial response (PR), stable disease (SD), and progressive disease (PD) results of Arms A (Mosun+Pola) and B (R-GemOx) described in Example 4.

FIG. 19A-FIG. 19D describes results comparing Arm A with Arm B when stratified by various demographics (FIG. 19A) and baseline characteristics (FIG. 19B-FIG. 19D) of the patients described in Example 4.

FIG. 20 is a Kaplan-Meier plot showing duration of response of patients, as well as summary statistics described in Example 4.

FIG. 21 is a Kaplan-Meier plot showing duration of complete response of patients, as well as summary statistics described in Example 4.

FIG. 22 summarizes the adverse events reported in Arms A (Mosun+Pola) and B (R-GemOx) described in Example 4.

FIG. 23 summarizes the death and primary causes in Arms A (Mosun+Pola) and B (R-GemOx) described in Example 4.

FIG. 24 summarizes the adverse events with occurrence >10% in Arms A (Mosun+Pola) and B (R-GemOx) described in Example 4.

FIG. 25 summarizes the serious adverse events reported in Arms A (Mosun+Pola) and B (R-GemOx) described in Example 4.

FIG. 26 summarizes the treatments used for CRS management in Arm A (Mosun+Pola) described in Example 4.

FIG. 27 summarizes the timing of CRS events and Grade of CRS events relative to mosunetuzumab dosing described in Example 4.

FIG. 28 summarizes the injection site reactions that occurred with mosunetuzumab SC administration described in Example 4.

FIG. 29 summarizes the infection/infestation events in Arms A (Mosun+Pola) and B (R-GemOx) described in Example 4.

FIG. 30 summarizes the adverse events of special interest (AESI) in Arms A (Mosun+Pola) and B (R-GemOx) described in Example 4.

FIG. 31A-FIG. 31C show efficacy of subcutaneous mosunetuzumab+intravenous polatuzumab vedotin in patients with R/R MCL in terms of PFS (FIG. 31A); OS (FIG. 31B); and response rates (FIG. 31C) in all patients and in high-risk subgroups. CAR: chimeric antigen receptor; CI: confidence interval; CR: complete response; MCL: mantle cell lymphoma; M+Pola: mosunetuzumab plus polatuzumab vedotin; NE: not evaluable; OS: overall survival; PFS: progression-free survival; PR: partial response; R/R: relapsed/refractory.

FIG. 32 shows time to event summary for PFS by IRS in ITT patients, excluding Chinese patients.

FIG. 33 shows a Kaplan-Meier plot of PFS determined by IRC in ITT population, excluding Chinese patients, with CCOD of Feb. 17, 2025.

FIG. 34A-FIG. 34E are forest plots showing subgroup analyses results of PFS in Arm A vs Arm B, excluding Chinese patients.

FIG. 35 shows time to event summary for OS by IRS in ITT patients, excluding Chinese patients.

FIG. 36 shows a Kaplan-Meier plot of OS determined by IRC in ITT population, excluding Chinese patients, with CCOD of Feb. 17, 2025.

FIG. 37 shows assessment of overall (complete and partial) responses by IRC up to first NALT date, excluding Chinese patients, with CCOD of Feb. 17, 2025.

FIG. 38 shows summary of adverse events in patients from Arm A and Arm B, excluding Chinese patients.

### DETAILED DESCRIPTION

The present invention relates to methods of treating a subject or a population of subjects having a CD20-positive cell proliferative disorder (e.g., a B cell proliferative disorder; e.g., a non-Hodgkin's lymphoma (NHL) (e.g., a large B cell lymphoma (LBCL), a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma; e.g., a relapsed and/or refractory NHL (R/R NHL; e.g., an R/R LBCL, an R/R DLBCL, an R/R FL, or an R/R MCL) or an aggressive NHL (aNHL, e.g., R/R diffuse large DLBCL, R/R HGBL, R/R trFL, and R/R Grade 3b FL)), by subcutaneous administration of mosunetuzumab and intravenous administration of polatuzumab vedotin as a combination treatment, wherein the combination treatment comprising subcutaneous administration of mosunetuzumab in combination with intravenous administration of polatuzumab vedotin exhibits non-inferiority or an improved outcome as measured using pharmacokinetics (PK), efficacy, safety, or a combination thereof, as compared to (i) a reference treatment comprising intravenous administration of mosunetuzumab and intravenous administration of polatuzumab vedotin; (ii) a reference treatment comprising rituximab and polatuzumab vedotin; or (iii) a combination treatment comprising rituximab, gemcitabine, and oxaliplatin (R-GemOx).

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. DEFINITIONS

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

The terms “cancer” and “cancerous” refer to or describe the physiological condition in mammals that is typically characterized by unregulated cell growth. Examples of cancer include, but are not limited to, hematologic cancers, such as mature B cell cancers, excluding Hodgkin's lymphoma, but including non-Hodgkin's lymphoma (NHL), such as large B cell lymphoma (LBCL), diffuse large B cell lymphoma (DLBCL), which may be relapsed and/or refractory DLBCL or a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high grade B-cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, γ heavy chain disease, u heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “B cell proliferative disorder” or “B cell malignancy” refer to disorders that are associated with some degree of abnormal B cell proliferation and include, for example, lymphomas, leukemias, myelomas, and myelodysplastic syndromes. In some instances, the B cell proliferative disorder is a lymphoma, such as non-Hodgkin's lymphoma (NHL), including, for example, follicular lymphoma (FL) (e.g., a relapsed and/or refractory FL or transformed FL (trFL)), large B cell lymphoma (LBCL), diffuse large B cell lymphoma (DLBCL) (e.g., a relapsed and/or refractory DLBCL or a Richter's transformation), mantle cell lymphoma (MCL), high grade B-cell lymphoma (HGBL), primary mediastinal (thymic) large B-cell lymphoma (PMLBCL), diffuse B cell lymphoma, small lymphocytic lymphoma, marginal zone lymphoma (MZL), Burkitt lymphoma, or lymphoplasmacytic lymphoma. In another embodiment, the B cell proliferative disorder is a leukemia, such as chronic lymphocytic leukemia (CLL). In one embodiment, the B-cell proliferative disorder is relapsed and/or refractory. In some embodiments, an NHL may be an aggressive NHL (aNHL), including, e.g., HGBL, DLBCL, trFL, and Grade 3b FL (see Swerdlow S H, et al. Blood 2016; 127:2375-90).

“Refractory disease” is defined as no complete remission to at least a first-line therapy. In one embodiment, refractory disease defined as no response to or relapse within 6 months of prior therapy. In one embodiment, refractory disease is characterized by one or more of the following: progressive disease (PD) as best response to first-line therapy, stable disease (SD) as best response after at least one first line therapy, or partial response (PR) as best response, and biopsy-proven residual disease or disease progression after the partial response. “Relapsed disease” is defined as complete remission to first-line therapy. In one embodiment, disease relapse is proven by biopsy. In one embodiment, subjects have relapsed after or failed to respond to at least one prior systemic treatment regimen.

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies and antibody drug conjugates of the invention are used to delay development of a disease or to slow the progression of a disease.

As used herein, “delaying progression” of a disorder or disease means to defer, hinder, slow, retard, stabilize, and/or postpone development of the disease or disorder (e.g., a CD20-positive cell proliferative disorder). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with mosunetuzumab using the step-up dosing regimen of the invention relative to unchanging, preset dosing with the target dose of mosunetuzumab. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the CD20-positive cell proliferative disorder (e.g., the B cell proliferative disorder) being treated, the presence or size of metastases, or the size of the primary tumor. In yet other embodiments, reducing or inhibiting cancer relapse means to reduce or inhibit tumor or cancer relapse, or tumor or cancer progression.

As used herein, “administering” is meant a method of giving a dosage of a compound (e.g., a bispecific antibody (e.g., mosunetuzumab) and/or an antibody drug conjugate (e.g., polatuzumab vedotin)) or a composition (e.g., a pharmaceutical composition, e.g., a pharmaceutical composition including a bispecific antibody (e.g., mosunetuzumab) and/or an antibody drug conjugate (e.g., polatuzumab vedotin)) to a subject. The compounds and/or compositions utilized in the methods described herein can be administered subcutaneously (e.g., by subcutaneous injection) or intravenously (e.g., by intravenous infusion).

A “fixed” or “flat” dose of a therapeutic agent (e.g., a bispecific antibody (e.g., mosunetuzumab) or an antibody drug conjugate (e.g., polatuzumab vedotin)) herein refers to a dose that is administered to a subject without regard for the weight or body surface area (BSA) of the subject. The fixed or flat dose is therefore not provided as a mg/kg dose or a mg/m² dose, but rather as an absolute amount of the therapeutic agent (e.g., mg).

A “subject” or an “individual” is a mammal. Mammals include, but are not limited to, primates (e.g., humans and non-human primates such as monkeys), domesticated animals (e.g., cows, sheep, cats, dogs, and horses), rabbits, and rodents (e.g., mice and rats). In particular embodiments, the subject or individual is a human.

“Individual response” or “response” can be assessed using any endpoint indicating a benefit to the subject, including, without limitation, (1) inhibition, to some extent, of disease progression (e.g., progression of a CD20-positive cell proliferative disorder (e.g., a B cell proliferative disorder), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the CD20-positive cell proliferative disorder (e.g., the B cell proliferative disorder); (6) increase or extend in the length of survival, including overall survival and progression-free survival; and/or (7) decreased mortality at a given point of time following treatment.

As used herein, “complete response” or “CR” refers to disappearance of all target lesions (i.e., all evidence of disease).

As used herein, “partial response” or “PR” refers to at least a 30% decrease in the sum of the longest diameters (SLD) of target lesions, taking as reference the baseline SLD, or at least a 50% decrease in the sum of the product of the diameters (SPD) of target lesions, taking as reference the baseline SPD.

As used herein, “objective response rate” or “ORR” refers to the sum of complete response (CR) rate and partial response (PR) rate.

As used herein, “duration of objective response”, “duration of response”, or “DOR” is defined as the time from the first occurrence of a documented objective response (CR or PR) to the time of disease progression, relapse, or death from any cause, whichever occurs first.

As used herein, “duration of complete response” or “DOCR” is defined as the time from the first occurrence of a documented complete response to disease progression, or death from any cause within 30 days of the last dose of a treatment, whichever occurs first.

As used herein, “tumor burden” refers to the total amount of tumor (e.g., tumor cells or tumor mass) in a subject (e.g., a human subject) having a cancer, e.g., a CD20-positive cell proliferative disorder (e.g., a B cell proliferative disorder). In some embodiments, tumor burden is defined as the sum of diameters of target lesions or the sum of the product of target lesions. In a particular embodiment, tumor burden is defined as the sum of the product of the diameters of (SPD) target lesions. In some embodiments, the diameter of target lesions is quantified by computed tomography (CT).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “progression-free survival” or “PFS” is defined as the time from first study treatment to the first occurrence of disease progression, relapse, or death from any cause, whichever occurs first.

As used herein, “event-free survival” or EFS″ is defined as the time from first study treatment to the first occurrence of any treatment failure including disease progression, relapse, initiation of NALT, or death.

As used herein, “overall survival” or “OS” is defined as the time from first study treatment to the date of death from any cause.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, “delaying progression” of a disorder or disease means to defer, hinder, slow, retard, stabilize, and/or postpone development of the disease or disorder (e.g., a CD20-positive cell proliferative disorder (e.g., a B cell proliferative disorder)). This delay can be of varying lengths of time, depending on the history of the disease and/or subject being treated. As is evident to one skilled in the art, a sufficient or significant delay can, in effect, encompass prevention, in that the subject does not develop the disease. For example, in a late-stage cancer, development of central nervous system (CNS) metastasis, may be delayed.

By “extending survival” is meant increasing overall or progression free survival in a treated subject relative to an untreated subject (e.g., relative to a subject not treated with the medicament), or relative to a subject who does not express a biomarker at the designated level, and/or relative to a subject treated with an approved anti-tumor agent. An objective response refers to a measurable response, including complete response (CR) or partial response (PR).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “full-length antibody,” “intact antibody,” and “whole antibody” are used herein interchangeably to refer to an antibody having a structure substantially similar to a native antibody structure or having heavy chains that contain an Fc region as defined herein.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “variable region” or “variable domain” refers to the domain of an antibody heavy or light chain that is involved in binding the antibody (i.e., mosunetuzumab) to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred embodiments, the antibody (i.e., mosunetuzumab) will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody (i.e., mosunetuzumab) as being obtained from a substantially homogeneous population of antibodies, and is not to be construed as requiring production of the antibody by any particular method. For example, the monoclonal antibodies (i.e., mosunetuzumab) to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10-13 M, e.g., from 10⁻⁹ M to 10-13 M). In certain embodiments, an anti-CD3 antibody binds to an epitope of CD3 that is conserved among CD3 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 108 M to 10-13 M, e.g., from 10⁻⁹ M to 10-13 M). In certain embodiments, an anti-CD20 antibody binds to an epitope of CD20 that is conserved among CD20 from different species. In some embodiments, the anti-CD20 antibody is a monoclonal antibody. In some embodiments, the anti-CD20 antibody or anti-CD20 monoclonal antibody is rituximab. In some embodiments, the anti-CD20 antibody or anti-CD20 monoclonal antibody is obinutuzumab.

As used herein, the term “rituximab” or “RITUXAN®” refers to an anti-CD20 antibody (e.g., anti-CD20 monoclonal antibody) having the Proposed International Nonproprietary Names for Pharmaceutical Substances (Proposed INN) List 77 (WHO Drug Information, Vol. 11, No. 2, 1997, p. 99), or the CAS Registry Number 174722-31-7.

As used herein, the term “obinutuzumab” or “GAZYVAR” refers to an anti-CD20 antibody (e.g., anti-CD20 monoclonal antibody) having the Proposed International Nonproprietary Names for Pharmaceutical Substances (Proposed INN) List 99 (WHO Drug Information, Vol. 22, No. 2, 2008, p. 396), Proposed International Nonproprietary Names for Pharmaceutical Substances (Proposed INN) List 108 (WHO Drug Information, Vol. 26, No. 4, 2012, p. 453), or the CAS Registry Number 949142-50-1.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2).

The terms “anti-CD20/anti-CD3 bispecific antibody,” “bispecific anti-CD20/anti-CD3 antibody,” and “antibody that binds to CD20 and CD3,” or variants thereof, refer to mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 303), or the CAS Registry Number 1905409-39-3.

The terms “anti-CD79b antibody” and “an antibody that binds to CD79b” refer to an antibody that is capable of binding CD79b with sufficient affinity such that the antibody is useful as a diagnostic and/or therapeutic agent in targeting CD79b. In one embodiment, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10-13 M, or e.g., from 10⁻⁹ M to 10-13 M). In certain embodiments, an anti-CD79b antibody binds to an epitope of CD79b that is conserved among CD79b from different species. As used herein, an anti-CD79b antibody is polatuzumab.

As used herein, the term “polatuzumab vedotin” refers to an antibody drug conjugate comprising an anti-CD79b antibody conjugated to monomethyl auristatin E (MMAE, i.e., vedotin). Polatuzumab vedotin is also referred to as IUPHAR/BPS Number 8404, the KEGG Number D10761, or by the CAS Registry Number 1313206-42-6. Polatuzumab vedotin-piiq is also interchangeably referred to as “polatuzumab vedotin-piiq”, “huMA79bv28-MC-vc-PAB-MMAE”, or “DCDS4501A” as described in U.S. U.S. Pat. No. 8,088,378).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 M, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10-11 M or lower, alternatively 10-12 M or lower or a Ko in the range of 10⁻⁴ M to 10⁻⁶ M or 10⁻⁶ M to 10-10 M or 10⁻⁷ M to 10⁻⁹ M. As will be appreciated by the skilled artisan, affinity and Ko values are inversely related. A high affinity for an antigen is measured by a low Ko value. In one embodiment, the term “specific binding” refers to binding where a molecule binds to a particular polypeptide or epitope on a particular polypeptide without substantially binding to any other polypeptide or polypeptide epitope.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “chemotherapeutic agent” refers to a compound useful in the treatment of a cancer, such as a CD20-positive cell proliferative disorder (e.g., a B cell proliferative disorder; e.g., a non-Hodgkin's lymphoma (NHL) (e.g., a large B cell lymphoma (LBCL), a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma; e.g., a relapsed and/or refractory NHL (R/R NHL; e.g., an R/R LBCL, an R/R DLBCL, an R/R FL, or an R/R MCL) or an aggressive NHL (aNHL, e.g., R/R diffuse large DLBCL, R/R HGBL, R/R trFL, and R/R Grade 3b FL)). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); ECB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl) methoxy]phenyl]-6 [5 [2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN@ cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, dacarbazine, mechlorethamine, cisplatin, methotrexate, 5-fluorouracil, and ara-C); and (x) pharmaceutically acceptable salts, acids, prodrugs, and derivatives of any of the above.

The term “chemo-immunotherapy” refers to combination therapy that includes both chemotherapy drugs and immunotherapeutic agents. In some embodiments, chemo-immunotherapy is used to treat a cancer, e.g., a CD20-positive cancer, e.g., a NHL, e.g., e.g., a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma. In some embodiments, immunotherapeutic agents include an antibody, e.g., an anti-CD20 antibody (e.g., an anti-CD20 monoclonal antibody). In some embodiments, the anti-CD20 antibody or anti-CD20 monoclonal antibody is rituximab. In some embodiments, chemo-immunotherapy includes R-GemOx.

The term “R-GemOx” as used herein refers to a treatment comprising rituximab (RITUXAN®; CAS #: 174722-31-7) plus gemcitabine (CAS #: 95058-81-4) and oxaliplatin (CAS #: 61825-94-3). In some embodiments, R-GemOx is a chemotherapy treatment or regimen used in the treatment of a cancer, optionally a B cell proliferative disorder (e.g., a non-Hodgkin's lymphoma; e.g., a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma). In some embodiments, R-GemOx is the standard of care (SOC) or standard therapy to be administered to a subject to treat the cancer, optionally the B cell proliferative disorder (e.g., the non-Hodgkin's lymphoma; e.g., the diffuse-large B cell lymphoma (DLBCL), a follicular lymphoma (FL), the high-grade B cell lymphoma (HGBL), the mantle cell lymphoma (MCL), the high-grade B cell lymphoma, the primary mediastinal (thymic) large B cell lymphoma (PMLBCL), the diffuse B cell lymphoma, the small lymphocytic lymphoma, the marginal zone lymphoma (MZL), the Burkitt lymphoma, the lymphoplasmacytic lymphoma). In some embodiments, R-GemOx is the standard therapy to be administered to subjects who are relapsed and/or refractory to prior therapies and/or subjects who are ineligible for autologous stem cell therapy (ASCT). In some embodiments, R-GemOx is administered every two weeks (in 14-day dosing cycles) for eight dosing cycles. In some embodiments, the dosing regimen for R-GemOx therapy comprises eight 14-day dosing cycles, wherein during each dosing cycle, the subject is administered 375 mg/m² rituximab intravenously (IV), 1000 mg/m² gemcitabine IV, and 100 mg/m² oxaliplatin IV.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “package insert” is used to refer to instructions customarily included in commercial packages of therapeutic products, that contain information about the indications, usage, dosage, administration, combination therapy, contraindications and/or warnings concerning the use of such therapeutic products.

The term “NALT” or “new anti-lymphoma treatment” refers to any non-protocol-specified therapy initiated after a progression event (like disease progression or relapse) in a clinical trial. NALTs may include radiotherapy, stem cell transplant (including autologous or allogenic stem cell transplants), chimeric antigen receptor T cell (CAR-T) therapy, polatuzumab vedotin-based therapy, or bispecific antibody treatment.

The term “dose intensity” refers to the amount of treatment (e.g., mosunetuzumab+polatuzumab vedotin (Mosun+Pola) or rituximab, gemcitabine, and oxaliplatin (R-GemOx) administered per time unit. In some embodiments, dose intensity used herein, particularly when expressed as a percentage, refers to relative dose intensity, calculated as (actual dose/actual days)/(planned dose/planned days)*100%.

The term “International Prognostic Index” or “IPI” refers to a system used to predict the prognosis of diffuse large B-cell lymphoma patients, primarily based on five factors: age (>60 years of age), Ann Arbor stage (III or IV), LDH level (serum LDH level >1× normal), ECOG performance status (2 or higher), and the number of extranodal sites (>1 extranodal site-bone marrow, GI tract, liver, lung, central nervous system (CNS), skin, testes, Waldeyer's ring). See The International Non-Hodgkin's Lymphoma Prognostic Factors Project, Shipp et al., N. Engl. J. Med. 329:987-994, 1993, which is incorporated herein by reference in its entirety.

As used herein, the terms “Ann Arbor staging” or “Ann Arbor stages” refers to a system for classification of stages of lymphoma (e.g., NHLs). Lymphomas (e.g., NHLs) can be classified as one of four Ann Arbor stages. Stage I refers to lymphomas exhibiting involvement of a single lymph node region or of a single extralymphatic organ or site. Stage II refers to lymphomas exhibiting involvement of 2 or more lymph node regions on the same side of the diaphragm. Stage III refers to lymphomas exhibiting involvement of lymph node regions on both sides of the diaphragm (III), which may also be accompanied by localized involvement of extralymphatic organ or site or by involvement of the spleen, or both. Stage IV refers to lymphomas exhibiting diffuse or disseminated involvement of 1 or more extralymphatic organs or tissues with or without associated lymph node enlargement. Liver involvement is always considered to be diffuse, and, thus, always considered Ann Arbor stage IV. Lymphatic structures include the lymph nodes, thymus, spleen, appendix, Waldeyer's ring, and Peyer's patches. See Carbone, P. P. et al., Cancer Res. 1971, 31 (11): 1860-1861, which is incorporated herein by reference in its entirety.

The term “double hit” refers to B cell lymphomas (e.g., HGBL) with rearrangements in MYC and B-cell lymphoma 2 (BCL2) genes.

The term “triple hit” refers to B cell lymphomas (e.g., HGBL) with rearrangements in MYC, B-cell lymphoma 2 (BCL2), and B-cell lymphoma 6 (BCL6) genes.

The term “activities of daily living score” or “ADL score” refers a scoring system of evaluating whether an individual is capable of performing activities that allows the individual to live. ADL activities include bathing, personal hygiene and grooming, toileting and continence, eating, dressing, and moving. ADL scoring gives 1 point for each ADL activity the individual is capable of.

The term “instrumental activities of daily living score” or “IADL score” refers to a scoring system of evaluating whether an individual is capable of performing activities that allows the individual to live independently in a community. IADL activities include managing money, managing a household, managing health, preparing meals, communicating with others, managing transportation, and shopping. IADL scoring gives 1 point for each IADL activity the individual is capable of.

The term “Cumulative Illness Rating Scale-Geriatric score” or “CIRS-G score” refers to a scoring system of evaluating chronic medical illness burden in geriatric patients. An individual evaluated on a scale of 0-4 points for each of heart; vascular; respiratory; eyes, ears, nose, throat, and larynx; upper gastrointestinal (GI) tract; lower GI tract; liver, pancreas, and biliary; genitourinary; musculoskeletal and skin; neurologic; endocrine and breast; and psychiatric function categories. The CIRS-G score of a patient refers to the total score (e.g., sum) across all categories. “Worst CIRS-G score” refers to the highest score in any function category. See Miller et al., Psychiatry Research. 41 (3): 237-248, which is incorporated herein by reference in its entirety.

The term “Mini Nutritional Assessment-Short Form score” or “MNA-SF score” refers to a scoring system of evaluating whether an individual is at risk of malnutrition. An MNA-SF score of 12-14 indicates normal nutritional status. An MNA-SF score of 8-11 indicates a risk of malnutrition, An MNA-SF score of 0-7 indicates malnutrition. See Rubenstein et al., J. Gerontol. A Giol. Sci. Med. Sci. 56: M366-M372, 2001, which is incorporated herein by reference in its entirety.

The term “Ki-67 proliferation index” refers to the percentage of cells in a sample that are stained positively for the Ki-67 protein, which is a biomarker for actively dividing cells. In some embodiments, the Ki-67 proliferation index indicates the percentages of cells in a sample (e.g., a biopsy sample) that are in a state of cell division (e.g., actively dividing).

The terms “blastoid variant” and “pleomorphic variant” of mantle cell lymphoma (MCL) refer to morphologically distinguishable subgroups of MCL. Blastoid variant MCL cells have blastic morphology. Pleomorphic variant MCL cells are larger than those in conventional MCL, and whose nuclear shape and chromatic structure resemble diffuse large B-cell lymphoma cells to some extend. See Dreyling et al., Blood. 132 (26): 2722-2729 (2018), which is incorporated herein by reference in its entirety.

The term “quality of life factor” as used herein refers to a patient-reported outcome, e.g., regarding a patient or subject's quality of life. In some instances, the quality of life factor is determined by a patient or subject's self-reported evaluation regarding their quality of life. In some embodiments, the quality of life factor is evaluated according to a European Organization for Research and Treatment of Cancer Quality of Life Questionnaire Core 30 (EORTC QLQ-C30) for global health status, functioning, and core symptom scales (see Aaronson et al. J. Natl Cancer Inst. 1993, 85 (5): 365-376; Fitzsimmons et al., Eur. J. Cancer. 1999, 35 (6): 939-941. In some embodiments, the quality of life factor includes appetite loss, cognitive functioning, constipation, diarrhea, dyspnea, emotional functioning, fatigue, financial difficulties, nausea and vomiting, pain, physical functioning, global health status, role functioning, social functioning, and insomnia.

The term “lymphoma factor” as used herein refers to a patient-reported outcome, e.g., regarding a patient or subject's experience of lymphoma symptoms. In some embodiments, the lymphoma factor is evaluated according to a Functional Assessment of Cancer Therapy-Lymphoma subscale (FACT Lym LymS); see Hlubocky et al., Leuk Lymphoma. 2013, 54 (9): 1942-1946. In some embodiments, the lymphoma factor includes fever, night sweats, weight loss, and lymphoma subscale (FACT Lym LymS).

The term “peripheral neuropathy factor” as used herein refers to a patient-reported outcome, e.g., regarding a patient or subject's experience of peripheral neuropathy symptoms. In some embodiments, the peripheral neuropathy factor is evaluated according to a Functional Assessment of Cancer Therapy-Gynecologic Oncology Group-Neurotoxicity (FACT/GOG-Ntx; Huang et al., Int. J. Gynecol. Cancer. 2007, 17 (2): 387-393). In some embodiments, the peripheral neuropathy factor includes neurotoxicity subscale, numbness or tingling in the hands or feet, discomfort in the hands or feet, joint pain or muscle cramps, trouble hearing, ringing or buzzing in ears, and difficulties in fine motor control in the hands.

### III. THERAPEUTIC METHODS

Provided herein are methods of treating a subject or a population of subjects having a CD20-positive cell proliferative disorder (e.g., a B cell proliferative disorder; e.g., a non-Hodgkin's lymphoma (NHL) (e.g., a large B cell lymphoma (LBCL), a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma; e.g., a relapsed and/or refractory NHL (R/R NHL; e.g., an R/R LBCL, an R/R DLBCL, an R/R FL, or an R/R MCL) or an aggressive NHL (aNHL, e.g., R/R diffuse large DLBCL, R/R HGBL, R/R trFL, and R/R Grade 3b FL)), by subcutaneous administration of mosunetuzumab and intravenous administration of polatuzumab vedotin as a combination treatment, wherein the combination treatment comprising subcutaneous administration of mosunetuzumab as a monotherapy or in combination with lenalidomide exhibits non-inferiority or an improved outcome as measured using pharmacokinetics (PK), efficacy, safety, or a combination thereof, as compared to a combination treatment comprising intravenous administration of mosunetuzumab and intravenous administration of polatuzumab vedotin or a combination treatment comprising rituximab, gemcitabine, and oxaliplatin (R-GemOx). In particular, provided herein are methods of treating a subject or a population of subjects having an aggressive NHL (e.g., a DLBCL, a transformed FL, or a Grade 3b FL) by subcutaneous administration of mosunetuzumab and intravenous administration of polatuzumab vedotin as a combination therapy. In some instances, the subject or the population of subjects are relapsed and/or refractory (R/R) to at least one line of prior therapy, while maintaining an acceptable safety profile (e.g., with respect to frequency and severity of adverse events, such as cytokine release syndrome (CRS)). In some instances, the subject or the population of subjects may have received two or more lines of prior therapy. In some instances, the subjects may be ineligible for autologous stem cell transplant (ASCT).

### A. Therapeutic Methods for Dosing of Mosunetuzumab and Polatuzumab Vedotin

In one aspect, the invention features a method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

In some embodiments, the efficacy response is a CRR, and wherein the CRR or reference CRR is the proportion of the subjects receiving the corresponding treatment whose best overall response is a complete response (CR).

In some embodiments, the improved response in CRR is an increase in CRR compared to the reference CRR of between 1% and 41% (e.g., between 1% and 30%, between 1% and 20%, between 1% and 10%, between 10% and 30%, between 10% and 20%, between 15% and 35%, between 15% and 25%, between 30% and 40%, or between 20% and 40%; e.g., about 5%, 10%, 15%, 20%, 21%, 22%, 23%, 25%, 30%, 35%, 40%, or 41%). In some embodiments, the improved response in CRR is an increase in CRR compared to the reference CRR of about 20.8%.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 3% and 47% (e.g., between 3% and 40%, between 3% and 35%, between 3% and 30%, between 3% and 25%, between 3% and 15%, between 3% and 10%, between 5% and 45%, between 5% and 45%, between 10% and 45%, between 15% and 45%, between 25% and 45%, between 30% and 45%, between 35% and 45%, between 40% and 45%, between 15% and 35%, or between 20% and 30%; e.g., about 3%, 5%, 10%, 15%, 20%, 25%, 26%, 30%, 35%, 40% or 45%). In some embodiments, the improved response in ORR is an increase in ORR compared to the reference ORR of about 25.6%.

In some embodiments, the efficacy response is a DOR, and wherein the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the efficacy response in median DOR is non-inferior compared to the reference median DOR. In some embodiments, the efficacy response in the rate of a DOR of 3 months is non-inferior compared to the reference rate of a DOR of 3 months. In some embodiments, the efficacy response in the rate of a DOR of 6 months is non-inferior compared to the reference rate of a DOR of 6 months. In some embodiments, the efficacy response in the rate of a DOR of 9 months is non-inferior compared to the reference rate of a DOR of 9 months.

In some embodiments, the efficacy response is a DOCR, and wherein the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the efficacy response in median DOCR is non-inferior compared to the reference median DOCR. In some embodiments, the efficacy response in the rate of a DOCR of 3 months is non-inferior compared to the reference rate of a DOCR of 3 months. In some embodiments, the efficacy response in the rate of a DOCR of 6 months is non-inferior compared to the reference rate of a DOCR of 6 months. In some embodiments, the efficacy response in the rate of a DOCR of 9 months is non-inferior compared to the reference rate of a DOCR of 9 months.

In some embodiments, the CR or PR determined by PET/computed tomography (CT). In some embodiments, the CR or PR is determined based on the Lugano Response Criteria for Malignant Lymphoma (Cheson et al., 2014).

In one aspect, the invention provides a method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

In some embodiments, the safety response is the rate of AE, and wherein the rate of AE or the reference rate of AE is the rate of AE in the plurality of subjects receiving the corresponding treatment. In some embodiments, the safety response in rate of AE is non-inferior compared to the reference rate of AE. In some embodiments, the safety response in rate of Grade 3-5 AE is non-inferior compared to the reference rate of Grade 3-5 AE. In some embodiments, the Grade of AE is determined based on the National Cancer Institute Common Terminology Criteria for Adverse Events (NCI CTCAE), v5.0.

In some embodiments, the safety response is the rate of SAE, and wherein the rate of SAE or the reference rate of SAE is the rate of SAE in the plurality of subjects receiving the corresponding treatment. In some embodiments, the safety response in rate of SAE is non-inferior compared to the reference rate of SAE.

In some embodiments, the combination treatment comprises subcutaneously administering mosunetuzumab and intravenously administered polatuzumab vedotin according to a dosing regimen comprising at least a first 21-day dosing cycle and a second 21-day dosing cycle, wherein: (a) the first dosing cycle comprises: (i) a first subcutaneous dose (scC1D1), a second subcutaneous dose (scC1D2), and a third subcutaneous dose (scC1D3) of mosunetuzumab administered on Days 1, 8 (±1 day), and 15 (±1 day), respectively, of the first dosing cycle, wherein the scC1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or +3 mg; e.g., 5 mg), the scC1D2 is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or +3 mg; e.g., 45 mg), and the scC1D3 is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and (ii) an intravenous dose of polatuzumab vedotin administered on Day 1 of the first dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and (ii) an intravenous dose of polatuzumab vedotin administered on Day 1 of the second dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg).

In some embodiments, the dosing regimen of the combination treatment further comprises one or more additional (e.g., 1, 2, 3, 4, 5, 6, 7, 8, 9, 10, 11, 12, or more) 21-day dosing cycles. In some embodiments, the dosing regimen of the combination treatment comprises six additional 21-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and an intravenous dose of polatuzumab vedotin administered on Day 1 of the additional dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); or (b) a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg). In some embodiments, (a) the first four additional dosing cycles each comprises a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and an intravenous dose of polatuzumab vedotin administered on Day 1 of the additional dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); and (b) the next two additional dosing cycles each comprises a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg).

In some embodiments, the control treatment comprises intravenously administering 375 mg/m² (e.g., 375 mg/m²±1 mg/m², ±2.5 mg/m², ±5 mg/m², ±7.5 mg/m², ±10 mg/m², ±20 mg/m², ±30 mg/m², +40 mg/m², or ±50 mg/m²; e.g., 375 mg/m²) rituximab, 1000 mg/m² (e.g., 1000 mg/m²±1 mg/m², ±2.5 mg/m², ±5 mg/m², ±7.5 mg/m², ±10 mg/m², ±20 mg/m², ±30 mg/m², ±40 mg/m², ±50 mg/m²; +100 mg/m²; +150 mg/m²; +200 mg/m²; +250 mg/m²; +300 mg/m²; e.g., 1000 mg/m²) gemcitabine, and 100 mg/m² (e.g., 100 mg/m²±1 mg/m², ±2.5 mg/m², ±5 mg/m², ±7.5 mg/m², ±10 mg/m², ±20 mg/m², or ±30 mg/m²; e.g., 100 mg/m²) oxaliplatin for eight 14-day dosing cycles, wherein rituximab, gemcitabine, and oxaliplatin are administered on Day 1 of each dosing cycle.

In one aspect, the invention features a method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

In some embodiments, the efficacy response is a CRR, and wherein the CRR or reference CRR is the proportion of the subjects receiving the corresponding treatment whose best overall response is a complete response (CR). In some embodiments, the improved response in CRR is an increase in CRR compared to the reference CRR of between 1% and 52% (e.g., between 1% and 40%, between 1% and 30%, between 1% and 20%, between 1% and 10%, between 10% and 40%, between 10% and 30%, between 10% and 20%, between 15% and 35%, between 15% and 25%, between 1% and 25%, between 25% and 50%, between 30% and 50%, between 20% and 50%, between 30% and 40%, between 20% and 40%, between 20% and 30%, or between 20% and 25%; e.g., about 5%, 10%, 15%, 20%, 21%, 22%, 23%, 24%, 25%, 30%, 35%, 40%, 45%, 50%, or 52%). In some embodiments, the improved response in CRR is an increase in CRR compared to the reference CRR of about 23%.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 1% and 55% (e.g., between 1% and 50%, between 1% and 40%, between 1% and 30%, between 1% and 20%, between 1% and 10%, between 10% and 40%, between 10% and 30%, between 10% and 20%, between 15% and 35%, between 15% and 25%, between 1% and 25%, between 25% and 50%, between 35% and 55%, between 25% and 35%, between 30% and 50%, between 20% and 50%, between 30% and 40%, between 20% and 40%, between 20% and 30%, between 20% and 25%, or between 25% and 30%; e.g., about 5%, 10%, 15%, 20%, 25%, 26%, 27%, 28%, 29%, 30%, 35%, 40%, 45%, 50%, or 55%). In some embodiments, the improved response in ORR is an increase in ORR compared to the reference ORR of about 28%.

In some embodiments, the efficacy response is a DOR, and wherein the DOR or the reference DOR is measured starting from the time from the first occurrence of a documented CR or PR to disease progression or relapse or death from any cause, whichever occurs first.

In some embodiments, the DOR or the reference DOR is the median DOR of the plurality of subjects receiving the corresponding treatment. In some embodiments, the improvement of the median DOR is an increase in the DOR compared to the reference DOR of between 1 and 21 months (e.g., between 1 month and 5 months, between 5 months and 21 months, between 10 months and 21 months, between 15 months and 21 months, between 5 months and 10 months, between 5 months and 15 months, between 10 months and 21 months, between 10 months and 15 months, or between 15 months and 21 months; e.g., about 1 months, 5 months, 10 months, 15 months, 20 months, or 21 months). In some embodiments, the improvement of the DOR is an increase in the rate of a DOR of 6 months compared to the rate of a reference DOR of 6 months of between 1% and 61% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 18%, 19%, 20%, 21%, 22%, 25%, 30%, 35%, 40%, 45%, 50%, 55%, 60%, or 61%). In some embodiments, the improvement of the DOR is an increase in the rate of a DOR of 6 months compared to the rate of a reference DOR of 6 months of about 20%. In some embodiments, the improvement of the DOR is an increase in the rate of a DOR of 9 months compared to the rate of a reference DOR of 9 months of between 1% and 72% (e.g., between 1% and 70%, between 1% and 60%, between 1% and 50%, between 1% and 40%, between 1% and 30%, between 1% and 20%, between 1% and 10%, between 10% and 70%, between 10% and 60%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 35% and 70%, between 55% and 70%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 30%, 35%, 40%, 45%, 50%, 55%, 60%, 65%, 70%, or 72%). In some embodiments, the improvement of the DOR is an increase in the rate of a DOR of 9 months compared to the rate of a reference DOR of 9 months of about 26%. In some embodiments, the improvement of the DOR is an increase in the rate of a DOR of 12 months compared to the rate of a reference DOR of 12 months of between 1% and 87% (e.g., between 1% and 85%, between 1% and 80%, between 1% and 70%, between 1% and 60%, between 1% and 50%, between 1% and 40%, between 1% and 30%, between 1% and 20%, between 1% and 10%, between 10% and 80%, between 10% and 60%, between between 10% and 40%, between 10% and 30%, between 10% and 20%, between 15% and 35%, between 15% and 25%, between 1% and 25%, between 25% and 50%, between 35% and 55%, between 30% and 80%, between 35% and 70%, between 55% and 70%, between 25% and 35%, between 20% and 50%, between 40% and 60%, between 20% and 60%, between 30% and 40%, between 20% and 40%, between 60% and 80%, between 10% and 50%, between 20% and 50%, between 30% and 50%, or between 35% and 45%; e.g., about 5%, 10%, 15%, 20%, 25%, 26%, 27%, 28%, 30%, 35%, 40%, 45%, 50%, 55%, 60%, 65%, 70%, 75%, 80%, 85%, or 87%). In some embodiments, the improvement of the DOR is an increase in the rate of a DOR of 12 months compared to the rate of a reference DOR of 12 months of about 39%. In some embodiments, the improvement of the DOR is an increase in the rate of a DOR of 18 months compared to the rate of a reference DOR of 18 months of between 43% and 92% (e.g., between 43% and 90%, between 43% and 80%, between 43% and 70%, between 43% and 60%, between 43% and 50%, between 50% and 80%, between 50% and 70%, between 50% and 60%, between 60% and 70%, between 60% and 80%, between 60% and 75%, between 55% and 65%, between 50% and 90%, between 80%, and 90%, between 75% and 90%, or between 65% and 70%; e.g., about 43%, 45%, 50%, 55%, 60%, 65%, 66%, 67%, 68%, 69%, 70%, 75%, 80%, 85%, 90%, or 92%). In some embodiments, the improvement of the DOR is an increase in the rate of a DOR of 18 months compared to the rate of a reference DOR of 18 months of about 67%.

In some embodiments, the CR or PR determined by PET/computed tomography (CT). In some embodiments, the CR or PR is determined based on the Lugano Response Criteria for Malignant Lymphoma (Cheson et al., 2014).

In some embodiments, the efficacy response is a PFS, and wherein the PFS or the reference PFS is measured starting from the time of receiving first dose of mosunetuzumab or polatuzumab vedotin to the time of a first occurrence of disease progression or death from any cause. In some embodiments, the PFS or the reference PFS is the median PFS of the plurality of subjects receiving the corresponding treatment. In some embodiments, the improvement of the median PFS is an increase in the PFS compared to the reference PFS of between 1 and 24 months. In some embodiments, the improvement of the PFS is an increase in the rate of a PFS of 6 months compared to the rate of a reference PFS of 6 months of between 1% and 53% (e.g., between 1% and 50%, between 1% and 40%, between 1% and 30%, between 1% and 20%, between 1% and 10%, between 10% and 40%, between 10% and 30%, between 10% and 20%, between 15% and 35%, between 15% and 25%, between 1% and 25%, between 25% and 50%, between 35% and 53%, between 25% and 35%, between 30% and 50%, between 20% and 50%, between 30% and 40%, between 20% and 40%, between 20% and 30%, between 20% and 25%, or between 25% and 30%; e.g., about 5%, 10%, 15%, 20%, 25%, 26%, 27%, 28%, 29%, 30%, 35%, 40%, 45%, 50%, or 53%). In some embodiments, the improvement of the PFS is an increase in the rate of a PFS of 6 months compared to the rate of a reference PFS of 6 months of about 21%. In some embodiments, the improvement of the PFS is an increase in the rate of a PFS of 9 months compared to the rate of a reference PFS of 9 months of between 1% and 62% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 29%, 30%, 35%, 40%, 45%, 50%, 55%, 60%, or 62%). In some embodiments, the improvement of the PFS is an increase in the rate of a PFS of 9 months compared to the rate of a reference PFS of 9 months of about 28%. In some embodiments, the improvement of the PFS is an increase in the rate of a PFS of 12 months compared to the rate of a reference PFS of 12 months of between 1% and 63% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 29%, 30%, 35%, 40%, 45%, 50%, 55%, 60%, or 63%). In some embodiments, the improvement of the PFS is an increase in the rate of a PFS of 12 months compared to the rate of a reference PFS of 12 months of about 27%. In some embodiments, the improvement of the PFS is an increase in the rate of a PFS of 18 months compared to the rate of a reference PFS of 18 months of between 1% and 68% (e.g., between 1% and 55%, between 1% and 50%, between 1% and 40%, between 1% and 30%, between 1% and 20%, between 1% and 10%, between 10% and 50%, between 10% and 40%, between 10% and 30%, between 10% and 20%, between 15% and 35%, between 15% and 25%, between 1% and 25%, between 25% and 50%, between 35% and 55%, between 30% and 60%, between 20% and 65%, between 40% and 65%, between 25% and 35%, between 30% and 50%, between 20% and 50%, between 40% and 60%, between 20% and 60%, between 30% and 40%, between 20% and 40%, between 20% and 30%, between 20% and 25%, or between 15% and 20%; e.g., about 5%, 10%, 15%, 20%, 25%, 26%, 27%, 28%, 29%, 30%, 35%, 40%, 45%, 50%, 55%, 60%, 65%, or 68%). In some embodiments, the improvement of the PFS is an increase in the rate of a PFS of 18 months compared to the rate of a reference PFS of 18 months of about 25%.

In one aspect, the invention features a method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

In some embodiments, (a) the safety response is the rate of AE, and wherein the rate of AE or the reference rate of AE is the rate of AE in the plurality of subjects receiving the corresponding treatment; or (b) the safety response is the rate of SAE, and wherein the rate of SAE or the reference rate of SAE is the rate of SAE in the plurality of subjects receiving the corresponding treatment. In some embodiments, (a) the safety response in rate of AE is non-inferior compared to the reference rate of AE; or (b) the safety response in rate of SAE is non-inferior compared to the reference rate of SAE.

In some embodiments, the control treatment comprises (a) intravenously administering 375 mg/m² (e.g., 375 mg/m²±1 mg/m², ±2.5 mg/m², ±5 mg/m², ±7.5 mg/m², ±10 mg/m², ±20 mg/m², ±30 mg/m², ±40 mg/m², or ±50 mg/m²; e.g., 375 mg/m²) rituximab and 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg) polatuzumab vedotin for six 21-day dosing cycles, wherein rituximab and polatuzumab vedotin are administered on Day 1 of each dosing cycle; and (b) intravenously administering 375 mg/m² (e.g., 375 mg/m²±1 mg/m², ±2.5 mg/m², ±5 mg/m², ±7.5 mg/m², ±10 mg/m², ±20 mg/m², ±30 mg/m², ±40 mg/m², or ±50 mg/m²; e.g., 375 mg/m²) rituximab for two 21-day dosing cycles, wherein rituximab is administered on Day 1 of each dosing cycle.

In one aspect, the invention features a method of achieving an objective response (OR), a complete response (CR), an overall survival (OS), or a progression free survival (PFS) in a subject having a relapsed and/or refractory non-Hodgkin's lymphoma (R/R NHL), wherein the subject is administered a combination treatment comprising subcutaneously administered mosunetuzumab and intravenously administered polatuzumab vedotin.

In some embodiments, the subject achieves an OR. In some embodiments, the OR is maintained for at least 6, 9, 12, or 18 months.

In some embodiments, the subject achieves a CR.

In some embodiments, the OS is maintained for at least 6, 9, 12, or 18 months.

In some embodiments, the PFS is maintained for at least 6, 9, 12, or 18 months.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall response rate of between 62% and 89% (e.g., between 65% and 85%, between 65% and 75%, or between 75% and 85%; e.g., about 62%, 65%, 70%, 75%, 80%, 85%, or 89%).

In some embodiments, the overall response rate is about 78%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a complete response rate of between 41% and 73% (e.g., between 45% and 70%, between 55% and 70%, between 65% and 70%, between 45% and 55%, between 45% and 65%, or between 55% and 65%; e.g., about 41%, 45%, 50%, 55%, 56%, 57%, 58%, 59%, 60%, 65%, 70%, or 73%).

In some embodiments, the complete response rate is about 58%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall survival rate at 9 months of between 66% and 92% (e.g., between 65% and 90%, between 65% and 80%, between 65% and 75%, between 75% and 85% between 75% and 90%, or between 70% and 85%; e.g., about 62%, 65%, 70%, 75%, 80%, 85%, 90%, or 92%).

In some embodiments, the overall survival rate at 9 months is about 79%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall survival rate at 12 months of between 60% and 88% (e.g., between 65% and 85%, between 65% and 80%, between 65% and 75%, between 75% and 85%, between 75% and 88%, or between 70% and 85%; e.g., about 62%, 65%, 70%, 73%, 74%, 75%, 80%, 85%, or 88%).

In some embodiments, the overall survival rate at 12 months is about 74%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a progression free survival rate at 9 months of between 57% and 87% (e.g., between 60% and 85%, between 65% and 85%, between 65% and 80%, between 60% and 75%, between 75% and 85%, between 75% and 87%, or between 70% and 85%; e.g., about 57%, 60%, 65%, 70%, 71%, 72%, 73%, 74%, 75%, 80%, 85%, or 87%).

In some embodiments, the progression free survival rate at 9 months is about 72%.

In one aspect, the invention features a method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a progression free survival rate at 12 months of between 47% and 81% (e.g., between 50% and 80%, between 50% and 70%, between 55% and 70%, between 65% and 70%, between 50% and 75%, between 60% and 80%, or between 60% and 65%; e.g., about 47%, 50%, 55%, 60%, 63%, 64%, 65%, 70%, 75%, 80%, or 81%).

In some embodiments, the progression free survival rate at 12 months is about 64%.

In some embodiments, the combination treatment comprises subcutaneously administering mosunetuzumab and intravenously administered polatuzumab vedotin according to a dosing regimen comprising at least a first 21-day dosing cycle and a second 21-day dosing cycle, wherein: (a) the first dosing cycle comprises: (i) a first subcutaneous dose (scC1D1), a second subcutaneous dose (scC1D2), and a third subcutaneous dose (scC1D3) of mosunetuzumab administered on Days 1, 8 (±1 day), and 15 (±1 day), respectively, of the first dosing cycle, wherein the scC1D1 is about 5 mg (e.g., 5 mg±0.01 mg,+0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 5 mg), the scC1D2 is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg), and the scC1D3 is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and (ii) an intravenous dose of polatuzumab vedotin administered on Day 1 of the first dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and (ii) an intravenous dose of polatuzumab vedotin administered on Day 1 of the second dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg).

In some embodiments, the dosing regimen of the combination treatment further comprises one or more (e.g., 1, 2, 3, 4, 5, 6, 7, 8, 9, 10, 11, 12, or more) additional 21-day dosing cycles. In some embodiments, the dosing regimen of the combination treatment comprises six additional 21-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and an intravenous dose of polatuzumab vedotin administered on Day 1 of the additional dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); or (b) a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg).

In some embodiments, (a) the first four additional dosing cycles each comprises a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg); and an intravenous dose of polatuzumab vedotin administered on Day 1 of the additional dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); and (b) the next two additional dosing cycles each comprises a subcutaneous dose of mosunetuzumab administered on Day 1 of the additional dosing cycle, wherein the subcutaneous dose of mosunetuzumab is about 45 mg (e.g., 45 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 45 mg).

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median overall survival of between 15 and 39 months (e.g., between 15 months and 35 months, between 15 months and 30 months, between 15 months and 25 months, between 15 months and 20 months, between 20 months and 35 months, between 20 months and 30 months, between 20 months and 25 months, between 25 months and 39 months, or between 25 months and 30 months; e.g., about 15, 20, 25, 26, 27, 28, 29, 30, 35, or 39 months). In some embodiments, the median overall survival is about 27 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 6 months of between 73% and 89% (e.g., between 75% and 89%, between 75% and 85%, between 75% and 80%, between 80% and 85%, or between 80% and 89%; e.g., about 73%, 75%, 80%, 81%, 82%, 85%, or 89%). In some embodiments, the overall survival rate at 6 months is about 81%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 9 months of between 61% and 80% (e.g., between 61% and 80%, between 65% and 80%, between 60% and 75%, between 75% and 80%, between 61% and 70%, between 70% and 80%, or between 65% and 75%; e.g., about 61%, 65%, 68%, 69%, 70%, 71%, 72%, 75%, or 80%). In some embodiments, the overall survival rate at 9 months is about 70%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 12 months of between 55% and 75% (e.g., between 55% and 70%, between 55% and 65%, between 55% and 60%, between 60% and 75%, between 65% and 75%, between 70% and 75%, or between 60% and 70%; e.g., about 55%, 60%, 65%, 70%, or 75%). In some embodiments, the overall survival rate at 12 months is about 65%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 18 months of between 45% and 65% (e.g., between 45% and 65%, between 45% and 60%, between 45% and 55%, between 45% and 50%, between 60% and 65%, between 50% and 65%, between 55% and 65%, or between 50% and 60%; e.g., about 45%, 50%, 55%, 60%, or 65%). In some embodiments, the overall survival rate at 18 months is about 55%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 24 months of between 40% and 61% (e.g., between 40% and 60%, between 40% and 55%, between 40% and 50%, between 40% and 45%, between 55% and 60%, between 50% and 60%, or between 45% and 55%; e.g., about 40%, 45%, 50%, 55%, 60%, or 61%). In some embodiments, the overall survival rate at 24 months is about 50%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median progression free survival of between 9 and 27 months (e.g., between 9 months and 25 months, between 15 months and 25 months, between 10 months and 25 months, between 15 months and 20 months, or between 10 months and 20 months; e.g., about 9, 10, 13, 14, 15, 20, 25, or 27 months). In some embodiments, the median progression free survival is about 14 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 6 months of between 51% and 72% (e.g., between 55% and 70%, between 55% and 65%, between 55% and 60%, between 60% and 72%, between 65% and 72%, between 70% and 72%, or between 60% and 70%; e.g., about 51%, 55%, 60%, 61%, 62%, 63%, 64%, 65%, 70%, or 72%). In some embodiments, the progression free survival rate at 6 months is about 62%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 9 months of between 50% and 71% (e.g., between 50% and 70%, between 50% and 65%, between 50% and 60%, between 50% and 55%, between 60% and 71%, between 65% and 71%, between 55% and 70%, or between 55% and 65%; e.g., about 50%, 55%, 60%, 61%, 62%, 63%, 64%, 65%, 70%, or 71%). In some embodiments, the progression free survival rate at 9 months is about 60%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 12 months of between 41% and 64% (e.g., between 45% and 64%, between 45% and 60%, between 45% and 55%, between 45% and 50%, between 60% and 64%, between 50% and 64%, between 55% and 64%, or between 50% and 60%; e.g., about 41%, 45%, 50%, 55%, 60%, or 64%). In some embodiments, the progression free survival rate at 12 months is about 52%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 18 months of between 34% and 57% (e.g., between 35% and 55%, between 35% and 50%, between 35% and 45%, between 35% and 40%, between 40% and 55%, between 40% and 50%, between 40% and 45%, or between 45% and 55%; e.g., about 34%, 35%, 40%, 45%, 46%, 47%, 48%, 50%, 55%, or 57%). In some embodiments, the progression free survival rate at 18 months is about 46%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 24 months of between 28% and 52% (e.g., between 30% and 50%, between 30% and 45%, between 30% and 40%, between 30% and 35%, between 35% and 50%, between 40% and 50%, between 40% and 45%, or between 35% and 45%; e.g., about 28%, 30%, 35%, 40%, 45%, 50%, or 52%). In some embodiments, the progression free survival rate at 24 months is about 40%.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median duration of response of between 16 and 39 months (e.g., between 16 months and 35 months, between 16 months and 25 months, between 16 months and 20 months, between 20 months and 35 months, or between 25 months and 30 months; e.g., about 16, 20, 25, 26, 27, 28, 29, 30, 35, or 39 months).

In some embodiments, the median duration of response is about 28 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 78% and 96% (e.g., between 80% and 95%, between 80% and 90%, between 80% and 85%, between 85% and 90%, or between 85% and 95%; e.g., about 78%, 80%, 85%, 86%, 87%, 88%, 89%, 90%, 95%, or 96%) of the population of subjects maintains a durable response for 6 months. In some embodiments, about 87% of the population of subjects maintains a durable response for 6 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 63% and 88% (e.g., between 65% and 85%, between 65% and 80%, between 65% and 75%, between 65% and 70%, between 70% and 75%, between 70% and 80%, between 70% and 85%, or between 75% and 85%; e.g., about 63%, 65%, 70%, 75%, 80%, 85%, or 88%) of the population of subjects maintains a durable response for 9 months. In some embodiments, about 75% of the population of subjects maintains a durable response for 9 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 58% and 84% (e.g., between 60% and 84%, between 65% and 84%, between 65% and 80%, between 60% and 75%, between 65% and 70%, between 65% and 75%, between 70% and 75%, between 70% and 80%, between 70% and 84%, or between 75% and 84%; e.g., about 58%, 60%, 65%, 70%, 71%, 72%, 73%, 75%, 80%, or 84%) of the population of subjects maintains a durable response for 12 months. In some embodiments, about 71% of the population of subjects maintains a durable response for 12 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 46% and 75% (e.g., between 46% and 75%, between 46% and 70%, between 46% and 60%, between 46% and 50%, between 50% and 75%, between 50% and 70%, between 60% and 70%, between 55% and 65%, between 50% and 60%, or between 55% and 75%; e.g., about 46%, 50%, 55%, 60%, 61%, 62%, 63%, 65%, 70%, or 75%) of the population of subjects maintains a durable response for 18 months. In some embodiments, about 61% of the population of subjects maintains a durable response for 18 months.

In one aspect, the invention features a method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 43% and 73% (e.g., between 45% and 73%, between 45% and 70%, between 45% and 60%, between 45% and 50%, between 50% and 73%, between 55% and 70%, between 50% and 70%, between 60% and 70%, between 55% and 65%, between 50% and 60%, or between 55% and 60%; e.g., about 43%, 45%, 50%, 55%, 56%, 57%, 58%, 59%, 60%, 65%, 70%, or 73%) of the population of subjects maintains a durable response for 24 months. In some embodiments, about 58% of the population of subjects maintains a durable response for 24 months.

In one aspect, the invention features a method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved patient-reported outcome of the plurality of subjects as compared to a reference patient-reported outcome, wherein the reference patient-reported outcome is the patient-reported outcome of a plurality of subjects who have received a control treatment comprising intravenously administered rituximab, gemcitabine, and oxaliplatin (R-GemOx), and wherein the patient-reported outcome is a quality of life factor, a lymphoma factor, or a peripheral neuropathy factor.

In some embodiments, the patient-reported outcome is the mean of the patient-reported outcome of the plurality of subjects. In some embodiments, the reference patient-reported outcome is the mean of the reference patient-reported outcome of the plurality of reference patients.

In some embodiments, the quality of life factor is evaluated according to a European Organization for Research and Treatment of Cancer Quality of Life Questionnaire Core 30 (EORTC QLQ-C30) for global health status, functioning, and core symptom scales (see Aaronson et al. J. Natl Cancer Inst. 1993, 85 (5): 365-376; Fitzsimmons et al., Eur. J. Cancer. 1999, 35 (6): 939-941. In some embodiments, the quality of life factor includes appetite loss, cognitive functioning, constipation, diarrhea, dyspnea, emotional functioning, fatigue, financial difficulties, nausea and vomiting, pain, physical functioning, global health status, role functioning, social functioning, and insomnia.

In some embodiments, the lymphoma factor is evaluated according to a Functional Assessment of Cancer Therapy-Lymphoma subscale (FACT Lym LymS; see Hlubocky et al., Leuk Lymphoma. 2013, 54 (9): 1942-1946)). In some embodiments, the lymphoma factor includes fever, night sweats, weight loss, and lymphoma subscale.

In some embodiments, the peripheral neuropathy factor is evaluated according to a Functional Assessment of Cancer Therapy-Gynecologic Oncology Group-Neurotoxicity (FACT/GOG-Ntx; Huang et al., Int. J. Gynecol. Cancer. 2007, 17 (2): 387-393). In some embodiments, the peripheral neuropathy factor includes neurotoxicity subscale.

In some embodiments, the patient-reported outcome is calculated as a change from baseline by subtracting the patient-reported outcome prior to administration of the combination treatment from the patient-reported outcome after administration of the combination treatment. In some embodiments, the patient-reported outcome is calculated as a change from baseline by subtracting the patient-reported outcome prior to administration of the combination treatment from the patient-reported outcome after administration of one or more (e.g., 1, 2, 3, 4, 5, 6, 7, 8, or more) dosing cycles of a dosing regimen described herein for the combination treatment. In a non-limiting example, a change from baseline of a subject's peripheral neuropathy factor after administration of seven dosing cycles of the combination treatment can be calculated by subtracting the subject's peripheral neuropathy factor prior to administration of the combination treatment from the subject's peripheral neuropathy factor after administration of seven dosing cycles of the combination treatment.

In some embodiments, the reference patient-reported outcome is calculated as a change from baseline by subtracting the reference patient-reported outcome prior to administration of the control treatment from the reference patient-reported outcome after administration of the control treatment. In some embodiments, the reference patient-reported outcome is calculated as a change from baseline by subtracting the reference patient-reported outcome prior to administration of the control treatment from the reference patient-reported outcome after administration of one or more (e.g., 1, 2, 3, 4, 5, 6, 7, 8, or more) dosing cycles of a dosing regimen described herein for the control treatment. In a non-limiting example, a change from baseline of a subject's peripheral neuropathy factor after administration of seven dosing cycles of the control treatment can be calculated by subtracting the subject's peripheral neuropathy factor prior to administration of the control treatment from the subject's peripheral neuropathy factor after administration of seven dosing cycles of the control treatment.

In some embodiments, an improved patient-reported outcome is a larger increase (e.g., a larger flat increase or percentage increase) in the change from baseline of the patient-reported outcome as compared to the change from baselines of the reference patient-reported outcome. In a non-limiting example, an improved patient-reported outcome may be a larger increase for a factor which is positively correlated or associated with a patient or subject's health and well-being; or ability to live or function independently, e.g., an increase in a patient's emotional functioning or physical functioning.

In some embodiments, an improved patient-reported outcome is a larger decrease (e.g., a larger flat decrease or percentage decrease) in the change from baseline of the patient-reported outcome as compared to the change from baseline of the reference patient-reported outcome. In a non-limiting example, an improved patient-reported outcome may be a larger decrease for a factor which is negatively correlated or associated with a patient or subject's health and well-being; or ability to live or function independently, e.g., a decrease in constipation or night sweats.

In some embodiments, an improved patient-reported outcome is an earlier (e.g., 1, 2, 3, 4, 5, 6, 7, or more dosing cycles earlier) increase in the change from baseline of the patient-reported outcome as compared to the change from baselines of the reference patient-reported outcome, e.g., for a factor which is positively correlated or associated with a patient or subject's health and well-being; or ability to live or function independently. In a non-limiting example, a patient or subject may report experiencing an increase in emotional function one or more dosing cycles earlier when administered the combination treatment as compared to a patient or subject who was administered the control treatment.

In some embodiments, an improved patient-reported outcome is a later (e.g., 1, 2, 3, 4, 5, 6, 7, or more dosing cycles earlier) increase in the change from baseline of the patient-reported outcome as compared to the change from baselines of the reference patient-reported outcome, e.g., for a factor which is negatively correlated or associated with a patient or subject's health and well-being; or ability to live or function independently. In a non-limiting example, a patient or subject may report experiencing fever or night sweats one or more dosing cycles later when administered the combination treatment as compared to a patient or subject who was administered the control treatment.

In some embodiments, an improved patient-reported outcome is a later (e.g., 1, 2, 3, 4, 5, 6, 7, or more dosing cycles later) decrease in the change from baseline of the patient-reported outcome as compared to the change from baseline of the reference patient-reported outcome, e.g., for a factor which is positively correlated or associated with a patient or subject's health and well-being; or ability to live or function independently. In a non-limiting example, a patient or subject may report experiencing a decrease in physical functioning one or more dosing cycles later when administered the combination treatment as compared to a patient or subject who was administered the control treatment.

In some embodiments, an improved patient-reported outcome is an earlier (e.g., 1, 2, 3, 4, 5, 6, 7, or more dosing cycles earlier) decrease in the change from baseline of the patient-reported outcome as compared to the change from baselines of the reference patient-reported outcome, e.g., for a factor which is negatively correlated or associated with a patient or subject's health and well-being; or ability to live or function independently. In a non-limiting example, a patient or subject may report experiencing a decrease in pain one or more dosing cycles earlier when administered the combination treatment as compared to a patient or subject who was administered the control treatment.

In some embodiments, the combination treatment comprises intravenously administering mosunetuzumab and intravenously administering polatuzumab vedotin according to a dosing regimen comprising eight 21-day dosing cycle, wherein: (a) the first dosing cycle comprises: (i) a first intravenous dose (ivC1D1), a second intravenous dose (ivC1D2), and a third intravenous dose (ivC1D3) of mosunetuzumab administered on Days 1, 8 (±1 day), and 15 (±1 day), respectively, of the first dosing cycle, wherein the ivC1D1 is about 1 mg (e.g., 1 mg±0.01 mg, ±0.025 mg, ±0.05 mg, ±0.075 mg, ±0.1 mg; e.g., 1 mg), the ivC1D2 is about 2 mg (e.g., 2 mg±0.01 mg, ±0.025 mg, ±0.05 mg, ±0.075 mg, ±0.1 mg, ±0.2 mg; e.g., 2 mg), and the ivC1D3 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, or ±6 mg; e.g., 60 mg); and (ii) an intravenous dose of polatuzumab vedotin administered on Day 1 of the first dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); (b) the second dosing cycle comprises: (i) a first intravenous dose (ivC2D1) of mosunetuzumab administered on Day 1 of the second dosing cycle, wherein the ivC2D1 is about 60 mg (e.g., 60 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, or ±6 mg; e.g., 60 mg); and (ii) an intravenous dose of polatuzumab vedotin administered on Day 1 of the second dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); (c) the third to sixth dosing cycles each comprises: (i) a first intravenous dose (ivC3D1-ivC6D1) of mosunetuzumab administered on Day 1 of each respective dosing cycle, wherein the ivC3D1-ivC6D1 is each about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg); and (ii) an intravenous dose of polatuzumab vedotin administered on Day 1 of the second dosing cycle, wherein the intravenous dose of polatuzumab vedotin is about 1.8 mg/kg (e.g., 1.8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, or ±1 mg/kg; e.g., 1.8 mg/kg); and (d) the seventh and eighth dosing cycles each comprises a first intravenous dose (ivC7D1-ivC8D1) of mosunetuzumab administered on Day 1 of each respective dosing cycle, wherein the ivC7D1-ivC8D1 is each about 30 mg (e.g., 30 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, or ±3 mg; e.g., 30 mg) and does not comprise administration of polatuzumab vedotin.

In some embodiments, the subject has an Eastern Cooperative Oncology Group (ECOG) Performance Status of 0, 1, or 2. In some embodiments, aNHL is a diffuse large B cell lymphoma (DLBCL), a high grade B cell lymphoma (HGBL), a Grade 3b follicular lymphoma (FL), or a transformed FL (trFL). In some embodiments, the R/R NHL is an R/R large B cell lymphoma (LBCL), an R/R diffuse large B cell lymphoma (DLBCL), an R/R follicular lymphoma (FL), or an R/R mantle cell lymphoma (MCL). In some embodiments, the R/R FL is a Grade 1, 2, 3a, or 3b FL, an in situ follicular neoplasia, a duodenal-type FL, or a pediatric-type FL. In some embodiments, the FL has relapsed after prior regimen(s) after having a documented history of response (CR, CR unconfirmed [CRu], or PR) of ≥6 months in duration from completion of regimen(s), and/or is refractory to any prior regimen. In some embodiments, the R/R FL was previously treated with at least one prior systemic treatment regimen containing an anti-CD20-directed therapy (e.g., an anti-CD20 antibody). In some embodiments, the R/R DLBCL is a DLBCL not otherwise specified (NOS) (including germinal center B-cell type and activated B-cell type), a T-cell/histiocyte-rich large B-cell lymphoma, a high-grade B-cell lymphoma, e.g., with MYC and BCL-2 and/or BCL-6 rearrangements, EBV+DLBCL NOS, NOS, HHV8+DLBCL NOS, anaplastic lymphoma kinase (ALK)+large B-cell lymphoma, a transformed FL, or a Grade 3b FL. In some embodiments, the R/R DLBCL is R/R to standard therapies for aggressive NHL, has relapsed after prior regimen(s) after having a documented history of response (CR, CR unconfirmed [CRu], or PR) of ≥6 months in duration from completion of regimen(s), and/or is refractory to any prior regimen. In some embodiments, the R/R DLBCL was previously treated with at least one prior systemic treatment regimen containing an anti-CD20-directed therapy (e.g., an anti-CD20 antibody). In some embodiments, the R/R MCL has relapsed after prior regimen(s) after having a documented history of response (CR, CR unconfirmed [CRu], or PR) of >6 months in duration from completion of regimen(s), and/or is refractory to any prior regimen. In some embodiments, the R/R MCL was previously treated using an anti-CD20-directed therapy, a Bruton's tyrosine kinase (BTK) inhibitor, and an anthracycline or bendamustine.

In some embodiments, the subject has relapsed after or is refractory to one or more prior lines of systemic therapy. In some embodiments, the subject is ineligible for autologous stem cell transplant (ASCT).

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the population of subjects has an overall response rate of between 74% and 100% (e.g., between 75% and 100%, between 75% and 90%, between 75% and 80%, between 80% and 100%, between 90% and 100%, between 80% and 90%, or between 85% and 95%; e.g., about 74%, 75%, 80%, 85%, 86%, 87%, 88%, 89%, 90%, 91%, 93%, 94%, 95%, or 100%). In some embodiments, the overall response rate is about 88%.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the population of subjects has a complete response rate of between 63% and 93% (e.g., between 65% and 93%, between 75% and 93%, between 85% and 93%, between 63% and 80%, between 63% and 70%, between 70% and 80%, between 65% and 75%, or between 75% and 90%; e.g., about 63%, 65%, 68%, 69%, 70%, 71%, 75%, 77%, 78%, 79%, 80%, 81%, 82%, 85%, 90%, 91%, 92%, or 93%). In some embodiments, the complete response rate is about 79%.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the median time to first response in the population of subjects is between 80 days to 100 days (e.g., about 2.5 months, about 3 months, between 80-90 days, between 90-100 days, between 85-95 days, about 80 days, about 90 days, or about 100 days). In some embodiments, the median time to first response in the population of subjects is about 3 months.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the median PFS in the population of subjects is at least 14 months (e.g., at least 18 months, at least 24 months, at least 30 months, at least 36 months, or more; e.g., 14, 15, 16, 17, 18, 19, 20, 21, 22, 23, 24, 25, 26, 27, 28, 29, 30, 31, 32, 33, 34, 35, or 36 months, or more). In some embodiments, the median PFS in the population of subjects is about 19 months.

In one aspect, the invention features a method of treating a population of subjects having a relapsed and/or refractory MCL, wherein each subject of the population is administered a combination treatment comprising subcutaneously administered mosunetuzumab and intravenously administered polatuzumab vedotin, and wherein the median OS in the population of subjects is at least 17 months (e.g., at least 18 months, at least 24 months, at least 30 months, at least 36 months, or more; e.g., 17, 18, 19, 20, 21, 22, 23, 24, 25, 26, 27, 28, 29, 30, 31, 32, 33, 34, 35, or 36 months, or more). In some embodiments, the median OS in the population of subjects is about 21 months.

In some embodiments, the MCL is R/R MCL. In some embodiments, the subjects of the population are R/R to Bruton's tyrosine kinase (BTK) inhibitor therapy. In some embodiments, the subjects of the population are R/R to prior chimeric antigen receptor T-cell (CAR T) therapy. In some embodiments, the subject is R/R to 2 or more (e.g., 2, 3, 4, 5, 6, 7, 8, or more) prior lines of therapy. In some embodiments, the subjects of the population have a high-risk factor comprising a Ki-67 proliferation index ≥50%, a blastoid/pleomorphic variants, or a TP53 mutation.

In one aspect, the invention features a method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a CRR, an ORR, a PFS, or an OS.

In some embodiments, the efficacy response is a CRR, and wherein the CRR or reference CRR is the proportion of the subjects receiving the corresponding treatment whose best overall response is a complete response (CR). In some embodiments, the improved response in CRR is an increase in CRR compared to the reference CRR of between 3% and 44% (e.g., between 5% and 44%, between 5% and 35%, between 5% and 25%, between 5% and 15%, between 5% and 10%, between 10% and 44%, between 15% and 44%, between 25% and 44%, between 35% and 44%, between 15% and 35%, between 25% and 35%, between 15% and 25%; e.g., about 3%, 5%, 10%, 15%, 20%, 30%, 35%, 40%, or 44%). In some embodiments, the improved response in CRR is an increase in CRR compared to the reference CRR of about 25%.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 15% and 46% (e.g., between 15% and 45%, between 15% and 35%, between 15% and 25%, between 25% and 45%, between 35% and 45%, between 15% and 35%, between 25% and 35%, between 15% and 25%; e.g., about 15%, 20%, 30%, 35%, 40%, 45%, or 46%). In some embodiments, the improved response in ORR is an increase in ORR compared to the reference ORR of about 31%.

In some embodiments, the efficacy response is a PFS, and wherein the PFS or the reference PFS is measured starting from the time of receiving first dose of mosunetuzumab or polatuzumab vedotin to the time of a first occurrence of disease progression or death from any cause. In some embodiments, the PFS or the reference PFS is the median PFS of the plurality of subjects receiving the corresponding treatment.

In some embodiments, the improvement of the median PFS is an increase in the PFS compared to the reference PFS of between 1 and 15 months (e.g., 1, 2, 3, 4, 5, 6, 7, 8, 9, 10, 11, 12, 13, 14, or 15 months). In some embodiments, the improvement of the median PFS is an increase in the PFS compared to the reference PFS of 7 months.

In some embodiments, the improvement of the PFS is an increase in the rate of a PFS of 3 months compared to the rate of a reference PFS of 3 months of between 7% and 37% (e.g., between 10% and 35%, 10% and 30%, between 10% and 25%, between 10% and 20%, between 10% and 15%, between 15% and 25%, between 15% and 30%, between 20% and 25%, between 15% and 35%, between 20% and 35%, or between 15% and 20%; e.g., about 7%, 10%, 15%, 20%, 25%, 30%, 35%, or 37%). In some embodiments, the improvement of the PFS is an increase in the rate of a PFS of 3 months compared to the rate of a reference PFS of 3 months of about 22%.

In some embodiments, the improvement of the PFS is an increase in the rate of a PFS of 6 months compared to the rate of a reference PFS of 6 months of between 11% and 44% (e.g., between 15% and 40%, 15% and 35%, 15% and 30%, between 15% and 25%, between 15% and 30%, between 20% and 40%, between 20% and 25%, between 25% and 35%, between 15% and 35%, between 20% and 35%, or between 15% and 20%; e.g., about 11%, 15%, 20%, 25%, 30%, 35%, 40%, or 44%). In some embodiments, the improvement of the PFS is an increase in the rate of a PFS of 6 months compared to the rate of a reference PFS of 6 months of about 27%.

In some embodiments, the improvement of the PFS is an increase in the rate of a PFS of 9 months compared to the rate of a reference PFS of 9 months of between 11% and 45% (e.g., between 15% and 45%, between 15% and 40%, 15% and 35%, 15% and 30%, between 15% and 25%, between 20% and 40%, between 20% and 25%, between 25% and 35%, between 20% and 35%, between 20% and 45%, between 30% and 45%, or between 15% and 20%; e.g., about 11%, 15%, 20%, 25%, 30%, 35%, 40%, or 45%). In some embodiments, the improvement of the PFS is an increase in the rate of a PFS of 9 months compared to the rate of a reference PFS of 9 months of about 28%.

In some embodiments, the improvement of the PFS is an increase in the rate of a PFS of 12 months compared to the rate of a reference PFS of 12 months of between 10% and 44% (e.g., between 10% and 40%, 10% and 35%, 10% and 30%, between 10% and 25%, between 10% and 20%, between 20% and 40%, between 20% and 25%, between 15% and 40%, between 20% and 30%, between 25% and 35%, between 15% and 35%, between 20% and 35%, or between 15% and 20%; e.g., about 10%, 15%, 20%, 25%, 30%, 35%, 40%, or 44%). In some embodiments, the improvement of the PFS is an increase in the rate of a PFS of 12 months compared to the rate of a reference PFS of 12 months of about 27%.

In some embodiments, the improvement of the PFS is an increase in the rate of a PFS of 18 months compared to the rate of a reference PFS of 18 months of between 5% and 39% (e.g., between 5% and 35%, 5% and 30%, between 5% and 5%, between 5% and 20%, between 5% and 15%, between 5% and 10%, between 10% and 20%, between 10% and 30%, between 10% and 15%, between 15% and 25%, between 15% and 30%, between 20% and 25%, between 15% and 35%, between 20% and 35%, or between 15% and 20%; e.g., about 5%, 10%, 15%, 20%, 25%, 30%, 35%, or 39%). In some embodiments, the improvement of the PFS is an increase in the rate of a PFS of 18 months compared to the rate of a reference PFS of 18 months of about 22%.

In some embodiments, the improvement of the PFS is an increase in the rate of a PFS of 24 months compared to the rate of a reference PFS of 24 months of between 5% and 38% (e.g., between 5% and 35%, 5% and 30%, between 5% and 5%, between 5% and 20%, between 5% and 15%, between 5% and 10%, between 10% and 20%, between 10% and 30%, between 10% and 15%, between 15% and 25%, between 15% and 30%, between 20% and 25%, between 15% and 35%, between 20% and 35%, or between 15% and 20%; e.g., about 5%, 10%, 15%, 20%, 25%, 30%, 35%, or 38%). In some embodiments, the improvement of the PFS is an increase in the rate of a PFS of 24 months compared to the rate of a reference PFS of 24 months of about 21%.

In some embodiments, the efficacy response is an OS, and wherein the OS or the reference OS is measured starting from the time of receiving first dose of mosunetuzumab or polatuzumab vedotin to the time of a first occurrence of disease progression or death from any cause. In some embodiments, the OS or the reference OS is the median OS of the plurality of subjects receiving the corresponding treatment.

In some embodiments, the improvement of the median OS is an increase in the OS compared to the reference OS. In some embodiments, the improvement of the median OS is an increase in the OS compared to the reference OS of 7.4 months.

In some embodiments, the improvement of the OS is an increase in the rate of a OS of 6 months compared to the rate of a reference OS of 6 months of between 1% and 26% (e.g., between 1% and 20%, between 1% and 15%, between 1% and 10%, between 1% and 5%, between 5% and 20%, between 10% and 20%, between 15% and 20%, between 5% and 10%, between 5% and 15%, between 15% and 25%, between 10% and 25%, between 5% and 25%, or between 10% and 20%; e.g., about 1%, 5%, 10%, 15%, 20%, 25%, or 26%). In some embodiments, the improvement of the OS is an increase in the rate of a OS of 6 months compared to the rate of a reference OS of 6 months of about 12%.

In some embodiments, the improvement of the OS is an increase in the rate of a OS of 9 months compared to the rate of a reference OS of 9 months of between 1% and 24% (e.g., between 1% and 20%, between 1% and 15%, between 1% and 10%, between 1% and 5%, between 5% and 20%, between 10% and 20%, between 10% and 24%, between 15% and 24%, between 15% and 20%, between 5% and 10%, between 5% and 15%, or between 10% and 20%; e.g., about 1%, 5%, 10%, 15%, 20%, or 24%). In some embodiments, the improvement of the OS is an increase in the rate of a OS of 9 months compared to the rate of a reference OS of 9 months of about 9%.

In some embodiments, the improvement of the OS is an increase in the rate of a OS of 12 months compared to the rate of a reference OS of 12 months of between 1% and 27% (e.g., between 1% and 20%, between 1% and 15%, between 1% and 10%, between 1% and 5%, between 5% and 20%, between 10% and 20%, between 15% and 20%, between 5% and 10%, between 5% and 15%, between 15% and 25%, between 10% and 25%, between 5% and 25%, or between 10% and 20%; e.g., about 1%, 5%, 10%, 15%, 20%, 25%, or 27%). In some embodiments, the improvement of the OS is an increase in the rate of a OS of 12 months compared to the rate of a reference OS of 12 months of about 11%.

In some embodiments, the improvement of the OS is an increase in the rate of a OS of 18 months compared to the rate of a reference OS of 18 months of between 1% and 28% (e.g., between 1% and 20%, between 1% and 15%, between 1% and 10%, between 1% and 5%, between 5% and 20%, between 10% and 20%, between 15% and 20%, between 5% and 10%, between 5% and 15%, between 15% and 25%, between 10% and 25%, between 5% and 25%, or between 10% and 20%; e.g., about 1%, 5%, 10%, 15%, 20%, 25%, or 28%). In some embodiments, the improvement of the OS is an increase in the rate of a OS of 18 months compared to the rate of a reference OS of 18 months of about 11%.

In some embodiments, the improvement of the OS is an increase in the rate of a OS of 24 months compared to the rate of a reference OS of 24 months of between 1% and 29% (e.g., between 1% and 20%, between 1% and 15%, between 1% and 10%, between 1% and 5%, between 5% and 20%, between 10% and 20%, between 15% and 20%, between 5% and 10%, between 5% and 15%, between 15% and 25%, between 10% and 25%, between 5% and 25%, or between 10% and 20%; e.g., about 1%, 5%, 10%, 15%, 20%, 25%, or 29%). In some embodiments, the improvement of the OS is an increase in the rate of a OS of 24 months compared to the rate of a reference OS of 24 months of about 13%.

In some embodiments, the CR or PR determined by PET/computed tomography (CT). In some embodiments, the CR or PR is determined based on the Lugano Response Criteria for Malignant Lymphoma (Cheson et al., 2014).

In one aspect, the invention features a method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a duration of response (DOR) or a duration of complete response (DOCR).

In some embodiments, the efficacy response is a DOR, and the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the efficacy response in median DOR is non-inferior compared to the reference median DOR. In some embodiments, the efficacy response in median DOR is increased compared to the reference median DOR.

In some embodiments, the efficacy response is a DOCR, and the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the efficacy response in median DOCR is non-inferior compared to the reference median DOCR. In some embodiments, the efficacy response in median DOCR is increased compared to the reference median DOCR.

In some embodiments, a subject is more likely to discontinue a reference treatment comprising R-GemOx due to disease progression (PD) than to discontinue the combination treatment comprising subcutaneously administered mosunetuzumab and intravenously administered polatuzumab vedotin due to PD. In some embodiments, the rate of PD is higher in subjects who receive the combination treatment compared to the rate of PD in subjects who receive the reference treatment. In some embodiments, the rate of discontinuation of the reference treatment due to PD in subjects receiving the reference treatment is higher than the rate of discontinuation of the combination treatment due to PD in subjects receiving the combination treatment.

In some embodiments, a subject is administered a new anti-lymphoma treatment (NALT) after discontinuing treatment with a combination treatment (e.g., comprising subcutaneously administered mosunetuzumab and intravenously administered polatuzumab vedotin) or a reference treatment (e.g., comprising R-GemOx). In some embodiments, more subjects are administered a NALT after discontinuation of a reference treatment than subjects who are administered a NALT after discontinuation of a combination treatment. In some embodiments, the NALT comprises a radiotherapy, a stem cell transplant (e.g., an autologous or allogenic stem cell transplant), a chimeric antigen receptor T cell (CAR-T) therapy, a polatuzumab vedotin-based therapy, or a bispecific antibody. In some embodiments, the NALT comprises CAR-T therapy or a bispecific antibody. In some embodiments, a subject is administered a NALT comprising a CAR-T therapy after discontinuation of a combination treatment. In some embodiments, a subject is administered a NALT comprising a bispecific antibody after discontinuation of a reference treatment.

In one aspect, the invention features a method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered R-GemOx, and wherein the safety response is the rate of AE or the rate of SAE.

In some embodiments, the safety response is the rate of AE, and wherein the rate of AE or the reference rate of AE is the rate of AE in the plurality of subjects receiving the corresponding treatment.

In some embodiments, the safety response is the rate of SAE, and wherein the rate of SAE or the reference rate of SAE is the rate of SAE in the plurality of subjects receiving the corresponding treatment. In some embodiments, the safety response in rate of SAE is non-inferior compared to the reference rate of SAE.

In some embodiments, a subject is administered the combination treatment comprising subcutaneously administered mosunetuzumab and intravenously administered polatuzumab vedotin with a dose intensity >90%. In some embodiments, subjects are administered the combination treatment with higher completion rate (e.g., completes all 8 dosing cycles of mosunetuzumab and all 6 dosing cycles of polatuzumab vedotin) than subjects administered a reference treatment comprising intravenously administered R-GemOx (e.g., does not complete all 8 dosing cycles of R-GemOx).

In some embodiments, the subject with R/R LBCL: has a positive baseline CD20 expression, has a negative baseline CD20 expression, has a baseline ECOG performance status of 0, has a baseline ECOG performance status of 0 or 1, has a baseline ECOG performance status of 1 or higher, has a baseline ECOG performance status of 2 or higher, has received 1 prior line of therapy, has received 1 or 2 prior lines of therapy, has received 2 or more prior lines of therapy, has received 3 or more prior lines of therapy, has relapsed after first prior therapy, is refractory to first prior therapy, has relapsed after the last prior therapy, is refractory to the last prior therapy, has received prior CAR-T therapy, has not received prior CAR-T therapy, has relapsed after prior CAR-T therapy, is refractory to prior CAR-T therapy, has relapsed after prior anti-CD20 antibody therapy, is refractory to prior anti-CD20 antibody therapy, has received prior anti-CD20 antibody therapy within the last 3 months, has received prior anti-CD20 antibody therapy more than 3 months ago, has 0-1 IPI factors, has 2 IPI factors, has 3 IPI factors, has 4-5 IPI factors, has received prior autologous stem cell transplant (ASCT), has not received prior ASCT, has relapsed early from prior ASCT, has relapsed after or is refractory to first line therapy, has relapsed after or is refractory to first line therapy within 12 months of receiving first line therapy, has bulky disease >7.5 cm, does not have bulky disease >7.5 cm, has bulky disease >10 cm, does not have bulky disease >10 cm, has DLBCL, has Grade 3B FL, has HGBL, has transformed follicular lymphoma, does not have transformed follicular lymphoma, has baseline LDH≤1× ULN, has baseline LDH >1× ULN, is Ann Arbor Stage I, is Ann Arbor Stage II, is Ann Arbor Stage III, is Ann Arbor Stage IV, is Ann Arbor Stage I-II, is Ann Arbor Stage III-IV, has LBCL with cell of origin (COO) category of germinal center B-cell-like (GCB), has LBCL with COO category of activated B-cell-like (ABC), has LBCL with COO category of unclassified, has LBCL with COO category of non-GCB, has double hit HGBL with rearrangements in MYC and BCL2 genes, has triple hit HGBL with rearrangements in MYC, BCL2, and BCL6 genes, has double hit or triple hit HGBL, has activities of daily living (ADL) score of $4, has ADL score of >4, has instrumental activities of daily living (IADL) score of ≤6, has IADL score of >6, has Cumulative Illness Rating Scale-Geriatric (CIRS-G) score <7, has CIRS-G score IRS-G score >7, has worst CIRS-G Score of 0, has worst CIRS-G Score of 1-2, has worst CIRS-G Score of 3-4, has mini nutritional assessment-short form (MNA-SF) score of 0-7, has MNA-SF score of 8-11, or has MNA-SF score of 12-14.

In some embodiments, the subject with R/R LBCL: is male, is female, is less than 65 years of age, is 65 years of age or older, is less than 75 years of age, is 75 years of age or older, is Hispanic, is Latino, is not Hispanic, is not Latino, is American Indian, is Alaska Native, is Asian, is Black, is African American, is Native Hawaiian, is Pacific Islander, is White, is below median BMI, or is at or above median BMI.

In some embodiments, the method comprises administering an additional therapeutic agent. In some embodiments, the additional therapeutic agent comprises a corticosteroid, an antihistamine, an antipyretic, or an IL-6R antagonist. In some embodiments, the additional therapeutic agent is a corticosteroid, and wherein the corticosteroid comprises prednisone, methylprednisolone, or dexamethasone. In some embodiments, the additional therapeutic agent is an antihistamine, and wherein the antihistamine comprises diphenhydramine hydrochloride or equivalent. In some embodiments, the additional therapeutic agent is an antipyretic, and wherein the antipyretic is acetaminophen. In some embodiments, the additional therapeutic agent is an IL-6R antagonist, wherein the IL-6R antagonist is tocilizumab.

### B. Dosing Strategies for Mitigating Adverse Events (e.g., Cytokine Release Syndrome)

The present invention relates to methods of treating a subject or a population of subjects having a CD20-positive cell proliferative disorder (e.g., a B cell proliferative disorder; e.g., a non-Hodgkin's lymphoma (NHL) (e.g., a large B cell lymphoma (LBCL), a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma; e.g., a relapsed and/or refractory NHL (R/R NHL; e.g., an R/R LBCL, an R/R DLBCL, an R/R FL, or an R/R MCL) or an aggressive NHL (aNHL, e.g., R/R diffuse large DLBCL, R/R HGBL, R/R trFL, and R/R Grade 3b FL)), by administration of mosunetuzumab and polatuzumab vedotin as a combination therapy. In particular, the present invention relates to methods of treating a subject or a population of subjects having an aggressive NHL (e.g., a DLBCL, a transformed FL, or a Grade 3b FL) by subcutaneous administration of mosunetuzumab and intravenous administration of polatuzumab vedotin as a combination therapy. In some instances, the subject or the population of subjects are relapsed and/or refractory (R/R) to at least one line of prior therapy, while maintaining an acceptable safety profile (e.g., with respect to frequency and severity of adverse events, such as cytokine release syndrome (CRS)). In some instances, the subject or the population of subjects may have received two or more lines of prior therapy. In some instances, the subjects may be ineligible for autologous stem cell transplant (ASCT). The therapies and dosing regimens described herein provide acceptable safety profiles in a subject or a population of subjects with R/R NHL treated with the described dosing regimens.

### 1. CRS Symptoms and Grading

Any of the methods described herein may involve monitoring a subject for cytokine release syndrome (CRS), e.g., a CRS event following commencement of any of the methods described above. Current clinical management focuses on treating the individual signs and symptoms, providing supportive care, and attempting to dampen the inflammatory response using a high dose of corticosteroids. However, this approach is not always successful, especially in the case of late intervention. The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biol Blood Marrow Transplantation. 25 (4): 625-638, 2019). The ASTCT criteria is intended to be objective, easy to apply, and more accurately categorize the severity of CRS. This CRS grading system is shown below in Table 1.

TABLE 1
CRS Grading System

| CRS | | | | |
|---|---|---|---|---|
| Parameter | Grade 1 | Grade 2 | Grade 3 | Grade 4 |
| Fever | Temperature ≥ 38° C. | Temperature ≥ 38° C. | Temperature ≥ 38° C. | Temperature ≥ 38° C. |
| | | with | | |
| Hypotension | None | Not requiring | Requiring a | Requiring multiple |
| | | vasopressors | vasopressor with or | vasopressors |
| | | | without vasopressin | (excluding |
| | | | | vasopressin) |
| | | and/or | | |
| Hypoxia | None | Requiring low-glow | Requiring high-flow | Requiring positive |
| | | nasal cannula or | nasal cannula, | pressure |
| | | blow-by | facemask, | (e.g., CPAP, BiPAP, |
| | | | nonrebreather mask | intubation and |
| | | | or Venturi mask | mechanical |
| | | | | ventilation) |

ASTCT = American Society for Transplantation and Cellular Therapy; BiPAP = bilevel positive airway pressure; CPAP = continuous positive airway pressure; CRS = cytokine release syndrome; CTCAE = Common Terminology Criteria for Adverse Events.

Fever is defined as a temperature ≥38° C. not attributable to any other cause. In subjects who have CRS then receive antipyretic or anti-cytokine therapy such as tocilizumab or steroids, fever is no longer required to grade subsequent CRS severity. In this case, CRS grading is determined by hypotension and/or hypoxia.

CRS grade is determined by the more severe event, hypotension or hypoxia not attributable to any other cause. For example, a subject with temperature of 39.5° C., hypotension requiring 1 vasopressor, and hypoxia requiring low-flow nasal cannula is classified as Grade 3 CRS.

Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

CRS is associated with elevations in a wide array of cytokines, including marked elevations in IFN-γ, IL-6, and TNF-α levels. Emerging evidence implicates IL-6, in particular, as a central mediator in CRS. IL-6 is a proinflammatory, multi-functional cytokine produced by a variety of cell types, which has been shown to be involved in a diverse array of physiological processes, including T cell activation. Regardless of the inciting agent, CRS is associated with high IL-6 levels (Nagorsen et al. Cytokine. 25 (1): 31-5, 2004; Lee et al. Blood. 124 (2): 188-95, 2014); Doesegger et al. Clin. Transl. Immunology. 4 (7): e39, 2015), and IL-6 correlates with the severity of CRS, with subjects who experience a Grade 4 or 5 CRS event having much higher IL-6 levels compared to subjects who do not experience CRS or experience milder CRS (Grades 0-3) (Chen et al. J. Immunol. Methods. 434:1-8, 2016).

Therefore, blocking the inflammatory action of IL-6 using an agent that inhibits IL-6-mediated signaling to manage CRS observed in subjects during the double-step fractionated, dose-escalation dosing regimen is an alternative to steroid treatment that would not be expected to negatively impact T cell function or diminish the efficacy or clinical benefit of mosunetuzumab therapy in the treatment of CD20-positive cell proliferative disorders (e.g., a B cell proliferative disorders).

If the subject has a CRS event that does not resolve or worsens within 24 hours of administering the IL-6R antagonist to treat the symptoms of the CRS event, and the method may further comprise administering to the subject one or more additional doses of the IL-6R antagonist to manage the CRS event. The subject may be administered a corticosteroid, such as methylprednisolone or dexamethasone if CRS event is not managed through administration of the IL-6R antagonist.

### 2. Other Adverse Events and Grading

Any of the methods described herein may involve monitoring a subject for additional non-CRS adverse events. Incidence, nature, and severity of physical findings and adverse events, with severity determined according to the National Cancer Institute Common Terminology Criteria for Adverse Events version 5 (NCI CTCAE v5.0). Other than CRS, one of the most common adverse events reported in subjects undergoing treatment with mosunetuzumab is neutropenia (e.g., febrile neutropenia).

Neutropenia is characterized by an abnormally low blood count of neutrophils, which are a type of white blood cells. Neutropenia may lead to an increased risk of infection. The generally accepted reference range for absolute neutrophil count (ANC) in adult humans is 1,500 to 8,000 cells/μL of blood. Mild neutropenia is characterized by ANC between 1,000-1,500 cells/μL (Grade 1-2); moderate neutropenia is characterized by ANC between 500 and 1,000 cells/μL (Grade 3), and severe neutropenia is characterized by ANC below 500 cells/μL (Grade 4). Febrile neutropenia (Grade 3+neutropenia) is characterized by ANC below 1,000 cells/μL in addition to either a single temperature measurement greater than 38.3° C. or sustained temperature measurements greater than 38° C. for more than one hour.

3. Dosing Regimens with Acceptable Safety Profiles

In some embodiments, administration of mosunetuzumab and/or polatuzumab vedotin to a subject according to the methods herein may result in adverse events in the subject. Described herein are methods for administering one or more additional therapeutic agents for mitigating (e.g., reducing the severity or duration of) and/or preventing adverse events in the subject.

In some embodiments, the first dosing cycle further comprises administration of a corticosteroid. In some embodiments, the second dosing cycle further comprises administration of a corticosteroid. In some embodiments, any of the one or more additional dosing cycles comprises administration of a corticosteroid. In some embodiments, a single dose of the corticosteroid is administered to the subject prior to the administration of any dose of mosunetuzumab. In some embodiments, the corticosteroid comprises dexamethasone or methylprednisolone. In some embodiments, the corticosteroid is administered intravenously or orally. In some embodiments, the corticosteroid comprises dexamethasone and is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg). In some embodiments, the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, ±10 mg, ±12 mg, ±14 mg, or ±16 mg; e.g., 80 mg).

In some embodiments, the first dosing cycle further comprises administration of an antihistamine. In some embodiments, the second dosing cycle further comprises administration of an antihistamine. In some embodiments, any of the one or more additional dosing cycles comprises administration of an antihistamine. In some embodiments, a single dose of the antihistamine is administered to the subject prior to (e.g., 30, 35, 40, 45, 50, 60 70, 80, 90, 120, 150, 180 minutes, or more prior to) the administration of any dose of mosunetuzumab. In some embodiments, the antihistamine is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the antihistamine is administered orally or intravenously. In some embodiments, the antihistamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-80 mg, 60-90 mg, 60-80 mg, 70-90 mg, or 65-85 mg; e.g., about 50 mg, about 55 mg, about 60 mg, about 65 mg, about 70 mg, about 75 mg, about 80 mg, about 85 mg, about 90 mg, about 95 mg, or about 100 mg).

In some embodiments, the first dosing cycle further comprises administration of an anti-pyretic. In some embodiments, the second dosing cycle further comprises administration of an anti-pyretic. In some embodiments, any of the one or more additional dosing cycles comprises administration of an anti-pyretic. In some embodiments, a single dose of the anti-pyretic is administered to the subject prior to the administration of any dose of mosunetuzumab. In some embodiments, the anti-pyretic is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the anti-pyretic is administered orally. In some embodiments, the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In some embodiments, the first dosing cycle further comprises administration of an initial dose of a prophylactic agent against tumor lysis syndrome (TLS). In some embodiments, the second dosing cycle further comprises administration of an initial dose of a prophylactic agent against TLS. In some embodiments, any of the one or more additional dosing cycles comprises administration of an initial dose of a prophylactic agent against TLS. In some embodiments, the initial dose of the prophylactic agent against TLS is administered to the subject prior to the administration of any dose of mosunetuzumab. In some embodiments, the prophylactic agent against TLS comprises allopurinol. In some embodiments, the initial dose of allopurinol is administered about 72 hours (e.g., 72±0.5 hours, +1 hours, +2 hours, +3 hours, +4 hours, +8 hours, +12 hours, or ±16 hours; e.g., 72 hours) prior to the administration of any dose of mosunetuzumab. In some embodiments, additional single doses of allopurinol are administered daily for 6-10 days (±1 day) after the administration of the initial dose. In some embodiments, the initial dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, each additional single dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, allopurinol is administered orally. In some embodiments, the prophylactic agent against TLS comprises rasburicase. In some embodiments, the initial dose of rasburicase is administered about 30 minutes (e.g., 30±0.5 minutes, 1 minutes, +2 minutes, +3 minutes, +4 minutes, +5 minutes, or ±6 minutes; e.g., 30 minutes) prior to the administration of any dose of mosunetuzumab. In some embodiments, additional single doses of rasburicase are administered daily for 1-5 days (±1 day) after the administration of the initial dose. In some embodiments, the initial dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, each additional single dose of rasburicase is about 0.2 mg/kg (e.g., 0.2 +0.005 mg/kg, ±0.01 mg/kg, ±0.02 mg/kg, ±0.03 mg/kg, or ±0.04 mg/kg; e.g., 0.2 mg/kg). In some embodiments, rasburicase is administered intravenously.

In some embodiments, an IL-6R antagonist may be administered to the subject treated with the methods of the invention, e.g., when the subject exhibits CRS, e.g., after being administered any dose of mosunetuzumab. In some embodiments, the IL-6R antagonist is tocilizumab. In some embodiments, tocilizumab is administered to the subject as a single dose of about 8 mg/kg (e.g., 8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 8 mg/kg), and wherein the single dose does not exceed 800 mg. In some embodiments, tocilizumab is administered to the subject as a single dose of about 12 mg/kg (e.g., 12 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 12 mg/kg), and wherein the single dose does not exceed 800 mg. In some embodiments, tocilizumab is administered intravenously.

The methods described herein may result in an acceptable safety profile for subjects having a CD20-positive cell proliferative disorder (e.g., a B cell proliferative disorder; e.g., an NHL or CLL) being treated with mosunetuzumab monotherapy or a combination therapy of mosunetuzumab and polatuzumab vedotin. In some instances, treatment using the methods described herein that include administration of mosunetuzumab (e.g., subcutaneous administration) and/or administration of polatuzumab vedotin results in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable adverse events, such as cytokine-driven toxicities (e.g., cytokine release syndrome (CRS)), infusion-related reactions (IRRs), macrophage activation syndrome (MAS), neurologic toxicities, tumor lysis syndrome (TLS), neutropenia, thrombocytopenia, elevated liver enzymes, and/or hepatotoxicities.

In some instances, subcutaneous administration of mosunetuzumab in combination with polatuzumab vedotin according to the methods described herein results in a non-inferiority or a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable adverse events, such as cytokine-driven toxicities (e.g., cytokine release syndrome (CRS)), infusion-related reactions (IRRs), macrophage activation syndrome (MAS), neurologic toxicities, tumor lysis syndrome (TLS), neutropenia, thrombocytopenia, elevated liver enzymes, and/or hepatotoxicities as compared to intravenous administration of mosunetuzumab in combination with intravenous administration of polatuzumab vedotin according to the methods described herein.

### IV. THERAPEUTIC AGENTS

### A. Mosunetuzumab

The invention provides mosunetuzumab, a bispecific antibody that binds to CD20 and CD3, useful for treating a CD20-positive cell proliferative disorder. In some instances, the CD20-positive cell proliferative disorder is a relapsed and/or refractory (R/R) non-Hodgkin's lymphomas (NHLs) (e.g., an R/R LBCL, an R/R DLBCL, an R/R FL, or an R/R MCL or an aggressive NHL (aNHL; e.g., R/R diffuse large DLBCL, R/R HGBL, R/R trFL, and R/R Grade 3b FL).

In some instances, mosunetuzumab includes an anti-CD20 arm having a first binding domain comprising at least one, two, three, four, five, or six hypervariable regions (HVRs) selected from (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6). In some instances, mosunetuzumab comprises an anti-CD20 arm comprising a first binding domain comprising at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively. In some instances, mosunetuzumab comprises an anti-CD20 arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). Accordingly, in some instances, the first binding domain comprises a VH domain comprising an amino acid sequence of SEQ ID NO: 7 and a VL domain comprising an amino acid sequence of SEQ ID NO: 8.

In some instances, mosunetuzumab includes an anti-CD3 arm having a second binding domain comprising at least one, two, three, four, five, or six HVRs selected from (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). In some instances, mosunetuzumab comprises an anti-CD3 arm comprising a second binding domain comprising at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, mosunetuzumab comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the second binding domain comprises a VH domain comprising an amino acid sequence of SEQ ID NO: 15 and a VL domain comprising an amino acid sequence of SEQ ID NO: 16.

In some instances, mosunetuzumab includes (1) an anti-CD20 arm having a first binding domain comprising at least one, two, three, four, five, or six HVRs selected from (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6); and (2) an anti-CD3 arm having a second binding domain comprising at least one, two, three, four, five, or six HVRs selected from (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). In some instances, mosunetuzumab comprises an anti-CD20 arm comprising a first binding domain comprising (1) at least one (e.g., 1, 2, 3, or 4) of heavy chain framework regions FR-H1, FR-H2, FR-H3, and FR-H4 comprising the sequences of SEQ ID NOs: 17-20, respectively, and/or an anti-CD3 arm comprising a second binding domain comprising at least one (e.g., 1, 2, 3, or 4) of the light chain framework regions FR-L1, FR-L2, FR-L3, and FR-L4 comprising the sequences of SEQ ID NOs: 21-24, respectively, and (2) at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, mosunetuzumab comprises (1) an anti-CD20 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). In some instances, mosunetuzumab comprises (1) a first binding domain comprising a VH domain comprising an amino acid sequence of SEQ ID NO: 7 and a VL domain comprising an amino acid sequence of SEQ ID NO: 8 and (2) a second binding domain comprising a VH domain comprising an amino acid sequence of SEQ ID NO: 15 and a VL domain comprising an amino acid sequence of SEQ ID NO: 16.

In some instances, mosunetuzumab has the International Nonproprietary Names for Pharmaceutical Substances (INN) List 117 (WHO Drug Information, Vol. 31, No. 2, 2017, p. 303), or CAS Registry No. 1905409-39-3, and having (1) an anti-CD20 arm comprising the heavy chain and light chain sequences of SEQ ID NOs: 33 and 34, respectively; and (2) an anti-CD3 arm comprising the heavy chain and light chain sequences of SEQ ID NOs: 35 and 36, respectively. In some instances, mosunetuzumab comprises (1) an anti-CD20 arm comprising a first binding domain comprising (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 33; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 34; or (c) a heavy chain as in (a) and a light chain as in (b), and (2) an anti-CD3 arm comprising a second binding domain comprising (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 35; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 36; or (c) a heavy chain as in (a) and a light chain as in (b). In some instances, mosunetuzumab comprises (1) an anti-CD20 arm comprising a first binding domain comprising a heavy chain comprising an amino acid sequence of SEQ ID NO: 33 and a light chain comprising an amino acid sequence of SEQ ID NO: 34 and (2) an anti-CD3 arm comprising a second binding domain comprising a heavy chain comprising an amino acid sequence of SEQ ID NO: 35 and a light chain comprising an amino acid sequence of SEQ ID NO: 36.

Amino acid sequences of mosunetuzumab are summarized in Table 2 below.

TABLE 2
Sequence IDs for Mosunetuzumab

| CD3 Arm | | CD20 Arm | |
|---|---|---|---|
| SEQ ID NO: | Description | SEQ ID NO: | Description |
| 9 | CD3 HVR-H1 | 1 | CD20 HVR-H1 |
| 10 | CD3 HVR-H2 | 2 | CD20 HVR-H2 |
| 11 | CD3 HVR-H3 | 3 | CD20 HVR-H3 |
| 12 | CD3 HVR-L1 | 4 | CD20 HVR-L1 |
| 13 | CD3 HVR-L2 | 5 | CD20 HVR-L2 |
| 14 | CD3 HVR-L3 | 6 | CD20 HVR-L3 |
| 15 | CD3 VH | 7 | CD20 VH |
| 16 | CD3 VL | 8 | CD20 VL |
| 35 | CD3 heavy chain | 33 | CD20 heavy chain |
| 36 | CD3 light chain | 34 | CD20 light chain |

Mosunetuzumab may be produced using recombinant methods and compositions, for example, as described in U.S. Pat. No. 4,816,567.

### B. Polatuzumab Vedotin

The invention provides polatuzumab vedotin, an anti-CD79b antibody drug conjugate useful for treating a CD20-positive cell proliferative disorder. In some instances, the CD20-positive cell proliferative disorder is a relapsed and/or refractory (R/R) non-Hodgkin's lymphomas (NHLs) (e.g., an R/R LBCL, an R/R DLBCL, an R/R FL, or an R/R MCL or an aggressive NHL (aNHL; e.g., R/R diffuse large DLBCL, R/R HGBL, R/R trFL, and R/R Grade 3b FL).

In some instances, the anti-CD79b antibody of polatuzumab vedotin (i.e., polatuzumab) includes an anti-CD79b binding domain comprising at least one, two, three, four, five, or six hypervariable regions (HVRs) selected from (a) an HVR-H1 comprising the amino acid sequence of SEQ ID NO: 37; (b) an HVR-H2 comprising the amino acid sequence of SEQ ID NO: 38; (c) an HVR-H3 comprising the amino acid sequence of SEQ ID NO: 39; (d) an HVR-L1 comprising the amino acid sequence of SEQ ID NO: 40; (e) an HVR-L2 comprising the amino acid sequence of SEQ ID NO: 41; and (f) an HVR-L3 comprising the amino acid sequence of SEQ ID NO: 42. In some instances, the anti-CD79b antibody drug conjugate includes an anti-CD79b binding domain comprising all six of the following HVRs: (a) an HVR-H1 comprising the amino acid sequence of GYTFSSYWIE (SEQ ID NO: 37); (b) an HVR-H2 comprising the amino acid sequence of GEILPGGGDTNYNEIFKG (SEQ ID NO: 38); (c) an HVR-H3 comprising the amino acid sequence of TARVPIRLDY (SEQ ID NO: 39); (d) an HVR-L1 comprising the amino acid sequence of KASQSVDYEGDSFLN (SEQ ID NO: 40); (e) an HVR-L2 comprising the amino acid sequence of AASNLES (SEQ ID NO: 41); and (f) an HVR-L3 comprising the amino acid sequence of QQSNEDPLT (SEQ ID NO: 42).

In some instances, the anti-CD79b antibody of polatuzumab vedotin (i.e., polatuzumab) includes at least one (e.g., 1, 2, 3, or 4) of heavy chain framework regions FR-H1, FR-H2, FR-H3, and FR-H4 comprising the sequences of SEQ ID NOs: 45-48, respectively, and/or at least one (e.g., 1, 2, 3, or 4) of the light chain framework regions FR-L1, FR-L2, FR-L3, and FR-L4 comprising the sequences of SEQ ID NOs: 49-52, respectively. In some instances, the anti-CD79b antibody drug conjugate comprises (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 43; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 44; or (c) a VH domain as in (a) and a VL domain as in (b). Accordingly, in some instances, the anti-CD79b antibody of polatuzumab vedotin (i.e., polatuzumab) comprises a VH domain comprising an amino acid sequence of SEQ ID NO: 43 and a VL domain comprising an amino acid sequence of SEQ ID NO: 44.

In some instances, the anti-CD79b antibody of polatuzumab vedotin (i.e., polatuzumab) includes (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 53; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 54; or (c) a heavy chain as in (a) and a light chain as in (b). Accordingly, in some instances, the anti-CD79b antibody of polatuzumab vedotin (i.e., polatuzumab) comprises a heavy chain comprising an amino acid sequence of SEQ ID NO: 53 and a light chain comprising an amino acid sequence of SEQ ID NO: 54.

The sequences of the anti-CD79b antibody of polatuzumab vedotin (i.e., polatuzumab) are summarized in Table 3 below.

TABLE 3
Sequence IDs for the Anti-CD79b
Antibody of Polatuzumab Vedotin

| | |
|---|---|
| Heavy Chain | Light Chain |

| SEQ ID NO: | Description | SEQ ID NO: | Description |
|---|---|---|---|
| 37 | HVR-H1 | 40 | HVR-L1 |
| 38 | HVR-H2 | 41 | HVR-L2 |
| 39 | HVR-H3 | 42 | HVR-L3 |
| 43 | VH | 44 | VL |
| 53 | Heavy Chain | 54 | Light Chain |

In some instances, the anti-CD79b antibody of polatuzumab vedotin (i.e., polatuzumab) is linked to monomethyl auristatin E (MMAE, i.e., vedotin). In some instances, polatuzumab vedotin (immunoglobulin G1-kappa auristatin E conjugate, anti-[Homo sapiens CD79b (immunoglobulin-associated CD79 beta)] is a humanized monoclonal antibody conjugated to auristatin E; gamma1 heavy chain (1-447) [humanized VH (Homo sapiens IGHV3-23*04 (76.50%)-(IGHD)-IGHJ4*01) [8.8.10] (1-117)-Homo sapiens IGHG1*03 (CH1 R120>K (214) (118-215), hinge (216-230), CH2 (231-340), CH3 (341-445), CHS (446-447)) (118-447)], (220-218′)-disulfide with kappa light chain (1′-218′) [humanized V-KAPPA (Homo sapiens IGKV1-39*01 (85.90%)-IGKJ1*01) [10.3.9] (1′-111′)-Homo sapiens IGKC*01 (112′-218′)]; dimer (226-226″: 229-229″)-bisdisulfide; conjugated, on an average of 3 to 4 cysteinyl, to monomethylauristatin E (MMAE), via a cleavable maleimidocaproyl-valyl-citrullinyl-p-aminobenzyloxycarbonyl (mc-val-cit-PABC) type linker; also known as RG-7596, or RO5541077-000)), as defined by International Nonproprietary Names for Pharmaceutical Substances (INN) List 110 (WHO Drug Information, Vol. 27, No. 4, 2016, p. 443). Polatuzumab vedotin is also referred to as IUPHAR/BPS Number 8404, the KEGG Number D10761, or the CAS #: 1313206-42-6. Polatuzumab vedotin is also interchangeably referred to as “polatuzumab vedotin-piiq”, “huMA79bv28-MC-vc-PAB-MMAE”, or “DCDS4501A.”

In some instances, polatuzumab vedotin comprises the formula:

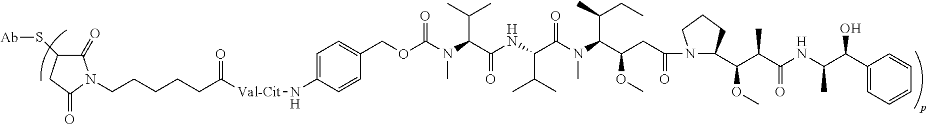

wherein Ab is polatuzumab described herein, and wherein p is between 1 and 8.

In some embodiments, polatuzumab vedotin comprises an anti-CD79b antibody (i.e., polatuzumab) that comprises a VH as in any of the embodiments provided herein, and a VL as in any of the embodiments provided herein. In some embodiments, polatuzumab vedotin comprises an anti-CD79b antibody (i.e., polatuzumab) that comprises the VH and VL sequences having the amino acid sequences of SEQ ID NO: 43 and SEQ ID NO: 44, respectively. In some embodiments, polatuzumab vedotin comprises an anti-CD79b antibody (i.e., polatuzumab) that comprises a heavy chain having the amino acid sequence of SEQ ID NO: 53 and a light chain having the amino acid sequence of SEQ ID NO: 54.

In some embodiments, polatuzumab vedotin comprises a substantially full length anti-CD79b antibody, e.g., an IgG₁ antibody or other antibody class or isotype as described elsewhere herein. Polatuzumab vedotin may be produced using recombinant methods and compositions, for example, as described in U.S. Pat. No. 4,816,567Polatuzumab vedotin (or huMA79bv28-MC-vc-PAB-MMA) is described in U.S. Pat. No. 8,088,378).

### C. Additional Therapeutic Agents

In some instances, the methods described herein include administering mosunetuzumab and polatuzumab vedotin in combination with one or more additional therapeutic agents.

In some instances, the one or more additional therapeutic agents may reduce the rate or the severity of cytokine release syndrome (CRS). In some instances, the one or more additional therapeutic agents may prevent symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #50-42-8), or methylprednisolone (CAS #: 83-43-2)) or an IL-6R antagonist (e.g., tocilizumab (CAS #: 375823-41-9), sarilumab (CAS #: 1189541-98-7), vobarilizumab (ALX-0061; CAS #: 1628814-88-9), satralizumab (SA-237; CAS #: 1535963-91-7), and variants thereof).

In some instances, the additional therapeutic agent is tocilizumab. In some instances, the additional therapeutic agent is a corticosteroid. In some instances, the corticosteroid is dexamethasone. In some instances, the corticosteroid is prednisone. In some instances, the corticosteroid is methylprednisolone.

In some instances, the one or more additional therapeutic agents is acetaminophen or paracetamol. Acetaminophen or paracetamol has the CAS #: 103-90-2.

In some instances, the one or more additional therapeutic agents is diphenhydramine. Diphenhydramine has the CAS #: 58-73-1.

In some instances, additional therapeutic agents useful in the present invention include therapeutic antibodies, such as alemtuzumab (CAMPATH®), bevacizumab (AVASTIN®, Genentech); cetuximab (ERBITUX®, Imclone); panitumumab (VECTIBIX®, Amgen), rituximab (RITUXAN®, Genentech/Biogen Idec; CAS #: 174722-31-7), pertuzumab (OMNITARG®, 2C4, Genentech), trastuzumab (HERCEPTIN®, Genentech), tositumomab (BEXXAR®, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, tafasitamab, talizumab, tefibazumab, tocilizumab, toralizumab, tucotuzumab celmoleukin, tucusituzumab, umavizumab, urtoxazumab, ustekinumab, visilizumab, and briakinumab.

In some instances, the one or more additional therapeutic agents may be used in the treatment of neutropenia. In some instances, the additional therapeutic agents may prevent symptoms associated with neutropenia. In some instances, the additional therapeutic agents may reduce the rate or severity of neutropenia. In particular instances, the additional therapeutic agent is granulocyte colony-stimulating factor (G-CSF or GCSF) or colony-stimulating factor 3 (CSF 3). The mRNA sequence of human G-CSF/CSF 3 includes, e.g., NCBI RefSeq No. NM_000759, NM_001178147, NM_172219, and NM_172220, and the protein amino acid sequence of human G-CSF/CSF 3 includes, e.g., NCBI RefSeq No. NP_000750, NP_001171618, NP_757373, and NP_757374.

### V. PHARMACEUTICAL COMPOSITIONS AND FORMULATIONS

Mosunetuzumab and/or polatuzumab vedotin described herein can be used in pharmaceutical compositions and formulations. Pharmaceutical compositions and formulations of mosunetuzumab, polatuzumab vedotin, and/or other therapeutic agents describe herein (e.g., dexamethasone, methylprednisolone, prednisone, acetaminophen, paracetamol, and diphenhydramine) can be prepared by mixing one, two, or all three agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Polatuzumab vedotin may also be formulated according to standard formulation and/or manufacturing practices. Dexamethasone, methylprednisolone, prednisone, acetaminophen, paracetamol, and diphenhydramine may also be formulated according to standard formulation and/or manufacturing practices. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a corticosteroid, a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

In some embodiments, mosunetuzumab is formulated for administration subcutaneously. In some embodiments, polatuzumab vedotin is formulated for administration intravenously. In some embodiments, dexamethasone is formulated for administration intravenously. In some embodiments, dexamethasone is formulated for administration orally. In some embodiments, methylprednisolone is formulated for administration intravenously. In some embodiments, prednisone is formulated for administration orally. In some embodiments, acetaminophen or paracetamol is formulated for administration orally. In some embodiments, diphenhydramine is formulated for administration orally.

### VI. KITS AND ARTICLES OF MANUFACTURE

In another aspect of the invention, a kit or an article of manufacture containing materials useful for the treatment, prevention, and/or diagnosis of the disorders described above is provided. The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is mosunetuzumab or polatuzumab vedotin described herein. The label or package insert indicates that the composition is used for treating a CD20-positive cell proliferative disorder (e.g., a B cell proliferative disorder; e.g., a non-Hodgkin's lymphoma (NHL) (e.g., a diffuse-large B cell lymphoma (DLBCL), a follicular lymphoma (FL), a high-grade B cell lymphoma (HGBL), a mantle cell lymphoma (MCL), a high-grade B cell lymphoma, a primary mediastinal (thymic) large B cell lymphoma (PMLBCL), a diffuse B cell lymphoma, a small lymphocytic lymphoma, a marginal zone lymphoma (MZL), a Burkitt lymphoma, or a lymphoplasmacytic lymphoma; e.g., an aggressive NHL (aNHL)) and further includes information related to at least one of the dosing regimens described herein. In some embodiments, the label or package insert indicates that the composition is used for treating a CD20-positive cell proliferative disorder in a subject who is relapsed and/or refractory (R/R) to at least one prior lines of therapy and is autologous stem cell transplant (ASCT) or a subject who is R/R to at least two prior lines of therapy. Moreover, the kit or article of manufacture may comprise (a) a first container with a composition contained therein, wherein the composition comprises mosunetuzumab, polatuzumab vedotin, or both mosunetuzumab and polatuzumab vedotin; and (b) a second container with a composition contained therein, wherein the composition comprises an additional therapeutic agent. Examples of additional therapeutic agents include dexamethasone, methylprednisolone, prednisone, acetaminophen, paracetamol, and diphenhydramine. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### Example 1. A Randomized, Open-Label, Multicenter Phase III Study Evaluating Efficacy and

Safety of Mosunetuzumab in Combination with Polatuzumab Vedotin in Comparison with Rituximab in Combination with Gemcitabine Plus Oxaliplatin (R-GemOx) in Subjects with Relapsed and/or Refractory Aggressive B-Cell Non-Hodgkin's Lymphoma (“SUNMO”)

### Study Design

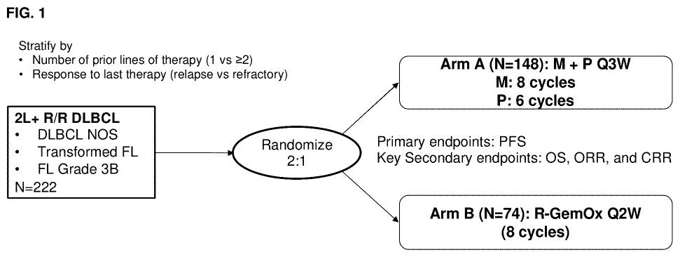

This is a Phase III, open-label, multicenter, randomized, controlled trial in subjects with R/R DLBCL, trFL, or FL Grade 3b, who are not candidates for ASCT. Approximately 222 eligible subjects are randomized in 2:1 ratio to receive either mosunetuzumab (M)+polatuzumab vedotin (P) (Arm A) or R-GemOx (rituximab, gemcitabine, oxaliplatin) (Arm B). This study evaluates the efficacy and safety of mosunetuzumab compared with R-GemOx in subjects with R/R aggressive non-Hodgkin's lymphoma (aNHL), including DLBCL, trFL, and FL Grade 3b, who received at least one prior systemic therapy and are not candidates for ASCT. The study schema is provided in FIG. 1.

### Randomization

Subjects in this trial are stratified at the time of randomization for the following 2 factors:
Number of previous lines of systemic therapy for aggressive lymphoma (1 vs. ≥2).
Outcome after last systemic therapy (relapsed vs. refractory)
   Relapsed disease in this study is defined as disease that has recurred >6 months after completion of the last treatment.
   Refractory disease is defined as disease that either progressed during therapy or progressed within 6 months (<6 months) of last treatment.

### Arm A Treatment (Mosunetuzumab+Polatuzumab Vedotin)

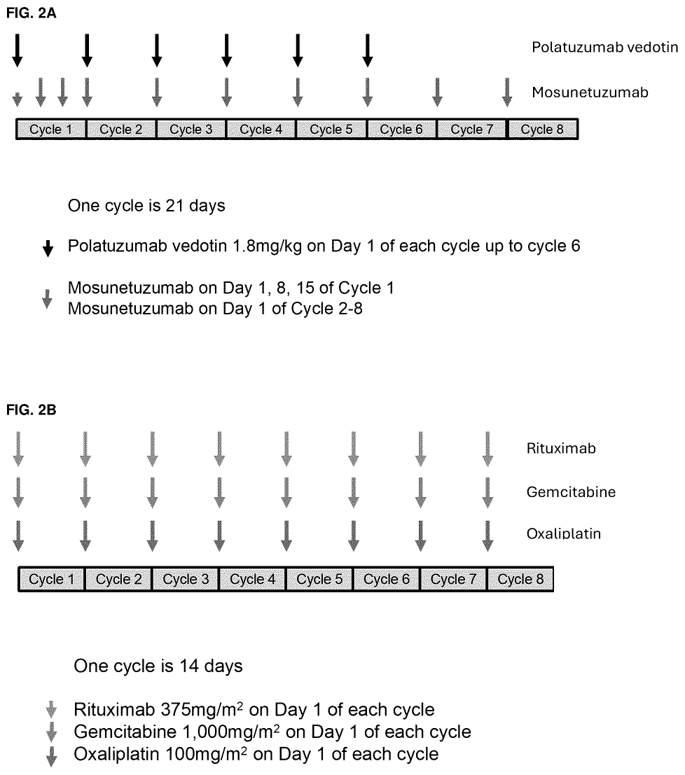

The Arm A treatment consists of mosunetuzumab administered subcutaneously (SC) and polatuzumab vedotin administered intravenously (IV) (see FIG. 2A). One cycle of treatment is 21 days (e.g., 21-day dosing cycles). Mosunetuzumab is administered 5 mg SC on Cycle 1, Day 1 (Cycle 1 Dose 1; i.e., C1D1); 45 mg on Cycle 1 (Cycle 1 Dose 2; i.e., C1D2), Day 8; Cycle 1, Day 15 (Cycle 1 Dose 3; i.e., C1D3); and Day 1 of Cycles 2-8 (Cycle 2-8 Dose 1; i.e., C2D1-C8D1). Polatuzumab vedotin is administered IV at 1.8 mg/kg on Day 1 of Cycles 1-6 (C1D1-C6D1). Prophylactic (preemptive) or therapeutic use of granulocyte colony-stimulating factor (G-CSF) is permitted. Dosing occurs if a subject's clinical assessment and laboratory test values are acceptable, including peripheral neuropathy Grade≤1, ANC ≥1000/mm3 and platelet count ≥75,000/mm3. Details of each drug administration is described in the Study Treatment section below.

### Arm B Treatment (Rituximab, Gemcitabine, Oxaliplatin)

The Arm B treatment consists of R-GemOx (see FIG. 2B). One cycle of treatment is 14 days (e.g., 14-day dosing cycles). Rituximab 375 mg/m² is administered IV on Day 1. Gemcitabine 1000 mg/m² is administered IV on Day 1. Oxaliplatin 100 mg/m² is administered IV on Day 1. Prophylactic (preemptive) or therapeutic use of G-CSF is permitted at the treating physician's discretion. Dosing occurs if a subject's clinical assessment and laboratory test values are acceptable, including ANC ≥1000/mm3 and platelet count >75,000/mm3. If these required hematologic parameters are not met within 2 weeks after the last treatment, the treatment is delayed. If such a delay occurs despite prophylactic (preemptive) use of G CSF, it is acceptable to change the treatment cycle to every 21 days (e.g., 21-day dosing cycles), instead of 14 days for the subsequent treatment. Treatment is administered for up to 8 cycles. Details of each drug administration is described in Study Treatment section below. In some instances, a reference population of subjects comprises subjects who are assigned to Arm B and/or who receive the Arm B treatment.

### Assessment During the Study

All subjects are monitored for adverse events, clinical laboratory test results and vital signs throughout the study and for at least 90 days after the final dose of study treatment. Adverse events are graded according to the National Cancer Institute Common Terminology Criteria for Adverse Events (NCI CTCAE) Version 5.0, except for cytokine release syndrome (CRS) severity, which is determined per the American Society for Transplantation and Cellular Therapy (ASTCT) CRS grading criteria CRS (Lee et al. Biol Blood Marrow Transplantation. 25 (4): 625-638, 2019). Response assessments are performed according to the 2014 Lugano Response Criteria (Cheson B D, et al. J Clin Oncol 2014; 32:1-9), as assessed on positron emission tomography (PET)/computed tomography scans. To characterize the pharmacokinetic (PK) profile and immune response in response to study treatment, blood samples are taken at various timepoints before and after dosing.

### Inclusion Criteria

Subjects in the study exhibit the following inclusion criteria:
Subjects who are age >18 years at the time of signing Informed Consent Form
Subjects who have an Eastern Cooperative Oncology Group (ECOG) Performance Status of 0, 1, or 2.
Subjects who have a life expectancy of at least 12 weeks
Subjects who have CD20-positive aggressive lymphoma (e.g., aggressive non-Hodgkin's lymphoma [aNHL]) as determined by the local hematopathology laboratory from the following diagnoses by 2016 World Health Organization classification of lymphoid neoplasms:
   Diffuse large B cell lymphoma (DLBCL), not otherwise specified (NOS)
   High-grade B-cell lymphoma (HGBL; NOS or double/triple hit)
   Transformed follicular lymphoma (trFL): The disease must be R/R to standard therapies for trFL
   Follicular lymphoma (FL) Grade 3b.
Subjects who have received at least one prior systemic therapy for aNHL.
Subjects who have either relapsed or have become refractory to a prior regimen must meet the following criteria:
   Relapsed to prior regimen(s) after having a documented history of response (CR or PR) of ≥6 months in duration from completion of regimen(s)
   Refractory to any prior regimen, defined as no response to the prior therapy, or progression within 6 months of completion of the last dose of therapy.
Subjects who have received only one prior line of therapy must be ineligible for ASCT.
Subjects who have measurable disease, defined as at least 1 bi-dimensionally measurable nodal lesion, defined as >1.5 cm in its longest dimension, or at least 1 bi-dimensionally measurable extra nodal lesion, defined as >1.0 cm in its longest dimension.
Subjects who have a pathology report for the initial histopathology diagnosis and the most recent histopathology diagnosis prior to entering the study
   Subjects with trFL must also have a pathology report completed at the time of disease transformation
Subjects whose representative tumor specimen and the corresponding pathology report are available for confirmation of diagnosis as well as for biomarker analysis
   Pretreatment sample of excisional, incisional, forceps, or at least 1 core-needle tumor biopsy is required. Cytological or fine-needle aspiration samples are not acceptable.
   Fresh biopsy is preferred. However, subjects who are unable to undergo biopsy procedures may be eligible for study enrollment if an archival tumor tissue sample obtained after the most recent systemic treatment as paraffin blocks or at least 10 slides (preferably 15) unstained can be sent to the Sponsor.
   Receipt of tumor samples or central review of diagnosis does not have to occur prior to study enrollment.
Subjects who have adequate hepatic, hematologic, and renal functions defined by laboratory values below:
   Hepatic function: AST and ALT≤2.5× upper limit of normal (ULN); total bilirubin≤1.5× ULN; subjects with a documented history of Gilbert syndrome and in whom total bilirubin elevations are accompanied by elevated indirect bilirubin are eligible.
   Hematologic function: platelet count >75,000/mm3 without transfusion within 14 days prior to first dose of study treatment; ANC >1000/mm3; total hemoglobin >9 g/dL without transfusion within 14 days prior to first dose of study treatment.
   Subjects with extensive marrow involvement of lymphoma and/or disease-related cytopenias (e.g., immune thrombocytopenia) may be enrolled if the following is met: platelet count ≥50,000/mm3 without transfusion within 14 days of study treatment; ANC ≥500/mm3; any hemoglobin but without transfusion within 7 days prior to first dose of study treatment.
   Renal function: estimated creatinine clearance (CrCI) ≥40 mL/min by Cockroft-Gault method (see Gault MH, Longerich LL, Harnett J D, et al., Nephron 1992, 62:249) or other institutional standard methods.
For women of childbearing potential: subjects who agree to remain abstinent (refrain from heterosexual intercourse) or use contraception and agree to refrain from donating eggs.
For men: subjects who agree to remain abstinent (refrain from heterosexual intercourse) or use a condom and agree to refrain from donating sperm.

### Exclusion Criteria:

Subjects in the study do not exhibit the following exclusion criteria:
Subjects who are pregnant, breastfeeding, or intending to become pregnant during the study or within 3 months after the final dose of mosunetuzumab, 9 months after the final dose of polatuzumab vedotin, 12 months after the final dose of rituximab, 6 months after the final dose of gemcitabine, 9 months after the final dose of oxaliplatin, and 3 months after the final dose of tocilizumab, as applicable. Women of childbearing potential must have a negative serum pregnancy test result within 7 days prior to initiation of study treatment.
Subjects who have received prior treatment with mosunetuzumab or other CD20-directed bispecific antibodies, prior treatment with polatuzumab vedotin, and/or prior treatment with R-GemOx or GemOx.
Subjects who have a contraindication to any component of the study treatment
Subjects with current Grade >1 peripheral neuropathy.
Subjects who have received anti-lymphoma treatments with monoclonal antibodies, radio-immunoconjugates or ADCs within 4 weeks before the first dose of study treatment.
Subjects who have received treatment with any chemotherapeutic agent, or treatment with any other anti-cancer agent (investigational or otherwise) within 4 weeks or 5 half-lives of the drug, whichever is shorter, prior to the first dose of study treatment.
Subjects who have received treatment with radiotherapy within 2 weeks prior to the first dose of study treatment
   Subjects who have received radiotherapy within 4 weeks prior to the first study treatment administration must have at least one measurable lesion outside of the radiation field.
   Subjects who have only one measurable lesion that was previously irradiated but subsequently progressed are eligible.
Subjects who have ASCT within 100 days prior to the first study treatment administration
Subjects who received prior treatment with CAR T therapy within 30 days before the first study treatment administration
Subjects who have had prior allogeneic SCT
Subjects who have had solid organ transplantation
Subjects who have a known or suspected history of HLH
Subjects who have a history of confirmed progressive multifocal leukoencephalopathy.
Subjects who have a history of severe allergic or anaphylactic reactions to monoclonal antibody therapy (or recombination antibody-related fusion proteins).
Subjects who have history of malignancy that has been treated with curative intent within ≥2 years prior to screening, with the exception of the cancer under investigation in this study and malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer
   Subjects who have prostate cancer with no evidence of metastatic disease and are not on active therapy except for anti-androgen therapy may be allowed study entry
   Subjects who have a history of curatively treated basal or squamous cell carcinoma of the skin or in situ carcinoma of the cervix are allowed
   Subjects who have a malignancy that has been in remission without treatment for ≥2 years prior to the first study treatment administration are allowed
Subjects who currently have or have had a past history of CNS involvement of lymphoma
Subjects who have a history of CNS disease which was symptomatic or required treatment in the past 1 year, such as stroke, epilepsy, CNS vasculitis or neurodegenerative disease
Subjects who have significant cardiovascular disease such as New York Heart Association Class Ill or IV cardiac disease, myocardial infarction within the last 6 months, unstable arrhythmias, or unstable angina
Subjects who have significant active pulmonary disease (e.g., bronchospasm and/or obstructive pulmonary disease)
Subjects who have a known active bacterial, viral, fungal, mycobacterial, parasitic, or other infection (excluding fungal infections of the nail beds) at study enrollment, or any major episode of infection requiring treatment with IV antibiotics or hospitalization (relating to the completion of the course of antibiotics) within 2 weeks prior to the first study treatment administration
Subjects who have a known or suspected chronic active Epstein-Barr virus (EBV) infection.
Subjects who have had a recent major surgery within 4 weeks prior to the first study treatment administration. Protocol-mandated procedures (e.g., tumor biopsies and bone marrow biopsies) are permitted
Subjects who have positive test results for chronic hepatitis B infection (defined as positive hepatitis B surface antigen [HBsAg] serology). Subjects with occult or prior hepatitis B infection (defined as positive total hepatitis B core antibody and negative HBsAg) may be included if hepatitis B virus (HBV) DNA is undetectable at the time of screening. These subjects should be considered for prophylactic antivirals (e.g., entecavir) before and throughout the treatment, and must be willing to undergo monthly DNA testing.
Subjects who have acute or chronic hepatitis C virus (HCV) infection. Subjects who are positive for HCV antibody must be negative for HCV by polymerase chain reaction (PCR) to be eligible for study participation.
Subjects who have a history of HIV infection
Subjects who have been administered a live, attenuated vaccine within 4 weeks before the first dose of study treatment administration or anticipation that such a live, attenuated vaccine is required during the study. Subjects must not receive live, attenuated vaccines (e.g., FluMist®) while receiving study treatment and after the last dose until B-cell recovery to the normal ranges. Killed vaccines or toxoids should be given at least 4 weeks prior to the first dose of study treatment to allow development of sufficient immunity.
Subjects with a history of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barre syndrome, multiple sclerosis, vasculitis, or glomerulonephritis
   Subjects with a history of autoimmune-related hypothyroidism on a stable dose of thyroid replacement hormone may be eligible
   Subjects with controlled Type 1 diabetes mellitus who are on an insulin regimen are eligible for the study
Subjects who have received systemic immunosuppressive medications (including, but not limited to, cyclophosphamide, azathioprine, methotrexate, thalidomide, and anti-TNF agents) with the exception of corticosteroid treatment≤10 mg/day prednisone or equivalent within 2 weeks prior to first dose of study treatment
   Subjects who have received acute, low-dose, systemic immunosuppressant medications (e.g., single dose of dexamethasone for nausea or B-symptoms) may be enrolled in the study
   The use of inhaled corticosteroids is permitted
   The use of mineralocorticoids for management of orthostatic hypotension is permitted
   The use of physiologic doses of corticosteroids for management of adrenal insufficiency is permitted.
Subjects who received investigational therapy, whether or not intended for lymphoma treatment, within 7 days prior to initiation of study treatment
Subjects who have a clinically significant history of liver disease, including viral or other hepatitis, or cirrhosis
Subjects who have any serious medical condition or abnormality in clinical laboratory tests that, precludes the subject's safe participation in and in the completion of the study, or which could affect compliance with the protocol or interpretation of results

### Study Treatments and Concomitant Therapy

The investigational medicinal products (IMPs) for this study are mosunetuzumab, polatuzumab vedotin, rituximab, gemcitabine, oxaliplatin, and tocilizumab. On days when 2 of the IMPs are given, the order of the administration in Arm A should be polatuzumab vedotin, followed by mosunetuzumab, and the interval between the end of polatuzumab vedotin infusion and the mosunetuzumab injection should be at least 60 minutes. In Arm B, gemcitabine should be administered before oxaliplatin. The administration of rituximab can be either before gemcitabine or after oxaliplatin.

### Polatuzumab Vedotin

The dose of polatuzumab vedotin is 1.8 mg/kg. Polatuzumab vedotin was administered intravenously (IV) on Day 1 in Cycles 1-6 (C1D1-C6D1). If the subject's weight within 96 hours prior to Day 1 of a given treatment cycle increases or decreases >10% from the weight obtained for Cycle 1, Day 1, the most recent weight is used to calculate the dose. The weight that triggered a dose adjustment is taken as the new reference weight for future dose adjustments. All subsequent doses are modified accordingly.

After reconstitution with sterile water for injection and dilution into IV bags that contain isotonic sodium chloride solution (0.9% NaCl), polatuzumab vedotin is administered by IV infusion with use of a dedicated standard administration set with 0.2 μM or 0.22 μM in line filters at a final polatuzumab vedotin concentration determined by the subject specific dose.

The initial dose is administered over 90 (+10) minutes to subjects who are well hydrated. Premedication (e.g., 500-1000 mg of oral acetaminophen or paracetamol and 50±100 mg diphenhydramine as per institutional standard practice) may be administered to an individual subject before administration of polatuzumab vedotin. Administration of corticosteroids is permitted at the discretion of the treating physician. If infusion-related reactions (IRRs) are observed with the first infusion in the absence of premedication, premedication must be administered before subsequent doses.

The polatuzumab vedotin infusion is slowed or interrupted for subjects experiencing infusion-associated symptoms. Following the initial dose, subjects are observed for 90 minutes for fever, chills, rigors, hypotension, nausea, or other infusion-associated symptoms. If prior infusions have been well-tolerated, subsequent doses of polatuzumab vedotin are administered over 30 (±10) minutes, followed by a 30-minute observation period after the infusion. The time interval between the end of infusion of polatuzumab vedotin and the start of mosunetuzumab injection is at least 60 minutes.

### Mosunetuzumab

Mosunetuzumab is administered subcutaneously (SC) with a Cycle 1 step-up-dosing regimen. The same dosing, independent of body weight, is used for mosunetuzumab. In Cycle 1, subjects receive mosunetuzumab on Day 1 (5 mg; Cycle 1 Dose 1 (C1D1)), Day 8 (45 mg; Cycle 1 Dose 2 (C1D2)), and Day 15 (45 mg; Cycle 1 Dose 3 (C1D3)). In Cycles 2-8, subjects receive mosunetuzumab on Day 1 (45 mg; Cycle 2 Dose 1 to Cycle 8 Dose 1 (C2D1-C8D1)).

Mosunetuzumab is delivered by standard medical syringe with a final volume not to exceed 2.0 mL.

Mosunetuzumab is administered to well-hydrated subjects. Corticosteroid premedication with dexamethasone 20 mg is administered prior the administration of each mosunetuzumab dose. The administration of corticosteroid premedication may be optional for Cycle 2 and beyond at the investigator's discretion. However, if the subject experiences CRS with prior administration of mosunetuzumab, premedication with steroids must be administered for subsequent doses until no additional CRS events are observed. In addition, premedication with oral acetaminophen or paracetamol (e.g., 500-1000 mg) and/or 50-100 mg diphenhydramine may be administered per standard institutional practice prior to administration of mosunetuzumab.

Mosunetuzumab is administered over 30 seconds to 2 minutes. Refer to the pharmacy manual for more details, including syringe size and preferred injection site. During Cycles 1 and 2, and also for later cycles if CRS occurred after the last mosunetuzumab, subjects are observed for at least 30 minutes after mosunetuzumab for fever, chills, rigors, hypotension, nausea, or other signs and symptoms of CRS. Vital signs are recorded pre-injection (within 30 minutes) and then 30 (+15) minutes after mosunetuzumab administration. In Cycle 3 and beyond, in the absence of CRS after the last dose of mosunetuzumab, the observation time after the mosunetuzumab injection is at least 15 minutes. Vital signs are assessed prior to the mosunetuzumab injection (within 30 minutes prior to injection) and then at least once after the injection during the observation period.

### Tocilizumab

Tocilizumab is administered intravenously (IV) only to those subjects who experience a CRS event for which tocilizumab is indicated. Subjects who weigh ≥30 kg receive tocilizumab 8 mg/kg in a 100-mL infusion bag or bottle, and subjects who weigh >30 kg receive tocilizumab 12 mg/kg in 50-mL infusion bag or bottle. Doses exceeding 800 mg per infusion are not recommended. The infusion is administered IV over 60 minutes. Treatment is repeated every 8 hours as necessary (for a maximum of 4 doses).

### Rituximab

Rituximab is administered at a dose of 375 mg/m² by intravenously (IV) infusion on Day 1 of each 14-day cycle (Cycles 1-8). Once the rituximab infusion is complete, subjects are to be observed for 30 minutes before the start of the other infusions. The infusion of rituximab may be split over 2 days if the subject is at increased risk for an infusion-related reaction (IRR) (high tumor burden or high peripheral lymphocyte count). For subjects who experience an adverse event during a rituximab infusion, administration of R-GemOx may be continued on the following day, if required. Rituximab can be given either before gemcitabine or after oxaliplatin on the same day.

### Gemcitabine

Gemcitabine is administered at 1000 mg/m² intravenously (IV) on Day 1 of each 14-day cycle (Cycles 1-8). Gemcitabine is administered before oxaliplatin on the same day.

### Oxaliplatin

Oxaliplatin is administered at 100 mg/m² intravenously (IV) on Day 1 of each 14-day cycle (Cycles 1-8). Oxaliplatin is administered after gemcitabine on the same day.

### Permitted Concomitant Therapy

In general, investigators may manage a subject's care (including preexisting conditions) through use of supportive therapies. Subjects who experience infusion-associated symptoms may be treated symptomatically with acetaminophen, ibuprofen, diphenhydramine, and/or H2-receptor antagonists (e.g., famotidine, cimetidine), or equivalent medications. Prophylactic use of hematopoietic growth factors or anti-infectives for viral, fungal, bacterial, or Pneumocystis infections are permitted. Premedication with antihistamines, antipyretics, and/or analgesics are also permitted.

### Cautionary Concomitant Therapy

Given the expected pharmacology of mosunetuzumab, the transient release of cytokines (most resolved within the first 24 hours of the Cycle 1, Day 1 dose) may suppress CYP450 enzymes and cause drug-drug interactions. Subjects who may be of highest risk of a drug-drug interaction are those receiving concomitant medications that are CYP450 substrates and have a narrow therapeutic index.

In vitro data suggest that unconjugated MMAE is mainly metabolized by CYP3A4 and, to a lesser extent, by CYP2D6. Based on a validated physiological-based PK model simulation (Chen et al. 2015), strong CYP3A4 inhibitors may increase the exposure (e.g., AUC) of unconjugated MMAE by approximately 50%, while antibody-conjugated monomethyl auristatin E (acMMAE; e.g., polatuzumab vedotin) PK is not affected. Concomitant medications that are strong CYP3A4 inhibitors should be considered cautionary as they may potentially lead to adverse reactions.

Cytochrome P450 enzymes in the liver are down-regulated by infection and inflammatory stimuli, including cytokines such as IL-6. Inhibition of IL-6 signaling in subjects with rheumatoid arthritis who are treated with tocilizumab may restore CYP450 activities to higher levels than those subjects not treated with tocilizumab, leading to increased metabolism of drugs that are CYP450 substrates. In vitro studies showed that tocilizumab has the potential to affect expression of multiple CYP enzymes, including CYP1A2, CY2B6, CYP2C9, CYP2C19, CYP2D6, and CYP3A4. The effects of tocilizumab on CYP2C8 or transporters are unknown. In vivo studies with omeprazole (metabolized by CYP2C19 and CYP3A4) and simvastatin (metabolized by CYP3A4) showed up to a 28% and 57% decrease in exposure 1 week following a single dose of tocilizumab, respectively. The effect of tocilizumab on CYP450 enzyme activity may persist for several weeks after stopping therapy.

### Prohibited Concomitant Therapy

Herbal therapies intended for the treatment of lymphoma are prohibited.
Concomitant therapy intended for the treatment of cancer (including, but not limited to, chemotherapy, hormonal therapy, immunotherapy, radiotherapy, and herbal therapy), whether health authority-approved or experimental, is prohibited for various time periods prior to starting study treatment, depending on the agent, and during study treatment, until disease progression is documented and the subject has discontinued study treatment
Intrathecal chemotherapy for CNS prophylaxis is permitted.
Adjuvant endocrine therapy for non-metastatic, hormone receptor positive breast cancer and anti-androgen therapy for non-metastatic prostate cancer are permitted.
Investigational therapy, whether intended for the treatment of lymphoma or not, is prohibited within 7 days prior to initiation of study treatment and during study treatment
Systemic immunosuppressive therapy is prohibited (except medications indicated per protocol, including corticosteroids and tocilizumab)
Live virus vaccines are prohibited for at least 4 weeks before initiation of or at any time during study treatment

### Efficacy Assessments

Subjects undergo tumor assessments at screening, every 8 weeks for the first 6 months following treatment initiation, and every 3 months, regardless of dose delays, until radiographic disease progression, study discontinuation or up to 2 years, whichever is earlier, per Lugano Criteria 2014 (Cheson B D, et al. J Clin Oncol 2014; 32:1-9). All measurable and/or evaluable lesions are assessed and documented at screening.

### Radiographic Assessments

Fluorodeoxyglucose (FDG) PET-CT scans, in conjunction with diagnostic-quality CT scans, are required at screening, the interim response assessment, and at the end-of-treatment. After the end-of-treatment, CT scans (preferred) or FDG PET-CT scans are performed. Diagnosis of disease progression based on clinical examination are confirmed radiographically by imaging (e.g., CT scan, FDG PET-CT scan) or histopathologically by biopsy as soon as feasible (within 30 days) and prior to initiation of non-protocol specified anti-cancer therapy.

All measurable and/or evaluable disease are documented at screening and re assessed at each subsequent tumor evaluation. Response is assessed by the investigator and the IRF on the basis of physical examinations, CT scans, FDG PET-CT scans.

### Bone Marrow Assessment

Subjects may use screening PET/CT scans to assess bone marrow involvement; bone marrow examinations are not required unless clinically indicated (Cheson B D, et al. J Clin Oncol 2014; 32:1-9).

### Response Evaluation

Objective response is determined at specified timepoints according to the Lugano Response Criteria (Cheson B D, et al. J Clin Oncol 2014; 32:1-9).

Endpoints (e.g., ORR, CRR, PFS, DOR, duration of complete response [DOCR]), are calculated programmatically.

### Safety Assessment

### Adverse Events

An adverse event is any untoward medical occurrence in a patient or clinical study subject temporally associated with the use of a study treatment, whether or not considered related to the study treatment.

The following events meet the definition of adverse event:
Any abnormal laboratory test results (hematology, clinical chemistry, or urinalysis) or other safety assessments (e.g., ECG, radiological scans, vital sign measurements), including those that worsen from baseline, or are considered clinically significant in the medical and scientific judgment of the investigator (i.e., not related to progression of underlying disease).
Exacerbation of a chronic or intermittent preexisting condition, including either an increase in frequency and/or intensity of the condition.
New condition detected or diagnosed after study treatment administration, even though it may have been present before the start of the study
Signs, symptoms, or clinical sequelae of a suspected drug-drug interaction
Signs, symptoms, or clinical sequelae of a suspected overdose of either study treatment or a concomitant medication

Overdose per se is noy reported as an adverse event or serious adverse event unless it is an intentional overdose taken with possible suicidal or self-harming intent. Such overdoses are reported regardless of sequelae.
“Lack of efficacy” or “failure of expected pharmacological action” per se is not reported as an adverse event or serious adverse event. Such instances are captured in the efficacy assessments. However, the signs, symptoms, and/or clinical sequelae resulting from lack of efficacy are reported as an adverse event or serious adverse event if they fulfill the definition of an adverse event or serious adverse event.

The investigator assesses the severity of each adverse event reported during the study through use of the NCI CTCAE (v5.0) grading scale.

### Serious Adverse Events

A serious adverse event is defined as any untoward medical occurrence that, at any dose: results in death; is life-threatening; requires in-patient hospitalization or prolongation of existing hospitalization; results in persistent disability or incapacity; is a congenital anomaly or birth defect; and other adverse events deemed to be serious adverse events, e.g. invasive or malignant cancers, intensive treatment in an emergency room or at home for allergic bronchospasm, blood dyscrasias or convulsions that do not result in hospitalization, or development of drug dependency or drug abuse.

The terms “severe” and “serious” are not synonymous. Severity refers to the intensity of an adverse event (e.g., rated as mild, moderate, or severe, or according to National Cancer Institute Common Terminology Criteria for Adverse Events [NCI CTCAE] (v5.0)); the event itself may be of relatively minor medical significance (such as severe headache without any further findings). Severity and seriousness need to be independently assessed for each adverse event.

### Adverse Events of Special Interest

Adverse events of special interest (AESI) for this study include cases of potential drug-induced liver injury that include an elevated ALT or AST in combination with either an elevated bilirubin or clinical jaundice and cases of suspected transmission of an infectious agent by a study treatment. In addition, AESI specific to mosunetuzumab or polatuzumab vedotin are as follows:

Mosunetuzumab:
Grade ≥2 CRS
Grade ≥2 neurologic adverse event
Grade ≥2 injection-site reaction.
Any suspected HLH or macrophage activation syndrome
Grade ≥3 TLS
Grade ≥3 febrile neutropenia
Grade ≥2 AST, ALT, or total bilirubin elevation.
Any Grade disseminated intravascular coagulation (minimum Grade 2 by definition)
Grade ≥2 tumor flare (e.g., manifestation of signs/symptoms associated with an increase in size of known nodal or extranodal lesions by clinical or radiographic assessment, new onset or worsening of preexisting pleural effusions).
Any Grade pneumonitis/interstitial lung disease (excluding pneumonia of infectious etiology)

Polatuzumab Vedotin:
TLS any grade (minimum Grade 3 by definition)
Second malignancies

### Pharmacokinetics

Serum/plasma samples are collected for measurement of serum/plasma concentrations of mosunetuzumab and polatuzumab vedotin. Samples are used to evaluate the pharmacokinetics of mosunetuzumab and polatuzumab vedotin. Samples collected for analyses of mosunetuzumab and polatuzumab vedotin concentration may also be used to evaluate safety or efficacy aspects related to concerns arising during or after the study. Also, these data are used to understand the relationship of PK exposure to dose and support characterization of dose/exposure-response relationships in the combination setting. In addition, these data are used to explore and characterize the potential PK interactions between mosunetuzumab and polatuzumab vedotin.

### Clinical Outcome Assessments

Subject-reported outcome (PRO) instruments are completed to assess the treatment benefit of mosunetuzumab and polatuzumab vedotin compared with R-GemOx. In addition, PRO instruments enable the capture of each subject's direct experience with mosunetuzumab and polatuzumab vedotin.

Subject-reported outcomes data are collected through use of the following instruments: the European Organization for Research and Treatment of Cancer Quality of Life-Core 30 Questionnaire (EORTC QLQ-C30; see Aaronson et al. J. Natl Cancer Inst. 1993, 85 (5): 365-376; Fitzsimmons et al., Eur. J. Cancer. 1999, 35 (6): 939-941; Functional Assessment of Cancer Therapy-Lymphoma subscale (FACT Lym LymS; see Hlubocky et al., Leuk Lymphoma. 2013, 54 (9): 1942-1946); Functional Assessment of Cancer Therapy-Gynecologic Oncology Group-Neurotoxicity (FACT/GOG-Ntx; Huang et al., Int. J. Gynecol. Cancer. 2007, 17 (2): 387-393); and EuroQol 5-Dimension, 5-Level Questionnaire (EQ-5D-5L; see EuroQol Group Health Policy. 1990, 16 (3): 199-208; Brooks Health Policy 1996, 37 (1): 53-72; Herdman et al. Qual. Life. Res. 2011, 20 (10): 1727-1736; Janssen et al. Qual. Life. Res. 2013, 22 (7): 1717-1727).

### Additional Samples

Additional samples may be obtained from consenting subjects. Additional sample types include tissue samples (e.g., body fluids, solid tissues, and derivatives thereof), blood samples, tumor biopsies. Samples may be used for exploratory biomarker research. Samples may be used for biomolecule extraction (e.g., DNA, RNA, and/or protein extraction).

### Example 2. An Open-Label, Randomized, Multicenter, Phase Ib/II Trial Evaluating the Safety, Tolerability, Pharmacokinetics, and Efficacy of Mosunetuzumab (BTCT4465A) in Combination with Polatuzumab Vedotin in Patients with B-Cell Non-Hodgkin Lymphoma

This study evaluates the safety, tolerability, pharmacokinetics, and efficacy of mosunetuzumab in combination with polatuzumab vedotin in patients with B-cell NHL. Specific objectives and corresponding endpoints for the study are outlined in Table 4.

TABLE 4
Objectives and Endpoints

| | |
|---|---|
| Objectives | Corresponding Endpoints |

Safety Objective:

| | |
|---|---|
| To evaluate the safety and tolerability of | Occurrence and severity of adverse events, |
| mosunetuzumab plus polatuzumab vedotin in patients | including DLTs, with severity determined |
| with R/R DLBCL or FL, including estimation of the MTD, | according to NCI CTCAE v5.0; for CRS, |
| determination of the RP2D, and characterization of | severity determined according to the |
| DLTs (Phase Ib) | ASTCT CRS Consensus Grading criteria |
| To evaluate the safety of mosunetuzumab plus | Change from baseline in targeted vital signs |
| polatuzumab vedotin (Arm I) in patients with R/R FL | Change from baseline in targeted clinical |
| (Phase II) | laboratory test results |
| To evaluate the safety of mosunetuzumab SC plus | |
| polatuzumab vedotin (Arm K) in patients with R/R MCL | |
| (Phase II) | |
| To evaluate the safety of mosunetuzumab SC plus | |
| polatuzumab vedotin (Arm L) compared to rituximab | |
| polatuzumab vedotin (Arm M) in patients with R/R | |
| DLBCL (Phase II) | |

Efficacy Objective:

| | |
|---|---|
| To make a preliminary assessment of the anti-tumor | CR rate at the time of PRA based on PET |
| activity of mosunetuzumab plus polatuzumab vedotin | and/or CT scan, as determined by the |
| (Phase Ib) | investigator using Lugano 2014 criteria |
| To evaluate the efficacy of mosunetuzumab plus | ORR, defined as CR or PR, at PRA based |
| polatuzumab vedotin (Arm I) in patients with R/R FL | on PET and/or CT scan, as determined by |
| (Phase II) | the investigator and IRC using Lugano 2014 |
| To evaluate the efficacy of mosunetuzumab plus | criteria |
| polatuzumab vedotin (Arm J) in patients with R/R | Best ORR (CR or PR at any time) on study |
| DLBCL (Phase II) | based on PET and/or CT scan, as |
| To evaluate the efficacy of mosunetuzumab SC plus | determined by the investigator using |
| polatuzumab vedotin (Arm K) in patients with R/R MCL | Lugano 2014 criteria |
| (Phase II) | DOR, defined as the time from the first |
| To evaluate the efficacy of mosunetuzumab SC plus | occurrence of a documented objective |
| polatuzumab vedotin (Arm L) compared to rituximab | response to disease progression or relapse, |
| plus polatuzumab vedotin (Arm M) in patients with R/R | as determined by the investigator using |
| DLBCL (Phase II) | Lugano 2014 criteria, or death from any |
| To evaluate the efficacy of mosunetuzumab IV plus | cause, whichever occurs first |
| polatuzumab vedotin (Arm I) in patients with R/R FL | PFS, defined as the time from first study |
| DLBCL (Phase II) | treatment to the first occurrence of disease |
| To evaluate the efficacy of mosunetuzumab IV plus | progression or relapse, as determined by |
| polatuzumab vedotin (Arm J) in patients with R/R | the investigator and IRC using Lugano 2014 |
| DLBCL (Phase II) | criteria, or death from any cause, whichever |
| To evaluate the efficacy of mosunetuzumab SC plus | occurs first |
| polatuzumab vedotin (Arm K) in patients with R/R MCL | EFS, defined as the time from first study |
| DLBCL (Phase II) | treatment to the first occurrence of disease |
| To evaluate the efficacy of mosunetuzumab SC plus | progression or relapse, as determined by |
| polatuzumab vedotin (Arm L) compared to rituximab | the investigator and IRC using Lugano 2014 |
| polatuzumab vedotin (Arm M) in patients with R/R | criteria, initiation of NALT, or death from any |
| DLBCL DLBCL (Phase II) | cause, whichever occurs first |
| | OS, defined as the time from first study |
| | treatment to death from any cause |
| | CIRS-G prognostic impact on PFS and OS |
| | for patients ≥65 years old (Arm J, Arm L, |
| | and Arm M only) |

Pharmacokinetic Objective:

| | |
|---|---|
| To characterize the pharmacokinetics of | For mosunetuzumab in combination with |
| mosunetuzumab (SC and IV) when administered in | polatuzumab vedotin: |
| combination with polatuzumab vedotin (Groups A, B, | Cₘₐₓ |
| and C; Arms I, J, K, and L) | Cₘᵢₙ |
| To characterize the pharmacokinetics of polatuzumab | Total exposure (AUC), CL, and volume of |
| vedotin when administered in combination with | distribution, as estimated by population PK |
| mosunetuzumab (Groups A, B, and C; Arms I, J, K, and | modeling, as appropriate, and supported by |
| L) or rituximab (Arm M) | data |
| To characterize the relationship between | For polatuzumab vedotin in combination |
| pharmacokinetics and safety, biomarkers, or efficacy | with mosunetuzumab or rituximab |
| To assess potential PK interactions between | C_{EOI} |
| mosunetuzumab and polatuzumab vedotin (Groups A, | C_{trough} |
| B, and C; Arms I, J, K, and L) | Total exposure (AUC), CL, and volume of |
| | distribution, as estimated by population PK |
| | modeling, as appropriate, and supported by |
| | data |
| | Relationship between pharmacokinetics and |
| | safety, biomarkers, or efficacy endpoints, as |
| | appropriate |
| | Concentrations of mosunetuzumab when |
| | administered in combination with |
| | polatuzumab vedotin compared with |
| | mosunetuzumab given as a single agent |
| | based on historical data |
| | Concentrations of polatuzumab vedotin |
| | analytes when administered in combination |
| | with mosunetuzumab compared with |
| | polatuzumab vedotin as a single agent |
| | based on historical data |

Immunogenicity Objective:

| | |
|---|---|
| To assess the incidence of ADAs to mosunetuzumab | Relationship between ADA status and |
| (Groups A, B, and C; Arms I, J, K, and L) | efficacy, safety, pharmacokinetics, and |
| To assess the incidence of ADAs to polatuzumab | biomarkers |
| vedotin (Groups A, B, and C; Arms I, J, K, M, and L) | |

Biomarker Objective:

| | |
|---|---|
| To identify biomarkers that are predictive of response to | Association between prognostic subtypes, |
| mosunetuzumab plus polatuzumab vedotin (i.e., | exploratory biomarkers, and PET-CT CR |
| predictive biomarkers), are associated with progression | rate, ORR, DOR, PFS, and EFS endpoints |
| to a more severe disease state (i.e., prognostic | Relationship over time between ctDNA and |
| biomarkers), are associated with acquired resistance to | tumor burden as measured by imaging |
| mosunetuzumab plus polatuzumab vedotin, are | |
| associated with susceptibility to developing adverse | |
| events, can provide evidence of mosunetuzumab plus | |
| polatuzumab vedotin activity, or can increase the | |
| knowledge and understanding of disease biology | |
| To make a preliminary assessment of response to | |
| mosunetuzumab plus polatuzumab vedotin in different | |
| clinical and biologic prognostic subgroups of NHL | |
| To make a preliminary assessment of MRD status | |
| following mosunetuzumab treatment in combination with | |
| polatuzumab vedotin (Groups A, B, and C; Arms I, J, K, | |
| L, and M) | |

Health Status Utility Objective:

| | |
|---|---|
| To assess health status of patients | Health status (EQ-5D-5L) |

ADA = anti-drug antibody; ASTCT = American Society for Transplantation and Cellular Therapy; AUC = area under the concentration-time curve; C_{EOI} = concentration at end of infusion; CIRS-G = Cumulative Illness Rating Scale-Geriatric (CIRS-G) (Miller et al., Psychiatry Research. 41(3): 237-248); CL = clearance; Cₘₐₓ = maximum serum concentration; Cₘᵢₙ = minimum serum concentration; CR = complete response; CRS = cytokine release syndrome; CT = computed tomography (scan); C_{trough} = trough concentration; ctDNA = circulating tumor DNA; DLBCL = diffuse large B-cell lymphoma; DLT = dose-limiting toxicity; DOR = duration of response; EFS = event-free survival; EORTC QLQ-C30 = European Organization for Research and Treatment of Cancer Quality of Life-Core 30 Questionnaire; EQ-5D-5L = EuroQol 5-Dimension, 5-Level (questionnaire); FACT/GOG-Ntx = Functional Assessment of Cancer Treatment/Gynecologic Oncology Group-Neurotoxicity; FACT-Lym = Functional Assessment of Cancer Therapy-Lymphoma (subscale); FL = follicular lymphoma; IRC = Independent Review Committee; MRD = minimal residual disease; MTD = maximum tolerated dose; NALT = new anti-lymphoma treatment; NCI CTCAE v5.0 = National Cancer Institute Common Terminology Criteria for Adverse Events, Version 5.0; NHL = non-Hodgkin lymphoma; ORR = objective response rate; OS = overall survival; PET = positron emission tomography (scan); PET-CR = complete response based on positron emission tomography scan; PET-CT = positron emission tomography-computed tomography (scan); PFS = progression-free survival; PK = pharmacokinetic; PR = partial response; PRA = primary response assessment; RP2D = recommended Phase II dose; R/R = relapsed or refractory; SCT = stem-cell transplantation.

### Study Design

This Phase Ib/II open-label multicenter study is designed to evaluate the safety, tolerability, pharmacokinetics, pharmacodynamics, and efficacy of escalating doses of intravenous (IV) or subcutaneous (SC) mosunetuzumab in combination with polatuzumab vedotin, in patients with R/R follicular lymphoma (FL), diffuse large B cell lymphoma (DLBCL), and mantle cell lymphoma (MCL); to determine a recommended Phase II dose (RP2D) and schedule of mosunetuzumab in combination with polatuzumab vedotin.

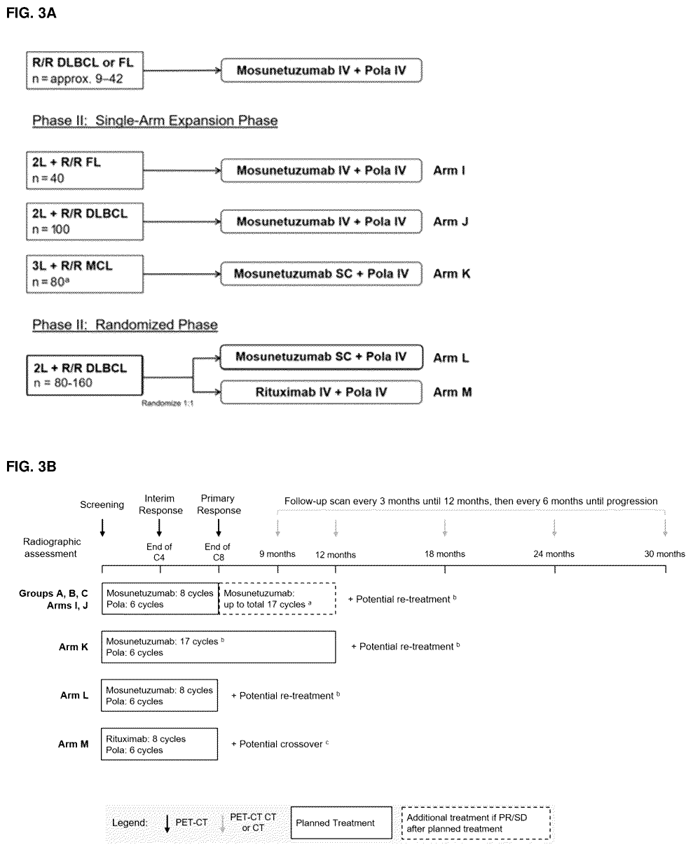

The study will include an initial dose-finding phase followed by a single-arm expansion phase for second line or later (2L+) patients with R/R DLBCL and 2L+R/R FL. In addition, mosunetuzumab SC dosing in combination with polatuzumab vedotin will be evaluated in patients with at least 2 prior lines of systemic therapy (3+) for the treatment of R/R MCL (Arm K), and in patients with 2L+R/R DLBCL in the randomized Phase II portion of the study (Arms L and M). FIG. 3A and FIG. 3B present an overview of the study design. This study includes up to 422 patients at approximately 40 investigative sites globally.

All patients are closely monitored for adverse events throughout the study and for at least 90 days after the last dose of study treatment or until the initiation of a next anti-lymphoma treatment, whichever is earlier. Adverse events are graded according to the National Cancer Institute Common Terminology Criteria for Adverse Events, Version 5.0 (NCI CTCAE v5.0), except for CRS events, which are graded according to the ASTCT CRS Consensus Grading criteria. An Internal Monitoring Committee (IMC) is responsible for monitoring patient safety throughout the study.

Blood samples are taken at various time-points before and during study treatment administration for biomarker analyses and to characterize the PK properties of mosunetuzumab and polatuzumab vedotin, as well as the immunogenicity of mosunetuzumab and polatuzumab vedotin when given in combination.

Response in the Phase II portion of the study (Arms I, J, K, L, and M) is determined by an IRC and investigators using the Lugano Response Criteria for Malignant Lymphoma (Cheson et al., J Clin Oncol. 2014. 32 (27): 3059-3067), hereinafter referred to as the “Lugano 2014 criteria.” Interim response is assessed between Day 15 of Cycle 4 and Day 21 of Cycle 4, prior to Cycle 5. Primary response assessment (PRA) for all cohorts at the end of Cycle 8 (Cycle 8 Day 21±1 week) for patients following either the Group A or C dosing schedule, or at the end of Cycle 9 (Cycle 9 Day 21±1 week) for patients following the Group B dosing schedule.

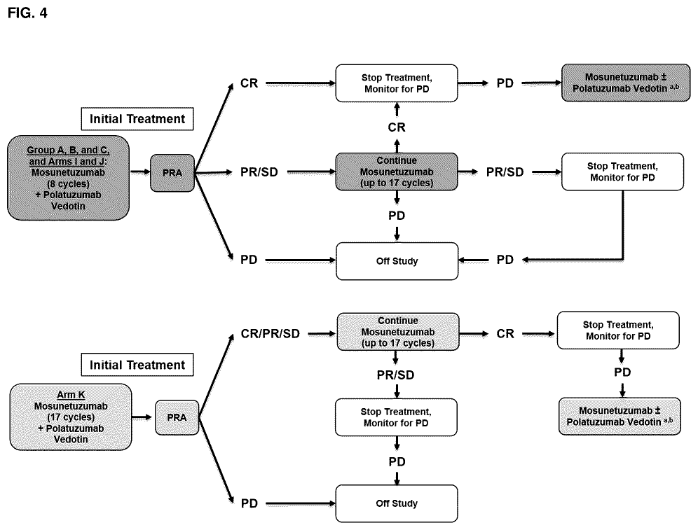

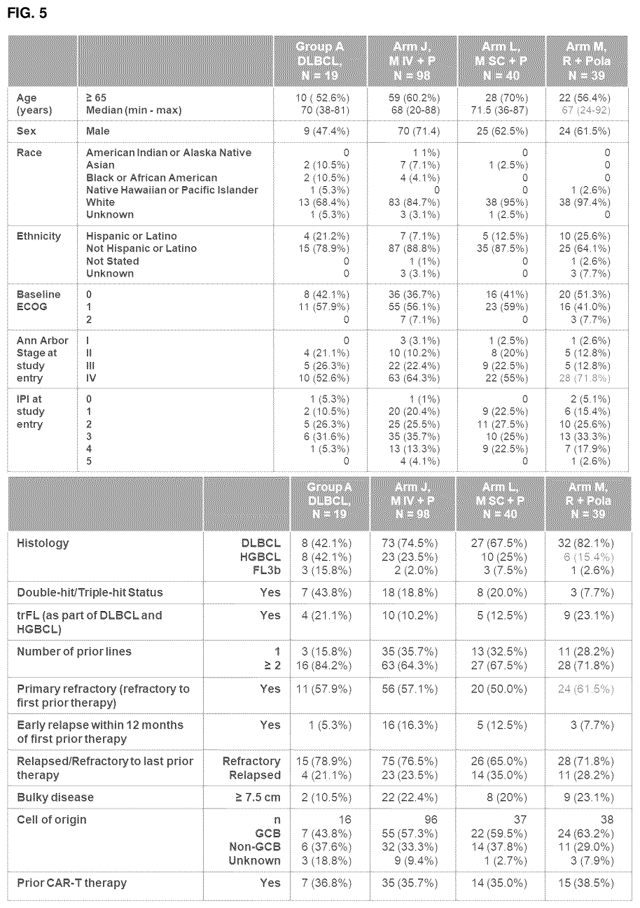

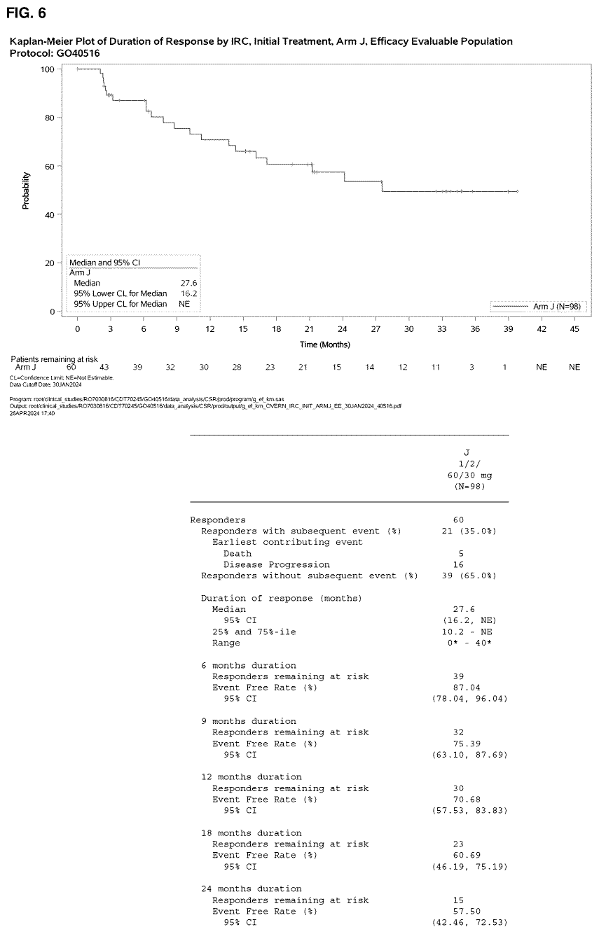

Patients are evaluated every 3 months (+2 weeks) by computed tomography (CT) scan or positron emission tomography (PET)-CT for the first year after C1D1, and then every 6 months (+2 weeks) until disease progression, death, withdrawal of consent, or initiation of another anti-cancer therapy (FIG. 4). Tumor assessments are performed to confirm clinical suspicion of relapse or disease progression.

Study treatments are administered every 21 days, with each 21-day period comprising a cycle. Mosunetuzumab is administered for 8 (Groups A, B, C, and Arms I, J, L, and M) or 17 cycles (Groups A, B, C, and Arms I, J, and K). Polatuzumab vedotin is administered for 6 cycles (Groups A, B, and C; Arms I, J, K, L, and M). Rituximab is administered on Day 1 of each cycle for 8 cycles (Arm M).

### Dose-Finding Phase

The purpose of the dose-finding phase is to determine the RP2D and schedule for mosunetuzumab when given in combination with fixed doses of polatuzumab vedotin (1.8 mg/kg) in patients with R/R DLBCL or FL. Approximately 9-42 patients with either R/R DLBCL or FL are enrolled in up to three dose-escalation treatment groups.
Group A: C1 step-up mosunetuzumab escalation with concurrent administration of polatuzumab vedotin starting in C1, both administered by IV infusion
Group B: C1 polatuzumab vedotin with delayed start C1 step-up mosunetuzumab escalation, both administered by IV infusion
Group C: C1 step-up mosunetuzumab escalation with concurrent administration of polatuzumab vedotin starting in C2, both administered by IV infusion

Dose escalation of Groups A, B, and C are run sequentially or in parallel at the discretion of the Sponsor. Dose escalation is performed based on a modified 3+3 design. Dose-escalation cohorts (within each group) consist of at least 3 patients, unless DLTs are observed in the first 2 patients prior to enrollment of a third patient. Approximately 6-12 patients are treated at the RP2D and schedule of mosunetuzumab in combination with polatuzumab vedotin prior to the expansion phase.

For each dose-escalation cohort in Groups A, B, and C, treatment is staggered such that the second patient enrolled in the cohort receives the first dose of study treatment at least 72 hours after the first enrolled patient receives the first dose of study treatment, to assess for any severe and unexpected acute drug or infusion-related toxicities. Dosing in subsequent patients in each cohort is staggered by at least 24 hours from the end of the prior patient's administration. In each scenario, the Sponsor must receive documentation of the status of the prior patient before the next patient receives the first dose of study treatment.

Patients are closely monitored for adverse events during a DLT assessment window. Adverse events meeting the criteria for DLT, will be reported to the Sponsor within 24 hours. Staggered patient enrollment is not required for enrollment of additional patients to acquire additional safety and PD data at a dose level that has been shown to not exceed the MTD. Patients exhibiting acceptable safety and evidence of clinical benefit continue to receive study treatment every 21 days for 8 cycles (or 17 cycles if PR or stable disease [SD] after 8 cycles) for mosunetuzumab, and for 6 cycles for polatuzumab vedotin, until confirmed objective disease progression or unacceptable toxicity, whichever occurs first.

Mosunetuzumab dose levels are independent of patient weight. The starting dose level of step-up mosunetuzumab is 1 mg (DL1, fixed for all schedules), 2 mg (DL2, fixed for all schedules, given 7 days after DL1), and 9 mg (DL3, initial mosunetuzumab test dose, given 7 days after DL2), for each initial cohort in Groups A, B, and C based on preliminary data from Study GO29781.

### Dose Limiting Toxicities (DLTs)

All adverse events, including DLTs, are reported and graded according to NCI CTCAE v5.0 unless otherwise indicated. Although CRS is graded according to the ASTCT CRS consensus grading criteria, for dose-escalation decisions, DLTs related to CRS are defined based on individual signs and symptoms and laboratory data according to NCI CTCAE v5.0. Decreases in B cells, lymphopenia, and/or leukopenia due to decreases in B cells are not considered DLTs as they are expected PD outcomes of mosunetuzumab and polatuzumab vedotin treatment. DLT assessment period is Cycle 1 Day 1 through Cycle 1 Day 21 for Group A; Cycle 1 Day 8 through Cycle 2 Day 21 for Group B, and Cycle 2 Day 1 through Cycle 2 Day 21 for Group C. In the dose finding phase, a DLT is defined as:
Any Grade ≥3 hematologic adverse event in the absence of another clearly identifiable cause, with the following exceptions:
   Grade 3 or 4 neutropenia that is not accompanied by temperature elevation (as a single oral temperature of ≥38.3° C. [101° F.] or an oral temperature of ≥38.0° C. [100.4° F.] sustained for ≥1 hour) and improves to Grade≤2 (or to >80% of the baseline value, whichever is lower) without a delay of the next scheduled cycle of study treatment exceeding 7 days
   Grade 3 or 4 lymphopenia, which is an expected outcome of therapy
   Grade 3 or 4 leukopenia, which is an expected outcome of therapy
   Grade 3 or 4 thrombocytopenia that improves to Grade≤2 (or to ≥80% of the baseline value, whichever is lower) before D1 of the next scheduled cycle of study treatment without platelet transfusion and is not associated with bleeding that is considered clinically significant by the investigator
   Grade 3 or 4 anemia that does not require an emergent transfusion.
Any Grade ≥3 non-hematologic adverse event not considered by the investigator to be attributable to another clearly identifiable cause, with the following exceptions:
   Grade 3 diarrhea that responds to standard-of-care therapy within 72 hours
   Grade 3 nausea or vomiting in the absence of premedication or that can be managed with resulting resolution to Grade≤2 with oral or IV anti-emetics within 72 hours
   Grade 3 nausea or vomiting that requires total parenteral nutrition or hospitalization are not excluded and should be considered a DLT.
   Grade 3 laboratory abnormality that is asymptomatic and deemed by the investigator not to be clinically significant
   Grade 3 fatigue lasting≤3 days
   Grade 3 (NCI CTCAE v5.0) individual signs and symptoms of CRS after mosunetuzumab infusion (Section 5.3.5.1) that occurs in the context of Grade≤2 CRS (as defined by the ASTCT CRS Consensus Grading criteria; and lasts <3 days will not be considered a DLT
   Grade 3 elevation in ALT or AST, provided the following criteria are met:
      Any Grade 3 AST or ALT elevation that lasts <3 days
      ALT or AST level is ≤8× the upper limit of normal (ULN)
      ALT or AST elevation resolves to Grade <2 (<5× ULN) within 7 days Total and direct bilirubin and other laboratory parameters of liver synthetic function (e.g., prothrombin time) are normal

There are no clinical signs or symptoms of hepatic injury.
Any case involving an increase in hepatic transaminase >3× baseline in combination with either an increase in direct bilirubin >2× ULN or clinical jaundice, without any findings of cholestasis or signs of hepatic dysfunction and in the absence of other contributory factors (e.g., worsening of metastatic disease or concomitant exposure to known hepatotoxic agent or of a documented infectious etiology) is suggestive of potential drug-induced liver injury (according to Hy's Law) and will be considered a DLT unless the following criteria are met:
   Any AST or ALT >3× the ULN and total bilirubin >2× ULN where no individual laboratory value exceeds Grade 3 and lasts <3 days will not be considered a DLT.

During dose finding in Groups A, B, and C, only the DL3 test dose may be escalated or de-escalate according to rules discussed below in detail. Mosunetuzumab dose levels may be rounded if the difference before and after the rounding is within 15% (e.g., 13.5 may be rounded to 14 mg, and 27 mg may be rounded to 30 mg). An example of dose escalation and de-escalation is shown in Table 5, though specific doses listed are for illustrative purposes only.

TABLE 5
Examples of Dose Escalation and De-Escalation for Groups A, B, and C

| Group | Cohort | DL₁ (mg) | DL₂(mg) | DL₃(mg) |
|---|---|---|---|---|
| A | Cohort A3 (escalation) | 1.0 | 2.0 | 40.0 |
| | Cohort A2 (escalation) | 1.0 | 2.0 | 20.0 |
| | Cohort A1 (initial cohort) | 1.0 | 2.0 | 9.0 |
| | Cohort A0 (de-escalation) | 1.0 | 2.0 | 6.0 |
| | | No DL₁ de-escalation | No DL₂ de-escalation | |
| | | allowed; assess | allowed; assess | |
| | | alternate schedule. | alternate schedule. | |
| B | Cohort B3 (escalation) | 1.0 | 2.0 | 40.0 |
| | Cohort B2 (escalation) | 1.0 | 2.0 | 20.0 |
| | Cohort B1 (initial cohort) | 1.0 | 2.0 | 9.0 |
| | Cohort B0 (de-escalation) | 1.0 | 2.0 | 6.0 |
| | | No DL₁ de-escalation | No DL₂ de-escalation | |
| | | allowed; assess | allowed; assess | |
| | | alternate schedule. | alternate schedule. | |
| C | Cohort C3 (escalation) | 1.0 | 2.0 | 40.0 |
| | Cohort C2 (escalation) | 1.0 | 2.0 | 20.0 |
| | Cohort C1 (initial cohort) | 1.0 | 2.0 | 9.0 |
| | Cohort C0 (de-escalation) | 1.0 | 2.0 | 6.0 |
| | | No DL₁ de-escalation | No DL₂ de-escalation | |
| | | allowed. | allowed. | |

DL = dose level.

Group A-Cycle 1 Double-Step Fractionated Mosunetuzumab Escalation with Concurrent Administration of Polatuzumab Vedotin Starting in Cycle 1

Group A evaluates mosunetuzumab and polatuzumab vedotin given concurrently starting on C1D1. Patients enrolled in dose finding Group A receive mosunetuzumab 1 mg (DL1) on C1D1, 2 mg (DL2) on C1D8, and the first DL3 test dose on C1D15 by IV infusion. In Cycle 2 and beyond (up to 8-17 cycles), the mosunetuzumab DL3 dose is given on Day 1 of each 21-day cycle, with Day 1 of Cycle 2 being 7 days after the C1D15 dose.

Patients receive polatuzumab vedotin 1.8 mg/kg by IV infusion on Day 1 of each 21-day cycle for up to a maximum of 6 cycles, starting on C1D1.

Schedule and dose level of mosunetuzumab plus polatuzumab vedotin are found in Table 6.

TABLE 6
Mosunetuzumab Plus Polatuzumab Vedotin Regimen (Group A)

| | | | | | | Cycles | Cycles | Cycles |
|---|---|---|---|---|---|---|---|---|
| | | | Cycle 1 | | | 2-6 | 7-8 | 9-17 ^{a} |
| Agent | Dose | Route | Day 1 | Day 8 | Day 15 | Day 1 | Day 1 | Day 1 |
| Polatuzumab | 1.8 | IV | x | | | x | | |
| vedotin | mg/kg | | | | | | | |
| Mosunetuzumab | DL₁ | IV | x | | | | | |
| | DL₂ | IV | | x | | | | |
| | DL₃ | IV | | | x | x | x | x |

DL = dose level.

Mosunetuzumab and polatuzumab vedotin may be given up to +1 day from the scheduled date for Cycle 2 (i.e., with a minimum of 6 days after C1D15 dosing), and +2 days from the scheduled date for Cycle 3 and beyond (i.e., with a minimum of 19 days between doses) for logistic/scheduling reasons.

Dose escalation in Group A uses a modified 3+3 design. The DLT assessment period for Group A is C1D1 through C1D21. Dose escalation of mosunetuzumab DL3 alone is based on recommendations by the IMC for each successive cohort based on set escalation rules. A minimum of 3 patients are enrolled into each cohort, unless the first 2 enrolled patients experience a protocol-defined DLT, in which case enrollment into the cohort is terminated. If none of the first 3 DLT-evaluable patients experiences a DLT, enrollment of the next cohort at the next highest dose level may proceed. If 1 of the first 3 DLT-evaluable patients experiences a DLT, the cohort is expanded to 6 patients, and all 6 patients are evaluated for DLTs before any dose-escalation decision. If no additional patient experiences a DLT in the 6 DLT-evaluable patients, enrollment of the next cohort at the next highest dose level may proceed.

Otherwise, if the cohort has expanded to 6 patients, the cumulative MTD of a cohort may be exceeded under 2 scenarios. In the first scenario, because DL1 and DL2 are fixed for each cohort in each group and are evaluated with each cohort during dose escalation, the assessment of MTD associated with these two doses reviews all DLTs occurring prior to the administration of the first DL3 test dose across all cohorts within a group. The MTD is exceeded if the number of DLTs prior to the administration of the first DL3 test dose across all applicable cohorts has >80% chance that the true DLT rate >20%, by the posterior probability approach (Thall and Simon Controlled Clinical Trials. 1994. 15 (6): 463-81). For example, there is an >80% chance that true DLT rate >20% if DLTs observed in 2/4, 2/5, 2/6, 3/7, 3/8, 3/9, 3/10, 4/11, 4/12, 4/13, 4/14, or 5/15 patients. If the MTD has been exceeded based on DLTs occurring prior to the administration of the first DL3 test dose, there are no dose reductions allowed for DL1 or DL2. Instead, an alternate schedule in other groups is tested. In the second scenario, if 2 or more out of 6 DLT-evaluable patients experience a DLT after the administration of the first DL3 test dose, the MTD is deemed to have been exceeded and dose escalation stops. An additional 3 patients are evaluated for DLTs at the preceding dose level, unless 6 patients have already been evaluated at that level. However, if the dose level at which the MTD is exceeded is >25% higher than the preceding dose level, 6 patients may be evaluated at an intermediate dose level.

If the MTD is exceeded at any dose level, the highest dose where fewer than 2 out of 6 DLT-evaluable patients (i.e., <33%) experience a DLT after the administration of the first DL3 test dose is declared the MTD. If the MTD is not exceeded at any dose level, the highest dose administered in this group is declared the maximum assessed dose. In the event that the initial mosunetuzumab DL3 test dose in combination with polatuzumab vedotin is above the MTD (i.e., >33% out of 6 DLT-evaluable patients experience a DLT after the administration of the first DL3 test dose), a reduced DL3 dose level that is at least 25% lower may be evaluated in an additional cohort of 3 to 6 patients. If this dose level is again above the MTD, further DL3 dose reductions of >25% of the preceding DL3 dose may be assessed in subsequent cohorts of 3-6 patients. The highest dose level where fewer than 2 out of 6 DLT-evaluable patients (i.e., <33%) experience DLTs is declared the MTD.

Group B-Cycle 1 Polatuzumab Vedotin with Delayed Start Cycle 1 Double-Step Fractionated Mosunetuzumab Escalation

Group B evaluates an alternate schedule of polatuzumab vedotin and mosunetuzumab, with polatuzumab starting on C1D1 and mosunetuzumab double-step fractionated doses starting on C1D8. Patients enrolled in dose-escalation Group B receive polatuzumab vedotin 1.8 mg/kg by IV infusion on Day 1 of each 21-day cycle for up to a maximum of 6 cycles, starting on C1D1.

Patients receive mosunetuzumab 1 mg (DL1) on C1D8, 2 mg (DL2) on C1D15, and the first DL3 test dose on C2D1 by IV infusion. In Cycle 3 and beyond (up to 9-17 cycles), the mosunetuzumab DL3 dose is given on Day 1 of each cycle.

Schedule and dose level of mosunetuzumab plus polatuzumab vedotin are found in Table 7.

TABLE 7
Mosunetuzumab Plus Polatuzumab Vedotin Regimen (Group B)

| | | | | | | Cycles | Cycles | Cycles |
|---|---|---|---|---|---|---|---|---|
| | | | Cycle 1 | | | 2-6 | 7-9 | 10-17 |
| Agent | Dose | Route | Day 1 | Day 8 | Day 15 | Day 1 | Day 1 | Day 1 |
| Polatuzumab | 1.8 | IV | x | | | x | | |
| vedotin | mg/kg | | | | | | | |
| Mosunetuzumab | DL₁ | IV | | x | | | | |
| | DL₂ | IV | | | x | | | |
| | DL₃ | IV | | | | x | x | x |

DL = dose level.

Mosunetuzumab and polatuzumab vedotin may be given up to ±2 days from the scheduled date for Cycle 3 and beyond (i.e., with a minimum of 19 days between doses) for logistic/scheduling reasons.

Dose escalation in Group B uses the same modified 3+3 design and dose-escalation and de-escalation rules as Group A. The DLT assessment period for Group B is C1D8 through C2D21. The main difference between Group A and Group B dose-escalation rules is the timing of the administration of the first DL3 test dose, which occurs on C2D1 for Group B.

Group C-Cycle 1 Double-Step Fractionated Mosunetuzumab Escalation with Concurrent Administration of Polatuzumab Vedotin Starting in Cycle 2

Group C evaluates an alternate schedule of polatuzumab vedotin and mosunetuzumab, with mosunetuzumab given on a double-step fractionated schedule starting on C1D1 and polatuzumab vedotin given starting on C2D1. Patients enrolled in Group C receive mosunetuzumab 1 mg (DL1) on C1D1, 2 mg (DL2) on C1D8, and DL3 test dose on C1D15 by IV infusion. In Cycle 2 and beyond (up to 8-17 cycles) the mosunetuzumab DL3 dose is given on Day 1 of each 21-day cycle.

Patients receive polatuzumab vedotin 1.8 mg/kg by IV infusion on Day 1 of each cycle up to a maximum of 6 cycles, starting on C2D1.

Schedule and dose level of mosunetuzumab plus polatuzumab vedotin are found in Table 8.

TABLE 8
Mosunetuzumab Plus Polatuzumab Vedotin Regimen (Group C)

| | | | | | | Cycles | | Cycles |
|---|---|---|---|---|---|---|---|---|
| | | | Cycle 1 | | | 2-7 | Cycle 8 | 9-17 |
| Agent | Dose | Route | Day 1 | Day 8 | Day 15 | Day 1 | Day 1 | Day 1 |
| Polatuzumab | 1.8 | IV | | | | x | | |
| vedotin | mg/kg | | | | | | | |
| Mosunetuzumab | DL₁ | IV | x | | | | | |
| | DL₂ | IV | | x | | | | |
| | DL₃ | IV | | | x | x | x | x |

DL = dose level.

For logistic/scheduling reasons, C2D1 administration of mosunetuzumab and polatuzumab vedotin may be given up to +1 day from the scheduled date for Cycle 2 (i.e., with a minimum of 6 days after C1D15 dosing), and +2 days from the scheduled date for Cycle 3 and beyond (with a minimum of 19 days between doses).

Dose escalation in Group C uses a 3+3 design. The DLT assessment window in Group C is from C2D1 through C2D21 (FIG. 8), because polatuzumab vedotin is given in combination with mosunetuzumab starting on C2D1. Between C1D1 and C1D21, if a patient experiences a treatment-emergent toxicity that does not completely resolve to baseline level by C2D1, the patient may be considered unevaluable for dose-escalation decisions and MTD determination and be replaced by an additional patient at that same dose level and schedule. Dose escalation of mosunetuzumab DL3 alone is based on recommendations by the IMC for each successive cohort based on set escalation rules. A minimum of 3 patients are enrolled into each cohort, unless the first 2 enrolled patients experience a protocol-defined DLT, in which case enrollment into the cohort is terminated. If none of the first 3 DLT-evaluable patients experiences a DLT, enrollment of the next cohort at the next highest dose level may proceed. If 1 of the first 3 DLT-evaluable patients experiences a DLT, the cohort is expanded to 6 patients, and all 6 patients are evaluated for DLTs before any dose-escalation decision. If no additional patient experiences a DLT in the 6 DLT-evaluable patients, enrollment of the next cohort at the next highest dose level may proceed.

Otherwise, if the cohort has expanded to 6 patients, and 2 or more out of 6 DLT-evaluable patients experience a DLT after the administration of the first DL3 test dose, the MTD is deemed to have been exceeded and dose escalation stops. An additional 3 patients are then evaluated for DLTs at the preceding dose level, unless 6 patients have already been evaluated at that level. However, if the dose level at which the MTD is exceeded is >25% higher than the preceding dose level, 6 patients may be evaluated at an intermediate dose level.

If the MTD is exceeded at any dose level, the highest dose where fewer than 2 out of 6 DLT-evaluable patients (i.e., <33%) experience a DLT after the administration of the first DL3 test dose is declared the MTD. If the MTD is not exceeded at any dose level, the highest dose administered in this group is declared the maximum assessed dose. In the event that the initial mosunetuzumab DL3 test dose in combination with polatuzumab vedotin is above the MTD (i.e., ≥33% out of 6 DLT-evaluable patients experience a DLT after the administration of the first DL3 test dose), a reduced DL3 dose level that is at least 25% lower may be evaluated in an additional cohort of 3 to 6 patients. If this dose level is again above the MTD, further DL3 dose reductions of >25% of the preceding DL3 dose may be assessed in subsequent cohorts of 3-6 patients. The highest dose level where fewer than 2 out of 6 DLT-evaluable patients (i.e., <33%) experience DLTs is declared the MTD.

### Continued Dosing Beyond the DLT Assessment Period

Patients are eligible to receive additional cycles of study treatment with mosunetuzumab given in combination with polatuzumab vedotin every 21 days (the day of infusion being Day 1 of each cycle) beyond the DLT assessment period if they have no clinical signs or symptoms of progressive disease, and have not experienced Grade 4 non-hematologic adverse events with the possible exception of Grade 4 TLS. Patients who experience Grade 4 TLS may be considered for continued study treatment provided TLS resolves completely within 14 days and with Medical Monitor approval. All other study treatment related adverse events from prior study treatment administration must have decreased to Grade≤1 or baseline grade by the next administration. Exceptions on the basis of ongoing overall clinical benefit may be allowed after a careful assessment and discussion of benefit-risk with the patient by the study investigator and with approval from the Medical Monitor. Any treatment delay for toxicities not attributed to study treatment may not require study treatment discontinuation but must be approved by the Medical Monitor. Within each treatment group, a lower dose level on Day 1 of Cycle 3 or subsequent cycles may be administered to assess whether a lower dose than the mosunetuzumab DL3 administered in the first two cycles is sufficient to maintain clinical efficacy during later cycles. Once an RP2D is declared, the IMC may permit patients receiving mosunetuzumab at doses below the RP2D to be dose escalated to the RP2D. A patient may be dose escalated to the RP2D provided that no prior DLTs or dose reductions have occurred, and provided that the treating physician views such dose escalation is in the patient's best interest. Patients who complete study treatment without disease progression are monitored according to the schedule for post-treatment follow-up, including regularly scheduled tumor assessments until discontinuation from the post-treatment follow-up (e.g., due to progression).

### Expansion Phase

The expansion phase is designed to further assess the safety and efficacy of mosunetuzumab and polatuzumab vedotin at the RP2D and schedule determined in the dose-finding phase. Mosunetuzumab dose and schedule in combination with polatuzumab vedotin to be assessed in the expansion phase is determined based on IMC recommendation and in consultation with the investigators following a review of cumulative safety data in dose finding. Patients with R/R DLBCL, R/R FL, or R/R MCL are enrolled during the expansion phase and treated as described below.

### Single Arm Phase

Approximately 220 patients will be treated with mosunetuzumab plus polatuzumab vedotin in the single-arm expansion phase. The patient cohorts will be assigned to the following Arms:
Arm I: R/R FL (Grade 1-3a); 40 patients (mosunetuzumab IV plus polatuzumab vedotin).
Arm J: R/R DLBCL, transformed FL, or Grade 3b FL; 100 patients (mosunetuzumab IV plus polatuzumab vedotin)
Arm K: R/R MCL; 80 patients (mosunetuzumab SC plus polatuzumab vedotin)

Dose expansion Arms I, J, and K may be run sequentially or in parallel and may also be prioritized or suspended, at the discretion of the Sponsor.

Arm K begins enrolling patients after the RP2D is declared in Study GO40516 and after an optimal mosunetuzumab SC step-up dose has been identified in Study GO29781 (Arm F). The mosunetuzumab dosing schedule in combination with polatuzumab vedotin (concurrent or staggered dosing) in Arm K follows the same dosing schedule as the RP2D in study GO40516 (e.g., Group A, in which both drugs are administered starting on Cycle 1 Day 1), and the mosunetuzumab SC step-up doses in Arm K do not exceed the highest subcutaneous step-up doses assessed in Study GO29781. The first 6 safety-evaluable patients in Arm K are considered as a safety run-in group for the R/R MCL cohort. After these patients have completed at least one cycle of mosunetuzumab in combination with polatuzumab vedotin, the IMC reviews cumulative safety data in these patients to recommend either allowing the enrollment of additional patients or halting the rest of the Arm K expansion enrollment. The IMC may also recommend modifications to the dose and/or dosing schedule for Arm K, and a second safety cohort of approximately 6 patients are enrolled, if needed. An additional IMC review takes place after these patients in the second safety run-in group have completed at least one cycle of mosunetuzumab in combination with polatuzumab vedotin.

### Randomized Phase II for R/R DLBCL

Approximately 80-160 patients with R/R DLBCL are randomized at 1:1 ratio to the treatment with mosunetuzumab SC plus polatuzumab vedotin (Arm L) and rituximab plus polatuzumab vedotin (Arm M):
Arm L: R/R DLBCL; 40-80 patients (mosunetuzumab SC plus polatuzumab vedotin)
Arm M: R/R DLBCL; 40-80 patients (rituximab IV plus polatuzumab vedotin)

Patients are randomized 1:1 with the use of stratified permuted blocks. Randomization is stratified by the number of prior treatment regimens (one prior line of therapy versus >2 prior lines of therapy). Patients assigned to Arm M are allowed to receive mosunetuzumab SC plus polatuzumab vedotin if they meet the criteria for the crossover before or at the end of 8 cycles of rituximab plus polatuzumab vedotin therapy (Arm M-crossover).

### Mosunetuzumab Treatment Duration and Re-Treatment

Patients who initially respond or have SD to mosunetuzumab combined with polatuzumab vedotin may benefit from additional cycles beyond the initial 8 cycles of mosunetuzumab treatment, depending on anti-tumor responses to initial treatment. Patients are eligible for mosunetuzumab re-treatment (for all patients) or continued study treatment beyond the initial 8 cycles (for patients in Groups A, B, and C, and Arms I and J), either as single-agent or combined with polatuzumab vedotin.

The dose and schedule of study treatment to be administered for patients receiving re-treatment will be a previously tested dose and schedule that has cleared the DLT assessment period. The schema of the duration of initial study treatment and options for re-treatment (for all patients) or continued study treatment beyond the initial 8 cycles of study treatment (for Groups A, B, C, Arms I and J) are described in FIG. 4. The dose and schedule of administration of mosunetuzumab with or without polatuzumab vedotin based on the nature and timing of study treatment is described below.

Patients in Groups A, B, and C, and Arms I and J who achieve CR at primary response assessment (PRA) as the best overall response after 8 cycles of mosunetuzumab treatment do not receive any additional cycles of mosunetuzumab. Patients in Groups A, B, and C, and Arms I and J who achieve a PR or maintain SD at PRA as the best overall response after receiving 8 cycles of mosunetuzumab treatment may continue single-agent mosunetuzumab for up to a total of 17 cycles unless progressive disease or unacceptable toxicity is observed. Patients in Arm K who complete 17 cycles of mosunetuzumab treatment do not receive any additional cycles of mosunetuzumab. Patients in Arm L or Arm M-crossover who complete 8 cycles of mosunetuzumab treatment do not receive any additional cycles of mosunetuzumab. Additional rounds of re-treatment with mosunetuzumab with or without polatuzumab vedotin are permitted, following the re-treatment rules described.

If the time between the last dose of initial treatment and first dose of re-treatment including step dose is >6 weeks, administer mosunetuzumab at previous schedule including Cycle 1 step-up. If the time between the last dose of initial treatment and first dose of re-treatment including step dose is <6 weeks, administer mosunetuzumab at previous schedule target dose every 21 days (e.g., Cycle 2 dose in double-step fractionated escalation dosing or Cycle 3 dose in step-load-base dosing); no mosunetuzumab step-up is needed. The formulation of mosunetuzumab for re-treatment must be the same as the original formulation administered during initial treatment for that individual patient (e.g., if SC was administered in the initial treatment phase for a patient, then SC must also be administered for re-treatment).

Crossover for Patients from Arm M (Rituximab plus Polatuzumab Vedotin) to Arm M-crossover (Mosunetuzumab SC plus Polatuzumab Vedotin)

Patients in Arm M have the option to receive treatment with mosunetuzumab SC plus polatuzumab vedotin (Arm M-crossover) within 6 weeks if either of the following criteria is met: the patient has disease progression before or at the end of 8 cycles of Arm M treatment or the disease assessment is stable disease at the end of 8 cycles of Arm M treatment.

Treatment cycles of polatuzumab vedotin in Arm M-crossover is reduced so that the total number of treatment cycles of polatuzumab vedotin in Arm M and Arm M-crossover do not exceed 8 cycles. Patients in Arm M-crossover will receive a total of 8 cycles of mosunetuzumab SC as in Arm L, regardless of the number of rituximab treatment cycles given in Arm M.

### Inclusion Criteria

Approximately up to 422 patients are enrolled in this study (approximately 9-42 patients with R/R DLBCL or FL in the dose-finding phase, and approximately 100 patients with R/R DLBCL, 40 patients with R/R FL, 80 patients with R/R MCL in the single-arm expansion phase, and 80-160 patients with R/R DLBCL in the randomized Phase II portion).

Patients meet the following criteria:
Signed Informed Consent Form
Age >18 years at time of signing Informed Consent Form
Able to comply with the study protocol and procedures in the investigator's judgment
ECOG PS of 0, 1, or 2
Life expectancy of at least 12 weeks
Histologically confirmed FL, DLBCL, or MCL from the following diagnoses by 2016 WHO classification of lymphoid neoplasms, that have either relapsed or have become refractory to a prior regimen as defined below:
   R/R FL (Groups A, B, C, and I)
      FL Grade 1, 2, or 3a
      In situ follicular neoplasia
      Duodenal-type FL
      Pediatric-type FL
      Relapsed to prior regimen(s) after having a documented history of response (CR, CR unconfirmed [CRu], or PR) of ≥6 months in duration from completion of regimen(s)
      Refractory to any prior regimen, defined as no response to the prior therapy, or progression within 6 months of completion of the last dose of therapy
      Must have received at least one prior systemic treatment regimen containing an anti-CD20-directed therapy
   R/R DLBCL (Groups A, B, and C; Arms J, L, and M)
      DLBCL not otherwise specified (NOS) (including germinal center B-cell type and activated B-cell type)
      T-cell/histiocyte-rich large B-cell lymphoma
      High-grade B-cell lymphoma with MYC and BCL-2 and/or BCL-6 rearrangements; must be R/R to standard therapies for aggressive NHL
      EBV+DLBCL, NOS
      HHV8+DLBCL, NOS.
      High grade B-cell lymphoma, NOS
      Anaplastic lymphoma kinase (ALK)+large B-cell lymphoma
      Grade 3b FL; Grade 3b FL is an eligible diagnosis for enrollment in the DLBCL cohort but must be R/R to standard therapies for aggressive NHL.
      Relapsed to prior regimen(s) after having a documented history of response (CR, CRu, or PR) of ≥6 months in duration from completion of regimen(s)
      Refractory to any prior regimen, defined as no response to the prior therapy, or progression within 6 months of completion of the last dose of therapy.
      Must have received at least one prior systemic treatment regimen containing an anti-CD20-directed therapy
      Transformed FL is an eligible diagnosis for enrollment in the DLBCL cohort but must be R/R to standard therapies for transformed FL. Patients with Patients with Richter's transformation are not eligible for study.
   R/R MCL
      Relapsed to prior regimen(s) after having a documented history of response (CR, CRu, or PR) of ≥6 months in duration from completion of regimen(s)
      Refractory to any prior regimen, defined as no response to the prior therapy, or progression within 6 months of completion of the last dose of therapy.
      Must have received at least two prior systemic treatment regimens, which includes all of an anti-CD20-directed therapy; a Bruton's tyrosine kinase (BTK) inhibitor; and an anthracycline or bendamustine
Measurable disease, defined as at least one bi-dimensionally measurable nodal lesion, defined as >1.5 cm in its longest dimension, or at least one bi-dimensionally measurable extranodal lesion, defined as >1.0 cm in its longest dimension.
Pathology report for the initial histopathology diagnosis and the most recent histopathology diagnosis prior to study entry must be provided.
   Patients with transformed FL must also provide the pathology report at the time of disease transformation.
   The results of all tests conducted on the tissue at initial diagnosis, including, but not limited to, tests assessing cell of origin, BCL2 and MYC abnormalities, are provided if done.
   For patients with MCL, results of all tests conducted on the tissue at initial diagnosis and/or relapse, including, but not limited to, the MCL subtype (nodular and blastoid), Ki-67 proliferation index, and TP53 mutation status, should be provided if done.
Tumor accessible for biopsy; Patients who are unable to undergo biopsy procedures may be eligible for study enrollment if archival tumor tissue samples (paraffin blocks or at least 20 unstained slides), in place of a fresh biopsy, can be sent to the Sponsor.
Adverse events from prior anti-cancer therapy resolved to ≤Grade 1
Laboratory values as follows:
   Hepatic Function

$$AST and ALT \leq 2.5 \times ULN$$

$$Total bilirubin \leq 1.5 \times ULN$$

Patients with a documented history of Gilbert syndrome and in whom total bilirubin elevations are accompanied by elevated indirect bilirubin are eligible.
Hematologic Function
   Platelet count ≥75,000/mm3 without transfusion within 14 days prior to first dose of study treatment

$$ANC \geq 1 \; 000 / {mm}^{3}$$

Total hemoglobin ≥10 g/dL without transfusion within 21 days prior to first dose of study treatment
INR≤1.5× ULN in the absence of therapeutic anticoagulation
PTT or aPTT≤1.5× ULN in the absence of lupus anticoagulant
Patients with extensive marrow involvement of NHL and/or disease-related cytopenias (e.g., immune thrombocytopenia) may be enrolled if Platelet count ≥50,000/mm3 without transfusion within 14 days; ANC >500/mm3; and any hemoglobin but without transfusion within 7 days.
Serum creatinine≤ULN or estimated creatinine CL >60 mL/min by Cockroft-Gault method or other institutional standard methods, e.g., based on nuclear medicine renal scan
For women of childbearing potential: agreement to remain abstinent (refrain from heterosexual intercourse) or use contraceptive measures, and agreement to refrain from donating eggs
For men: agreement to remain abstinent (refrain from heterosexual intercourse) or use a condom, and agreement to refrain from donating sperm

### Exclusion Criteria

Patients with any of the following criteria are excluded:
Inability to comply with protocol-mandated hospitalization and activity restrictions
Pregnant or breastfeeding, or intending to become pregnant during the study or within 3 months after the final dose of mosunetuzumab, 12 months after the final dose of polatuzumab vedotin, 12 months after the final dose of rituximab, and 3 months after the final dose of tocilizumab, as applicable.
   Women of childbearing potential must have a negative serum pregnancy test result within 7 days prior to initiation of study treatment.
Prior treatment with mosunetuzumab or other CD20-directed bispecific antibodies
Prior treatment with polatuzumab vedotin
Current >Grade 1 peripheral neuropathy
Prior use of any monoclonal antibody, radioimmunoconjugate or ADC within 4 weeks before first dose of study treatment.
Treatment with any chemotherapeutic agent, or treatment with any other anti-cancer agent (investigational or otherwise) within 4 weeks or 5 half-lives of the drug, whichever is shorter, prior to first dose of study treatment
Treatment with radiotherapy within 2 weeks prior to the first dose of study treatment
   If patients have received radiotherapy within 4 weeks prior to the first study treatment administration, patients must have at least one measurable lesion outside of the radiation field.
   Patients who have only one measurable lesion that was previously irradiated but subsequently progressed are eligible.
Autologous SCT within 100 days prior to first study treatment administration
Prior treatment with CAR-T therapy within 30 days before first study treatment administration.
Current eligibility for autologous SCT in patients with R/R DLBCL, R/R transformed FL, or R/R Grade 3b FL
Prior allogeneic SCT
Prior solid organ transplantation
Patients with history of confirmed progressive multifocal leukoencephalopathy (PML)
History of severe allergic or anaphylactic reactions to monoclonal antibody therapy (or recombinant antibody-related fusion proteins)
History of other malignancy that could affect compliance with the protocol or interpretation of results
   Patients with a history of curatively treated basal or squamous cell carcinoma of the skin or in situ carcinoma of the cervix are allowed.
   Patients with a malignancy that has been treated with curative intent will also be allowed if the malignancy has been in remission without treatment for >2 years prior to first study treatment administration.
Current or past history of CNS lymphoma
Current or past history of CNS disease, such as stroke, epilepsy, CNS vasculitis, or neurodegenerative disease
   Patients with a history of stroke who have not experienced a stroke or transient ischemic attack in the past 2 years and have no residual neurologic deficits as judged by the investigator are allowed.
   Patients with a history of epilepsy who have had no seizures in the past 2 years while not receiving any anti-epileptic medications are allowed in the expansion cohorts only.
Significant cardiovascular disease such as New York Heart Association Class Ill or IV cardiac disease, myocardial infarction within the last 6 months, unstable arrhythmias, or unstable angina
Significant active pulmonary disease (e.g., bronchospasm and/or obstructive pulmonary disease)
Known active bacterial, viral, fungal, mycobacterial, parasitic, or other infection (excluding fungal infections of nail beds) at study enrollment, or any major episode of infection requiring treatment with IV antibiotics or hospitalization (relating to the completion of the course of antibiotics) within 4 weeks prior to first study treatment administration
Known or suspected chronic active EBV infection
Recent major surgery within 4 weeks prior to first study treatment administration
   Protocol-mandated procedures (e.g., tumor biopsies and bone marrow biopsies) are permitted.
Positive test results for chronic hepatitis B infection (defined as positive hepatitis B surface antigen [HBsAg] serology)
   Patients with occult or prior hepatitis B infection (defined as positive total hepatitis B core antibody and negative HBsAg) may be included if HBV DNA is undetectable at the time of screening. These patients must be willing to undergo monthly DNA testing and appropriate antiviral therapy as indicated.
Acute or chronic HCV infection
   Patients who are positive for HCV antibody must be negative for HCV by PCR to be eligible for study participation.
Positive serologic test results for HIV infection
Administration of a live, attenuated vaccine within 4 weeks before first dose of study treatment administration or anticipation that such a live, attenuated vaccine will be required during the study
   Patients must not receive live, attenuated vaccines (e.g., FluMist®) while receiving study treatment and after the last dose until B-cell recovery to the normal ranges. Killed vaccines or toxoids should be given at least 4 weeks prior to the first dose of study treatment to allow development of sufficient immunity.
   Inactivated influenza vaccination should be given during local influenza season only. Investigators should review the vaccination status of potential study patients being considered for this study and follow the U.S. Centers for Disease Control and Prevention guidelines for adult vaccination with any other non-live vaccines intended to prevent infectious diseases prior to study.
History of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barre syndrome, multiple sclerosis, vasculitis, or glomerulonephritis
   Patients with a history of autoimmune-related hypothyroidism on a stable dose of thyroid replacement hormone may be eligible.
   Patients with controlled Type 1 diabetes mellitus who are on an insulin regimen are eligible for the study.
   Patients with a history of disease-related immune thrombocytopenia purpura, autoimmune hemolytic anemia, or other stable autoimmune diseases may be eligible after review and approval by the Medical Monitor.
Received systemic immunosuppressive medications (including, but not limited to, cyclophosphamide, azathioprine, methotrexate, thalidomide, and anti-tumor necrosis factor agents) with the exception of corticosteroid treatment≤10 mg/day prednisone or equivalent within 2 weeks prior to first dose of study treatment
   Patients who received acute, low-dose, systemic immunosuppressant medications (e.g., single dose of dexamethasone for nausea or B symptoms) may be enrolled in the study after discussion with and approval of the Medical Monitor.
   The use of inhaled corticosteroids is permitted.
   The use of mineralocorticoids for management of orthostatic hypotension is permitted.
   The use of physiologic doses of corticosteroids for management of adrenal insufficiency is permitted.
Clinically significant history of liver disease, including viral or other hepatitis, current alcohol abuse, or cirrhosis.
Any serious medical condition or abnormality in clinical laboratory tests that, in the investigator's or Medical Monitor's judgment, precludes the patient's safe participation in and completion of the study, or which could affect compliance with the protocol or interpretation of results

### Method of Administration

Mosunetuzumab is administered by IV infusion (Groups A, B, C; and Arms I, and J) or SC injection (Arms K and L) in combination with polatuzumab vedotin or as a single agent, depending on the assigned treatment regimen. Compatibility testing has shown that mosunetuzumab is stable in infusion sets and IV bags. When administered intravenously, mosunetuzumab is delivered by syringe pump via an IV infusion set or an IV bag with a final mosunetuzumab volume determined by the dose, and an in-line filter should not be used. When administered subcutaneously, mosunetuzumab will be delivered by medical syringe with a final mosunetuzumab volume not to exceed 2.0 mL. Mosunetuzumab is administered to well-hydrated patients.

For IV infusion of mosunetuzumab: corticosteroid premedication consisting of dexamethasone 20 mg IV or methylprednisolone 80 mg IV are administered 1 hour prior to the administration of each mosunetuzumab dose. In addition, premedication with oral acetaminophen or paracetamol (e.g., 500-1000 mg) and/or 50-100 mg diphenhydramine may be administered per standard institutional practice prior to administration of mosunetuzumab. Initially, mosunetuzumab is infused over 4 hours±15 minutes. The infusion may be slowed or interrupted for patients experiencing infusion-associated symptoms. In the absence of infusion-related adverse events, the infusion time of mosunetuzumab in Cycle 2 (Group A) or Cycle 3 (Groups B and C) and beyond may be reduced to 2 hours (+15 minutes), after consultation with the Medical Monitor.

For SC injection of mosunetuzumab: corticosteroid premedication consisting of dexamethasone 20 mg IV or PO (orally) or methylprednisolone 80 mg IV or PO are administered 1 hour prior to the administration of each mosunetuzumab dose. In addition, premedication with oral acetaminophen or paracetamol (e.g., 500-1000 mg) and/or 50-100 mg diphenhydramine may be administered per standard institutional practice prior to administration of mosunetuzumab. Initially, mosunetuzumab is administered by injection over 30 seconds to 2 minutes.

Polatuzumab vedotin is administered by IV infusion in combination with mosunetuzumab or in combination with bendamustine and rituximab, depending on the assigned treatment regimen. The dose of polatuzumab vedotin for each patient is 1.8 mg/kg. The total dose of polatuzumab vedotin for each patient depends on the patient's weight on C1D1 (or within 96 hours before C1D1). If the patient's weight within 96 hours prior to Day 1 of a given treatment cycle increases or decreases >10% from the weight obtained during screening, the new weight is used to calculate the dose. The weight that triggered a dose adjustment is taken as the new reference weight for future dose adjustments. All subsequent doses are modified accordingly. The initial dose is administered over 90 (+10) minutes to patients who are well hydrated. Premedication (e.g., 500-1000 mg of oral acetaminophen or paracetamol and 50-100 mg diphenhydramine as per institutional standard practice) may be administered to an individual patient before administration of polatuzumab vedotin. Administration of corticosteroids is permitted at the discretion of the treating physician. If prior infusions have been well tolerated, subsequent doses of polatuzumab vedotin may be administered over 30 (±10) minutes, followed by a 30-minute observation period after the infusion.

On days when mosunetuzumab is given in combination with polatuzumab vedotin, polatuzumab vedotin is administered first, followed by mosunetuzumab. Polatuzumab vedotin and mosunetuzumab are administered as described above. The time interval between the end of infusion of polatuzumab vedotin and the start of mosunetuzumab infusion/injection is at least 90 minutes for Cycles 1 and 2. For Cycle 3 and beyond, if prior polatuzumab vedotin infusions have been well tolerated, the time interval between the end of infusion of polatuzumab vedotin and the start of mosunetuzumab infusion should be at least 60 minutes. Patients receive a total of 6 cycles of polatuzumab vedotin. Patients receive a total of 8 cycles of mosunetuzumab, with the exception of patients in Group B, who may receive 9 cycles because of a delayed start of mosunetuzumab. Patients in Group B receive the same number of mosunetuzumab doses as patients in Groups A and B. Patients in Groups A, B, and C, and Arms I and J may be treated with mosunetuzumab beyond 8 dosing cycles for a total of 17 dosing cycles. Dosing cycles are 21 days in length.

Rituximab is administered by IV infusion in combination with polatuzumab vedotin and bendamustine. Rituximab 375 mg/m² is administered by IV infusion. No dose modifications of rituximab are allowed. The rituximab administration is completed at least 30 minutes before administration of other study treatments. The infusion of rituximab can be split over two days if the patient is at increased risk for an IRR (high tumor burden, high peripheral lymphocyte count). Administration of rituximab can be continued on the following day, if needed, for patients who experience an adverse event during the rituximab infusion. If a dose of rituximab is split over 2 days, both infusions must occur with appropriate premedication and at the first infusion rate. All rituximab infusions are administered to patients after premedication with oral acetaminophen (e.g., 650-1000 mg) and an antihistamine such as diphenhydramine hydrochloride (50-100 mg) 30-60 minutes before starting each infusion (unless contraindicated). An additional glucocorticoid (e.g., 100 mg IV prednisone or prednisolone or equivalent) is allowed at the investigator's discretion. For patients who did not experience infusion-related symptoms with their previous infusion, premedication at subsequent infusions may be omitted at the investigator's discretion. During the treatment period, rituximab must be administered to patients in a setting where full emergency resuscitation facilities are immediately available. Rituximab is administered as a slow IV infusion through a dedicated line. IV infusion pumps (such as the Braun Infusomat Space) are used to control the infusion rate of rituximab. After the end of the first infusion, the IV line or central venous catheter remains in place for >2 hours in order to administer IV drugs if necessary.

Tocilizumab is administered when necessary. Any overdose or incorrect administration of tocilizumab is noted on the Study Drug Administration eCRF. Adverse events associated with an overdose or incorrect administration of study drug is recorded on the Adverse Event eCRF.

### Assessments and Monitoring

Patients are closely monitored for safety and tolerability throughout the study. FLIPI and FLIPI2 clinical factors obtained at diagnosis and at enrollment are collected for patients with FL. IPI clinical factors at diagnosis and at enrollment are collected for patients with DLBCL or transformed FL. All evaluable or measurable disease are documented at screening and re-assessed at each subsequent tumor evaluation. Response is assessed by the IRC and the investigator on the basis of physical examinations, CT scans, PET-CT scans, and bone marrow examinations (if applicable) using the Lugano 2014 criteria.

PET and diagnostic-quality CT scans are required at screening, at the interim response assessment, and at the PRA visit. Additionally, if disease progression or relapse is suspected before the PRA, both PET and diagnostic-quality CT scans are performed for tumor assessment using the Lugano 2014 criteria to assess overall response to study treatment. PET-CT scans include skull-base to mid-thigh. Full-body PET-CT scan is performed when clinically appropriate. CT scans with oral and IV contrast include chest, abdomen, and pelvic scans; CT scans of the neck are if clinically indicated. CT scans for response assessment may be limited to areas of prior involvement only if required by local regulatory authorities. A baseline brain MRI with gadolinium contrast is obtained in all patients unless medically contraindicated as part of the baseline neurologic assessment.

Bone marrow examinations are required at screening for staging purposes for patients with FL. For patients with DLBCL, screening PET/CT scans may be utilized to assess bone marrow involvement; bone marrow examinations are not required unless clinically indicated. The screening bone marrow may be obtained within 28 days before the start of study treatment. In addition, the definition of CR for CT-based response requires clearing of previously infiltrated bone marrow. Bone marrow examinations include a biopsy for morphology and an aspirate for local hematology (local flow studies are optional). Repeat bone marrow examination is required at the PRA visit for patients who achieve a CR for CT-based response if there was bone marrow infiltration at screening or at time of relapse or transformation if bone marrow involvement is suspected. In patients with a PR and continued bone marrow involvement, a subsequent bone marrow examination may be required to confirm a CR for CT-based response at a later timepoint. For patients with DLBCL, PET/CT scans may be utilized to assess bone marrow involvement, and repeat bone marrow examinations are not required unless clinically indicated.

### Laboratory, Biomarker, and Other Biological Samples

Exploratory biomarker research in tumor tissue and blood may include, but is limited to, analysis of genes or gene signatures associated with tumor immunobiology, prognostic or predictive markers associated with response to mosunetuzumab and polatuzumab vedotin, markers associated with T-cell activation, localization, and activation status of immune cells and their subsets, and may involve extraction of DNA, circulating tumor DNA or RNA, analysis of somatic mutations, and use of next-generation sequencing (NGS). Assays for exploratory analysis include, but are not limited to, IHC, immunofluorescence, and RNA sequencing. Additional exploratory biomarkers may be assessed based on evolving clinical and nonclinical data.

Samples for the following laboratory tests are sent to the study site's local laboratory for analysis:
Hematology: CBC (including hemoglobin, hematocrit, RBC, WBC), platelet count, ANC, absolute lymphocyte count, and other cells
Coagulation: aPTT, PT, INR, and fibrinogen (e.g., collected when monitoring systemic immune activation events (e.g., MAS/HLH, severe CRS)
Quantitative Igs (IgA, IgG, and IgM)
Serum chemistry: sodium, potassium, chloride, bicarbonate, glucose, BUN or urea, creatinine, calcium, magnesium, phosphorous, total and direct bilirubin, total protein, albumin, ALT, AST, ALP, gamma-glutamyl transferase, LDH, and uric acid
Beta-2 microglobulin
C-reactive protein
Serum ferritin
Viral serology and detection (e.g., hepatitis B (HBsAg, hepatitis B surface antibody [HBsAb], and hepatitis B core antibody [HBcAb]; HBV DNA by PCR if acute or chronic HBV infection cannot be ruled out by serology results [www.cdc.gov/hepatitis/hbv/pdfs/serologicchartv8.pdf]), HCV antibody; HCV RNA by PCR if the patient is HCV antibody positive, and/or EBV and CMV by quantitative PCR using peripheral blood samples, HIV serology)
Pregnancy test
All women of childbearing potential undertake a serum pregnancy test at screening (within 7 days prior to C1D1). Urine or serum pregnancy tests are performed at specified subsequent visits. If a urine pregnancy test is positive, it must be confirmed by a serum pregnancy test.
Samples for the following laboratory tests are sent to one or several central laboratories or to the Sponsor or designee for analysis:
Whole blood samples for flow cytometry and PBMC isolation.
Plasma (e.g., for cytokines, including but not limited to IL-6 and IFN-γ, circulating CD20) for assessment of minimal residual disease status (expansion cohorts only)
Serum samples for measurement of mosunetuzumab, polatuzumab vedotin, rituximab, obinutuzumab, and/or tocilizumab concentrations using validated PK assay
Lithium plasma for polatuzumab vedotin acMMAE and unconjugated MMAE using a validated PK assay
Blood samples are collected for viral infection test for quantitative PCR detection of viral infection that may include, but is not limited to, EBV and CMV
Serum samples for measurement of ADAs to mosunetuzumab, polatuzumab vedotin, and/or tocilizumab using validated assays
Tumor biopsies from safely accessible tumor sites (i.e., without unacceptable risk of a major procedural complication(s) per investigator assessment). Samples collected via resection, core-needle biopsy, or excisional, incisional, punch, or forceps biopsies are preferred. The specimen must contain adequate evaluable tumor cells (≥20% for excisional biopsy and ≥50% for core biopsy). Tumor biopsies are required at the following time-points: pretreatment, on-treatment, and re-treatment.

### Efficacy Analyses

The primary efficacy endpoint for Arms I, J, K, L and M is best ORR, defined as the percentage of patients with CR or PR at any time based on PET-CT and/or CT scan and as determined by the IRC using the Lugano 2014 criteria (Cheson et al. 2014). Patients with missing or no response assessments are classified as non-responders. The best ORRs between Arms L and M are compared approximately 6 months after the last patient is enrolled in the randomized Phase II portion. A best ORR delta of ≥10% between Arm L over Arm M demonstrates clinical benefits of mosunetuzumab in combination with polatuzumab vedotin over rituximab in combination with polatuzumab vedotin.

Comparison with respect to ORR between the treated patient population and historical controls will be tested for Arm J (R/R DLBCL/trFL/FL3b) and Arm K (R/R MCL). The control ORR is assumed to be 42% for Arm J and 30% for Arm K.

Secondary efficacy endpoints of response rates are analyzed using the Lugano 2014 criteria as follows. Patients with missing or no response assessments are classified as non-responders.
CR rate at PRA as measured by PET-CT scan and as determined by the investigator and by the IRC
ORR, defined as the percentage of patients with PR or CR at PRA as measured by PET-CT scan and as determined by the IRC and by the investigator
Best ORR, defined as the percentage of patients with any PR or CR while in the study as measured by PET-CT and/or CT only and as determined by the investigator
Best CR rate, defined as the percentage of patients with any CR while in the study as measured by PET-CT and/or CT only and as determined by the IRC and by the investigator

DOR is defined as the time from the first occurrence of a documented objective response (CR or PR) to the time of disease progression, relapse, or death from any cause, whichever occurs first. For patients who do not experience death or disease progression, DOR is censored at the date of last evaluable tumor assessment. DOR is assessed per IRC and per investigator, using the Lugano 2014 criteria (Cheson et al. 2014; see Appendix 7). Analyses of DOR includes only patients with objective responses (CR or PR) at any time in the study. The Kaplan-Meier method (Kaplan and Meier 1958) is used to estimate the distribution of DOR and median DOR (if analytically possible) for each treatment arm, with 95% CI for the median DOR constructed using the Brookmeyer-Crowley method (Brookmeyer and Crowley 1982). DOR is also summarized for the subgroups of patients whose best objective response is PR and patients whose best objective response is CR.

PFS is defined as the time from first study treatment to the first occurrence of disease progression, relapse, or death from any cause, whichever occurs first. For patients who do not experience disease progression, relapse or death, PFS is censored at the date of last evaluable tumor assessment. For patients who do not have a post-baseline evaluable tumor assessment, PFS is censored at D1. PFS is assessed per IRC and per investigator, using the Lugano 2014 criteria (Cheson et al. 2014). The Kaplan-Meier method (Kaplan and Meier 1958) is used to estimate the distribution of PFS, median (if analytically possible), and 6-month and 1-year PFS for each treatment arm. The Brookmeyer-Crowley method (Brookmeyer and Crowley 1982) is used to construct the 95% CI for the median PFS. The Greenwood's formula is used to provide standard errors and the corresponding 95% Cls for 6-month PFS and 1-year PFS.

EFS is defined as the time from first study treatment to the first occurrence of any treatment failure including disease progression, relapse, initiation of NALT, or death. For patients who do not experience the specified event (disease progression/relapse, death, start of an NALT), EFS is censored at the date of last evaluable tumor assessment. For patients who do not have a post-baseline evaluable tumor assessment or documentation of NALT, EFS is censored at D1. EFS is assessed per IRC and per investigator, using the Lugano 2014 criteria (Cheson et al. 2014). Analyses of EFS is identical to those outlined previously for PFS.

OS is defined as the time from first study treatment to the date of death from any cause. Patients who have not died will be censored at the last date known to be alive. Analyses of OS will be identical to those outlined previously for PFS.

### Safety Analyses

All safety analyses is based on the safety-evaluable population (i.e., patients who received any study treatment), according to the actual treatment received. Safety is assessed through summaries of adverse events, changes in laboratory test results, changes in ECGs, changes in ADAs, and changes in vital signs. All collected adverse event data are listed by phase of the study, assigned treatment arm and dose level, and patient number. All adverse events occurring on or after first study treatment are summarized by mapped terms, appropriate thesaurus levels, and toxicity grade per NCI CTCAE v5.0 (or, for CRS events, per ASTCT consensus grading criteria). In addition, all serious adverse events, including deaths, are listed separately and summarized. DLTs and adverse events leading to treatment discontinuation will also be separately listed. Selected laboratory data are listed, with values outside of normal ranges identified.

### Pharmacokinetic Analysis

Individual and mean serum concentration of mosunetuzumab versus time data are tabulated and plotted by dose level. The Cₘₐₓ and Cₘᵢₙ, of mosunetuzumab and polatuzumab vedotin are summarized. For patients with dense PK sampling scheme, additional PK parameters are calculated including area AUC, CL, and Vₛₛ, as appropriate for data collected. Estimates for these parameters are tabulated and summarized. Serum trough and maximum concentrations for rituximab, where applicable, are summarized, as appropriate and as data allow. Compartmental, non-compartmental, and/or population methods may be considered. Pre-dose rituximab and obinutuzumab concentrations are summarized for patients who received prior rituximab or obinutuzumab treatments. Additional PK analyses may be conducted as appropriate.

### Immunogenicity Analysis

Validated screening, titering, and confirmatory assays are employed to assess ADAs before, during, and after treatment with mosunetuzumab and polatuzumab vedotin. The immunogenicity analysis population consists of all patients with at least one ADA assessment. Patients are considered to be negative for ADAs if they are ADA negative at all time-points. Patients are considered to be treatment unaffected if they are ADA positive at baseline but do not have any postbaseline samples with a titer that is at least 4-fold greater than the titer of the baseline sample. Patients are considered to have treatment-induced ADA responses if they are ADA negative or missing data at baseline and then develop an ADA response following study drug administration. Patients are considered to have treatment-enhanced ADA responses if they are ADA positive at baseline and the titer of one or more postbaseline samples is at least 4-fold greater (i.e., at least 0.60 titer unit) than the titer of the baseline sample. The relationship between ADA status and safety, efficacy, PK, and biomarker endpoints may also be assessed as appropriate and reported in a descriptive manner via subgroup analyses.

### Biomarker Analysis

Exploratory analyses of biomarkers related to tumor and disease biology as well as the mechanisms of action of polatuzumab vedotin and mosunetuzumab are conducted. The association between candidate biomarkers and ORR rate and other measures of efficacy and safety, with treatment and independent of treatment, are explored to assess potential predictive and prognostic value, respectively. The effects of baseline prognostic characteristics, including NHL subtypes and mutation profiles on efficacy, are evaluated using univariate and/or multivariate statistical methods such as Cox regression and logistic regression. Exploratory PD analyses may include assessments of cytokines, T-cell activation and proliferation, NK cells, B cells as well as other assessments of biomarkers in both tumor tissue and blood when available.

### Example 3. Results for an Open-Label, Multicenter, Phase Ib/II Trial Evaluating the Safety, Tolerability, Pharmacokinetics, and Efficacy of Mosunetuzumab (BTCT4465A) in Combination with Polatuzumab Vedotin in Patients with B-Cell Non-Hodgkin Lymphoma

GO40516 pivotal Arm J met its primary endpoint of ORR by IRC in adult patients with relapsed or refractory large B-cell lymphoma (R/R LBCL) who are not candidates for autologous stem-cell transplantation (ASCT). Arm J (mosunetuzumab “Mosun” IV+polatuzumab vedotin (“Pola”), n=98) overall response rate (ORR) by IRC=61.2% [50.9%, 70.9%], p-value <0.0001 vs. historical control of 42%. CCOD of Jan. 30, 2024. Median (range) follow-up for pivotal Arm J (in months): 31.3 (0-43).

### Efficacy Results

Secondary efficacy endpoints showed consistent trend:
Arm J ORR by INV=62.2% [51.9%, 71.8%] (61 responders out of 98)
Arm J complete response rate (CRR) by IRC=49% [38.7%, 59.3%] (48 complete responders out of 98)
   Duration of response (DOR) by IRC (months): median DOR (mDOR)=27.6 [16.2, NE].
9m DOR (rate of DOR at 9 months)=75.4% [63.10, 87.7]. 12m DOR=70.7% [57.5, 83.8]. 24m DOR=57.5% [42.5, 72.5]
Progression-free survival (PFS) by IRC (months): median PFS (mPFS)=14.1 [8.8, 26.9].
   9m PFS=60.4% [49.7, 71.1]. 12m PFS=52.4% [41.1, 63.8]. 24m PFS=40.1% [28.2, 52.0]
Overall survival (OS) (months): median OS (mOS)=26.9 [14.8, NE].
   9m OS=70.3% [61.1, 79.6]. 12m OS=64.9% [55.2, 74.5]. 24m OS=50.5% [40.3, 60.7]

Randomized Arms L & M (Mosun SC+Pola vs. R+Pola) showed consistent trend of efficacy with IV Mosun formulation, and meaningful results to demonstrate contribution of effect (AORR ≥10% between Arm L over Arm M) for Mosun:
Arm L ORR by IRC=77.5% [61.6%, 89.2%] vs Arm M ORR by IRC=50.0% [33.8%, 66.2%]
DOR by IRC: HR=0.4 [0.13, 1.19], p-value=0.087. Arm L 9m DOR=78.7% [62.1, 95.4]
PFS by IRC: HR*=0.48 [0.24, 0.99], p-value=0.043. Arm L 9m PFS=71.7% [56.6, 86.8]. 12m PFS=64.2% [47.4, 80.9]
OS: HR*=0.87 [0.41, 1.85], p-value=0.71. Arm L 9m OS=79.1% [66.2, 92.0]. 12m OS=73.8% [59.9, 87.8]

Group A1 patients were administered 1/2/9 mg dosing regimen (e.g., C1D1 dose of 1 mg on Day 1 of Cycle 1; C1D2 dose of 2 mg on Day 8 of Cycle 1; C1D3 dose of 9 mg on Day 15 of Cycle 1; and C2D1+dose of 9 mg on Day 1 of Cycle 2 onwards). Group A2 patients were administered Jan. 2, 2020 mg dosing regimen (e.g., C1D1 dose of 1 mg on Day 1 of Cycle 1; C1D2 dose of 2 mg on Day 8 of Cycle 1; C1D3 dose of 20 mg on Day 15 of Cycle 1; and C2D1+dose of 20 mg on Day 1 of Cycle 2 onwards). Group A3 patients were administered Jan. 2, 1940 mg dosing regimen (e.g., C1D1 dose of 1 mg on Day 1 of Cycle 1; C1D2 dose of 2 mg on Day 8 of Cycle 1; C1D3 dose of 40 mg on Day 15 of Cycle 1; and C2D1+dose of 40 mg on Day 1 of Cycle 2 onwards). Group A4 patients were administered Jan. 2, 1960/30 mg dosing regimen (e.g., C1D1 dose of 1 mg on Day 1 of Cycle 1; C1D2 dose of 2 mg on Day 8 of Cycle 1; C1D3 dose of 60 mg on Day 15 of Cycle 1; C2D1 dose of 60 mg on Day 1 of Cycle 2; and C3D1+dose of 30 mg on Day 1 of Cycle 3 onwards). Expansion cohort patients were administered Jan. 2, 1960/30 mg dosing regimen. Hospitalization was not required for expansion cohort patients.

Patients who participated in this study are summarized below in Table 9.

TABLE 9
Patient Population

| | Number of patients | | | | |
|---|---|---|---|---|---|
| | | | Arm J, | Arm L, | |
| | Group A, | Group A, | Mosun IV + | Mosun SC + | Arm M, |
| | all patients | DLBCL pts | Pola | Pola | R + Pola |
| Efficacy-evaluable | 22 | 19 | 98 | 40 | 40 |
| Safety-evaluable | 22 | 19 | 98 | 40 | 39* |

Patient demographics and baseline characteristics are summarized in FIG. 5. The benchmark/control ORR for the primary efficacy of IV Mosun+Pola was determined based on prior studies DCS4968g (1.8 mg/kg or 2.4 mg/kg polatuzumab vedotin monotherapy) and GO27834 (1.8 mg/kg or 2.4 mg/kg in combination with obinutuzumab or rituximab, respectively). ORR was about 50% for 1.8 mg/kg polatuzumab vedotin monotherapy in DCS4968g and about 41% for 1.8 mg/kg polatuzumab vedotin+obinutuzumab in GO27834. Arm J (1/2/60/30 mg dosing) met its primary efficacy endpoint of ORR. The ORR was 61.2% (50.85%, 70.90% CI). p<0.0001. CRR was 49% (38.74%, 59.28% CI).

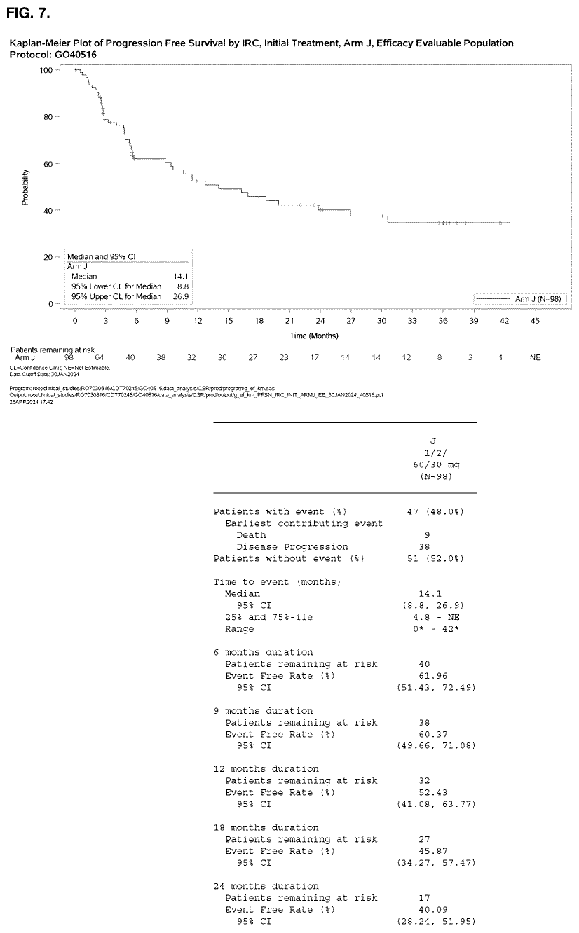

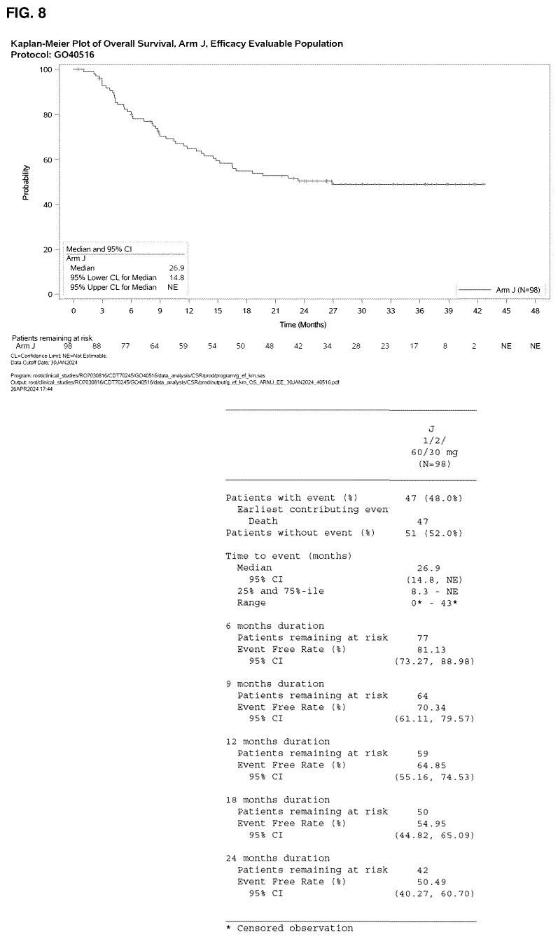

DOR results are described in FIG. 6. PFS results are described in FIG. 7. OS results are described in FIG. 8.

### Safety Results

The combination of Mosun+Pola demonstrated a manageable safety profile as an outpatient regimen. The combination of Mosun+Pola demonstrated a manageable safety profile as an outpatient regimen. The safety profile of Mosun+Pola was consistent with the known risk of individual study drugs. Median dose intensity was close to 100% and low treatment discontinuation rate due to AE. Fatigue and diarrhea were the most common AEs, and most cytokine release syndrome AEs were low grade: Arm J: 18.4% any grade CRS; 10.2% Grade 1 CRS; 5.1% Grade 2 CRS; and 3.1% Grade 3 CRS. Adverse events in Group A and Arms J, L, and M are summarized below in Table 10.

TABLE 10
Adverse Events

| | | Arm J, | Arm L, | |
|---|---|---|---|---|
| | Group A | Mosun | Mosun | Arm M, |
| | DLBCL, | IV + Pola | SC + Pola | R + Pola |
| | N = 19 | N = 98 | N = 40 | N = 39 |
| Pts with at least one AE, n (%) | 19 (100%) | 97 (99%) | 40 (100%) | 39 (100%) |
| SAEs, n (%) | 5 (26.3%) | 34 (34.7%) | 13 (32.5%) | 10 (25.6%) |
| Grade 3+ AEs, n (%) | 13 (68.4%) | 52 (53.1%) | 24 (60.0%) | 21 (53.8%) |
| Grade 5 AEs, n (%) | 2 (10.5%) | 3 (3.1%) | 2 (5.0%) | 1 (2.6%) |
| AE leading to treatment | 3 (15.8%) | 9 (9.2%) | 3 (7.5%) | 2 (5.1%) |
| withdrawal, n (%) | | | | |

Exposure of patients to mosunetuzumab/rituximab is summarized below in Table 11.

TABLE 11
Exposure to mosunetuzumab/rituximab

| | Mosunetuzumab/Rituximab | | | |
|---|---|---|---|---|
| | Group A | Arm J, | Arm L, | Arm M, |
| | DLBCL, | Mosun IV + Pola | Mosun SC + Pola | R + Pola |
| | N = 19 | N = 98 | N = 40 | N = 39 |
| Treatment Duration in days: | | | | |
| Median (range) | 147 (15-267) | 148 (15-246) | 148 (15-246) | 71 (1-371) |
| Number of treatment cycles: | | | | |
| Less than 8 cycles | 9 (47.4%) | 39 (39.8%) | 14 (35%) | 24 (61.5%) |
| 8 cycles | 7 (36.8%) | 51 (52.0%) | 26 (65.0%) | 15 (38.5%) |
| >8 cycles | 3 (15.8%) | 8 (8.2%) | 0 | 0 |
| Number of cycles: | | | | |
| Median (range) | 8 (1-13) | 8 (1-17) | 8 (1-8) | 4 (1-8) |
| Dose intensity (%) | | | | |
| Median (range) | 98.5 (83.6-100.7) | 98.7 (46.7-195.2) | 96.6 (53.3-100.7) | 96.4 (39.3-108.2) |

Exposure of patients to polatuzumab vedotin is summarized below in Table 12.

TABLE 12
Exposure to polatuzumab vedotin

| | Polatuzumab vedotin | | | |
|---|---|---|---|---|
| | Group A | Arm J, | Arm L, | Arm M, |
| | DLBCL, | Mosun IV + Pola | Mosun SC + Pola | R + Pola |
| | N = 19 | N = 98 | N = 40 | N = 39 |
| Treatment Duration in days: | | | | |
| Median (range) | 105 (1-123) | 106 (1-182) | 106 (1-199) | 71 (1-329) |
| Number of treatment cycles: | | | | |
| Less than 6 cycles | 10 (52.6%) | 39 (39.8%) | 12 (30%) | 22 (56.4%) |
| 6 cycles | 9 (47.4%) | 59 (60.2%) | 28 (70%) | 17 (43.6%) |
| Number of cycles: | | | | |
| Median (range) | 5 (1-6) | 6 (1-6) | 6 (1-6) | 4 (1-6) |
| Dose intensity (%) | | | | |
| Median (range) | 98.5 (72.8-101) | 99 (46.7-123.3) | 97.4 (53.3-101.9) | 95.7 (30.7-111.5) |

An overview of the safety results is described below in Table 13.

TABLE 13
Summary of adverse events

| | | Arm J, | Arm L, | |
|---|---|---|---|---|
| | Group A | Mosun | Mosun | Arm M, |
| | DLBCL, | IV + Pola | SC + Pola | R + Pola |
| | N = 19 | N = 98 | N = 40 | N = 39 |
| Any AE, n (%) | 19 (100%) | 97 (99.0%) | 40 (100%) | 39 (100%) |
| SAE, n (%) | 5 (26.3%) | 34 (34.7%) | 13 (32.5%) | 10 (25.6%) |
| Grade 3+ AEs, n (%) | 13 (68.4%) | 52 (53.1%) | 24 (60.0%) | 21 (53.8%) |
| Grade 5 AEs, n (%) | 2 (10.5%) | 3 (3.1%) | 2 (5.0%) | 1 (2.6%) |
| Treatment-related AEs, n (%) | 17 (89.5%) | 89 (90.8%) | 37 (92.5%) | 33 (84.6%) |
| AE leading to study drug | n = 3 | n = 9 | n = 3 | n = 2 |
| discontinuation, n (%) | | | | |
| Mosun discontinuation | 2 (10.5%) | 5 ( 5.1%) | 1 ( 2.5%) | n/a |
| Pola discontinuation | 3 (15.8%) | 8 ( 8.2%) | 3 ( 7.5%) | 2 ( 5.1%) |
| Ritux discontinuation | n/a | n/a | n/a | 1 ( 2.6%) |
| AE leading to dose | | | | |
| modification, n (%) | | | | |
| Mosun modification | 0 | 1 ( 1.0%) | 0 | n/a |
| Pola modification | 3 (15.8%) | 7 ( 7.1%) | 1 ( 2.5%) | 1 ( 2.6%) |
| Ritux modification | n/a | n/a | n/a | 0 |
| AE leading to dose | | | | |
| interruption, n (%) | | | | |
| Mosun interruption | 6 (31.6%) | 35 (35.7%) | 14 (35.0%) | n/a |
| Pola interruption | 4 (21.1%) | 25 (25.5%) | 11 (27.5%) | 15 (38.5%) |
| Ritux interruption | n/a | n/a | n/a | 14 (35.9%) |

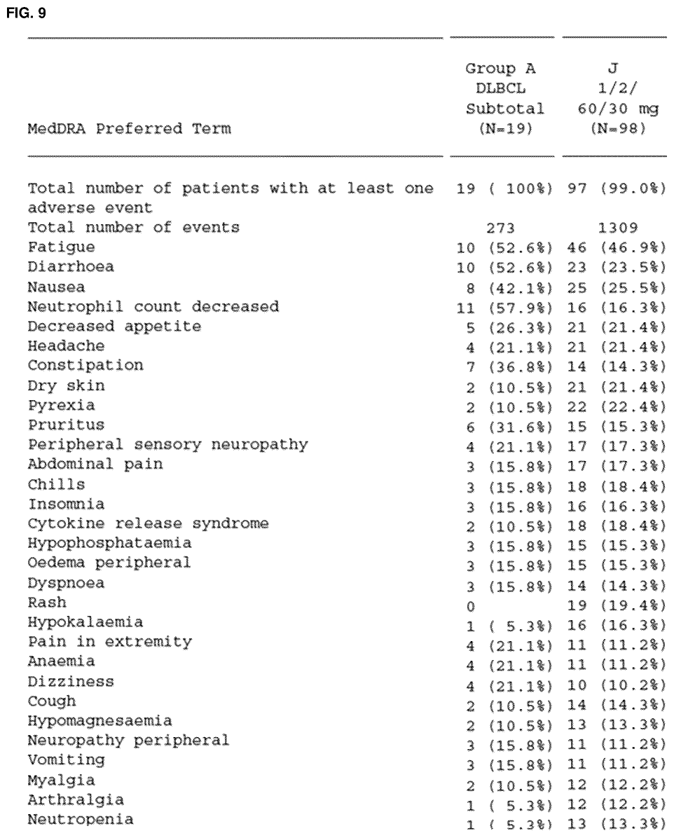

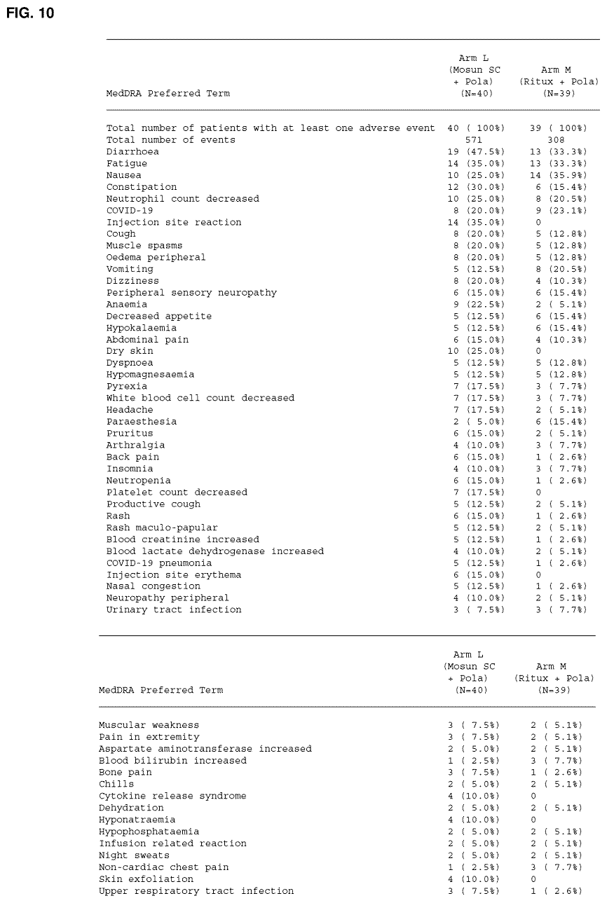

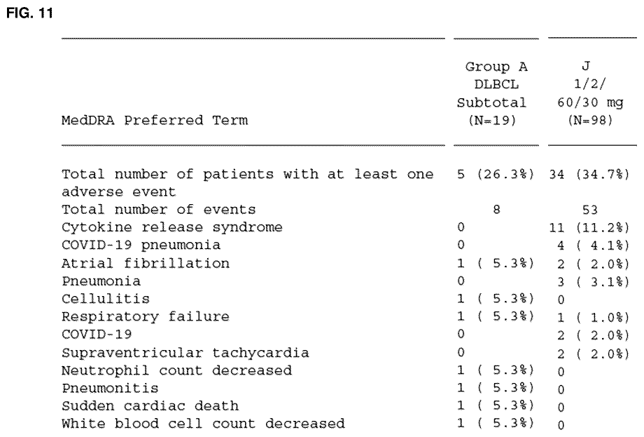

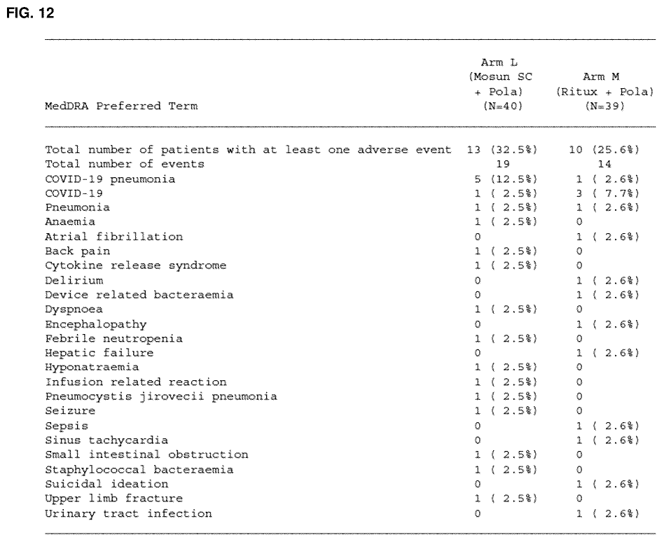

Adverse events (AE) in Group A and Arm J are summarized in FIG. 9. Adverse events in Arms L and M are summarized in FIG. 10. Serious adverse events (SAEs) in Group A and Arm J are summarized in FIG. 11. Serious adverse events in Arms L and M are summarized in FIG. 12. Cytokine release syndrome (CRS) events resulting from mosunetuzumab treatment in Group A and Arms J and L are summarized below in Table 14.

TABLE 14
Cytokine release syndrome by dose cycle

| | Group A | Arm J, | Arm L, |
|---|---|---|---|
| | DLBCL, | M IV + Pola | M SC + Pola |
| | N = 19 | N = 98 | N = 40 |
| Pts with at least one CRS | | | |
| Any grade | 2 (10.5%) | 18 (18.4%) | 4 (10%) |
| 1 | 2 (10.5%) | 10 (10.2%) | 3 (7.5%) |
| 2 | 0 | 5 (5.1%) | 1 (2.5%) |
| 3 | 0 | 3 (3.1%) | 0 |
| C1D1-C1D7 | n = 19 | n = 98 | n = 40 |
| Any grade | 2 (10.5%) | 6 (6.1%) | 4 (10%) |
| 1 | 2 (10.5%) | 4 (4.1%) | 3 (7.5%) |
| 2 | 0 | 1 (1%) | 1 (2.5%) |
| 3 | 0 | 1 (1%) | 0 |
| C1D8-C1D14 | n = 19 | n = 98 | n = 40 |
| Any grade | 0 | 2 (2%) | 0 |
| 1 | 0 | 2 (2%) | 0 |
| 2 | 0 | 0 | 0 |
| 3 | 0 | 0 | 0 |
| C1D15-C1D21 | N = 19 | n = 97 | n = 39 |
| Any grade | 0 | 11 (11.3%) | 0 |
| 1 | 0 | 5 (5.2%) | 0 |
| 2 | 0 | 5 (5.2%) | 0 |
| 3 | 0 | 1 (1%) | 0 |

CRS was manageable in all cases, and few patients required use of tocilizumab and/or ICU admission (Table 15).

TABLE 15
CRS management

| | Management in patients with CRS | | |
|---|---|---|---|
| | | Arm J, | Arm L |
| | Group A, | Mosun IV + | Mosun SC + |
| | DLBCL | Pola | Pola, |
| | N = 2 | N = 18 | N = 4 |
| Tocilizumab | 0 | 3 (16.7%) | 1 (25%) |
| Fluids | 0 | 4 (22.2%) | 0 |
| Single pressor | 0 | 2 (11.1%) | 0 |
| Oxygen low flow | 0 | 3 (16.7%) | 1 (25%) |
| Oxygen high flow | 0 | 1 (5.6%) | 0 |
| Steroids | 0 | 6 (33.3%) | 1 (25%) |
| Steroids + Tocilizumab | 0 | 2 (11.1%) | 1 (25%) |
| ICU Admission | 0 | 2 (11.1%) | 0 |

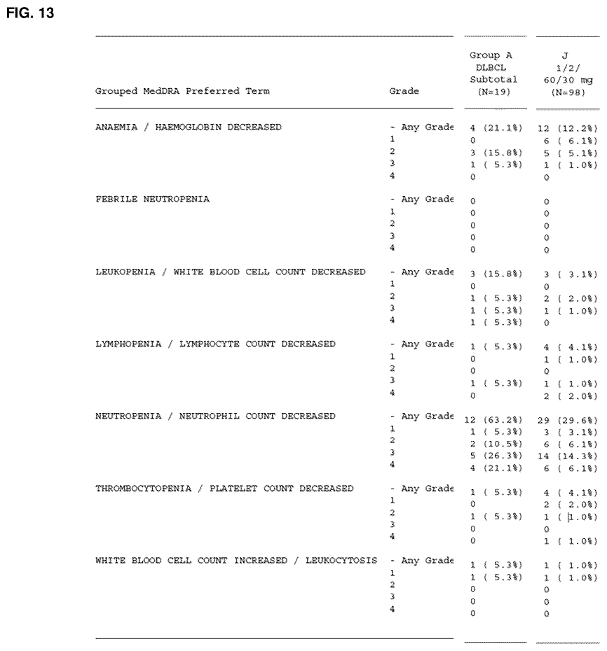

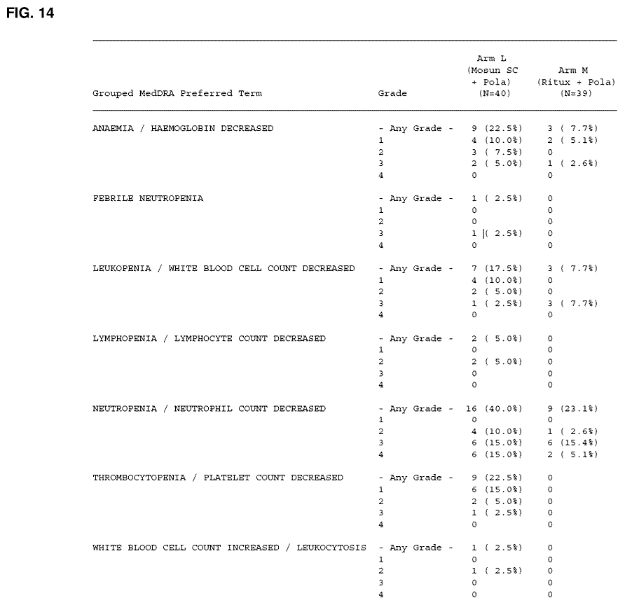

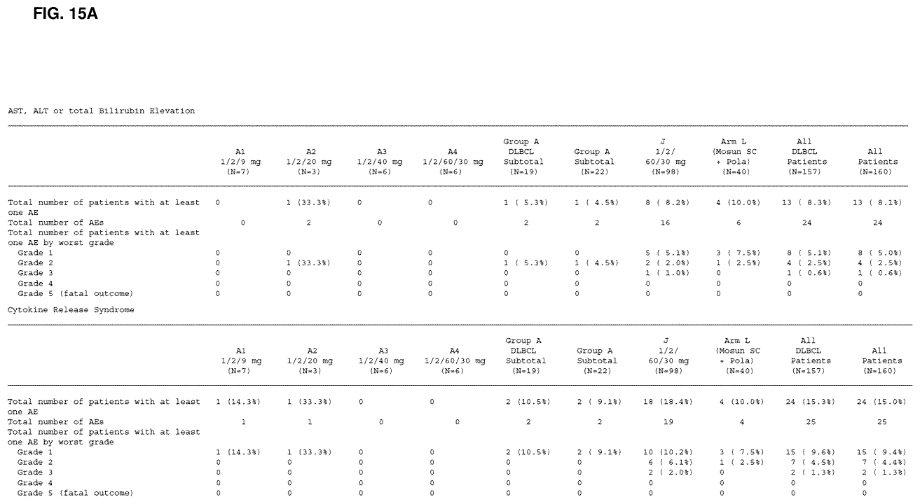

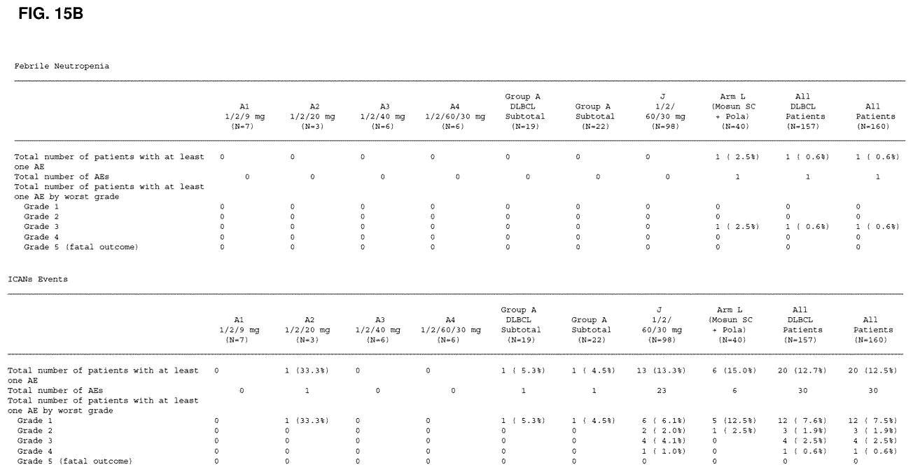

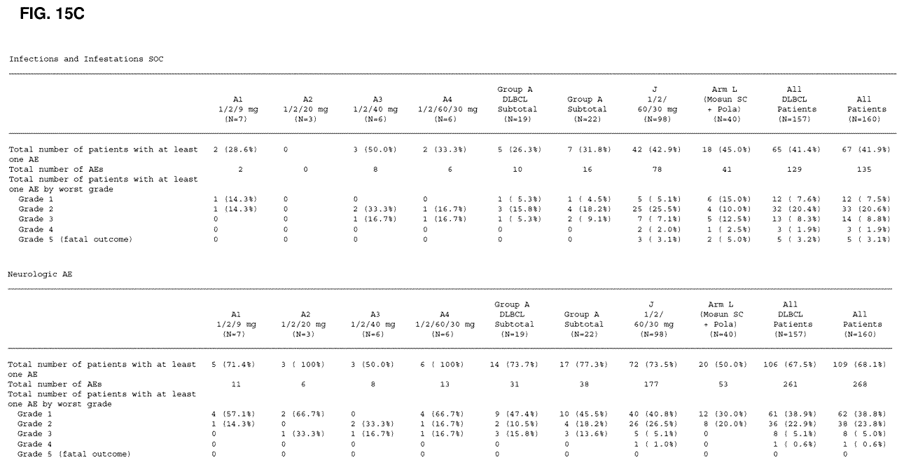

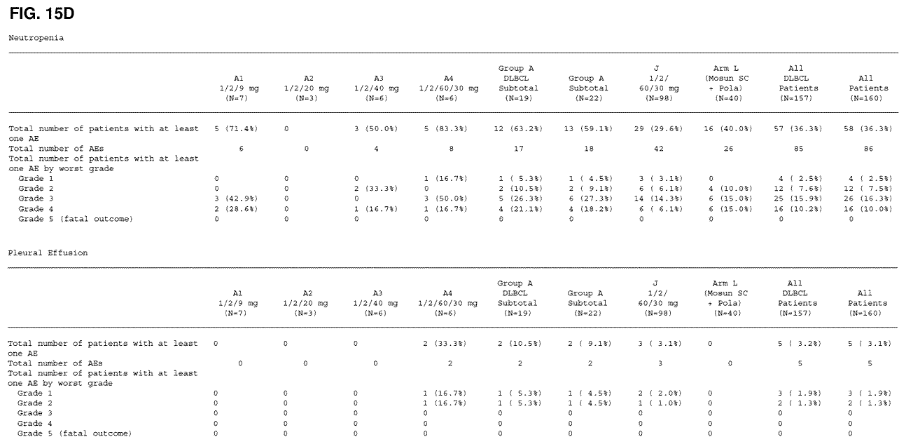

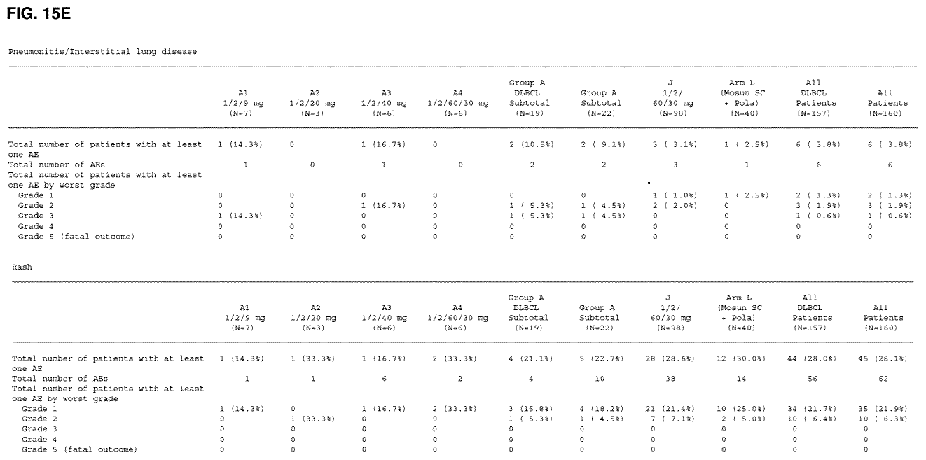

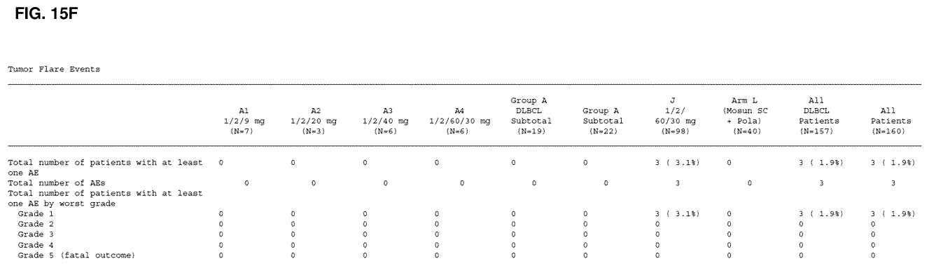

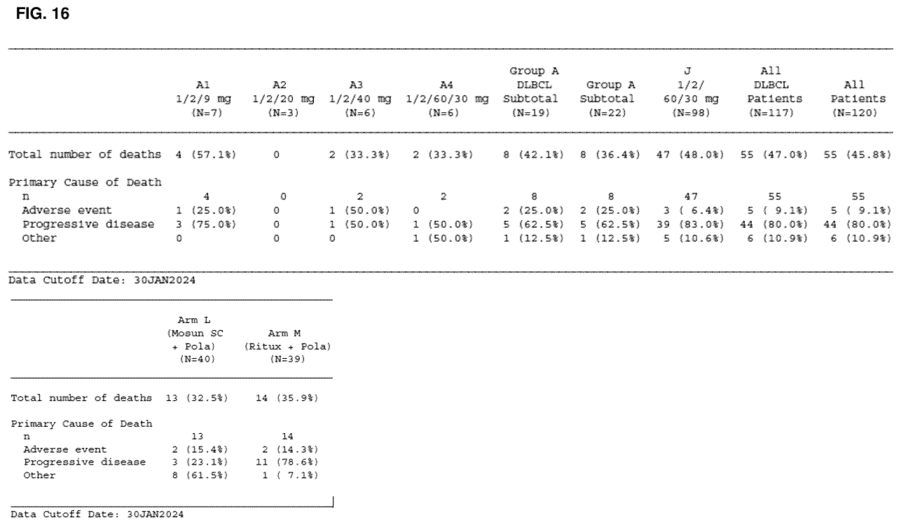

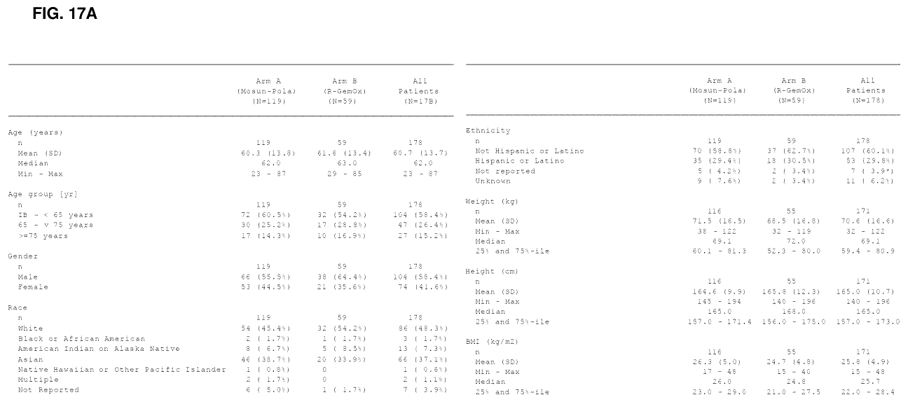

Hematologic AE in Group A and Arm J are summarized in FIG. 13. Hematologic AE in Arms L and M are summarized in FIG. 14. AE of special interest (AESI) are summarized in FIG. 15A-FIG. 15F. Deaths that occurred in the study are summarized in FIG. 16.

### Example 4. Results for a Randomized, Open-Label, Multicenter Phase III Study Evaluating Efficacy and Safety of Mosunetuzumab in Combination with Polatuzumab Vedotin in Comparison with Rituximab in Combination with Gemcitabine Plus Oxaliplatin (R-GemOx) in Subjects with Relapsed and/or Refractory Aggressive B-Cell Non-Hodgkin's Lymphoma (“SUNMO”)

SUNMO met its IA dual primary endpoint of ORR by IRC in adult patients with relapsed or refractory large B-cell lymphoma (R/R LBCL) who were not candidates for autologous stem-cell transplantation (ASCT). The efficacy-evaluable population included 178 participants with R/R LBCL who are not candidates for ASCT randomized (2:1) either in the Mosun+Pola arm or the R-GemOx arm based on the intention to treat principle (119 patients received Mosun+Pola and 59 patients received R-GemOx). The safety-evaluable population included any of the randomized 178 participants who were treated with any study treatment (116 patients received Mosun+Pola and 56 patients received R-GemOx). CCOD of the study was 19 Apr. 2024; Snapshot: 6 Jul. 2024. Median time on study was 12.6 months, range (0 -22 months) in Arm A and 10.8 months, range (0-21 months) in Arm B.

In Arm A, polatuzumab vedotin was administered intravenously (IV) on Day 1 in Cycles 1-6 (C1D1-C6D1) and mosunetuzumab was administered subcutaneously (SC) with a Cycle 1 step-up-dosing regimen (FIG. 2A). In Cycle 1, patients receive mosunetuzumab on Day 1 (5 mg; Cycle 1 Dose 1 (C1D1)), Day 8 (45 mg; Cycle 1 Dose 2 (C1D2)), and Day 15 (45 mg; Cycle 1 Dose 3 (C1D3)). In Cycles 2-8, patients receive mosunetuzumab on Day 1 (45 mg; Cycle 2 Dose 1 to Cycle 8 Dose 1 (C2D1-C8D1)). In Arm B, one cycle of treatment was 14 days (e.g., 14-day dosing cycles). During each cycle, rituximab 375 mg/m² was administered IV on Day 1; gemcitabine 1000 mg/m² was administered IV on Day 1; and oxaliplatin 100 mg/m² was administered IV on Day 1 (FIG. 2B).

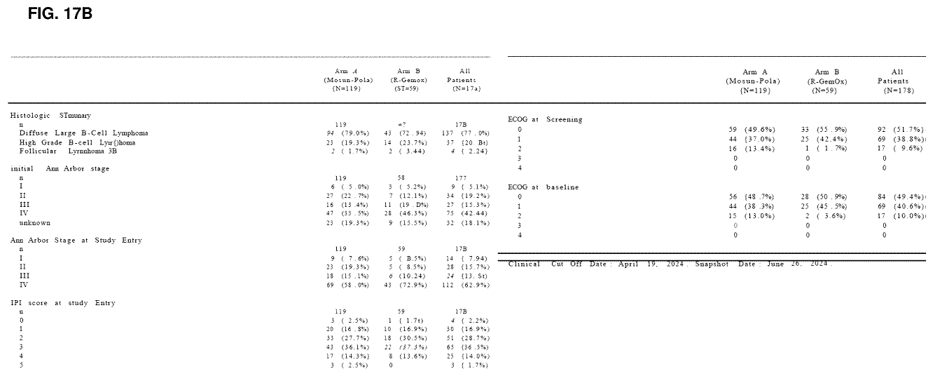

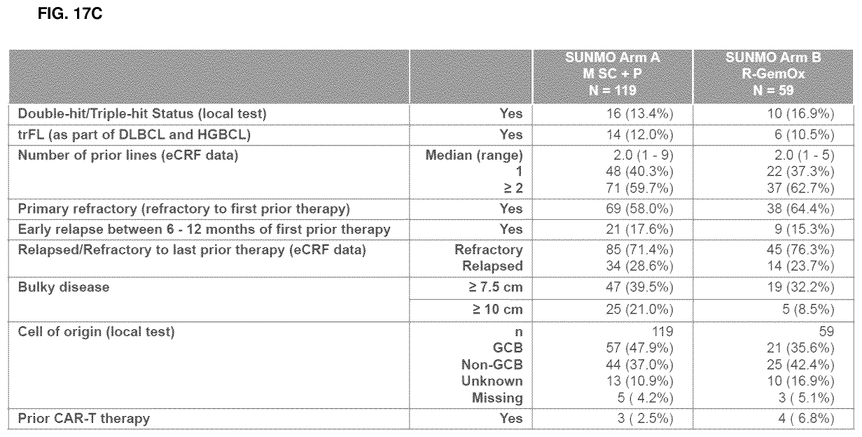

Patient demographics and baseline characteristics are summarized in FIG. 17A-FIG. 17C.

### Efficacy Results

SUNMO met its IA dual primary endpoint of ORR by IRC in adult patients with relapsed or refractory large B-cell lymphoma (R/R LBCL) who are not candidates for autologous stem-cell transplantation (ASCT).
Arm A (M+P) ORR by IRC=69.7% [95% CI: 60.7, 77.8] vs. Arm B (R-GemOx) ORR by IRC=44.1% [95% CI: 31.2, 57.6]
Delta ORR by IRC=25.7% [95% CI: 9.6, 41.8], p-value=0.0008. Consistent findings from Delta ORR by INV=30.8% [95% CI: 14.9, 46.6].
Secondary efficacy endpoints showed consistent trend favoring Arm A (M+P)
   Arm A CRR by IRC=49.6% [95% CI: 40.3%, 58.9%] vs. Arm B CRR by IRC=28.8% [95% CI: 17.8%, 42.1%]
   DOR by IRC: HR=0.72 [95% CI: 0.36, 1.46].
      Arm A mDOR=12.7 [95% CI: 9.5, NE]. Arm B mDOR=9.5 [95% CI: 3.7, NE]
      Arm A 6-month DOR: 69.0% [95% CI: 58.9, 79.1]. Arm B 6-month DOR: 52.8% [95% CI: 27.6, 78.0]
   DOCR by IRC: HR=0.41 [95% CI: 0.16, 1.07].
      Arm A mDOCR=NE [95% CI: 15.6, NE]. Arm B mDOCR=9.5 [95% CI: 3.9, NE]
      Arm A 6-month DOCR: 85.8% [95% CI: 76.6, 94.9]. Arm B 6-month DOCR: 66.1% [95% CI: 38.8, 93.4]
Benefit of M+P observed in most subgroups with minor variations in select subgroups

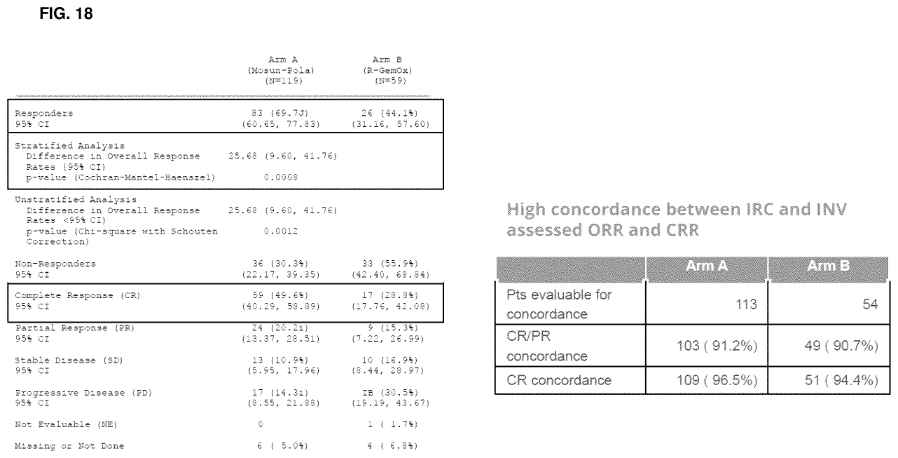

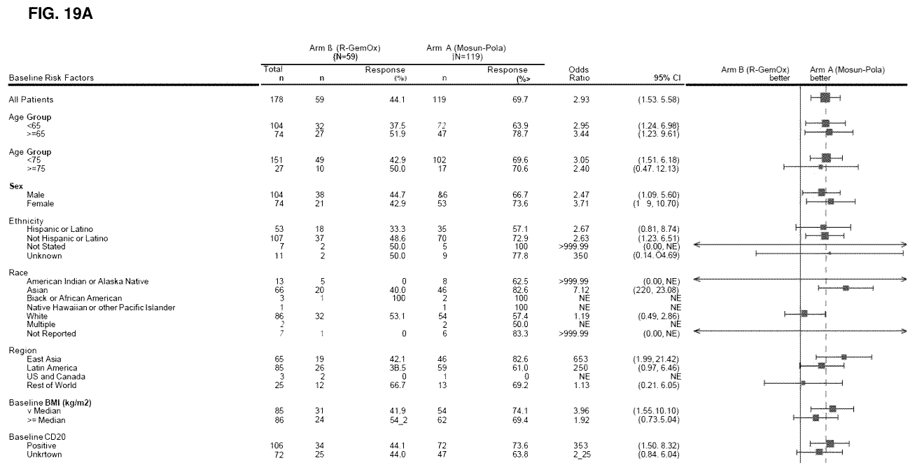

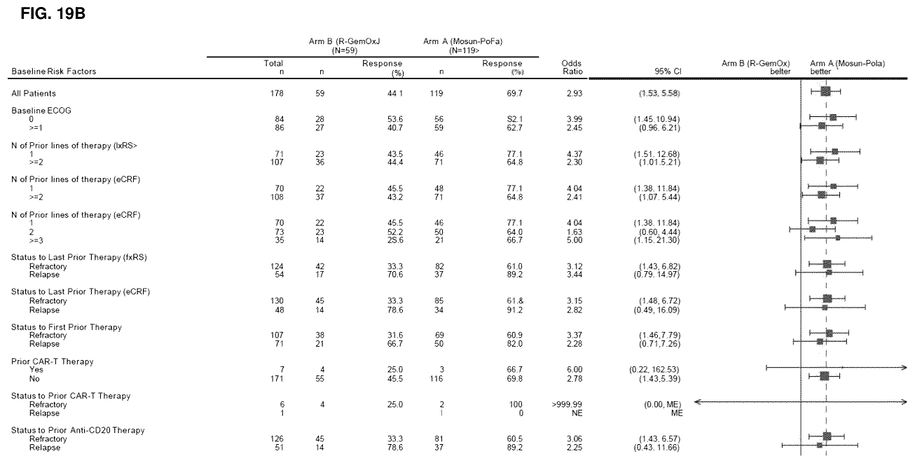

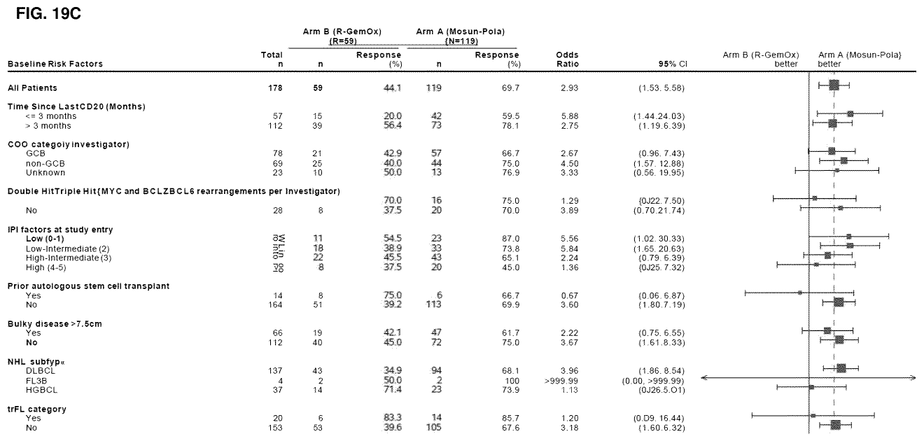

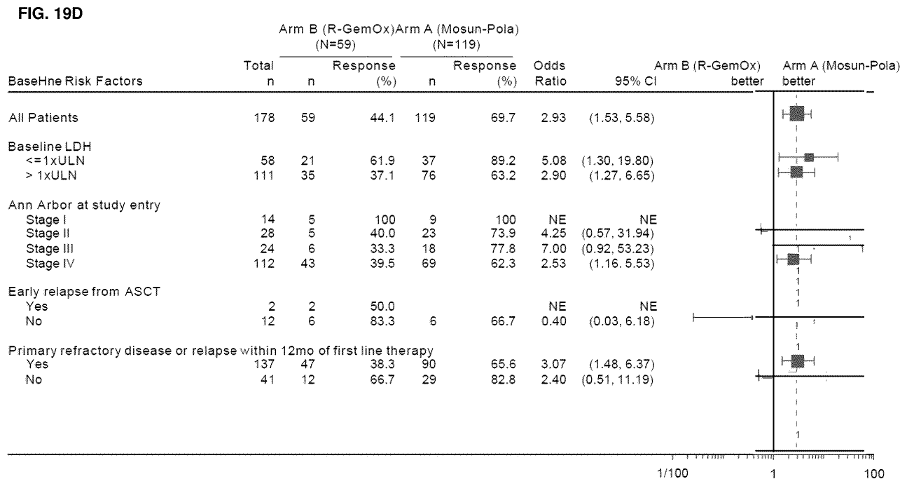

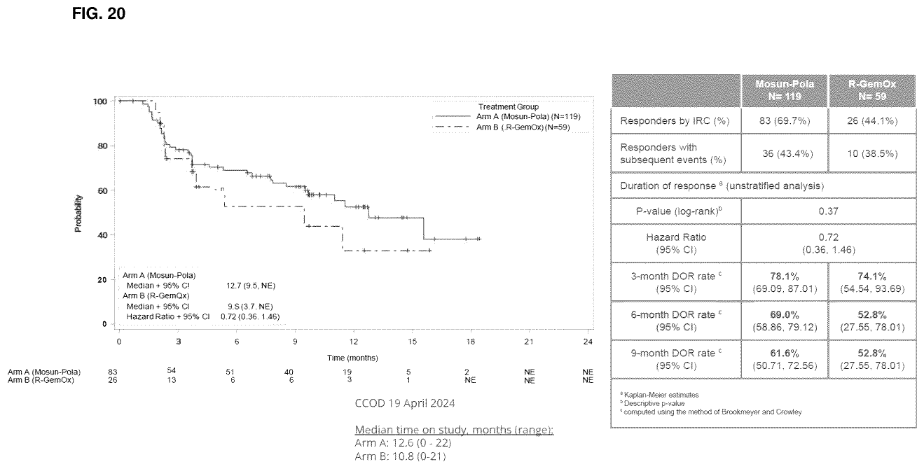

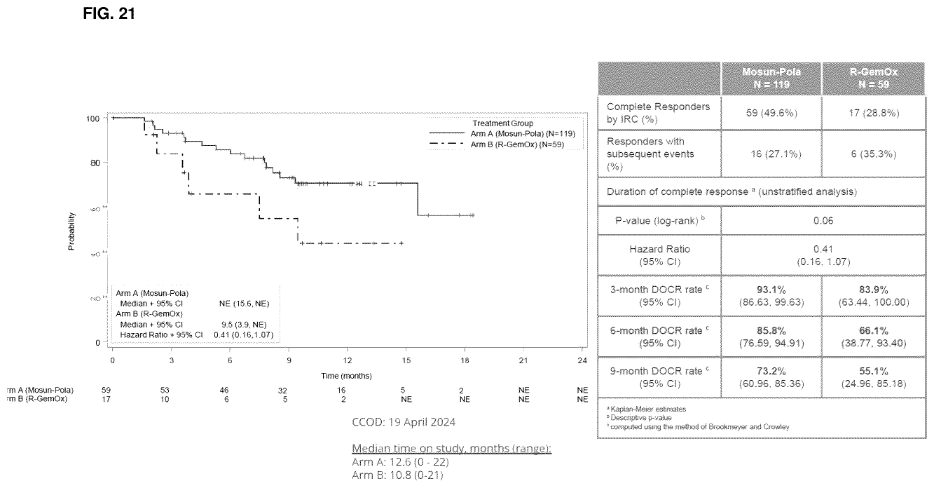

Response results are described in FIG. 18. Results determined by independent review committee (IRC) correlated strongly with those determined by investigators (INV). Response results, when stratified by various patient characteristics, consistently favored treatment by mosunetuzumab and polatuzumab vedotin as compared to R-GemOx (FIG. 19A-FIG. 19D). DOR results are described in FIG. 20. DOCR results are described in FIG. 21.

Safety Results
AESI and safety profile consistent with known risks of Mosun and Pola individually
Compared to patients treated with R-GemOx, patients treated with Mosun+Pola exhibited more any-grade infections (including COVID-19) and pneumonitis, but less cytopenias, and classic chemotherapy related toxicities (e.g., fatigue, nausea, vomiting, diarrhea, and neuropathy)
Combination of Mosun+Pola is manageable without mandatory hospitalizations
CRS events occurred in 25% (29 of 116 safety-evaluable patients in Arm A), and mostly Grade 1 (Grade 1:19.8%, Grade 2:4.3%, Grade 3:0.9%),
At least 74% (Mosun+Pola) and 54% (R-GemOx) of patients had dose intensity ≥90%
Treatment discontinuation rates due to AE were low (2.6% Mosun+Pola vs 5.4% R-GemOx)
Consistent with previous studies; no suspected ICANs events identified

For the 116 patients treated with mosunetuzumab+polatuzumab vedotin, the median duration of treatment with mosunetuzumab was 148 days (1-221 days range), and the median duration of treatment with polatuzumab vedotin was 106 days (1-155 days range). Patients were treated for a median of 8 dosing cycles of mosunetuzumab (1-8 cycles range) and 6 dosing cycles of polatuzumab vedotin (1-6 cycles range). Patients were treated with 98.6% (20.0%-110.5%) dose intensity of mosunetuzumab and 97.3% (37.9%-111.0%) dose intensity of polatuzumab vedotin, and 90 patients (77.6%) were treated with >90% dose intensity of mosunetuzumab and 86 patients (74.1%) were treated with ≥90% dose intensity of polatuzumab vedotin. Majority of patients with dose intensity >90% and high treatment completion rate supports Mosun+Pola being a tolerable regimen.

In contrast, for the 56 patients treated with R-GemOx, the median duration of treatment with rituximab was 67 days (1-155 days range), the median duration of treatment with gemcitabine was 66.5 days (1-155 days range), and the median duration of treatment with oxaliplatin was 66.5 days (1-155 days range). Patients were treated for a median of 5 dosing cycles of R-GemOx (1-8 cycles range). Patients were treated with 93.9% (49.0%-109.8%) dose intensity of rituximab, 96.1% (53.0%-124.7%) dose intensity of gemcitabine, and 92.6% (53.0%-124.6%) dose intensity of oxaliplatin; and 31 patients (55.4%) were treated with >90% dose intensity of rituximab, 34 patients (60.7%) were treated with ≥90% dose intensity of gemcitabine, and 30 patients (53.6%) were treated with ≥90% dose intensity of oxaliplatin. Compared with mosun+pola, less patients completed R-GemOx treatment and less patients were treated with dose intensity ≥90%.

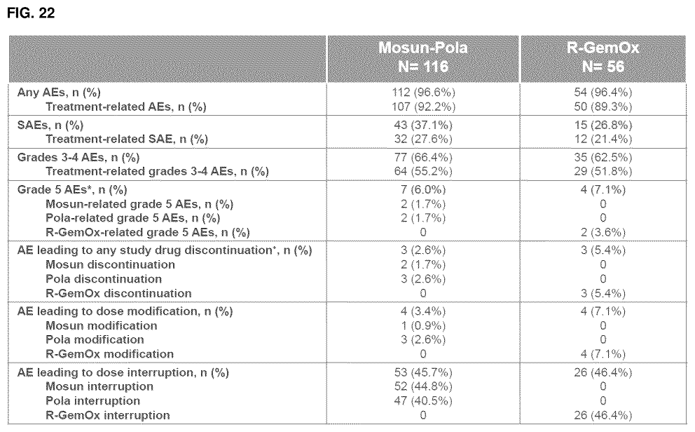

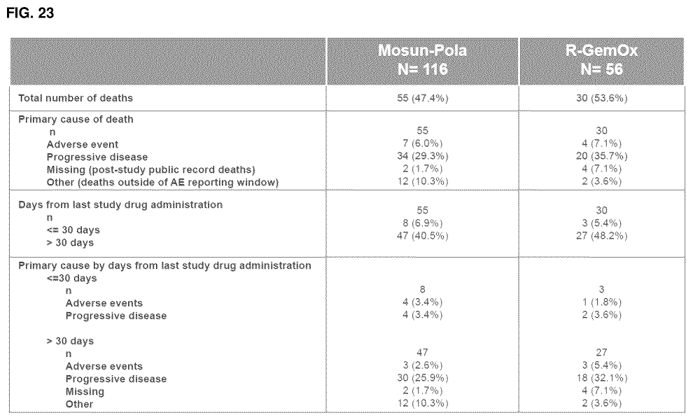

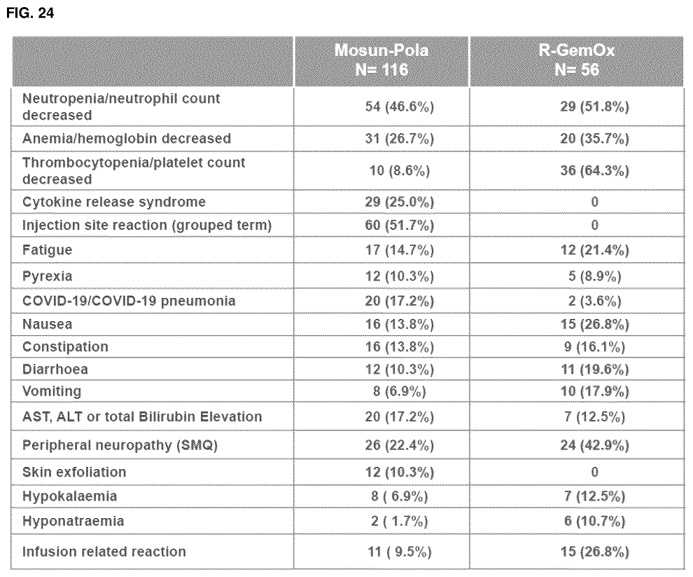

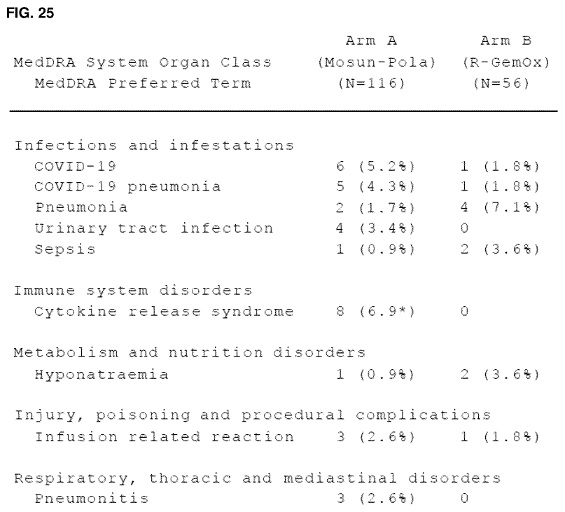

AEs observed in the study are summarized in FIG. 22. Deaths in the study are summarized in FIG. 23. Fatal AEs in Arm A (mosun+pola) included pulmonary embolism, septic shock, cardiac arrest, CMV infection, COVID-19, and COVID-19 pneumonia (n=2). Fatal AEs in Arm B (R-GemOx) included pneumonia, sceptic shock, sepsis, and COVID-19 pneumonia. AEs with incidence rate of at least 10% in either treatment arm are summarized in FIG. 24. Injection site reaction (ISR), CRS, COVID-19, and skin exfoliation were >5% more prevalent in Arm A compared to Arm B. Anaemia, neutropenia/neutrophil count decrease, thrombocytopenia/platelet count decrease, fatigue, nausea, diarrhea, vomiting, peripheral neuropathy, infusion related reaction (IRR), hypokalemia, and hyponatremia were >5% more prevalent in Arm B compared to Arm A. Serious adverse events are summarized in FIG. 25.

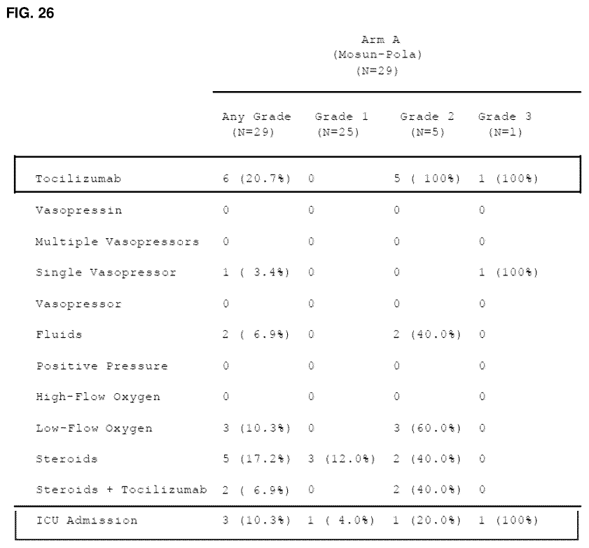

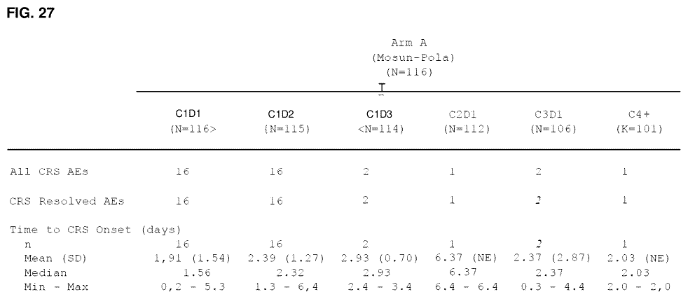

CRS events occurred in 25% (29 of 116 safety-evaluable patients in Arm A), and mostly Grade 1 (Grade 1:19.8%, Grade 2:4.3%, Grade 3:0.9%). No Grade 4 or 5 CRS events occurred. Median time to first CRS onset was 2 days (0-6 days range). Median duration was 3 days (1-11 days range). CRS was manageable in all cases, and few patients required use of tocilizumab and/or ICU admission (FIG. 26). Timing of CRS is summarized in FIG. 27. CRS mostly occurred after C1D1 (administered on Cycle 1 Day 1) and C1D2 (administered on Cycle 1 Day 8) doses.

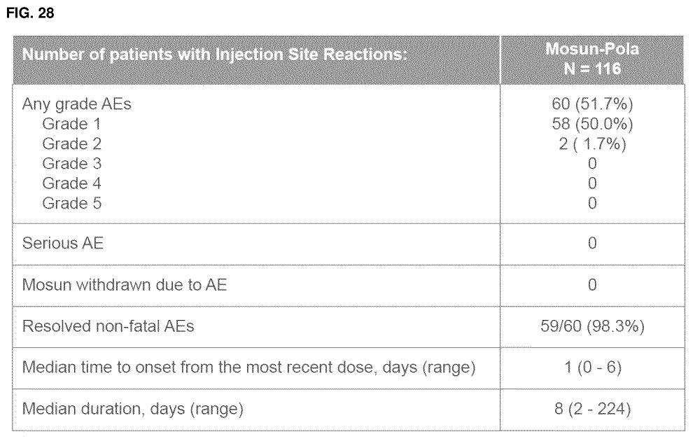

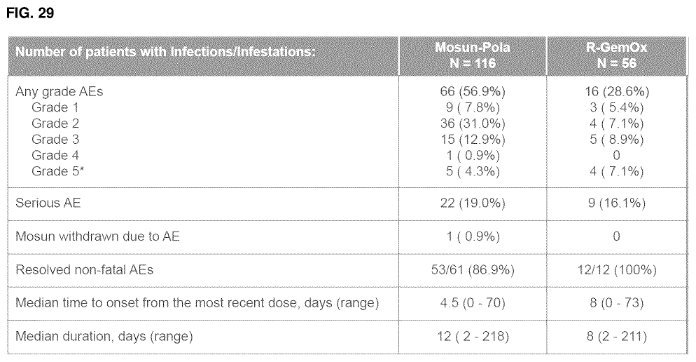

Injection site reactions for mosunetuzumab administration were all Grade 1 or 2 and majority were resolved (FIG. 28). Infections and Infestations that occurred were within the known safety profile of the individual study drugs (FIG. 29).

Adverse events of special interest were consistent with understanding of mosunetuzumab and polatuzumab vedotin (FIG. 30).

### Example 5. Fixed-Duration Outpatient Subcutaneous Mosunetuzumab (M)+Polatuzumab Vedotin (Pola) Shows Robust Efficacy in a Phase II Study of Relapsed/Refractory Post-BTKi Mantle Cell Lymphoma

Patients (pts) with mantle cell lymphoma (MCL) who progress after Bruton tyrosine kinase inhibitor (BTKi) therapy have suboptimal outcomes, and thus new treatment options are needed. Primary data from the Phase II MCL cohort are presented.

Methods: Pts with R/R MCL and >2 prior therapies received subcutaneous M with step-up dosing on Days 1 (5 mg), 8 (45 mg), and 15 (45 mg) of Cycle (C) 1, then 45 mg on D1 of each subsequent cycle every 3 weeks for 17 cycles. Intravenous Pola (1.8 mg/kg) was given on D1 of C1-6. Corticosteroid premedication was required for C1 (optional for C2+). Hospitalization to monitor for cytokine release syndrome (CRS) was not mandatory. Response was assessed by an independent review committee (IRC) using Lugano criteria.

Results: As of Nov. 8, 2024, 42 pts received M+Pola. Median age was 68 (range 44-82) years, 91% had Ann Arbor stage III/IV, and 48% had a simplified MIPI score of ≥6. Pts had high-risk factors including: Ki-67 proliferation index>50%, 67%; blastoid/pleomorphic variants, 38%; and TP53 mutation, 31%. Median prior lines of therapy was 3 (range 2-9). All pts received prior BTKi; 26% received prior chimeric antigen receptor (CAR) T-cell therapy and 93% were refractory to last therapy.

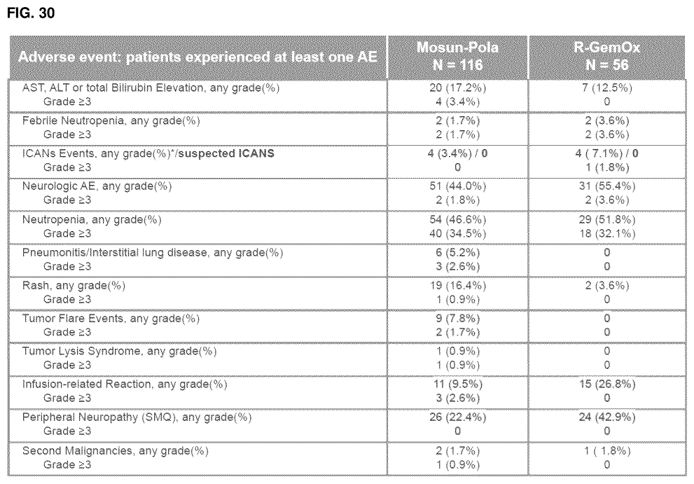

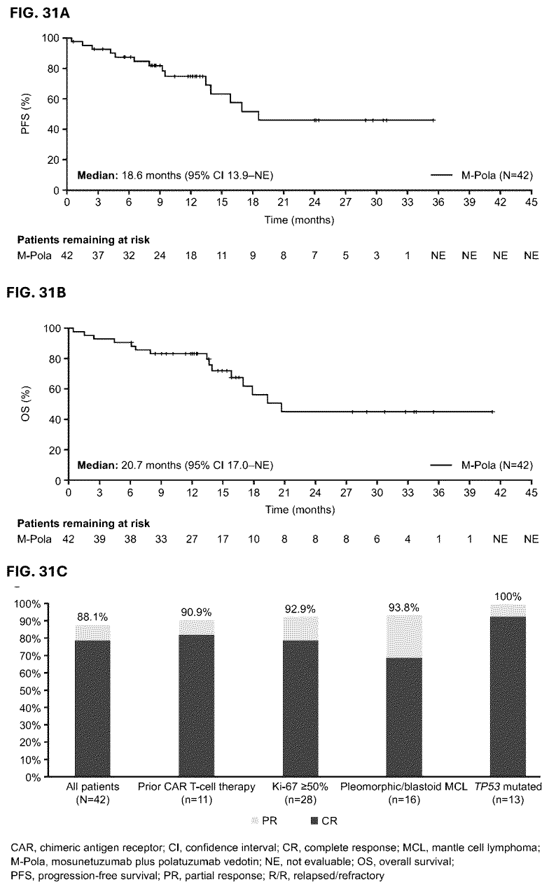

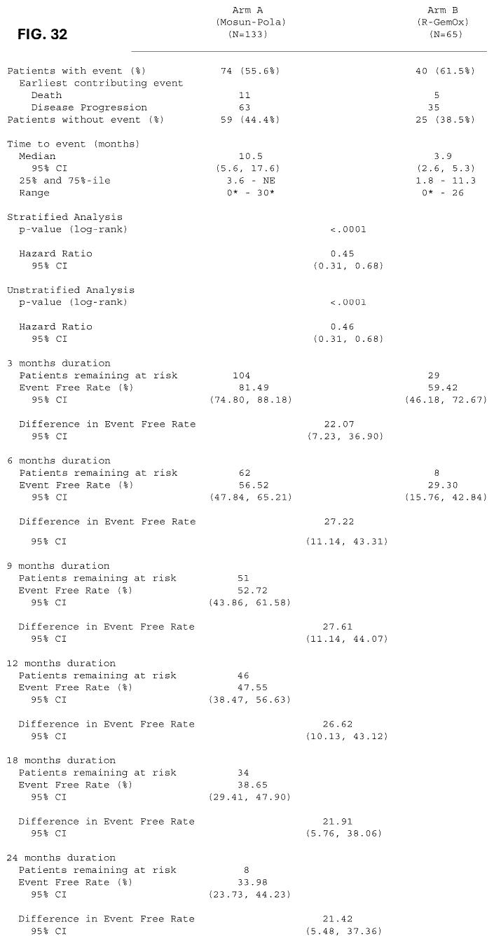

Median time on study was 16 (range 0-41) months. IRC-assessed best overall response and complete response (CR) rates were 88% (95% CI 74-96) and 79% (95% CI 63-90), respectively. Median time to first response was 3 months. Median duration of response and of CR were both not reached. Median PFS and OS were 19 months (95% CI 14—not evaluable [NE]) and 21 months (95% CI 17-NE), respectively; efficacy was consistent across high-risk subgroups (FIG. 31A-FIG. 31C).

Overall, 100%, 81% and 62% of pts reported ≥1 adverse event (AE), Grade (Gr) ≥3 AE and serious AE, respectively. The most common (>30%) treatment-related AEs were injection site reaction (57%), fatigue (50%), CRS (43%) and neutrophil count decreased (33%). CRS occurred in 18 pts (43%; Gr 1, n=12; Gr 2, n=6; no Gr >3; all resolved). There were five Gr 5 AEs (pneumonia [n=1], encephalopathy due to West Nile Virus [n=1], and COVID-19 pneumonia [n=3]). Gr 2 immune effector cell-associated neurotoxicity syndrome occurred in one patient (2%; resolved). Five pts (12%) had treatment-related AEs leading to any treatment discontinuation (Gr 2: upper respiratory tract infection [n=1], peripheral neuropathy [n=2]; Gr 3: uveitis, pneumonitis/Clostridium difficile (C. diff) colitis [n=1 each]).

Conclusions: Fixed-duration, outpatient M+Pola demonstrates robust efficacy and manageable safety in R/R MCL, including in high-risk subgroups and the post-CAR T-cell setting. M+Pola appears to be a suitable regimen for pts with R/R MCL in need of rapid disease control.

### Example 6. Updated Results for a Randomized, Open-Label, Multicenter Phase III Study Evaluating Efficacy and Safety of Mosunetuzumab (Mosun) in Combination with Polatuzumab Vedotin (Pola) in Comparison with Rituximab in Combination with Gemcitabine Plus Oxaliplatin (R-GemOx) in Subjects with Relapsed and/or Refractory Aggressive B-Cell Non-Hodgkin's Lymphoma (“SUNMO”)

Described below are updated results from the SUNMO study described in Examples 1 and 4, comparing the combination treatment of Mosun+Pola to the reference treatment of R-GemOx in adult patients with relapsed or refractory large B-cell lymphoma (R/R LBCL) who were not candidates for autologous stem-cell transplantation (ASCT). The efficacy-evaluable population (excluding Chinese patients) included 198 participants with R/R LBCL who are not candidates for ASCT randomized (2:1) either in the Mosun+Pola arm or the R-GemOx arm based on the intention to treat principle (133 patients received Mosun+Pola and 65 patients received R-GemOx). The safety-evaluable population included any of the randomized 189 participants who were treated with any study treatment (130 patients received Mosun+Pola and 59 patients received R-GemOx). CCOD of the study was Feb. 17, 2025; Data snapshot date: Apr. 9, 2025.

In Arm A, polatuzumab vedotin was administered intravenously (IV) on Day 1 in Cycles 1-6 (C1D1-C6D1) and mosunetuzumab was administered subcutaneously (SC) with a Cycle 1 step-up-dosing regimen (FIG. 2A). In Cycle 1, patients receive mosunetuzumab on Day 1 (5 mg; Cycle 1 Dose 1 (C1D1)), Day 8 (45 mg; Cycle 1 Dose 2 (C1D2)), and Day 15 (45 mg; Cycle 1 Dose 3 (C1D3)). In Cycles 2-8, patients receive mosunetuzumab on Day 1 (45 mg; Cycle 2 Dose 1 to Cycle 8 Dose 1 (C2D1-C8D1)). In Arm B, one cycle of treatment was 14 days (e.g., 14-day dosing cycles). During each cycle, rituximab 375 mg/m² was administered IV on Day 1; gemcitabine 1000 mg/m² was administered IV on Day 1; and oxaliplatin 100 mg/m² was administered IV on Day 1 (FIG. 2B).

SUNMO met its dual primary endpoint of PFS by IRC in adult patients with R/R LBCL who are not candidates for ASCT. The PFS HR was 0.45 (0.31, 0.68), and the p-value <0.0001.

Key secondary efficacy endpoint of OS was not met at this interim OS analysis; however, it consistently favors Mosun+Pola. OS HR=0.74 (0.49, 1.10); stratified p-value=0.1376. Median OS for Mosun+Pola is 18.7 mo (14.0, NE) vs R-GemOx 11.3 mo (9.2, 20.1).

PFS benefit of Mosun+Pola was observed in most subgroups with minor variations in select subgroups.

Supportive efficacy endpoints show consistent results favoring Mosun+Pola:
ORR by IRC: Mosun+Pola 70.7% (62.16%, 78.25%) vs R-GemOx 40.0% (28.04%, 52.90%).
CRR by IRC: Mosun+Pola 51.1% (42.32%, 59.89%) vs R-GemOx 26.2% (16.03%, 38.54%)

No new safety signals were detected.
Safety profile was consistent with known risks of Mosun and Pola individually.
Compared to R-GemOx, Mosun+Pola has less cytopenia, peripheral neuropathy, and fewer toxicities related to cytotoxic chemotherapies.
CRS events occurred in 24.6% of Mosun+Pola patients (32 out of 130 safety-evaluable patients), and mostly Grade 1 (Grade 1:20.0%, Grade 2:3.8%, Grade 3:0.8%), consistent with previous studies; no suspected ICANs events identified.

Mosun+Pola was demonstrated to provide a favorable benefit/risk with statistically significant and clinically meaningful improvement in PFS and ORR, and favorable safety profile suitable for outpatient administration in the target population of transplant ineligible (e.g., ASCT-ineligible) R/R LBCL (e.g., excluding Chinese patients).

Overall, more patients in the R-GemOx Arm received new anti-lymphoma treatment (NALT). NALT imbalance may impact OS.

### Efficacy Results

Progression free survival (PFS) as determined by independent review committee (IRC) in the intent to treat (ITT) population is shown in FIG. 32.

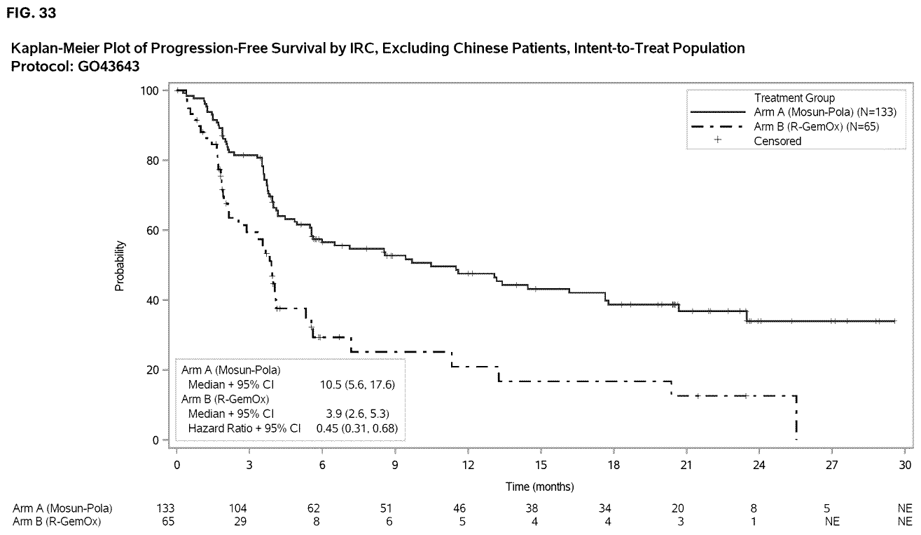

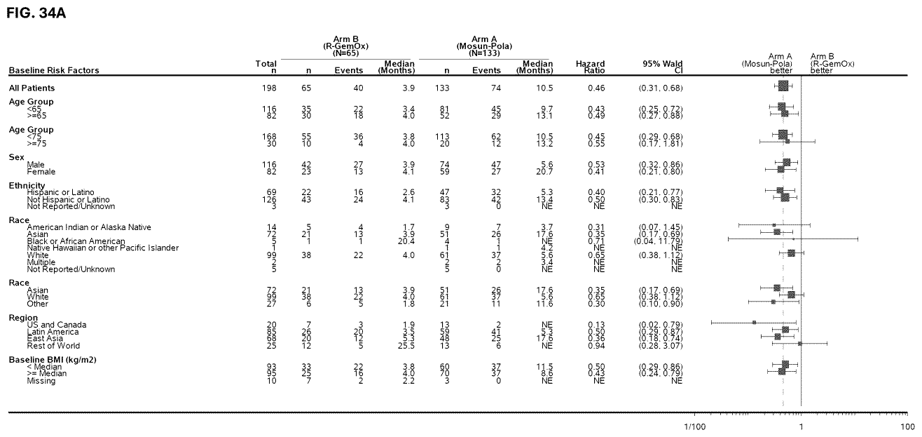

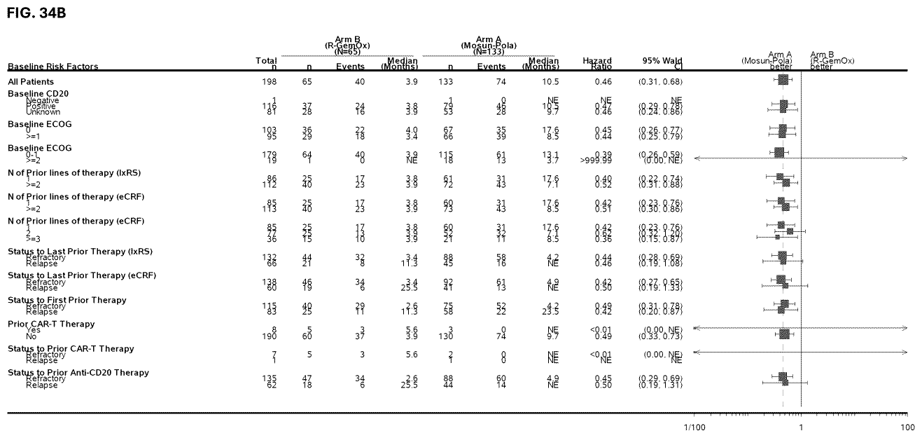

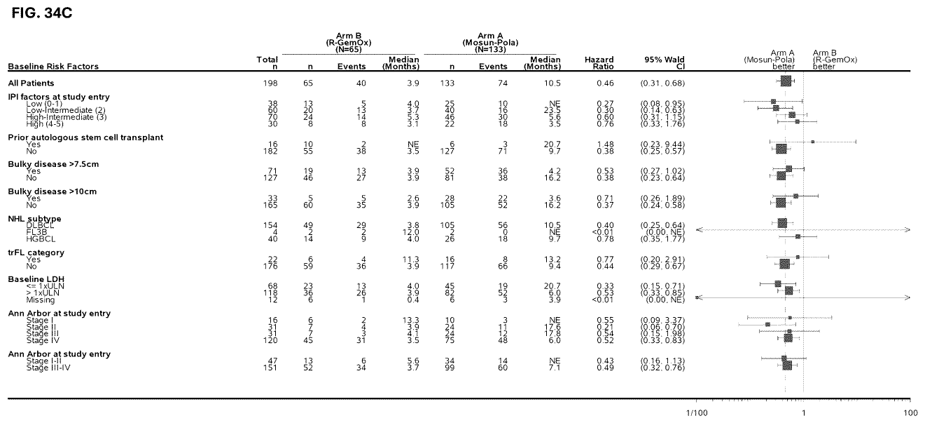

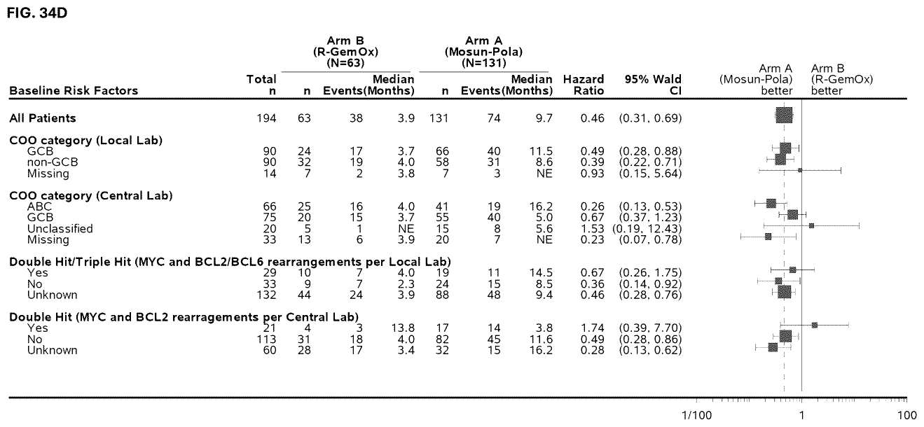

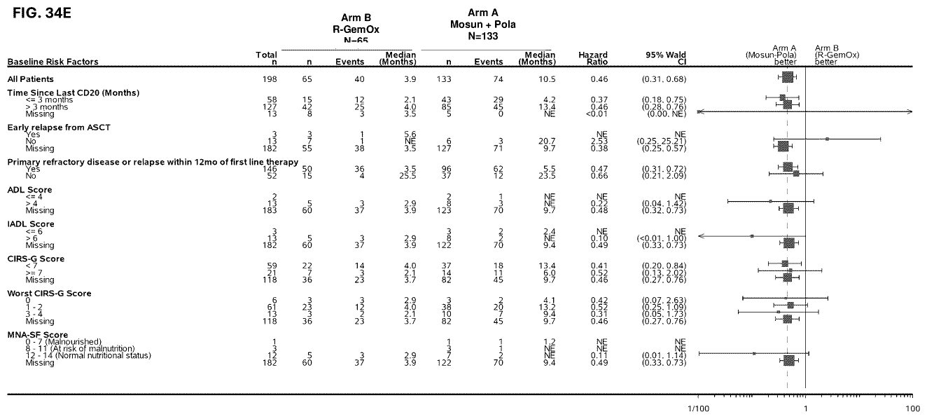

A Kaplan-Meier plot of PFS by IRC in ITT patients is shown in FIG. 33. Subgroup analysis of IRC-assessed PFS was performed for subgroups divided by baseline characteristics. Results are shown in FIG. 34A-FIG. 34E as forest plots. Subgroups analysis consistently favors Mosun+Pola compared over R-GemOx.

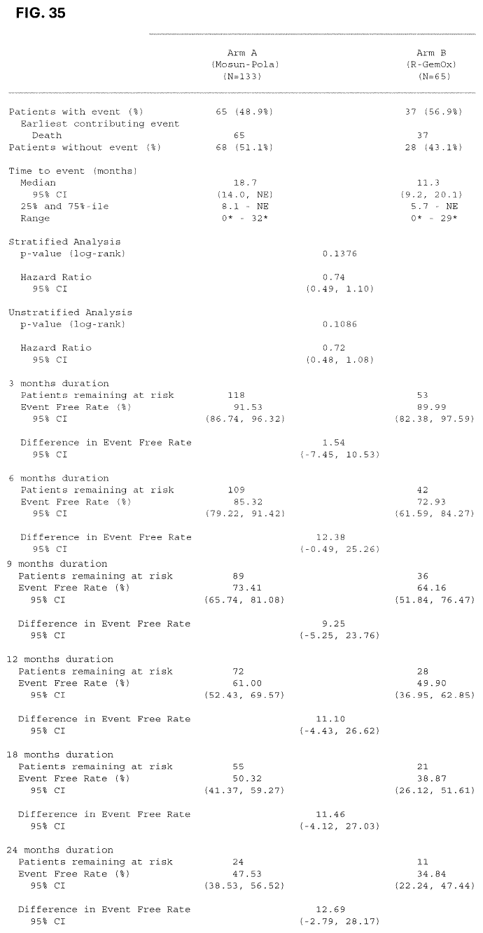

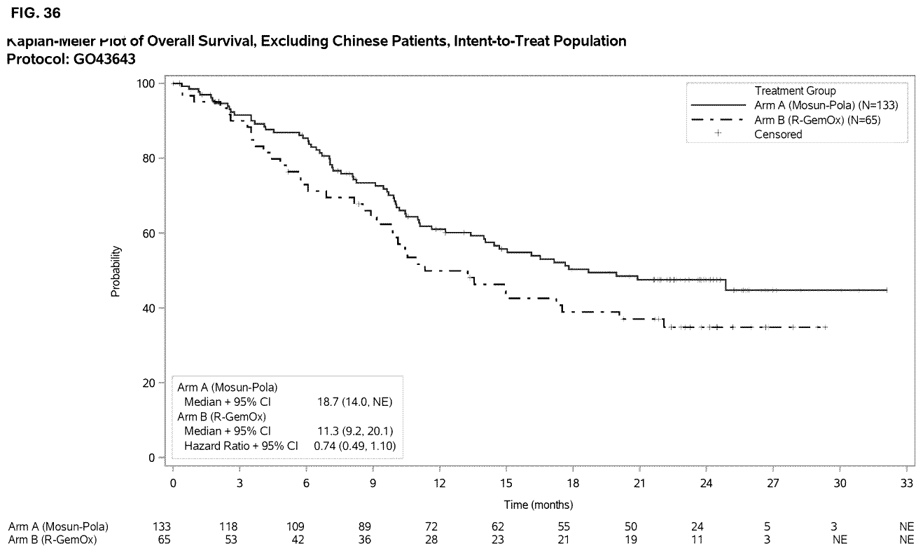

Interim OS did not meet statistical significance. OS analysis results are shown in FIG. 35. A Kaplan-Meier plot of OS by IRC in ITT patients is shown in FIG. 36.

After adjusting for potential baseline imbalances (using multivariate analysis (MVA)) and NALT (using inverse probability of censoring weighting (IPCW)), Mosun+Pola trended towards an OS benefit. Bayesian shrinkage model improves subgroup HR estimates, especially when data are limited or noisy, by pulling extreme or uncertain values closer to a central, more reliable value. This helps reduce overfitting and makes subgroup estimates more stable. Baseline covariates used in MVA and IPCW models are bulky disease (>10 cm), ECOG, LDH, sex, transformed FL (yes vs no), international prognostic index (IPI) factors at study entry, region, Ann Arbor Stage at study entry, cell of origin (COO) per Central Lab, histology, and status to first prior therapy.

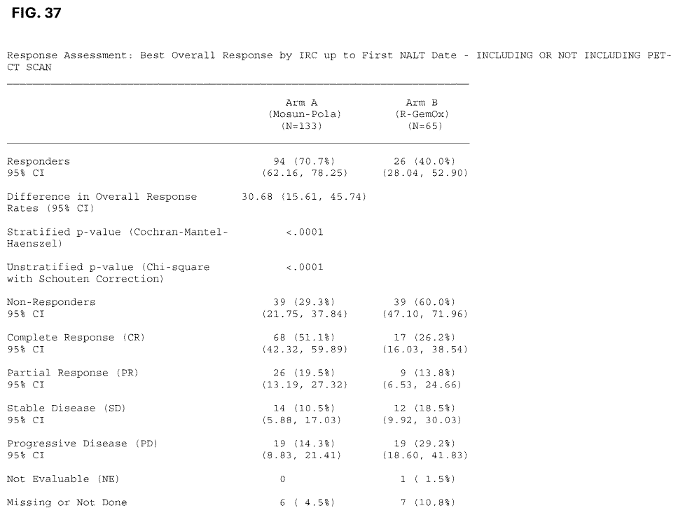

Objective response (complete response (CR)+partial response (PR)) rate (ORR) and complete response (CR only) rate (CRR) results are shown in FIG. 37. Concordance was found between IRC and INV assessed ORR (assessed CR/PR) and CRR (assessed CR).

### Safety Results

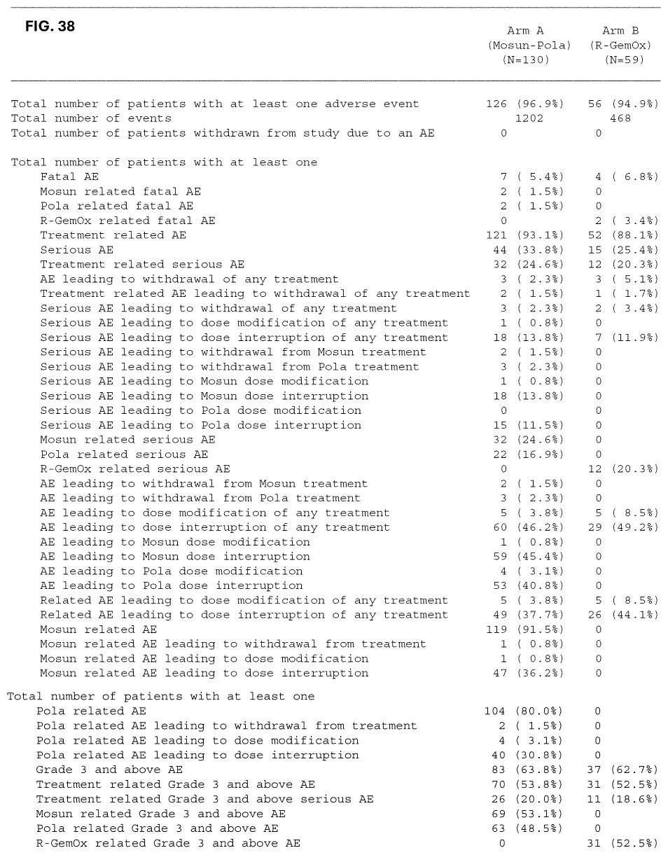

The combination of Mosun+Pola demonstrated a manageable safety profile as an outpatient regimen. Adverse events of special interest (AESI) and safety profile of Mosun+Pola were consistent with the known risk of individual study drugs. No new fatal AEs or AEs leading to treatment discontinuation occurred since the interim analysis. Most cytokine release syndrome AEs were Grade 1 (24.6% any grade CRS (32 patients): 20.0% Grade 1 (26 patients); 3.8% Grade 2 (5 patients); 0.8% Grade 3 (1 patient)) and most commonly occur in Cycle 1. There were no suspected immune effector cell-associated neurotoxicity syndrome (ICANS) events. Compared to R-GemOx, Mosun+Pola had more any-grade infections (including COVID-19) and pneumonitis and fewer cytopenia and classic chemotherapy-related toxicities (fatigue, nausea, vomiting, diarrhea, neuropathy). A summary of select AEs is show in FIG. 38. Generally comparable AE rates were observed between Mosun-Pola and R-GemOx.

Injection site reactions (e.g., for subcutaneously administered mosunetuzumab) of any Grade occurred in 70 patients (53.8%). Of these, 68 (52.3%) were Grade 1, and 2 (1.5%) were Grade 2. Infections and infestations of any Grade occurred in 69 (53.1%) of Mosun+Pola patients and 17 (28.8%) of R-GemOx patients. Grade 1 infections and infestations occurred in 9 (6.9%) of Mosun+Pola patients and 3 (5.1%) of R-GemOx patients; Grade 2 infections and infestations occurred in 39 (30.0%) of Mosun+Pola patients and 5 (8.5%) of R-GemOx patients; Grade 3 infections and infestations occurred in 15 (11.5%) of Mosun+Pola patients and 5 (8.5%) of R-GemOx patients; Grade 4 infections and infestations occurred in 1 (0.8%) of Mosun+Pola patients and 0 of R-GemOx patients; and Grade 5 infections and infestations occurred in 5 (3.8%) of Mosun+Pola patients and 4 (6.8%) of R-GemOx patients. Grade 5 infections and infestations in Mosun+Pola arm were: Septic Shock (n=1), COVID-19 pneumonia (n=2), COVID-19 (n=1), Cytomegalovirus infection reactivation (n=1); and in R-GemOx arm were: pneumonia (n=1), Septic Shock (n=1), Sepsis (n=1), COVID-19 pneumonia (n=1).

Other adverse events of special interest (AESIs) or AEs of note are shown below in Table 16.

TABLE 16
Adverse Events of Special Interest and Other AEs of Note

| | Mosun-Pola | R-GemOx |
|---|---|---|
| Adverse event: patients experienced at least one AE | N = 135 | N = 64 |
| Anemia/Hemoglobin Decreased, any grade(%) | 38 (29.2%) | 23 (39.0%) |
| Grade ≥3 | 7 (5.4%) | 12 (20.3%) |
| Bacterial Infections, any grade(%) | 8 (6.2%) | 5 (8.5%) |
| Grade ≥3 | 3 (2.3%) | 2 (3.4%) |
| Febrile Neutropenia, any grade(%) | 3 (2.3%) | 2 (3.4%) |
| Grade ≥3 | 3 (2.3%) | 2 (3.4%) |
| Fungal Infections, any grade(%) | 4 (3.1%) | 3 (5.1%) |
| Grade ≥3 | 0 | 1 (1.7%) |
| Hepatic Events, any grade(%) | 24 (18.5%) | 9 (15.3%) |
| Grade ≥3 | 4 (3.1%) | 0 |
| ICANs Events, any grade(%) | 5 (3.8%) | 4 (6.8%) |
| Grade ≥3 | 0 | 1 (1.7%) |
| Infections and Infestations, any grade(%) | 69 (53.1%) | 17 (28.8%) |
| Grade ≥3 | 21 (16.1%) | 9 (15.3%) |
| Infections with Pathogen Unspecified, any grade(%) | 49 (37.7%) | 10 (16.9%) |
| Grade ≥3 | 9 (7%) | 5 (8.5%) |
| Leukocytosis, any grade(%) | 0 | 1 (1.7%) |
| Grade ≥3 | 0 | 0 |
| Neurological AE, any grade(%) | 60 (46.2%) | 32 (54.2%) |
| Grade ≥3 | 2 (1.6%) | 2 (3.4%) |
| Neutropenia, any grade(%) | 59 (45.4%) | 30 (50.8%) |
| Grade ≥3 | 45 (34.6%) | 18 (30.5%) |
| Opportunistic Infections, any grade(%) | 4 (3.1%) | 1 (1.7%) |
| Grade ≥3 | 1 (0.8%) | 1 (1.7%) |
| Pneumonitis/Interstitial Lung Disease, any grade(%) | 6 (4.6%) | 0 |
| Grade ≥3 | 3 (2.3%) | 0 |
| Rash, any grade(%) | 22 (16.9%) | 3 (5.1%) |
| Grade ≥3 | 1 (0.8%) | 0 |
| Thrombocytopenia / Platelet Ct. Decrease, any grade(%) | 12 (9.2%) | 38 (64.4%) |
| Grade ≥3 | 3 (2.3%) | 22 (37.3%) |
| Tumor Flare Events, any grade(%) | 9 (6.9%) | 0 |
| Grade ≥3 | 2 (1.5%) | 0 |
| Tumor Lysis Syndrome, any grade(%) | 1 (0.8%) | 0 |
| Grade ≥3 | 1 (0.8%) | 0 |
| Viral Infections, any grade(%) | 34 (26.2%) | 5 (8.5%) |
| Grade ≥3 | 12 (9.3%) | 3 (5.1%) |

MedDRA coded AE terms of “RASH”, “RASH ERYTHEMATOUS”, “EXFOLIATIVE RASH”, “RASH MACULAR”, “RASH MACULO-PAPULAR”, “RASH PRURITIC”, “RASH PUSTULAR”, “ERYTHEMA”, “PALMAR ERYTHEMA”, “DERMATITIS”, “DERMATITIS ACNEIFORM”, “DERMATITIS CONTACT”, “PALMAR-PLANTAR ERYTHRODYSAESTHESIA”, “RASH MORBILLIFORM”, “RASH PAPULAR” are combined to Rash.
Neutropenia includes “Neutrophil Count Decreased” and “Neutropenia”.

Mosun-Pola was a tolerable dosing regimen as determined by treatment completion rate (e.g., number of completed dosing cycles out of the planned 8 dosing cycles of mosunetuzumab and 6 dosing cycles of polatuzumab vedotin) and dose intensity (e.g., planned dose amount vs given dose amount). A number of patients administered R-GemOx did not complete all 8 planned cycles.

### Example 7. Improvements in Health-Related Quality of Life (HRQOL) in the SUNMO Study: Subcutaneous (SC) Mosunetuzumab Plus Polatuzumab Vedotin (Mosun-Pola) Versus Rituximab, Gemcitabine and Oxaliplatin (R-GemOx) in Patients (pts) with Relapsed/Refractory (R/R) Large B-Cell Lymphoma (LBCL) After At Least One Prior Therapy

Described below are results of analyses of patient-reported outcomes from the SUNMO study described in Examples 1, 4, and 6, comparing the combination treatment of Mosun+Pola to the reference treatment of R-GemOx in adult patients with relapsed or refractory large B-cell lymphoma (R/R LBCL) who were not candidates for autologous stem-cell transplantation (ASCT). Reported below are patient-reported outcomes (PROs) from SUNMO to assess the impact of treatment on HRQoL.

### Methods

Patients (Pts) (excluding Chinese patients) with autologous stem cell transplant-ineligible R/R LBCL were randomly assigned (2:1) to receive Mosun-Pola or R-GemOx. HRQOL was measured using European Organisation for Research and Treatment of Cancer Quality of Life Questionnaire Core 30 (EORTC QLQ-C30) for global health status, functioning, and core symptom scales; Functional Assessment of Cancer Treatment-Lymphoma (FACT-Lym) for lymphoma symptoms; and FACT Gynecologic Oncology Group-Neurotoxicity (FACT/GOG-NTX) for peripheral neuropathy (PN). Pts completed questionnaires on Day 1 of Cycles [C] 1-3, 5 and 7, at end of treatment (EOT), and at 3, 6, and 12 months post-EOT. Questionnaire completion rates, time to deterioration (TTD), change from baseline (CfB) and summary statistics were estimated at each assessment. Minimal clinically important difference (MCID) was defined as a 10-point change for EORTC QLQ-C30 and 3-point change for FACT-Lym and FACT/GOG-NTX, based on validated thresholds. Higher scores in EORTC QLQ-C30 functioning and global health scales, and FACT-Lym and FACT/GOG-NTX scales, indicate improvement; higher EORTC QLQ-C30 symptom scores indicate worsening.

### Results

Overall, 198 pts were randomized (Mosun-Pola, n=133; R-GemOx, n=65). PRO questionnaire completion rates were >95% in both arms. At baseline, pts in both arms reported low-to-mild symptom severity, moderate global health status/QoL, moderate-to-high functioning, mild-to-moderate lymphoma symptoms, and low rates of PN. For Mosun-Pola, median TTD in physical functioning was delayed by >1 year compared with R-GemOx (17.4 [95% CI 5.3—not estimable (NE)] vs 5.3 [1.9-NE] months), by >3 months for lymphoma symptoms (7.2 [95% CI 4.6-18.3] vs 3.8 [1.9-NE] months), and by >2 months for PN (4.3 [95% CI 2.9-6.3] vs 1.9 [1.0-6.4] months), suggesting greater durability in maintaining baseline scores. Stratified hazard ratios (HR [95% CI]) favored Mosun-Pola vs R-GemOx, with reduction in risk of deterioration by 31% for physical functioning (HR 0.7 [0.4-1.1]), 34% for PN (HR 0.7 [0.4-1.0]), 27% for fatigue (HR 0.7 [0.5-1.1]), and 18% for lymphoma symptoms (HR 0.8 [0.5-1.3]). Although mean baseline fatigue scores were higher in the Mosun-Pola arm (34.7 vs 26.25), fatigue improved with Mosun-Pola, while remaining stable in the R-GemOx arm. Improvements in lymphoma symptoms were similar in responding pts from both arms; pts with Mosun-Pola had clinically meaningful improvements in mean scores two cycles earlier for pain (CfB by EORTC QLQ-C30: Mosun-Pola: Cycle 5 (C5),-10.6; C7,-12.5; R-GemOx: C5,-6.7; C7,-16.7) and two cycles later for lymphoma symptoms (CfB by FACT-Lym: Mosun-Pola: C3, +2.6; C5, +4.9; C7, +4.9; R-GemOx: C3, +3.5; C5, +4.0; C7, +5.0) compared with R-GemOx. Pts with Mosun-Pola had clinically meaningful improvements in mean scores by EORTC QLQ-C30 for financial difficulties (C5,-9.1), emotional functioning (C5, +10.9) and constipation (C7,-10.7), while mean scores remained at baseline levels for pts in the R-GemOx arm.

### CONCLUSIONS

In SUNMO, Mosun-Pola provided benefits across multiple aspects of HRQOL compared with R-GemOx, particularly in maintaining/improving physical functioning, fatigue, lymphoma symptoms, and PN. Pts (e.g., with high disease burden and refractory status of the pts at baseline) treated with Mosun-Pola achieved clinically meaningful improvements in PROs, with scores exceeding the MCID in several domains and exhibiting a delay in deterioration of >1 year for physical functioning, >3 months for lymphoma symptoms, and >2 months for PN, and. Improved PROs with Mosun-Pola vs R-GemOx suggest benefits in HRQOL with chemotherapy-free bispecific antibody combinations.

### Embodiments

Some embodiments of the technology described herein can be defined according to any of the following numbered embodiments:

1. A method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

2. Use of mosunetuzumab in combination with polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

3. Use of polatuzumab vedotin in combination with mosunetuzumab for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

4. Use of mosunetuzumab and polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

5. Use of mosunetuzumab in the manufacture of a medicament for use in combination with polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

6. Use of polatuzumab vedotin in the manufacture of a medicament for use in combination with mosunetuzumab for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

7. Use of mosunetuzumab and polatuzumab vedotin in the manufacture of a medicament for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

8. Mosunetuzumab for use in combination with polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

9. Polatuzumab vedotin for use in combination with mosunetuzumab for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

10. Use of mosunetuzumab and polatuzumab vedotin for use for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

11. The method, use, mosunetuzumab for use, polatuzumab vedotin for use, or mosunetuzumab and polatuzumab vedotin for use of any one of embodiments 1-10, wherein the efficacy response is a CRR, and wherein the CRR or reference CRR is the proportion of the subjects receiving the corresponding treatment whose best overall response is a complete response (CR).

12. The method, use, mosunetuzumab for use, polatuzumab vedotin for use, or mosunetuzumab and polatuzumab vedotin for use of embodiment 11, wherein the improved response in CRR is an increase in CRR compared to the reference CRR of between 1% and 41%.

13. The method, use, mosunetuzumab for use, polatuzumab vedotin for use, or mosunetuzumab and polatuzumab vedotin for use of embodiment 12, wherein the improved response in CRR is an increase in CRR compared to the reference CRR of about 20.8%.

14. The method, use, mosunetuzumab for use, polatuzumab vedotin for use, or mosunetuzumab and polatuzumab vedotin for use of any one of embodiments 1-10, wherein the efficacy response is an ORR, and wherein the ORR or reference ORR is the proportion of the subjects receiving the corresponding treatment whose best overall response is a CR or a partial response (PR).

15. The method, use, mosunetuzumab for use, polatuzumab vedotin for use, or mosunetuzumab and polatuzumab vedotin for use of embodiment 14, wherein the improved response in ORR is an increase in ORR compared to the reference ORR of between 3% and 47%.

16. The method, use, mosunetuzumab for use, polatuzumab vedotin for use, or mosunetuzumab and polatuzumab vedotin for use of embodiment 15, wherein the improved response in ORR is an increase in ORR compared to the reference ORR of about 25.6%.

17. The method, use, mosunetuzumab for use, polatuzumab vedotin for use, or mosunetuzumab and polatuzumab vedotin for use of any one of embodiments 1-10, wherein the efficacy response is a DOR, and wherein the DOR or the reference DOR is measured starting from the time from the first occurrence of a documented CR or PR to disease progression or relapse or death from any cause, whichever occurs first.

18. The method, use, mosunetuzumab for use, polatuzumab vedotin for use, or mosunetuzumab and polatuzumab vedotin for use of embodiment 17, wherein the DOR or the reference DOR is the median DOR of the plurality of subjects receiving the corresponding treatment.

19. The method, use, mosunetuzumab for use, polatuzumab vedotin for use, or mosunetuzumab and polatuzumab vedotin for use of embodiment 17 or 18, wherein the efficacy response in median DOR is non-inferior compared to the reference median DOR.

20. The method, use, mosunetuzumab for use, polatuzumab vedotin for use, or mosunetuzumab and polatuzumab vedotin for use of embodiment 17, wherein the efficacy response in the rate of a DOR of 3 months is non-inferior compared to the reference rate of a DOR of 3 months.

21. The method, use, mosunetuzumab for use, polatuzumab vedotin for use, or mosunetuzumab and polatuzumab vedotin for use of embodiment 17, wherein the efficacy response in the rate of a DOR of 6 months is non-inferior compared to the reference rate of a DOR of 6 months.

22. The method, use, mosunetuzumab for use, polatuzumab vedotin for use, or mosunetuzumab and polatuzumab vedotin for use of embodiment 17, wherein the efficacy response in the rate of a DOR of 9 months is non-inferior compared to the reference rate of a DOR of 9 months.

23. The method, use, mosunetuzumab for use, polatuzumab vedotin for use, or mosunetuzumab and polatuzumab vedotin for use of any one of embodiments 1-10, wherein the efficacy response is a DOCR, and wherein the DOCR or the reference DOCR is measured starting from the time from the first occurrence of a documented CR to disease progression or relapse or death from any cause, whichever occurs first.

24. The method, use, mosunetuzumab for use, polatuzumab vedotin for use, or mosunetuzumab and polatuzumab vedotin for use of embodiment 23, wherein the DOCR or the reference DOCR is the median DOCR of the plurality of subjects receiving the corresponding treatment.

25. The method, use, mosunetuzumab for use, polatuzumab vedotin for use, or mosunetuzumab and polatuzumab vedotin for use of embodiment 23 or 24, wherein the efficacy response in median DOCR is non-inferior compared to the reference median DOCR.

26. The method, use, mosunetuzumab for use, polatuzumab vedotin for use, or mosunetuzumab and polatuzumab vedotin for use of embodiment 23, wherein the efficacy response in the rate of a DOCR of 3 months is non-inferior compared to the reference rate of a DOCR of 3 months.

27. The method, use, mosunetuzumab for use, polatuzumab vedotin for use, or mosunetuzumab and polatuzumab vedotin for use of embodiment 23, wherein the efficacy response in the rate of a DOCR of 6 months is non-inferior compared to the reference rate of a DOCR of 6 months.

28. The method, use, mosunetuzumab for use, polatuzumab vedotin for use, or mosunetuzumab and polatuzumab vedotin for use of embodiment 23, wherein the efficacy response in the rate of a DOCR of 9 months is non-inferior compared to the reference rate of a DOCR of 9 months.

29. The method, use, mosunetuzumab for use, polatuzumab vedotin for use, or mosunetuzumab and polatuzumab vedotin for use of any one of embodiments 5-19, wherein the CR or PR determined by PET/computed tomography (CT).

30. The method, use, mosunetuzumab for use, polatuzumab vedotin for use, or mosunetuzumab and polatuzumab vedotin for use of embodiment 29, wherein the CR or PR is determined based on the Lugano Response Criteria for Malignant Lymphoma (Cheson et al., 2014).

31. A method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

32. Use of mosunetuzumab in combination with polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

33. Use of polatuzumab vedotin in combination with mosunetuzumab for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

34. Use of mosunetuzumab and polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

35. Use of mosunetuzumab in the manufacture of a medicament for use in combination with polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

36. Use of polatuzumab vedotin in the manufacture of a medicament for use in combination with mosunetuzumab for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

37. Use of mosunetuzumab and polatuzumab vedotin in the manufacture of a medicament for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

38. Mosunetuzumab for use in combination with polatuzumab vedotin for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

39. Polatuzumab vedotin for use in combination with mosunetuzumab for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

40. Use of mosunetuzumab and polatuzumab vedotin for use for treating an aggressive non-Hodgkin's lymphoma (aNHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

41. The method, use, mosunetuzumab for use, polatuzumab vedotin for use, or mosunetuzumab and polatuzumab vedotin for use of any one of embodiments 31-40, wherein the safety response is the rate of AE, and wherein the rate of AE or the reference rate of AE is the rate of AE in the plurality of subjects receiving the corresponding treatment.

42. The method, use, mosunetuzumab for use, polatuzumab vedotin for use, or mosunetuzumab and polatuzumab vedotin for use of embodiment 41, wherein the safety response in rate of AE is non-inferior compared to the reference rate of AE.

43. The method, use, mosunetuzumab for use, polatuzumab vedotin for use, or mosunetuzumab and polatuzumab vedotin for use of embodiment 41, wherein the safety response in rate of Grade 3-5 AE is non-inferior compared to the reference rate of Grade 3-5 AE.

44. The method, use, mosunetuzumab for use, polatuzumab vedotin for use, or mosunetuzumab and polatuzumab vedotin for use of embodiment 43, wherein the Grade of AE is determined based on the National Cancer Institute Common Terminology Criteria for Adverse Events (NCI CTCAE), v5.0.

45. The method, use, mosunetuzumab for use, polatuzumab vedotin for use, or mosunetuzumab and polatuzumab vedotin for use of any one of embodiments 31-40, wherein the safety response is the rate of SAE, and wherein the rate of SAE or the reference rate of SAE is the rate of SAE in the plurality of subjects receiving the corresponding treatment.

46. The method, use, mosunetuzumab for use, polatuzumab vedotin for use, or mosunetuzumab and polatuzumab vedotin for use of embodiment 45, wherein the safety response in rate of SAE is non-inferior compared to the reference rate of SAE.

47. The method, use, mosunetuzumab for use, polatuzumab vedotin for use, or mosunetuzumab and polatuzumab vedotin for use of any one of embodiments 1-46, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
   (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (ii) an intravenous dose of polatuzumab vedotin administered on Day 1 of the first dosing cycle, wherein the intravenous dose of polatuzumab vedotin is about 1.8 mg/kg; and

(b) the second dosing cycle comprises:
(i) a first subcutaneous dose (scC2D1) of mosunetuzumab administered on Day 1 of the second dosing cycle, wherein the scC2D1 is about 45 mg; and
(ii) an intravenous dose of polatuzumab vedotin administered on Day 1 of the second dosing cycle, wherein the intravenous dose of polatuzumab vedotin is about 1.8 mg/kg.

48. The method, use, mosunetuzumab for use, polatuzumab vedotin for use, or mosunetuzumab and polatuzumab vedotin for use of embodiment 47, wherein the dosing regimen of the combination treatment further comprises one or more additional 21-day dosing cycles.

49. The method, use, mosunetuzumab for use, polatuzumab vedotin for use, or mosunetuzumab and polatuzumab vedotin for use of embodiment 48, wherein the dosing regimen of the combination treatment comprises six additional 21-day dosing cycles.

50. The method, use, mosunetuzumab for use, polatuzumab vedotin for use, or mosunetuzumab and polatuzumab vedotin for use of embodiment 48 or 49, wherein each additional dosing cycle comprises:
(a) a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; or
(b) a subcutaneous dose of mosunetuzumab administered on Day 1 of the additional dosing cycle, wherein the subcutaneous dose of mosunetuzumab is about 45 mg.

51. The method, use, mosunetuzumab for use, polatuzumab vedotin for use, or mosunetuzumab and polatuzumab vedotin for use of embodiment 49, wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg.

52. The method, use, mosunetuzumab for use, polatuzumab vedotin for use, or mosunetuzumab and polatuzumab vedotin for use of any one of embodiments 1-51, wherein the control treatment comprises intravenously administering 375 mg/m² rituximab, 1000 mg/m² gemcitabine, and 100 mg/m² oxaliplatin for eight 14-day dosing cycles, wherein rituximab, gemcitabine, and oxaliplatin are administered on Day 1 of each dosing cycle.

53. A method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

54. Use of mosunetuzumab in combination with polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

55. Use of polatuzumab vedotin in combination with mosunetuzumab for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

56. Use of mosunetuzumab and polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

57. Use of mosunetuzumab in the manufacture of a medicament for use in combination with polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

58. Use of polatuzumab vedotin in the manufacture of a medicament for use in combination with mosunetuzumab for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

59. Use of mosunetuzumab and polatuzumab vedotin in the manufacture of a medicament for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

60. Mosunetuzumab for use in combination with polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

61. Polatuzumab vedotin for use in combination with mosunetuzumab for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

62. Use of mosunetuzumab and polatuzumab vedotin for use for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

63. The method, use, mosunetuzumab for use, polatuzumab vedotin for use, or mosunetuzumab and polatuzumab vedotin for use of any one of embodiments 53-62, wherein the efficacy response is a CRR, and wherein the CRR or reference CRR is the proportion of the subjects receiving the corresponding treatment whose best overall response is a complete response (CR).

64. The method, use, mosunetuzumab for use, polatuzumab vedotin for use, or mosunetuzumab and polatuzumab vedotin for use of embodiment 63, wherein the improved response in CRR is an increase in CRR compared to the reference CRR of between 1% and 52%.

65. The method, use, mosunetuzumab for use, polatuzumab vedotin for use, or mosunetuzumab and polatuzumab vedotin for use of embodiment 64, wherein the improved response in CRR is an increase in CRR compared to the reference CRR of about 23%.

66. The method, use, mosunetuzumab for use, polatuzumab vedotin for use, or mosunetuzumab and polatuzumab vedotin for use of any one of embodiments 53-62, wherein the efficacy response is an ORR, and wherein the ORR or reference ORR is the proportion of the subjects receiving the corresponding treatment whose best overall response is a CR or a partial response (PR).

67. The method, use, mosunetuzumab for use, polatuzumab vedotin for use, or mosunetuzumab and polatuzumab vedotin for use of embodiment 66, wherein the improved response in ORR is an increase in ORR compared to the reference ORR of between 1% and 55%.

68. The method, use, mosunetuzumab for use, polatuzumab vedotin for use, or mosunetuzumab and polatuzumab vedotin for use of embodiment 67, wherein the improved response in ORR is an increase in ORR compared to the reference ORR of about 28%.

69. The method, use, mosunetuzumab for use, polatuzumab vedotin for use, or mosunetuzumab and polatuzumab vedotin for use of any one of embodiments 53-62, wherein the efficacy response is a DOR, and wherein the DOR or the reference DOR is measured starting from the time from the first occurrence of a documented CR or PR to disease progression or relapse or death from any cause, whichever occurs first.

70. The method, use, mosunetuzumab for use, polatuzumab vedotin for use, or mosunetuzumab and polatuzumab vedotin for use of embodiment 69, wherein the DOR or the reference DOR is the median DOR of the plurality of subjects receiving the corresponding treatment.

71. The method, use, mosunetuzumab for use, polatuzumab vedotin for use, or mosunetuzumab and polatuzumab vedotin for use of embodiment 69 or 67, wherein the improvement of the median DOR is an increase in the DOR compared to the reference DOR of between 1 and 21 months.

72. The method, use, mosunetuzumab for use, polatuzumab vedotin for use, or mosunetuzumab and polatuzumab vedotin for use of embodiment 69, wherein the improvement of the DOR is an increase in the rate of a DOR of 6 months compared to the rate of a reference DOR of 6 months of between 1% and 61%.

73. The method, use, mosunetuzumab for use, polatuzumab vedotin for use, or mosunetuzumab and polatuzumab vedotin for use of embodiment 72, wherein the improvement of the DOR is an increase in the rate of a DOR of 6 months compared to the rate of a reference DOR of 6 months of about 20%.

74. The method, use, mosunetuzumab for use, polatuzumab vedotin for use, or mosunetuzumab and polatuzumab vedotin for use of embodiment 69, wherein the improvement of the DOR is an increase in the rate of a DOR of 9 months compared to the rate of a reference DOR of 9 months of between 1% and 72%.

75. The method, use, mosunetuzumab for use, polatuzumab vedotin for use, or mosunetuzumab and polatuzumab vedotin for use of embodiment 73, wherein the improvement of the DOR is an increase in the rate of a DOR of 9 months compared to the rate of a reference DOR of 9 months of about 26%.

76. The method, use, mosunetuzumab for use, polatuzumab vedotin for use, or mosunetuzumab and polatuzumab vedotin for use of embodiment 69, wherein the improvement of the DOR is an increase in the rate of a DOR of 12 months compared to the rate of a reference DOR of 12 months of between 1% and 87%.

77. The method, use, mosunetuzumab for use, polatuzumab vedotin for use, or mosunetuzumab and polatuzumab vedotin for use of embodiment 76, wherein the improvement of the DOR is an increase in the rate of a DOR of 12 months compared to the rate of a reference DOR of 12 months of about 39%.

78. The method, use, mosunetuzumab for use, polatuzumab vedotin for use, or mosunetuzumab and polatuzumab vedotin for use of embodiment 69, wherein the improvement of the DOR is an increase in the rate of a DOR of 18 months compared to the rate of a reference DOR of 18 months of between 43% and 92%.

79. The method, use, mosunetuzumab for use, polatuzumab vedotin for use, or mosunetuzumab and polatuzumab vedotin for use of embodiment 78, wherein the improvement of the DOR is an increase in the rate of a DOR of 18 months compared to the rate of a reference DOR of 18 months of about 67%.

80. The method, use, mosunetuzumab for use, polatuzumab vedotin for use, or mosunetuzumab and polatuzumab vedotin for use of any one of embodiments 63-79, wherein the CR or PR determined by PET/computed tomography (CT).

81. The method, use, mosunetuzumab for use, polatuzumab vedotin for use, or mosunetuzumab and polatuzumab vedotin for use of embodiment 80, wherein the CR or PR is determined based on the Lugano Response Criteria for Malignant Lymphoma (Cheson et al., 2014).

82. The method, use, mosunetuzumab for use, polatuzumab vedotin for use, or mosunetuzumab and polatuzumab vedotin for use of any one of embodiments 53-62, wherein the efficacy response is a PFS, and wherein the PFS or the reference PFS is measured starting from the time of receiving first dose of mosunetuzumab or polatuzumab vedotin to the time of a first occurrence of disease progression or death from any cause.

83. The method, use, mosunetuzumab for use, polatuzumab vedotin for use, or mosunetuzumab and polatuzumab vedotin for use of embodiment 82, wherein the PFS or the reference PFS is the median PFS of the plurality of subjects receiving the corresponding treatment.

84. The method, use, mosunetuzumab for use, polatuzumab vedotin for use, or mosunetuzumab and polatuzumab vedotin for use of embodiment 82 or 83, wherein the improvement of the median PFS is an increase in the PFS compared to the reference PFS of between 1 and 24 months.

85. The method, use, mosunetuzumab for use, polatuzumab vedotin for use, or mosunetuzumab and polatuzumab vedotin for use of embodiment 82, wherein the improvement of the PFS is an increase in the rate of a PFS of 6 months compared to the rate of a reference PFS of 6 months of between 1% and 53%.

86. The method, use, mosunetuzumab for use, polatuzumab vedotin for use, or mosunetuzumab and polatuzumab vedotin for use of embodiment 85, wherein the improvement of the PFS is an increase in the rate of a PFS of 6 months compared to the rate of a reference PFS of 6 months of about 21%.

87. The method, use, mosunetuzumab for use, polatuzumab vedotin for use, or mosunetuzumab and polatuzumab vedotin for use of embodiment 82, wherein the improvement of the PFS is an increase in the rate of a PFS of 9 months compared to the rate of a reference PFS of 9 months of between 1% and 62%.

88. The method, use, mosunetuzumab for use, polatuzumab vedotin for use, or mosunetuzumab and polatuzumab vedotin for use of embodiment 87, wherein the improvement of the PFS is an increase in the rate of a PFS of 9 months compared to the rate of a reference PFS of 9 months of about 28%.

89. The method, use, mosunetuzumab for use, polatuzumab vedotin for use, or mosunetuzumab and polatuzumab vedotin for use of embodiment 82, wherein the improvement of the PFS is an increase in the rate of a PFS of 12 months compared to the rate of a reference PFS of 12 months of between 1% and 63%.

90. The method, use, mosunetuzumab for use, polatuzumab vedotin for use, or mosunetuzumab and polatuzumab vedotin for use of embodiment 89, wherein the improvement of the PFS is an increase in the rate of a PFS of 12 months compared to the rate of a reference PFS of 12 months of about 27%.

91. The method, use, mosunetuzumab for use, polatuzumab vedotin for use, or mosunetuzumab and polatuzumab vedotin for use of embodiment 82, wherein the improvement of the PFS is an increase in the rate of a PFS of 18 months compared to the rate of a reference PFS of 18 months of between 1% and 68%.

92. The method, use, mosunetuzumab for use, polatuzumab vedotin for use, or mosunetuzumab and polatuzumab vedotin for use of embodiment 91, wherein the improvement of the PFS is an increase in the rate of a PFS of 18 months compared to the rate of a reference PFS of 18 months of about 25%. 93. A method of treating a relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

94. Use of mosunetuzumab in combination with polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved efficacy response of the plurality of subjects as compared to a reference efficacy response, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

95. Use of polatuzumab vedotin in combination with mosunetuzumab for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

96. Use of mosunetuzumab and polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

97. Use of mosunetuzumab in the manufacture of a medicament for use in combination with polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

98. Use of polatuzumab vedotin in the manufacture of a medicament for use in combination with mosunetuzumab for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

99. Use of mosunetuzumab and polatuzumab vedotin in the manufacture of a medicament for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

100. Mosunetuzumab for use in combination with polatuzumab vedotin for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

101. Polatuzumab vedotin for use in combination with mosunetuzumab for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

102. Use of mosunetuzumab and polatuzumab vedotin for use for treating a relapsed and/or refractory non-Hodgkin's lymphoma (R/R NHL) in a subject in need thereof, wherein mosunetuzumab is to be administered subcutaneously and polatuzumab vedotin is to be administered intravenously, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

103. The method, use, mosunetuzumab for use, polatuzumab vedotin for use, or mosunetuzumab and polatuzumab vedotin for use of any one of embodiments 93-102, wherein (a) the safety response is the rate of AE, and wherein the rate of AE or the reference rate of AE is the rate of AE in the plurality of subjects receiving the corresponding treatment; or (b) the safety response is the rate of SAE, and wherein the rate of SAE or the reference rate of SAE is the rate of SAE in the plurality of subjects receiving the corresponding treatment.

104. The method, use, mosunetuzumab for use, polatuzumab vedotin for use, or mosunetuzumab and polatuzumab vedotin for use of embodiment 103, wherein (a) the safety response in rate of AE is non-inferior compared to the reference rate of AE; or (b) the safety response in rate of SAE is non-inferior compared to the reference rate of SAE.

105. The method, use, mosunetuzumab for use, polatuzumab vedotin for use, or mosunetuzumab and polatuzumab vedotin for use of any one of embodiments 53-104, wherein the control treatment comprises (a) intravenously administering 375 mg/m² rituximab and 1.8 mg/kg polatuzumab vedotin for six 21-day dosing cycles, wherein rituximab and polatuzumab vedotin are administered on Day 1 of each dosing cycle; and (b) intravenously administering 375 mg/m² rituximab for two 21-day dosing cycles, wherein rituximab is administered on Day 1 of each dosing cycle.

106. A method of achieving an objective response (OR), a complete response (CR), an overall survival (OS), or a progression free survival (PFS) in a subject having a relapsed and/or refractory non-Hodgkin's lymphoma (R/R NHL), wherein the subject is administered a combination treatment comprising subcutaneously administered mosunetuzumab and intravenously administered polatuzumab vedotin.

107. Use of mosunetuzumab in combination with polatuzumab vedotin for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

108. Use of polatuzumab vedotin in combination with mosunetuzumab for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

109. Use of mosunetuzumab and polatuzumab vedotin for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

110. Use of mosunetuzumab in the manufacture of a medicament for use in combination with polatuzumab vedotin for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

111. Use of polatuzumab vedotin in the manufacture of a medicament for use in combination with mosunetuzumab for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

112. Use of mosunetuzumab and polatuzumab vedotin in the manufacture of a medicament for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

113. Mosunetuzumab for use in combination with polatuzumab vedotin for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

114. Polatuzumab vedotin for use in combination with mosunetuzumab for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

115. Use of mosunetuzumab and polatuzumab vedotin for use for achieving an objective response (OR), a complete response (CR), an overall survival (OS), or a progression free survival (PFS) in a subject having a relapsed and/or refractory non-Hodgkin's lymphoma (R/R NHL), wherein the subject is to be administered a combination treatment comprising subcutaneously administered mosunetuzumab and intravenously administered polatuzumab vedotin.

116. The method, use, mosunetuzumab for use, polatuzumab vedotin for use, or mosunetuzumab and polatuzumab vedotin for use of any one of embodiments 106-115, wherein the subject achieves an OR.

117. The method, use, mosunetuzumab for use, polatuzumab vedotin for use, or mosunetuzumab and polatuzumab vedotin for use of embodiment 116, wherein the OR is maintained for at least 6, 9, 12, or 18 months.

118. The method, use, mosunetuzumab for use, polatuzumab vedotin for use, or mosunetuzumab and polatuzumab vedotin for use of any one of embodiments 106-115, wherein the subject achieves a CR.

119. The method, use, mosunetuzumab for use, polatuzumab vedotin for use, or mosunetuzumab and polatuzumab vedotin for use of any one of embodiments 106-115, wherein the OS is maintained for at least 6, 9, 12, or 18 months.

120. The method, use, mosunetuzumab for use, polatuzumab vedotin for use, or mosunetuzumab and polatuzumab vedotin for use of any one of embodiments 106-115, wherein the PFS is maintained for at least 6, 9, 12, or 18 months.

121. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall response rate of between 62% and 89%.

122. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

123. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

124. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

125. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

126. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

127. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

128. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

129. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

130. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall response rate of between 62% and 89%.

131. The method, use, mosunetuzumab for use, polatuzumab vedotin for use, or mosunetuzumab and polatuzumab vedotin for use of any one of embodiments 121-130, wherein the overall response rate is about 78%.

132. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a complete response rate of between 41% and 73%.

133. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

134. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

135. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

136. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

137. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

138. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

139. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

140. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

141. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a complete response rate of between 41% and 73%.

142. The method, use, mosunetuzumab for use, polatuzumab vedotin for use, or mosunetuzumab and polatuzumab vedotin for use of any one of embodiments 132-141, wherein the complete response rate is about 58%.

143. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall survival rate at 9 months of between 66% and 92%.

144. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

145. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

146. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

147. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

148. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

149. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

150. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

151. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

152. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 9 months of between 66% and 92%.

153. The method, use, mosunetuzumab for use, polatuzumab vedotin for use, or mosunetuzumab and polatuzumab vedotin for use of any one of embodiments 143-152, wherein the overall survival rate at 9 months is about 79%.

154. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has an overall survival rate at 12 months of between 60% and 88%.

155. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

156. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

157. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

158. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

159. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

160. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

161. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

162. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

163. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has an overall survival rate at 12 months of between 60% and 88%.

164. The method, use, mosunetuzumab for use, polatuzumab vedotin for use, or mosunetuzumab and polatuzumab vedotin for use of any one of embodiments 154-163, wherein the overall survival rate at 12 months is about 74%.

165. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a progression free survival rate at 9 months of between 57% and 87%.

166. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

167. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

168. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

169. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

170. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

171. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

172. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

173. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

174. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 9 months of between 57% and 87%.

175. The method, use, mosunetuzumab for use, polatuzumab vedotin for use, or mosunetuzumab and polatuzumab vedotin for use of any one of embodiments 165-174, wherein the progression free survival rate at 9 months is about 72%.

176. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects has a progression free survival rate at 12 months of between 47% and 81%.

177. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

178. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

179. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

180. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

181. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

182. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

183. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

184. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

185. Use of mosunetuzumab and polatuzumab vedotin for treating a population of subjects having a relapsed and/or refractory non-Hodgkin's lymphoma (R/R NHL), wherein each subject of the population is to be administered a combination treatment comprising subcutaneously administered mosunetuzumab and intravenously administered polatuzumab vedotin, and wherein the population of subjects has a progression free survival rate at 12 months of between 47% and 81%.

186. The method, use, mosunetuzumab for use, polatuzumab vedotin for use, or mosunetuzumab and polatuzumab vedotin for use of any one of embodiments 176-185, wherein the progression free survival rate at 12 months is about 64%.

187. The method, use, mosunetuzumab for use, polatuzumab vedotin for use, or mosunetuzumab and polatuzumab vedotin for use of any one of embodiments 53-186, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
   (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
   (i) a first subcutaneous dose (scC2D1) of mosunetuzumab administered on Day 1 of the second dosing cycle, wherein the scC2D1 is about 45 mg; and
   (ii) an intravenous dose of polatuzumab vedotin administered on Day 1 of the second dosing cycle, wherein the intravenous dose of polatuzumab vedotin is about 1.8 mg/kg.

188. The method, use, mosunetuzumab for use, polatuzumab vedotin for use, or mosunetuzumab and polatuzumab vedotin for use of embodiment 187, wherein the dosing regimen of the combination treatment further comprises one or more additional 21-day dosing cycles.

189. The method, use, mosunetuzumab for use, polatuzumab vedotin for use, or mosunetuzumab and polatuzumab vedotin for use of embodiment 188, wherein the dosing regimen of the combination treatment comprises six additional 21-day dosing cycles.

190. The method, use, mosunetuzumab for use, polatuzumab vedotin for use, or mosunetuzumab and polatuzumab vedotin for use of embodiment 188 or 189, wherein each additional dosing cycle comprises:
(a) a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; or
(b) a subcutaneous dose of mosunetuzumab administered on Day 1 of the additional dosing cycle, wherein the subcutaneous dose of mosunetuzumab is about 45 mg.

191. The method, use, mosunetuzumab for use, polatuzumab vedotin for use, or mosunetuzumab and polatuzumab vedotin for use of embodiment 190, wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg.

192. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median overall survival of between 15 and 39 months.

193. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

194. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

195. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

196. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

197. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

198. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

199. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

200. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

201. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median overall survival of between 15 and 39 months.

202. The method, use, mosunetuzumab for use, polatuzumab vedotin for use, or mosunetuzumab and polatuzumab vedotin for use of any one of embodiments 192-201, wherein the median overall survival is about 27 months.

203. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 6 months of between 73% and 89%.

204. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

205. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

206. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

207. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

208. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

209. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

210. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

211. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

212. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 6 months of between 73% and 89%.

213. The method, use, mosunetuzumab for use, polatuzumab vedotin for use, or mosunetuzumab and polatuzumab vedotin for use of any one of embodiments 203-212, wherein the overall survival rate at 6 months is about 81%.

214. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 9 months of between 61% and 80%.

215. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

216. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

217. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

218. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

219. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

220. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

221. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

222. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

223. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 9 months of between 61% and 80%.

224. The method, use, mosunetuzumab for use, polatuzumab vedotin for use, or mosunetuzumab and polatuzumab vedotin for use of any one of embodiments 214-223, wherein the overall survival rate at 9 months is about 70%.

225. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 12 months of between 55% and 75%.

226. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

227. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

228. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

229. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

230. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

231. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

232. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

233. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

234. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 12 months of between 55% and 75%.

235. The method, use, mosunetuzumab for use, polatuzumab vedotin for use, or mosunetuzumab and polatuzumab vedotin for use of any one of embodiments 225-234, wherein the overall survival rate at 12 months is about 65%.

236. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 18 months of between 45% and 65%.

237. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

238. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

239. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

240. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

241. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

242. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

243. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

244. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

245. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 18 months of between 45% and 65%.

246. The method, use, mosunetuzumab for use, polatuzumab vedotin for use, or mosunetuzumab and polatuzumab vedotin for use of any one of embodiments 236-245, wherein the overall survival rate at 18 months is about 55%.

247. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has an overall survival rate at 24 months of between 40% and 61%.

248. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

249. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

250. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

251. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

252. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

253. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

254. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

255. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

256. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has an overall survival rate at 24 months of between 40% and 61%.

257. The method, use, mosunetuzumab for use, polatuzumab vedotin for use, or mosunetuzumab and polatuzumab vedotin for use of any one of embodiments 247-256, wherein the overall survival rate at 24 months is about 50%.

258. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median progression free survival of between 9 and 27 months.

259. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

260. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

261. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

262. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

263. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

264. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

265. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

266. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

267. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median progression free survival of between 9 and 27 months.

268. The method, use, mosunetuzumab for use, polatuzumab vedotin for use, or mosunetuzumab and polatuzumab vedotin for use of any one of embodiments 258-267, wherein the median progression free survival is about 14 months.

269. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 6 months of between 51% and 72%.

270. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

271. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

272. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

273. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

274. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

275. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

276. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

277. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

278. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 6 months of between 51% and 72%.

279. The method, use, mosunetuzumab for use, polatuzumab vedotin for use, or mosunetuzumab and polatuzumab vedotin for use of any one of embodiments 269-278, wherein the progression free survival rate at 6 months is about 62%.

280. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 9 months of between 50% and 71%.

281. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

282. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

283. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

284. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

285. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

286. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

287. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

288. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

289. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 9 months of between 50% and 71%.

290. The method, use, mosunetuzumab for use, polatuzumab vedotin for use, or mosunetuzumab and polatuzumab vedotin for use of any one of embodiments 280-289, wherein the progression free survival rate at 9 months is about 60%.

291. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 12 months of between 41% and 64%.

292. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

293. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

294. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

295. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

296. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

297. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

298. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

299. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

300. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 41% and 64%.

301. The method, use, mosunetuzumab for use, polatuzumab vedotin for use, or mosunetuzumab and polatuzumab vedotin for use of any one of embodiments 291-300, wherein the progression free survival rate at 12 months is about 52%.

302. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 18 months of between 34% and 57%.

303. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

304. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

305. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

306. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

307. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

308. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

309. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

310. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

311. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 18 months of between 34% and 57%.

312. The method, use, mosunetuzumab for use, polatuzumab vedotin for use, or mosunetuzumab and polatuzumab vedotin for use of any one of embodiments 302-311, wherein the progression free survival rate at 18 months is about 46%.

313. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a progression free survival rate at 24 months of between 28% and 52%.

314. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

315. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

316. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

317. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

318. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

319. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

320. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

321. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

322. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 24 months of between 28% and 52%.

323. The method, use, mosunetuzumab for use, polatuzumab vedotin for use, or mosunetuzumab and polatuzumab vedotin for use of any one of embodiments 313-322, wherein the progression free survival rate at 24 months is about 40%.

324. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein the population of subjects has a median duration of response of between 16 and 39 months.

325. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

326. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

327. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

328. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

329. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

330. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

331. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

332. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

333. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a median duration of response of between 16 and 39 months.

334. The method, use, mosunetuzumab for use, polatuzumab vedotin for use, or mosunetuzumab and polatuzumab vedotin for use of any one of embodiments 324-333, wherein the median duration of response is about 28 months.

335. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 78% and 96% of the population of subjects maintains a durable response for 6 months.

336. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

337. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

338. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

339. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

340. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

341. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

342. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

343. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 78% and 96% of the population of subjects maintains a durable response for 6 months.

344. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein the population of subjects has a progression free survival rate at 12 months of between 47% and 81%.

345. The method, use, mosunetuzumab for use, polatuzumab vedotin for use, or mosunetuzumab and polatuzumab vedotin for use of any one of embodiments 335-344, wherein about 87% of the population of subjects maintains a durable response for 6 months.

346. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 63% and 88% of the population of subjects maintains a durable response for 9 months.

347. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

348. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

349. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

350. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

351. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

352. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

353. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

354. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

355. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 63% and 88% of the population of subjects maintains a durable response for 9 months.

356. The method, use, mosunetuzumab for use, polatuzumab vedotin for use, or mosunetuzumab and polatuzumab vedotin for use of any one of embodiments 346-355, wherein about 75% of the population of subjects maintains a durable response for 9 months.

357. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 58% and 84% of the population of subjects maintains a durable response for 12 months.

358. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

359. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

360. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

361. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

362. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

363. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

364. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

365. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

366. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 58% and 84% of the population of subjects maintains a durable response for 12 months.

367. The method, use, mosunetuzumab for use, polatuzumab vedotin for use, or mosunetuzumab and polatuzumab vedotin for use of any one of embodiments 357-366, wherein about 71% of the population of subjects maintains a durable response for 12 months.

368. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 46% and 75% of the population of subjects maintains a durable response for 18 months.

369. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

370. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

371. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

372. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

373. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

374. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

375. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

376. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

377. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 46% and 75% of the population of subjects maintains a durable response for 18 months.

378. The method, use, mosunetuzumab for use, polatuzumab vedotin for use, or mosunetuzumab and polatuzumab vedotin for use of any one of embodiments 368-377, wherein about 61% of the population of subjects maintains a durable response for 18 months.

379. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein between 43% and 73% of the population of subjects maintains a durable response for 24 months.

380. Use of mosunetuzumab in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

381. Use of polatuzumab vedotin in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

382. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

383. Use of mosunetuzumab in the manufacture of a medicament for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

384. Use of polatuzumab vedotin in the manufacture of a medicament for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

385. Use of mosunetuzumab and polatuzumab vedotin in the manufacture of a medicament for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

386. Mosunetuzumab for use in combination with polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

387. Polatuzumab vedotin for use in combination with mosunetuzumab for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

388. Use of mosunetuzumab and polatuzumab vedotin for treating a population of subjects having a relapsed and/or refractory non-Hodgkin's lymphoma (R/R NHL), wherein each subject of the population is to be administered a combination treatment comprising intravenously administered mosunetuzumab and intravenously administered polatuzumab vedotin, and wherein between 43% and 73% of the population of subjects maintains a durable response for 24 months.

389. The method, use, mosunetuzumab for use, polatuzumab vedotin for use, or mosunetuzumab and polatuzumab vedotin for use of any one of embodiments 379-388, wherein about 58% of the population of subjects maintains a durable response for 24 months.

390. The method, use, mosunetuzumab for use, polatuzumab vedotin for use, or mosunetuzumab and polatuzumab vedotin for use of any one of embodiments 192-389, wherein the combination treatment comprises intravenously administering mosunetuzumab and intravenously administering polatuzumab vedotin according to a dosing regimen comprising eight 21-day dosing cycle, wherein:
(a) the first dosing cycle comprises:
   (i) a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg; and
   (ii) an intravenous dose of polatuzumab vedotin administered on Day 1 of the first dosing cycle, wherein the intravenous dose of polatuzumab vedotin is about 1.8 mg/kg;
(b) the second dosing cycle comprises:
   (i) a first intravenous dose (ivC2D1) of mosunetuzumab administered on Day 1 of the second dosing cycle, wherein the ivC2D1 is about 60 mg; and
   (ii) an intravenous dose of polatuzumab vedotin administered on Day 1 of the second dosing cycle, wherein the intravenous dose of polatuzumab vedotin is about 1.8 mg/kg;
(c) the third to sixth dosing cycles each comprises:
   (i) a first intravenous dose (ivC3D1-ivC6D1) of mosunetuzumab administered on Day 1 of each respective dosing cycle, wherein the ivC3D1-ivC6D1 is each about 30 mg; and
   (ii) an intravenous dose of polatuzumab vedotin administered on Day 1 of the second dosing cycle, wherein the intravenous dose of polatuzumab vedotin is about 1.8 mg/kg; and
(d) the seventh and eighth dosing cycles each comprises a first intravenous dose (ivC7D1-ivC8D1) of mosunetuzumab administered on Day 1 of each respective dosing cycle, wherein the ivC7D1-ivC8D1 is each about 30 mg and does not comprise administration of polatuzumab vedotin.

391. The method, use, mosunetuzumab for use, polatuzumab vedotin for use, or mosunetuzumab and polatuzumab vedotin for use of any one of embodiments 1-390, wherein the subject has an Eastern Cooperative Oncology Group (ECOG) Performance Status of 0, 1, or 2.

392. The method, use, mosunetuzumab for use, polatuzumab vedotin for use, or mosunetuzumab and polatuzumab vedotin for use of any one of embodiments 1-52 and 391, wherein aNHL is a diffuse large B cell lymphoma (DLBCL), a high grade B cell lymphoma (HGBL), a Grade 3b follicular lymphoma (FL), or a transformed FL (trFL).

393. The method, use, mosunetuzumab for use, polatuzumab vedotin for use, or mosunetuzumab and polatuzumab vedotin for use of any one of embodiments 53-391, wherein the R/R NHL is an R/R large B cell lymphoma (LBCL), an R/R diffuse large B cell lymphoma (DLBCL), an R/R follicular lymphoma (FL), or an R/R mantle cell lymphoma (MCL).

394. The method, use, mosunetuzumab for use, polatuzumab vedotin for use, or mosunetuzumab and polatuzumab vedotin for use of any one of embodiments 1-393, wherein the subject has relapsed after or is refractory to one or more prior lines of systemic therapy.

395. The method, use, mosunetuzumab for use, polatuzumab vedotin for use, or mosunetuzumab and polatuzumab vedotin for use of any one of embodiments 1-394, wherein the subject is ineligible for autologous stem cell transplant (ASCT).

396. The method, use, mosunetuzumab for use, polatuzumab vedotin for use, or mosunetuzumab and polatuzumab vedotin for use of any one of embodiments 1-395, wherein the method comprises administering an additional therapeutic agent.

397. The method, use, mosunetuzumab for use, polatuzumab vedotin for use, or mosunetuzumab and polatuzumab vedotin for use of embodiment 396, wherein the additional therapeutic agent comprises a corticosteroid, an antihistamine, an antipyretic, or an IL-6R antagonist.

398. The method, use, mosunetuzumab for use, polatuzumab vedotin for use, or mosunetuzumab and polatuzumab vedotin for use of embodiment 397, wherein the additional therapeutic agent is a corticosteroid, and wherein the corticosteroid comprises prednisone, methylprednisolone, or dexamethasone.

399. The method, use, mosunetuzumab for use, polatuzumab vedotin for use, or mosunetuzumab and polatuzumab vedotin for use of embodiment 397, wherein the additional therapeutic agent is an antihistamine, and wherein the antihistamine comprises diphenhydramine hydrochloride or equivalent.

400. The method, use, mosunetuzumab for use, polatuzumab vedotin for use, or mosunetuzumab and polatuzumab vedotin for use of embodiment 397, wherein the additional therapeutic agent is an antipyretic, and wherein the antipyretic is acetaminophen.

401. The method, use, mosunetuzumab for use, polatuzumab vedotin for use, or mosunetuzumab and polatuzumab vedotin for use of embodiment 397, wherein the additional therapeutic agent is an IL-6R antagonist, wherein the IL-6R antagonist is tocilizumab.

402. A method of treating a population of subjects having a relapsed and/or refractory MCL, wherein each subject of the population is administered a combination treatment comprising subcutaneously administered mosunetuzumab and intravenously administered polatuzumab vedotin, and wherein the population of subjects has an overall response rate of between 74% and 100%.

403. The method of embodiment 402, wherein the overall response rate is about 88%.

404. A method of treating a population of subjects having a relapsed and/or refractory MCL, wherein each subject of the population is administered a combination treatment comprising subcutaneously administered mosunetuzumab and intravenously administered polatuzumab vedotin, and wherein the population of subjects has a complete response rate of between 63% and 93%.

405. The method of embodiment 404, wherein the complete response rate is about 79%.

406. A method of treating a population of subjects having a relapsed and/or refractory MCL, wherein each subject of the population is administered a combination treatment comprising subcutaneously administered mosunetuzumab and intravenously administered polatuzumab vedotin, and wherein the median time to first response in the population of subjects is between 80 days to 100 days.

407. The method of embodiment 406, wherein the median time to first response in the population of subjects is about 3 months.

408. A method of treating a population of subjects having a relapsed and/or refractory MCL, wherein each subject of the population is administered a combination treatment comprising subcutaneously administered mosunetuzumab and intravenously administered polatuzumab vedotin, and wherein the median PFS in the population of subjects is at least 14 months.

409. The method of embodiment 408, wherein the median PFS in the population of subjects is about 19 months.

410. A method of treating a population of subjects having a relapsed and/or refractory MCL, wherein each subject of the population is administered a combination treatment comprising subcutaneously administered mosunetuzumab and intravenously administered polatuzumab vedotin, and wherein the median OS in the population of subjects is at least 17 months.

411. The method of embodiment 410, wherein the median OS in the population of subjects is about 21 months.

412. The method of any one of embodiments 402-411, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
   (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
   (i) a first subcutaneous dose (scC2D1) of mosunetuzumab administered on Day 1 of the second dosing cycle, wherein the scC2D1 is about 45 mg; and
   (ii) an intravenous dose of polatuzumab vedotin administered on Day 1 of the second dosing cycle, wherein the intravenous dose of polatuzumab vedotin is about 1.8 mg/kg.

413. The method of embodiment 412, wherein the dosing regimen of the combination treatment further comprises one or more additional 21-day dosing cycles.

414. The method of embodiment 413, wherein the dosing regimen of the combination treatment comprises six additional 21-day dosing cycles.

415. The method of any one of embodiments 402-414, wherein the MCL is R/R MCL.

416. The method of embodiment 415, wherein the subject is R/R to Bruton's tyrosine kinase (BTK) inhibitor therapy.

417. The method of embodiment 415, wherein the subject is R/R to chimeric antigen receptor T cell (CAR-T) therapy.

418. The method of embodiment 415, wherein the subject is R/R to 2 or more prior lines of therapy.

419. The method of any one of embodiments 402-418, wherein the subjects of the population have a high-risk factor comprising a Ki-67 proliferation index>50%. a blastoid/pleomorphic variants, or a TP53 mutation.

420. A method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a CRR, an ORR, a PFS, or an OS.

421. The method of embodiment 420, wherein the efficacy response is a CRR, and wherein the CRR or reference CRR is the proportion of the subjects receiving the corresponding treatment whose best overall response is a complete response (CR).

422. The method of embodiment 421, wherein the improved response in CRR is an increase in CRR compared to the reference CRR of between 3% and 44%.

423. The method of embodiment 422, wherein the improved response in CRR is an increase in CRR compared to the reference CRR of about 25%.

424. The method of embodiment 420, wherein the efficacy response is an ORR, and wherein the ORR or reference ORR is the proportion of the subjects receiving the corresponding treatment whose best overall response is a CR or a partial response (PR).

425. The method of embodiment 424, wherein the improved response in ORR is an increase in ORR compared to the reference ORR of between 15% and 46%.

426. The method of embodiment 425, wherein the improved response in ORR is an increase in ORR compared to the reference ORR of about 31%.

427. The method of embodiment 420, wherein the efficacy response is a PFS, and wherein the PFS or the reference PFS is measured starting from the time of receiving first dose of mosunetuzumab or polatuzumab vedotin to the time of a first occurrence of disease progression or death from any cause.

428. The method of embodiment 427, wherein the PFS or the reference PFS is the median PFS of the plurality of subjects receiving the corresponding treatment.

429. The method of embodiment 427 or 428, wherein the improvement of the median PFS is an increase in the PFS compared to the reference PFS of between 1 and 15 months.

430. The method of embodiment 429, wherein the improvement of the median PFS is an increase in the PFS compared to the reference PFS of 7 months.

431. The method of embodiment 427, wherein the improvement of the PFS is an increase in the rate of a PFS of 3 months compared to the rate of a reference PFS of 3 months of between 7% and 37%.

432. The method of embodiment 431, wherein the improvement of the PFS is an increase in the rate of a PFS of 3 months compared to the rate of a reference PFS of 3 months of about 22%.

433. The method of embodiment 427, wherein the improvement of the PFS is an increase in the rate of a PFS of 6 months compared to the rate of a reference PFS of 6 months of between 11% and 44%.

434. The method of embodiment 433, wherein the improvement of the PFS is an increase in the rate of a PFS of 6 months compared to the rate of a reference PFS of 6 months of about 27%.

435. The method of embodiment 427, wherein the improvement of the PFS is an increase in the rate of a PFS of 9 months compared to the rate of a reference PFS of 9 months of between 11% and 45%.

436. The method of embodiment 435, wherein the improvement of the PFS is an increase in the rate of a PFS of 9 months compared to the rate of a reference PFS of 9 months of about 28%.

437. The method of embodiment 427, wherein the improvement of the PFS is an increase in the rate of a PFS of 12 months compared to the rate of a reference PFS of 12 months of between 10% and 44%.

438. The method of embodiment 437, wherein the improvement of the PFS is an increase in the rate of a PFS of 12 months compared to the rate of a reference PFS of 12 months of about 27%.

439. The method of embodiment 427, wherein the improvement of the PFS is an increase in the rate of a PFS of 18 months compared to the rate of a reference PFS of 18 months of between 5% and 39%.

440. The method of embodiment 439, wherein the improvement of the PFS is an increase in the rate of a PFS of 18 months compared to the rate of a reference PFS of 18 months of about 22%.

441. The method of embodiment 427, wherein the improvement of the PFS is an increase in the rate of a PFS of 24 months compared to the rate of a reference PFS of 24 months of between 5% and 38%.

442. The method of embodiment 441, wherein the improvement of the PFS is an increase in the rate of a PFS of 24 months compared to the rate of a reference PFS of 24 months of about 21%.

443. The method of embodiment 420, wherein the efficacy response is an OS, and wherein the OS or the reference OS is measured starting from the time of receiving first dose of mosunetuzumab or polatuzumab vedotin to the time of a first occurrence of disease progression or death from any cause.

444. The method of embodiment 443, wherein the OS or the reference OS is the median OS of the plurality of subjects receiving the corresponding treatment.

445. The method of embodiment 443 or 444, wherein the improvement of the median OS is an increase in the OS compared to the reference OS.

446. The method of embodiment 445, wherein the improvement of the median OS is an increase in the OS compared to the reference OS of 7.4 months.

447. The method of embodiment 443, wherein the improvement of the OS is an increase in the rate of a OS of 6 months compared to the rate of a reference OS of 6 months of bet ween 1% and 26%.

448. The method of embodiment 447, wherein the improvement of the OS is an increase in the rate of a OS of 6 months compared to the rate of a reference OS of 6 months of about 12%.

449. The method of embodiment 443, wherein the improvement of the OS is an increase in the rate of a OS of 9 months compared to the rate of a reference OS of 9 months of between 1% and 24%.

450. The method of embodiment 449, wherein the improvement of the OS is an increase in the rate of a OS of 9 months compared to the rate of a reference OS of 9 months of about 9%.

451. The method of embodiment 443, wherein the improvement of the OS is an increase in the rate of a OS of 12 months compared to the rate of a reference OS of 12 months of between 1% and 27%.

452. The method of embodiment 451, wherein the improvement of the OS is an increase in the rate of a OS of 12 months compared to the rate of a reference OS of 12 months of about 11%.

453. The method of embodiment 443, wherein the improvement of the OS is an increase in the rate of a OS of 18 months compared to the rate of a reference OS of 18 months of between 1% and 28%.

454. The method of embodiment 453, wherein the improvement of the OS is an increase in the rate of a OS of 18 months compared to the rate of a reference OS of 18 months of about 11%.

455. The method of embodiment 443, wherein the improvement of the OS is an increase in the rate of a OS of 24 months compared to the rate of a reference OS of 24 months of between 1% and 29%.

456. The method of embodiment 455, wherein the improvement of the OS is an increase in the rate of a OS of 24 months compared to the rate of a reference OS of 24 months of about 13%.

457. The method of any one of embodiments 421-456, wherein the CR or PR determined by PET/computed tomography (CT).

458. The method of embodiment 457, wherein the CR or PR is determined based on the Lugano Response Criteria for Malignant Lymphoma (Cheson et al., 2014).

459. A method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered R-GemOx, and wherein the safety response is the rate of AE or the rate of SAE.

460. The method of embodiment 459, wherein the safety response is the rate of AE, and wherein the rate of AE or the reference rate of AE is the rate of AE in the plurality of subjects receiving the corresponding treatment.

461. The method of embodiment 460, wherein the safety response in rate of AE is non-inferior compared to the reference rate of AE.

462. The method of embodiment 460, wherein the safety response in rate of Grade 3-5 AE is non-inferior compared to the reference rate of Grade 3-5 AE.

463. The method of embodiment 462, wherein the Grade of AE is determined based on the National Cancer Institute Common Terminology Criteria for Adverse Events (NCI CTCAE), v5.0.

464. The method of embodiment 459, wherein the safety response is the rate of SAE, and wherein the rate of SAE or the reference rate of SAE is the rate of SAE in the plurality of subjects receiving the corresponding treatment.

465. The method of embodiment 464, wherein the safety response in rate of SAE is non-inferior compared to the reference rate of SAE.

466. The method of any one of embodiments 420-465, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
   (i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
   (i) a first subcutaneous dose (scC2D1) of mosunetuzumab administered on Day 1 of the second dosing cycle, wherein the scC2D1 is about 45 mg; and
   (ii) an intravenous dose of polatuzumab vedotin administered on Day 1 of the second dosing cycle, wherein the intravenous dose of polatuzumab vedotin is about 1.8 mg/kg.

467. The method of embodiment 466, wherein the dosing regimen of the combination treatment further comprises six additional 21-day dosing cycles.

468. The method of embodiment 467, wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg.

469. The method of any one of embodiments 420-468, wherein the control treatment comprises intravenously administering 375 mg/m² rituximab, 1000 mg/m² gemcitabine, and 100 mg/m² oxaliplatin for eight 14-day dosing cycles, wherein rituximab, gemcitabine, and oxaliplatin are administered on Day 1 of each dosing cycle.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a complete response rate (CRR), an objective response rate (ORR), a duration of response (DOR), a duration of complete response (DOCR), a progression free survival (PFS), or an overall survival (OS).

2. The method of claim 1, wherein:
(a) the efficacy response is a CRR, and wherein the CRR or reference CRR is the proportion of the subjects receiving the corresponding treatment whose best overall response is a complete response (CR);
(b) the efficacy response is an ORR, and wherein the ORR or reference ORR is the proportion of the subjects receiving the corresponding treatment whose best overall response is a CR or a partial response (PR);
(c) the efficacy response is a DOR, and wherein the DOR or the reference DOR is measured starting from the time from the first occurrence of a documented CR or PR to disease progression or relapse or death from any cause, whichever occurs first; or
(d) the efficacy response is a DOCR, and wherein the DOCR or the reference DOCR is measured starting from the time from the first occurrence of a documented CR to disease progression or relapse or death from any cause, whichever occurs first.

3. The method of claim 2, wherein:
(a) the improved response in CRR is an increase in CRR compared to the reference CRR of between 1% and 41% or of about 20.8%;
(b) the improved response in ORR is an increase in ORR compared to the reference ORR of between 3% and 47% or of about 25.6%;
(c) the DOR or the reference DOR is the median DOR of the plurality of subjects receiving the corresponding treatment, and =the efficacy response in median DOR is non-inferior compared to the reference median DOR;
(d) the efficacy response in the rate of a DOR of:
(i) 3 months is non-inferior compared to the reference rate of a DOR of 3 months;
(ii) 6 months is non-inferior compared to the reference rate of a DOR of 6 months; or
(iii) 9 months is non-inferior compared to the reference rate of a DOR of 9 months;
(e) the DOCR or the reference DOCR is the median DOCR of the plurality of subjects receiving the corresponding treatment, and the efficacy response in median DOCR is non-inferior compared to the reference median DOCR; or
(f) the efficacy response in the rate of a DOCR of:
(i) 3 months is non-inferior compared to the reference rate of a DOCR of 3 months;
(ii) 6 months is non-inferior compared to the reference rate of a DOCR of 6 months; or
(iii) 9 months is non-inferior compared to the reference rate of a DOCR of 9 months.

4-19. (canceled)

20. The method of claim 2, wherein the CR or PR determined by PET/computed tomography (CT); and/or the CR or PR is determined based on the Lugano Response Criteria for Malignant Lymphoma (Cheson et al., 2014).

21. (canceled)

22. A method of treating an aggressive non-Hodgkin's lymphoma (aNHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab, gemcitabine, and oxaliplatin (R-GemOx), and wherein the safety response is the rate of adverse event (AE) or the rate of serious adverse events (SAE).

23. The method of claim 22, wherein:
(a) the safety response is the rate of AE, and wherein the rate of AE or the reference rate of AE is the rate of AE in the plurality of subjects receiving the corresponding treatment; or
(b) the safety response is the rate of SAE, and wherein the rate of SAE or the reference rate of SAE is the rate of SAE in the plurality of subjects receiving the corresponding treatment.

24. The method of claim 23, wherein:
(a) the safety response in rate of AE is non-inferior compared to the reference rate of AE;
(b) the safety response in rate of Grade 3-5 AE is non-inferior compared to the reference rate of Grade 3-5 AE; and/or the Grade of AE is determined based on the National Cancer Institute Common Terminology Criteria for Adverse Events (NCI CTCAE), v5.0; or
(c) the safety response in rate of SAE is non-inferior compared to the reference rate of SAE.

25-28. (canceled)

29. The method of claim 1, wherein:
(I) the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
(i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
(ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
(i) a first subcutaneous dose (scC2D1) of mosunetuzumab administered on Day 1 of the second dosing cycle, wherein the scC2D1 is about 45 mg; and
(ii) an intravenous dose of polatuzumab vedotin administered on Day 1 of the second dosing cycle, wherein the intravenous dose of polatuzumab vedotin is about 1.8 mg/kg; and/or
(II) the control treatment comprises intravenously administering 375 mg/m² rituximab, 1000 mg/m² gemcitabine, and 100 mg/m² oxaliplatin for eight 14-day dosing cycles, wherein rituximab, gemcitabine, and oxaliplatin are administered on Day 1 of each dosing cycle.

30. The method of claim 29, wherein the dosing regimen of the combination treatment further comprises one or more additional 21-day dosing cycles.

31. The method of claim 30, wherein:
(I) the dosing regimen of the combination treatment comprises six additional 21-day dosing cycles, wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg; or
(II) each additional dosing cycle comprises:
(a) a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; or
(b) a subcutaneous dose of mosunetuzumab administered on Day 1 of the additional dosing cycle, wherein the subcutaneous dose of mosunetuzumab is about 45 mg.

32-34. (canceled)

35. A method of treating relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab and polatuzumab vedotin, and wherein the efficacy response is a complete response rate (CRR), an objective response rate (ORR), a duration of response (DOR), or a progression free survival (PFS).

36. The method of claim 35, wherein:
(a) the efficacy response is a CRR, and wherein the CRR or reference CRR is the proportion of the subjects receiving the corresponding treatment whose best overall response is a complete response (CR);
(b) the efficacy response is an ORR, and wherein the ORR or reference ORR is the proportion of the subjects receiving the corresponding treatment whose best overall response is a CR or a partial response (PR);
(c) the efficacy response is a DOR, and wherein the DOR or the reference DOR is measured starting from the time from the first occurrence of a documented CR or PR to disease progression or relapse or death from any cause, whichever occurs first; or
(d) the efficacy response is a PFS, and wherein the PFS or the reference PFS is measured starting from the time of receiving first dose of mosunetuzumab or polatuzumab vedotin to the time of a first occurrence of disease progression or death from any cause.

37. The method of claim 36, wherein:
(a) the improved response in CRR is an increase in CRR compared to the reference CRR of between 1% and 52% or of about 23%;
(b) the improved response in ORR is an increase in ORR compared to the reference ORR of between 1% and 55% or of about 28%;
(c) the DOR or the reference DOR is the median DOR of the plurality of subjects receiving the corresponding treatment, and the improvement of the median DOR is an increase in the DOR compared to the reference DOR of between 1 and 21 months;
(d) the improvement of the DOR is:
(i) an increase in the rate of a DOR of 6 months compared to the rate of a reference DOR of 6 months of between 1% and 61% or of about 20%;
(ii) an increase in the rate of a DOR of 9 months compared to the rate of a reference DOR of 9 months of between 1% and 72% or of about 26%;
(iii) an increase in the rate of a DOR of 12 months compared to the rate of a reference DOR of 12 months of between 1% and 87% or of about 39%; or
(iv) an increase in the rate of a DOR of 18 months compared to the rate of a reference DOR of 18 months of between 43% and 92% or of about 67%
(e) the PFS or the reference PFS is the median PFS of the plurality of subjects receiving the corresponding treatment, and the improvement of the median PFS is an increase in the PFS compared to the reference PFS of between 1 and 24 months; or
(f) the improvement of the PFS is:
(i) an increase in the rate of a PFS of 6 months compared to the rate of a reference PFS of 6 months of between 1% and 53% or of about 21%;
(ii) an increase in the rate of a PFS of 9 months compared to the rate of a reference PFS of 9 months of between 1% and 62% or of about 28%;
(iii) an increase in the rate of a PFS of 12 months compared to the rate of a reference PFS of 12 months of between 1% and 63% or of about 27%; or
(iv) an increase in the rate of a PFS of 18 months compared to the rate of a reference PFS of 18 months of between 1% and 68% or of about 25%.

38-52. (canceled)

53. The method of claim 36, wherein the CR or PR determined by PET/computed tomography (CT); and/or the CR or PR is determined based on the Lugano Response Criteria for Malignant Lymphoma (Cheson et al., 2014).

54-65. (canceled)

66. A method of treating relapsed and/or refractory non-Hodgkin's lymphoma (R/R NHL) in a subject in need thereof, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered rituximab and polatuzumab vedotin, and wherein the safety response is the rate of adverse event (AE) or the rate of serious adverse event (SAE).

67. The method of claim 66, wherein (a) the safety response is the rate of AE, and wherein the rate of AE or the reference rate of AE is the rate of AE in the plurality of subjects receiving the corresponding treatment, and the safety response in rate of AE is non-inferior compared to the reference rate of AE; or (b) the safety response is the rate of SAE, and wherein the rate of SAE or the reference rate of SAE is the rate of SAE in the plurality of subjects receiving the corresponding treatment, and the safety response in rate of SAE is non-inferior compared to the reference rate of SAE.

68. (canceled)

69. The method of claim 35, wherein;
(I) the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
(i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
(ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
(i) a first subcutaneous dose (scC2D1) of mosunetuzumab administered on Day 1 of the second dosing cycle, wherein the scC2D1 is about 45 mg; and
(ii) an intravenous dose of polatuzumab vedotin administered on Day 1 of the second dosing cycle, wherein the intravenous dose of polatuzumab vedotin is about 1.8 mg/kg; and/or
(II) the control treatment comprises (a) intravenously administering 375 mg/m² rituximab and 1.8 mg/kg polatuzumab vedotin for six 21-day dosing cycles, wherein rituximab and polatuzumab vedotin are administered on Day 1 of each dosing cycle; and (b) intravenously administering 375 mg/m² rituximab for two 21-day dosing cycles, wherein rituximab is administered on Day 1 of each dosing cycle.

70. A method of achieving an objective response (OR), a complete response (CR), an overall survival (OS), or a progression free survival (PFS) in a subject having a relapsed and/or refractory non-Hodgkin's lymphoma (R/R NHL), wherein the subject is administered a combination treatment comprising subcutaneously administered mosunetuzumab and intravenously administered polatuzumab vedotin.

71. The method of claim 70, wherein:
(a) the subject achieves an OR, and the OR is maintained for at least 6, 9, 12, or 18 months;
(b) the subject achieves a CR;
(c) the OS is maintained for at least 6, 9, 12, or 18 months; or
(d) the PFS is maintained for at least 6, 9, 12, or 18 months.

72-75. (canceled)

76. A method of treating a population of subjects having a relapsed and/or refractory non-Hodgkin's lymphoma (R/R NHL), wherein each subject of the population is administered a combination treatment comprising subcutaneously administered mosunetuzumab and intravenously administered polatuzumab vedotin, and wherein the population of subjects:
(a) has an overall response rate of between 62% and 89%, or about 78%;
(b) has a complete response rate of between 41% and 73%, or about 58%;
(c) has an overall survival rate at 9 months of between 66% and 92%, or about 79%;
(d) has an overall survival rate at 12 months of between 60% and 88%, or about 74%;
(e) has a progression free survival rate at 9 months of between 57% and 87%, or about 72%; or
(f) has a progression free survival rate at 12 months of between 47% and 81%, or about 64%.

77-87. (canceled)

88. The method of claim 35, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
(i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
(ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
(i) a first subcutaneous dose (scC2D1) of mosunetuzumab administered on Day 1 of the second dosing cycle, wherein the scC2D1 is about 45 mg; and
(ii) an intravenous dose of polatuzumab vedotin administered on Day 1 of the second dosing cycle, wherein the intravenous dose of polatuzumab vedotin is about 1.8 mg/kg.

89. The method of claim 88, wherein the dosing regimen of the combination treatment further comprises one or more additional 21-day dosing cycles.

90. The method of claim 89, wherein;
(I) the dosing regimen of the combination treatment comprises six additional 21-day dosing cycles, wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg; or
(II) the each additional dosing cycle comprises:
(a) a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; or
(b) a subcutaneous dose of mosunetuzumab administered on Day 1 of the additional dosing cycle, wherein the subcutaneous dose of mosunetuzumab is about 45 mg.

91-92. (canceled)

93. A method of treating a population of subjects having a relapsed and/or refractory non-Hodgkin's lymphoma (R/R NHL), wherein each subject of the population is administered a combination treatment comprising intravenously administered mosunetuzumab and intravenously administered polatuzumab vedotin, and wherein:
(a) the population of subjects has a median overall survival of between 15 and 39 months, or about 27 months;
(b) the population of subjects has an overall survival rate:
(i) at 6 months of between 73% and 89%, or about 81%;
(ii) at 9 months of between 61% and 80%, or about 70%;
(iii) at 12 months of between 55% and 75%, or about 65%;
(iv) at 18 months of between 45% and 65%, or about 55%; or
(v) at 24 months of between 40% and 61%, or about 50%;
(c) the population of subjects has a median progression free survival of between 9 and 27 months, or about 14 months;
(d) the population of subjects has a progression free survival rate:
(i) at 6 months of between 51% and 72%, or about 62%;
(ii) at 9 months of between 50% and 71%, or about 60%;
(iii) at 12 months of between 41% and 64%, or about 52%;
(iv) at 18 months of between 34% and 57%, or about 46%; or
(v) at 24 months of between 28% and 52%, or about 40%;
(e) the population of subjects has a median duration of response of between 16 and 39 months, or about 28 months;
(f) between:
(i) 78% and 96% of the population of subjects maintains a durable response for 6 months, or about 87% of the population of subjects maintains a durable response for 6 months;
(ii) 63% and 88% of the population of subjects maintains a durable response for 9 months, or about 75% of the population of subjects maintains a durable response for 9 months;
(iii) 58% and 84% of the population of subjects maintains a durable response for 12 months, or about 71% of the population of subjects maintains a durable response for 12 months;
(iv) 46% and 75% of the population of subjects maintains a durable response for 18 months, or about 61% of the population of subjects maintains a durable response for 18 months; or
(v) 43% and 73% of the population of subjects maintains a durable response for 24 months; or
(g) about 58% of the population of subjects maintains a durable response for 24 months.

94-128. (canceled)

129. The method of claim 93, wherein the combination treatment comprises intravenously administering mosunetuzumab and intravenously administering polatuzumab vedotin according to a dosing regimen comprising eight 21-day dosing cycle, wherein:
(a) the first dosing cycle comprises:
(i) a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg; and
(ii) an intravenous dose of polatuzumab vedotin administered on Day 1 of the first dosing cycle, wherein the intravenous dose of polatuzumab vedotin is about 1.8 mg/kg;
(b) the second dosing cycle comprises:
(i) a first intravenous dose (ivC2D1) of mosunetuzumab administered on Day 1 of the second dosing cycle, wherein the ivC2D1 is about 60 mg; and
(ii) an intravenous dose of polatuzumab vedotin administered on Day 1 of the second dosing cycle, wherein the intravenous dose of polatuzumab vedotin is about 1.8 mg/kg;
(c) the third to sixth dosing cycles each comprises:
(i) a first intravenous dose (ivC3D1-ivC6D1) of mosunetuzumab administered on Day 1 of each respective dosing cycle, wherein the ivC3D1-ivC6D1 is each about 30 mg; and
(ii) an intravenous dose of polatuzumab vedotin administered on Day 1 of the second dosing cycle, wherein the intravenous dose of polatuzumab vedotin is about 1.8 mg/kg; and
(d) the seventh and eighth dosing cycles each comprises a first intravenous dose (ivC7D1-ivC8D1) of mosunetuzumab administered on Day 1 of each respective dosing cycle, wherein the ivC7D1-ivC8D1 is each about 30 mg and does not comprise administration of polatuzumab vedotin.

130. The method of claim 1, wherein;
(a) the subject has an Eastern Cooperative Oncology Group (ECOG) Performance Status of 0, 1, or 2;
(b) the aNHL is a diffuse large B cell lymphoma (DLBCL), a high grade B cell lymphoma (HGBL), a Grade 3b follicular lymphoma (FL), or a transformed FL (trFL);
(c) the subject has relapsed after or is refractory to one or more prior lines of systemic therapy;
(d) the subject is ineligible for autologous stem cell transplant (ASCT); and/or
(e) wherein the method comprises administering an additional therapeutic agent.

131. (canceled)

132. The method of claim 35, wherein the R/R NHL is an R/R large B cell lymphoma (LBCL), an R/R diffuse large B cell lymphoma (DLBCL), an R/R follicular lymphoma (FL), or an R/R mantle cell lymphoma (MCL).

133-135. (canceled)

136. The method of claim 130, wherein the additional therapeutic agent comprises;
(a) a corticosteroid, wherein the corticosteroid is prednisone, methylprednisolone, or dexamethasone;
(b) an antihistamine, wherein the antihistamine is diphenhydramine hydrochloride or an equivalent thereof;
(c) an antipyretic, wherein the antipyretic is acetaminophen; or
(d) an IL-6R antagonist, wherein the IL-6R antagonist is tocilizumab.

137-140. (canceled)

141. A method of treating a population of subjects having a relapsed and/or refractory MCL, wherein each subject of the population is administered a combination treatment comprising subcutaneously administered mosunetuzumab and intravenously administered polatuzumab vedotin, and wherein:
(a) the population of subjects has an overall response rate of between 74% and 100%, or about 88%;
(b) wherein the population of subjects has a complete response rate of between 63% and 93%, or about 79%;
(c) the median time to first response in the population of subjects is between 80 days to 100 days, or about 3 months;
(d) the median PFS in the population of subjects is at least 14 months, or about 19 months; or
(e) the median OS in the population of subjects is at least 17 months, or about 21 months.

142-150. (canceled)

151. The method of claim 141, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
(i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
(ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
(i) a first subcutaneous dose (scC2D1) of mosunetuzumab administered on Day 1 of the second dosing cycle, wherein the scC2D1 is about 45 mg; and
(ii) an intravenous dose of polatuzumab vedotin administered on Day 1 of the second dosing cycle, wherein the intravenous dose of polatuzumab vedotin is about 1.8 mg/kg.

152. The method of claim 151, wherein the dosing regimen of the combination treatment further comprises six additional 21-day dosing cycles, and wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg.

153. (canceled)

154. The method of claim 141, wherein:
(a) the MCL is R/R MCL; and/or
(b) the subjects of the population have a high-risk factor comprising a Ki-67 proliferation index>50%, a blastoid/pleomorphic variant, or a TP53 mutation.

155. The method of claim 154, wherein the is subjects of the population are:
(a) R/R to Bruton's tyrosine kinase (BTK) inhibitor therapy;
(b) R/R to prior chimeric antigen receptor T cell (CAR-T) therapy; and/or
(c) R/R to 2 or more prior lines of therapy; and/or.

156-158. (canceled)

159. A method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered rituximab, gemcitabine, and oxaliplatin (R-GemOx), and wherein the efficacy response is a CRR, an ORR, a PFS, or an OS.

160. The method of claim 159, wherein;
(a) the efficacy response is a CRR, and wherein the CRR or reference CRR is the proportion of the subjects receiving the corresponding treatment whose best overall response is a complete response (CR);
(b) the efficacy response is an ORR, and wherein the ORR or reference ORR is the proportion of the subjects receiving the corresponding treatment whose best overall response is a CR or a partial response (PR);
(c) the efficacy response is a PFS, and wherein the PFS or the reference PFS is measured starting from the time of receiving first dose of mosunetuzumab or polatuzumab vedotin to the time of a first occurrence of disease progression or death from any cause; or
(d) the efficacy response is an OS, and wherein the OS or the reference OS is measured starting from the time of receiving first dose of mosunetuzumab or polatuzumab vedotin to the time of a first occurrence of disease progression or death from any cause.

161. The method of claim 160, wherein:
(a) the improved response in CRR is an increase in CRR compared to the reference CRR of between 3% and 44%, or about 25%;
(b) the improved response in ORR is an increase in ORR compared to the reference ORR of between 15% and 46%, or about 31%;
(c) the PFS or the reference PFS is the median PFS of the plurality of subjects receiving the corresponding treatment, and wherein the improvement of the median PFS is an increase in the PFS compared to the reference PFS of between 1 and 15 months, or about 7 months;
(d) the improvement of the PFS is:
(i) an increase in the rate of a PFS of 3 months compared to the rate of a reference PFS of 3 months of between 7% and 37%, or about 22%;
(ii) an increase in the rate of a PFS of 6 months compared to the rate of a reference PFS of 6 months of between 11% and 44%, or about 27%;
(iii) an increase in the rate of a PFS of 9 months compared to the rate of a reference PFS of 9 months of between 11% and 45%, or about 28%;
(iv) an increase in the rate of a PFS of 12 months compared to the rate of a reference PFS of 12 months of between 10% and 44%, or about 27%;
(v) an increase in the rate of a PFS of 18 months compared to the rate of a reference PFS of 18 months of between 5% and 39%, or about 22%; or
(vi) an increase in the rate of a PFS of 24 months compared to the rate of a reference PFS of 24 months of between 5% and 38%, or about 21%;
(e) the OS or the reference OS is the median OS of the plurality of subjects receiving the corresponding treatment, and wherein the improvement of the median OS is an increase in the OS compared to the reference OS of about 7.4 months; or
(f) the improvement of the OS is:
(i) an increase in the rate of an OS of 6 months compared to the rate of a reference OS of 6 months of between 1% and 26%, or about 12%;
(ii) an increase in the rate of a OS of 9 months compared to the rate of a reference OS of 9 months of between 1% and 24%, or about 9%;
(iii) an increase in the rate of a OS of 12 months compared to the rate of a reference OS of 12 months of between 1% and 27%, or about 11%;
(iv) an increase in the rate of a OS of 18 months compared to the rate of a reference OS of 18 months of between 1% and 28%, or about 11%; or
(v) an increase in the rate of a OS of 24 months compared to the rate of a reference OS of 24 months of between 1% and 29%, or about 13%.

162-195. (canceled)

196. The method of claim 160, wherein the CR or PR determined by PET/computed tomography (CT); and/or the CR or PR is determined based on the Lugano Response Criteria for Malignant Lymphoma (Cheson et al., 2014).

197. (canceled)

198. A method of treating an R/R LBCL in a subject in need thereof who is ineligible for ASCT, comprising administering to the subject a combination treatment comprising subcutaneously administered mosunetuzumab and intravenously administered polatuzumab vedotin, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered R-GemOx, and wherein the safety response is the rate of AE or the rate of SAE.

199. The method of claim 198, wherein:
(a) the safety response is the rate of AE, and wherein the rate of AE or the reference rate of AE is the rate of AE in the plurality of subjects receiving the corresponding treatment; or
(b) the safety response is the rate of SAE, and wherein the rate of SAE or the reference rate of SAE is the rate of SAE in the plurality of subjects receiving the corresponding treatment.

200. The method of claim 199, wherein:
(a) the safety response in rate of AE is non-inferior compared to the reference rate of AE;
(b) the safety response in rate of Grade 3-5 AE is non-inferior compared to the reference rate of Grade 3-5 AE, wherein the Grade of AE is determined based on the National Cancer Institute Common Terminology Criteria for Adverse Events (NCI CTCAE), v5.0; or
(c) the safety response in rate of SAE is non-inferior compared to the reference rate of SAE.

201-204. (canceled)

205. The method of any one of claims 159-204, claim 159, wherein the combination treatment comprises subcutaneously administering mosunetuzumab and intravenously administered polatuzumab vedotin according to a dosing regimen comprising at least a first 21-day dosing cycle and a second 21-day dosing cycle, wherein:
(a) the first dosing cycle comprises:
(i) a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
(ii) an intravenous dose of polatuzumab vedotin administered on Day 1 of the first dosing cycle, wherein the intravenous dose of polatuzumab vedotin is about 1.8 mg/kg; and
(b) the second dosing cycle comprises:
(i) a first subcutaneous dose (scC2D1) of mosunetuzumab administered on Day 1 of the second dosing cycle, wherein the scC2D1 is about 45 mg; and
(ii) an intravenous dose of polatuzumab vedotin administered on Day 1 of the second dosing cycle, wherein the intravenous dose of polatuzumab vedotin is about 1.8 mg/kg.

206. The method of claim 205, wherein;
(I) the dosing regimen of the combination treatment further comprises six additional 21-day dosing cycles, and wherein:
(a) the first four additional dosing cycles each comprises a subcutaneous dose of mosunetuzumab administered on Day 1 of the additional dosing cycle, wherein the subcutaneous dose of mosunetuzumab is about 45 mg; and an intravenous dose of polatuzumab vedotin administered on Day 1 of the additional dosing cycle, wherein the intravenous dose of polatuzumab vedotin is about 1.8 mg/kg; and
(b) the next two additional dosing cycles each comprises a subcutaneous dose of mosunetuzumab administered on Day 1 of the additional dosing cycle, wherein the subcutaneous dose of mosunetuzumab is about 45 mg; and/or
(II) the control treatment comprises intravenously administering 375 mg/m² rituximab, 1000 mg/m² gemcitabine, and 100 mg/m² oxaliplatin for eight 14-day dosing cycles, wherein rituximab, gemcitabine, and oxaliplatin are administered on Day 1 of each dosing cycle.

207-208. (canceled)


SEQUENCE LISTING (54 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 25 aa, synthetic construct [sequence omitted]

SEQ ID NO: 18 - protein, 14 aa, synthetic construct
WVRQAPGKGLEWVG

SEQ ID NO: 19 - protein, 32 aa, synthetic construct
RFTISVDKSKNTLYLQMNSLRAEDTAVYYCAR

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WYQQKPGKAPKPLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRQAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 10 aa, synthetic construct
GYTFSSYWIE

SEQ ID NO: 38 - protein, 18 aa, synthetic construct
GEILPGGGDTNYNEIFKG

SEQ ID NO: 39 - protein, 10 aa, synthetic construct
TRRVPIRLDY

SEQ ID NO: 40 - protein, 15 aa, synthetic construct
KASQSVDYEGDSFLN

SEQ ID NO: 41 - protein, 7 aa, synthetic construct
AASNLES

SEQ ID NO: 42 - protein, 9 aa, synthetic construct
QQSNEDPLT

SEQ ID NO: 43 - protein, 117 aa, synthetic construct [sequence omitted]

SEQ ID NO: 44 - protein, 112 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 45 - protein, 25 aa, synthetic construct [sequence omitted]

SEQ ID NO: 46 - protein, 13 aa, synthetic construct
WVRQAPGKGLEWI

SEQ ID NO: 47 - protein, 30 aa, synthetic construct
RATFSADTSKNTAYLQMNSLRAEDTAVYYC

SEQ ID NO: 48 - protein, 11 aa, synthetic construct
WGQGTLVTVSS

SEQ ID NO: 49 - protein, 23 aa, synthetic construct
DIQLTQSPSSLSASVGDRVTITC

SEQ ID NO: 50 - protein, 15 aa, synthetic construct
WYQQKPGKAPKLLIY

SEQ ID NO: 51 - protein, 32 aa, synthetic construct; identical to SEQ ID NO: 23

SEQ ID NO: 52 - protein, 11 aa, synthetic construct
FGQGTKVEIKR

SEQ ID NO: 53 - protein, 447 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 218 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC